(19) United States
(12) Patent Application Publication
(10) Pub. No.: US 2023/0026789 A1
(43) Pub. Date: Jan. 26, 2023

(54) METHODS, SYSTEMS, AND TOOLS FOR LONGEVITY-RELATED APPLICATIONS

(71) Applicant: SPRING DISCOVERY, INC., San Carlos, CA (US)

(72) Inventors: Brandon WHITE, San Carlos, CA (US); Ben KOMALO, San Francisco, CA (US); Rachel JACOBSON, Belmont, CA (US); Christian ELABD, Belmont, CA (US); Wendy COUSIN, Belmont, CA (US); Ben KAMENS, Palo Alto, CA (US); Charles MARSH, Brooklyn, NY (US); Lauren NICOLAISEN, Redwood City, CA (US)

(21) Appl. No.: 17/833,719

(22) Filed: Jun. 6, 2022

Related U.S. Application Data
(63) Continuation of application No. PCT/US2020/067648, filed on Dec. 31, 2020.
(60) Provisional application No. 63/057,274, filed on Jul. 27, 2020.
(60) Provisional application No. 63/008,601, filed on Apr. 10, 2020.
(60) Provisional application No. 62/956,581, filed on Jan. 2, 2020.

(51) Int. Cl. G16B 15/30; G16B 40/20; G06N 20/00

(52) U.S. Cl. CPC G16B 15/30; G16B 40/20; G06N 20/00

(57) ABSTRACT

Disclosed herein are methods and systems for identifying a drug capable of changing a cell's state, function, and/or predicted age, which is useful in, at least, drug discovery. Further disclosed herein are methods and systems for identifying an in vitro cell's state, function, and/or predicted age.

### CROSS-REFERENCE

This application is a continuation of U.S. International Patent Application No. PCT/US2020/067648 which claims the benefit of U.S. Provisional Application No. 62/956,581, filed Jan. 2, 2020, U.S. Provisional Application No. 63/008,601, filed Apr. 10, 2020, and U.S. Provisional Application No. 63/057,274, filed Jul. 27, 2020, each of which is incorporated by reference herein in its entirety.

### BACKGROUND

Throughout history, drugs and methods that promote longevity, improve immune function, and/or treat disease have been eagerly sought. However, even now, such drug discovery is time consuming and costly. Indeed, the drug discovery industry may benefit from methods that detect changes in a treated cell's state, function, and/or predicted age upon contact with a potential drug. Unfortunately, methods that accurately and rapidly identify a cell's state, function, and/or predicted age are currently unavailable. Accordingly, there remains an unmet need for methods that can be used for efficient discovery of drugs and methods that promote longevity, improve immune function, and/or treat disease.

### SUMMARY

The present disclosure provides methods and systems for identifying a drug capable of changing a cell's state, function, and/or predicted age, which is useful in, at least, drug discovery. Further, the present disclosure provides methods and systems for identifying an in vitro cell's state, function, and/or predicted age.

An aspect of the present disclosure provides a method for identifying an in vitro cell's state, function, and/or predicted age. The method comprising: contacting an in vitro cell with a first binding reagent capable of recognizing and binding a first marker of the in vitro cell, and contacting the in vitro cell with an at least second binding reagent capable of recognizing and binding an at least second marker of the in vitro cell; determining the in vitro cell's morphological signature and/or functional signature based on the intensity of a signal associated with the first binding reagent and the intensity of a signal associated with the at least second binding reagent; and identifying the in vitro cell's state, function, and/or predicted age based upon the morphological signature and/or functional signature.

In some embodiments, the in vitro cell's morphological signature comprises the in vitro cell's size and/or the in vitro cell's shape and/or morphological properties (e.g., eccentricity, form factor, and solidity). In some embodiments, the in vitro cell's functional signature comprises one or more of the presence or absence of a marker characteristic of a specific cell organelle, the presence or absence of a marker characteristic of a specific cell type, the presence or absence of a marker characteristic of a specific cell activity, the presence or absence of a marker characteristic of an active cell, the presence or absence of a marker characteristic of an inactive cell, and/or the presence or absence of a marker indicating gene expression, and/or the ability of the in vitro cell to interact with another cell or fragment of another cell. In some embodiments, the first marker and/or the at least second marker is located on or in the in vitro cell's nucleus, cell membrane, cytoplasm, cytoskeleton, chromatin, mitochondrion, and/or extracellular matrix. In some embodiments, the first binding reagent and/or the at least second binding reagent are each respectively directly or indirectly labeled with a first fluorescent molecule and/or an at least second fluorescent molecule. In some embodiments, the at least second marker comprises a third marker, a fourth marker, a fifth marker, a sixth marker, a seventh marker, an eighth marker, a ninth marker, a tenth marker, or more markers. In some embodiments, determining the in vitro cell's morphological signature and/or functional signature further comprises contacting the in vitro cell with a third binding reagent, a fourth binding reagent, a fifth binding reagent, a sixth binding reagent, a seventh binding reagent, and/or an eighth binding reagent each respectively capable of recognizing and binding a third marker, a fourth marker, a fifth marker, a sixth marker, a seventh marker, am eighth marker, a ninth eighth marker, and/or an at least tenth marker. In some embodiments, the third binding reagent, the fourth binding reagent, the fifth binding reagent, the sixth binding reagent, the seventh binding reagent, the eighth binding reagent, the ninth binding reagent, and/or the at least tenth binding reagent are each respectively directly or indirectly labeled with a third fluorescent molecule, a fourth fluorescent molecule, a fifth fluorescent molecule, a sixth fluorescent molecule, a seventh fluorescent molecule, an eighth fluorescent molecule, a ninth fluorescent molecule and/or an at least tenth fluorescent molecule.

In some embodiments, the intensity of a signal associated with the first binding reagent and the intensity of a signal associated with the at least second binding reagent comprises visualization of the in vitro cell or a portion thereof by light microscopy, e.g., fluorescent microscopy. In some embodiments, the intensity of a signal associated with the first binding reagent and the intensity of a signal associated with the at least second binding reagent are produced by each of the first fluorescent molecule and/or the at least second fluorescent molecule. In some embodiments, determining an in vitro cell's morphological signature and/or functional signature further comprises normalizing the signal intensity produced by each of the first fluorescent molecule and/or the at least second fluorescent molecule. In some embodiments, the in vitro cell's morphological signature and/or functional signature is determined based upon the normalized signal intensity produced by each of the first fluorescent molecule and/or the at least second fluorescent molecule.

In some embodiments, the in vitro cell's predicted age is determined by a machine learning technique selected from convolutional neural networks and gradient boosted trees to compute the in vitro cell's age (e.g., as a continuous value between 0 and 100). For example, the machine learning technique may comprise a supervised machine learning algorithm or supervised machine learning classifier configured to analyze a morphological signature, a functional signature, a marker, etc. of an in vitro cell. In some embodiments, an age of the in vitro cell that is over 65 indicates that the in vitro cell is dying, old, and/or senescent. In some embodiments, an age of the in vitro cell that is under 30 indicates that the in vitro cell is young, healthy, and/or active.

In some embodiments, the marker indicating gene expression is methylated DNA.

In some embodiments, the marker characteristic of a specific cell type is differentially expressed by a T cell (e.g., memory T cell or an effector T cell), a B cell, an Natural Killer (NK) cell, a monocyte/macrophage (e.g., an M1 macrophage or by an M2 macrophage), and a monocyte/dendritic cell.

In some embodiments, the in vitro cell's morphological signature and/or functional signature comprises characterizing the in vitro cell's mitochondrial shape using one or more of spatial pattern analysis (e.g., analysis of spatial point pattern), texture analysis, intensity changes, and convolutional neural networks.

In some embodiments, the in vitro cell's size and/or the in vitro cell's shape and/or morphological properties (e.g., eccentricity, form factor, and solidity) is quantified using convolutional neural networks.

In some embodiments, the marker characteristic of an active cell identifies T cell activation.

In some embodiments, the marker characteristic of an inactive cell identifies T cell exhaustion.

In some embodiments, the marker characteristic of a specific cell type is differentially expressed by an M1 macrophage and by an M2 macrophage. In some embodiments, the marker characteristic of an M1 macrophage is Calprotectin and/or the marker characteristic of an M2 macrophage is Mannose Receptor. In some embodiments, the marker is identified by an antigen binding domain that recognizes Calprotectin or Mannose Receptor.

In some embodiments, the in vitro cell's ability to interact with another cell or fragment of another cell is quantified using a spatial pattern analysis method.

In some embodiments, the in vitro cell's morphological signature and/or functional signature comprises a characterization based upon the presence or absence of cell motility, genomic instability, telomere attrition, epigenetic alterations, loss of proteostasis, deregulated nutrient-sensing, mitochondrial dysfunction, cellular senescence, stem cell exhaustion, and/or altered intercellular communication.

In some embodiments, the first binding reagent and/or the at least second binding reagent is a lectin. In some embodiments, the lectin is Wheat Germ Agglutinin (WGA) or Concanavalin (ConA).

In some embodiments, the first binding reagent and/or the at least second binding reagent comprises an antigen binding domain. In some embodiments, the binding reagent that comprises an antigen binding domain is an antibody. In some embodiments, the binding reagent that comprises an antigen binding domain recognizes total Histone H3, Acetyl-Histone H3, or Tri-Methyl-Histone H3. In some embodiments, the binding reagent that comprises an antigen binding domain recognizes CD3, CD11c, CD14, CD16, CD19, CD45, CD56, or CD68.

In some embodiments, the first binding reagent and/or the at least second binding reagent is a nuclear stain, e.g., Hoechst 33342, or a mitochondrial stain, e.g., a MitoTracker®.

In some embodiments, the first binding reagent and/or the at least second binding reagent is Phalloidin.

In some embodiments, the first binding reagent and/or the at least second binding reagent is capable of determining if a cell is alive or dead (e.g., one or more of DAPI, propidium iodide (PI), and a Zombie dye).

In some embodiments, the first binding reagent, the second binding reagent, the third binding reagent, the fourth binding reagent, and/or the fifth binding reagent each comprises one of Hoechst 33342; Wheat Germ Agglutinin (WGA); Phalloidin; Concanavalin (ConA); and a binding reagent capable of determining if a cell is alive or dead (e.g., one or more of DAPI, propidium iodide (PI), and a Zombie dye). In some embodiments, the method further comprises determine the in vitro cell's morphological signature using brightfield microscopy. The combination of these five binding reagents along with brightfield imaging may be referred to herein as the CellPainting Membrane (CPM) palette.

In some embodiments, the first binding reagent, the second binding reagent, the third binding reagent, the fourth binding reagent, and/or the fifth binding reagent each comprises one of Hoechst 33342; an antigen binding domain that recognizes Acetyl-Histone H3; an antigen binding domain that recognizes Tri-Methyl-Histone H3; MitoTracker® Orange; and a binding reagent capable of determining if a cell is alive or dead (e.g., one or more of DAPI, propidium iodide (PI), and a Zombie dye). In some embodiments, the method further comprises determine the in vitro cell's morphological signature using brightfield microscopy. The combination of these five binding reagents along with brightfield imaging may be referred to herein as the CellPainting Epigenetic (CPE) palette.

In some embodiments, the first binding reagent, the second binding reagent, the third binding reagent, the fourth binding reagent, the fifth binding reagent, and/or the sixth binding reagent each comprises one of Hoechst 33342; an antigen binding domain that recognizes CD3; an antigen binding domain that recognizes CD19; an antigen binding domain that recognizes CD14; an antigen binding domain that recognizes CD11c; and an antigen binding domain that recognizes CD56. In some embodiments, the method further comprises determine the in vitro cell's morphological signature using brightfield microscopy. The combination of these six binding reagents along with brightfield imaging may be referred to herein as the Cell Specific (CPS) palette.

In some embodiments, the first binding reagent, the second binding reagent, the third binding reagent, the fourth binding reagent, and/or the fifth binding reagent each comprises one of Hoechst 33342; an antigen binding domain that recognizes CD3; Phalloidin; Concanavalin (ConA); and an antigen binding domain that recognizes CD11c. In some embodiments, the method further comprises determine the in vitro cell's morphological signature using brightfield microscopy. The combination of these five binding reagents along with brightfield imaging may be referred to herein as the CellPainting Membrane+Cell Specific (CPMS) palette.

In some embodiments, the first binding reagent, the second binding reagent, the third binding reagent, the fourth binding reagent, and/or the fifth binding reagent each comprises one of Hoechst 33342; WGA; MitoTracker® Orange; Concanavalin (ConA); and Phalloidin. In some embodiments, the method further comprises determine the in vitro cell's morphological signature using brightfield microscopy. The combination of these five binding reagents along with brightfield imaging may be referred to herein as the CPMM palette.

In some embodiments, the first binding reagent, the second binding reagent, the third binding reagent, and/or the fourth binding reagent each comprises one of Hoechst 33342; Wheat germ agglutinin (WGA); Phalloidin; and Concanavalin (ConA). In some embodiments, the method further comprises determine the in vitro cell's morphological signature using brightfield microscopy. The combination of these four binding reagents along with brightfield imaging may be referred to herein Cell Painting Palette 1.

In some embodiments, the first binding reagent, the second binding reagent, the third binding reagent, and/or the fourth binding reagent each comprises one of Hoechst 33342 (e.g., using a Hoechst fluorophore); an antigen binding domain that recognizes CD16 (e.g., using a FITC fluorophore); an antigen binding domain that recognizes CD14 (e.g., using a Texas Red fluorophore); and a lectin capable of identifying cell morphology, e.g., Wheat Germ Agglutinin (WGA) or Concanavalin (ConA)(e.g., using Cy5 fluorophore). In some embodiments, the method further comprises determine the in vitro cell's morphological signature using brightfield microscopy. The combination of these four binding reagents along with brightfield imaging may be referred to herein Cell Painting Palette 3. For example, Cell Painting Palette 3 may comprise a combination of 250 nM Hoechst 33342, 1:50 anti-mouse CD16, 1:50 anti-rabbit CD14, and Wheat Germ Agglutinin (WGA) or Concanavalin (ConA).

In some embodiments, the first binding reagent, the second binding reagent, the third binding reagent, and/or the fourth binding reagent each comprises one of Hoechst 33342 (e.g., using a Hoechst fluorophore); an antigen binding domain directed against Calprotectin (e.g., using a FITC(488) fluorophore); an antigen binding domain directed against mannose receptor (MR)(e.g., using a Texas Red(568) fluorophore); and a lectin capable of identifying cell morphology, e.g., Wheat Germ Agglutinin (WGA) or Concanavalin (ConA) (e.g., using Cy5 fluorophore). In some embodiments, the method further comprises determine the in vitro cell's morphological signature using brightfield microscopy. The combination of these four binding reagents along with brightfield imaging may be referred to herein Cell Painting Palette 4. For example, Cell Painting Palette 4 may comprise a combination of 250 nM Hoechst 33342, 1:50 anti-mouse Calprotectin antibody, 1:800 anti-rabbit mannose receptor antibody, and WGA or ConA.

In some embodiments, the first binding reagent, the second binding reagent, the third binding reagent, the fourth binding reagent, and/or the fifth binding reagent each comprises one of Hoechst 33342; Wheat germ agglutinin (WGA); MitoTracker® Orange or mCherry; Concanavalin (ConA); and Phalloidin (iFluor700). In some embodiments, the method further comprises determine the in vitro cell's morphological signature using brightfield microscopy. For cells infected with rVSV-AG-mCherry, the mChenry signal may be detected. The combination of these binding reagents along with brightfield imaging (and/or mCherry) may be referred to herein Cell Painting Palette 2.

In some embodiments, the first binding reagent, the second binding reagent, the third binding reagent, the fourth binding reagent, and/or the fifth binding reagent, or higher numbered binding reagents comprise any combination of markers selected from the aforementioned palettes.

In some embodiments, the in vitro cell's morphological signature and/or functional signature is determined by measurements of the cell membrane, the in vitro cell's epigenetic signature, the in vitro cell's mitochondrial signature, and cell specific protein markers.

In some embodiments, the method further comprises contacting the in vitro cell with an agent known to affect a cell's morphological signature and/or functional signature. In some embodiments, the agent known to affect a cell's morphological signature and/or functional signature comprises one or more of Vesicular Stomatitis virus (replication deficient VSV); lipopolysaccharide (LPS); Phytohaemagglutinin (PHA); ConcanavalinA (ConA); Pokeweed Mitogen (PWM); Carbonyl cyanide 4-(trifluoromethoxy)phenylhydrazone (FCCP); phorbol myristate acetate (PMA) with Ionomycin; trichostatin A (TSA); C646; 2-Hydroxyglutarate; Colchicine; Cytochalasine A; Jasplakinolide; Paclitaxol (Taxol); Phalloidin; and/or a receptor/antigen blocking antibody; e.g., an anti-MHC antibody.

In some embodiments, the in vitro cell is a peripheral blood mononuclear cell (PBMC).

In some embodiments, identifying the in vitro cell's state, function, and/or predicted age further predicts T cell activation, T cell fate, and T cell exhaustion when the identifying comprises use of a supervised machine learning algorithm.

In some embodiments, identifying the in vitro cell's state, function, and/or predicted age further provides for identification of therapeutically-relevant subsets of immune cells. In some embodiments, the identification of therapeutically-relevant subsets of immune cells comprises use of an unsupervised machine learning algorithm selected from t-SNE, Autoencoder, principal component analysis, and/or k-means clustering.

In some embodiments, identifying the in vitro cell's state, function, and/or predicted age further predicts cytokine and chemokine release by a PBMC. In some embodiments, the prediction of cytokine and chemokine release by a PBMC comprises use of a supervised machine learning algorithm.

In some embodiments, the in vitro cell's state is a characterization of the immune system of a subject. In some embodiments, the subject is an elderly subject. In some embodiments, the elderly subject is at least about 60, 65, or 70 years old.

In some embodiments, the method comprises determining or obtaining a characterization of the immune system of a young subject by identifying the state of cells from the young subject. In some embodiments, the young subject is about 35, 30, or 25 years old or younger. In some embodiments, the method comprises comparing the immune system of the elderly subject and the young subject.

In some embodiments, the in vitro cells of the elderly subject have been treated with a vaccine. In some embodiments, the vaccine is for SARS-CoV-2, influenza, diphtheria, tetanus, pertussis, hepatitis B, poliomyelitis, or Haemophilus influenzae type b, or a combination thereof.

In some embodiments, the in vitro cells of the elderly subject have been treated with a virus. In some embodiments, the virus comprises VSV, influenza, severe acute respiratory syndrome coronavirus 2 (SARS-CoV-2), HIV, or Ebola virus, or a combination or portion thereof (e.g., a protein of the virus).

In some embodiments, the in vitro cells of the elderly subject have been treated with an adjuvant. In some embodiments, the in vitro cells of the elderly subject have been treated with a test agent.

In some embodiments, the method comprises selecting the test agent as a therapeutic if a phenotype of the immune system of the elderly subject's cells is similar to a phenotype of the immune system of the young subject's cells after treatment of the elderly subject's cells with the test agent.

In some embodiments, immune cells from the subject have been treated with a first adjuvant. In some embodiments, the first adjuvant comprises AS01, M1F59, or GLA, or a combination thereof.

In some embodiments, the method comprises determining or obtaining a characterization of the immune system of a reference subject by identifying the state of in vitro cells from the reference subject, wherein the in vitro cells from the reference subject have been treated with a second adjuvant. In some embodiments, the second adjuvant comprises AS01, MF59, or GLA, or a combination thereof. In some embodiments, the method comprises comparing the immune cells from the subject treated with the first adjuvant, to the immune cells of the reference subject treated with the second adjuvant. In some embodiments, if a phenotype of the immune cells from the subject treated with the first adjuvant are similar to a phenotype of the immune cells of the reference subject treated with the second adjuvant, the first adjuvant is selected as a potential adjuvant for improving vaccine response.

In some embodiments, the method comprises determining or obtaining a second characterization of the immune system of the subject by identifying a second state of cells from the subject, wherein the in vitro cells from the subject in the second state have been treated with a second adjuvant. In some embodiments, the second adjuvant comprises AS01, MF59, or GLA, or a combination thereof. In some embodiments, the comprises comparing the immune cells from the subject treated with the first adjuvant, to the immune cells of the subject treated with the second adjuvant. In some embodiments, if a phenotype of the immune cells from the subject treated with the first adjuvant are similar to a phenotype of the immune cells of the subject treated with the second adjuvant, the first adjuvant is selected as a potential adjuvant for improving vaccine response in the subject.

In some embodiments, the in vitro cells from the subject are treated with one or more concentrations of the first adjuvant. In some embodiments, the in vitro cells from the subject are treated with a low concentration of the first adjuvant and a high concentration of first adjuvant, wherein the high concentration of the first adjuvant is at least about 2, 3, 4, 5, 6, 7, 8, 9, or 10 times as concentrated as the low concentration of the first adjuvant, up to about 50 times as concentrated as the low concentration.

In the above aspect or in any of the above embodiments, the method further comprising identifying the state, function, and/or predicted age of an at least second cell. The method comprising: contacting an at least second in vitro cell with a first binding reagent capable of recognizing and binding a first marker of the in vitro cell and contacting the at least second in vitro cell with an at least second binding reagent capable of recognizing and binding an at least second marker of the in vitro cell; determining the at least second in vitro cell's morphological signature and/or functional signature for the at least second in vitro cell based on the intensity of a signal associated with the first binding reagent and the intensity of a signal associated with the at least second binding reagent; and identifying the at least second in vitro cell's state, function, and/or predicted age based upon the morphological signature and/or functional signature.

In some embodiments, cells and cell culture materials are used to promote cell adhesion, e.g., for cells that are not naturally adherent. In some embodiments, the in vitro cells, during the initial cell plating preparation, are kept in cell culture medium depleted from protein supplements and/or plated onto substrates that are treated to promote adhesion.

It shall be understood that different aspects and/or embodiments of the invention can be appreciated individually, collectively, or in combination with each other. Various aspects and/or embodiments of the present disclosure may be applied to any other aspect and/or embodiment of the present disclosure. Any description herein concerning a specific composition and/or method apply to and may be used for any other specific composition and/or method as disclosed herein. In other words, any aspect or embodiment described herein can be combined with any other aspect or embodiment as disclosed herein.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 includes immunofluorescent images showing identification of specific cellular structures via a fluorescent-molecule associated binding reagent that recognizes an epitope on the specific cellular structure. Shown are labeled nuclei/DNA, chromatin, cell membranes, mitochondria, and cytoskeletons. Also included is a brightfield image which shows general morphology of a cell.

FIG. 2 includes an immunofluorescent image showing cells that have been labeled with multiple fluorescent-molecule associated binding reagents.

FIG. 3 is a graph showing the distribution of cell type (lymphocyte, monocyte, dead lymphocytes) from sixteen different donors.

FIG. 4 is a graph showing the distribution of CD antigens among lymphocytes from sixteen different donors.

FIG. 5 shows illustrative normal and abnormal cell type distributions using forward scatter/side scatter flow cytometry.

FIG. 6A shows illustrative cell distributions and staining from a population of T cells. FIG. 6B shows illustrative cell distribution and staining from a population of monocytes. FIG. 6C shows illustrative cell distribution and staining from a population of macrophages. FIG. 6D shows illustrative cell distribution and staining from a population of NK cells. FIG. 6E shows illustrative cell distribution and staining from a population of dendritic cells. FIG. 6F shows illustrative cell distribution and staining from a population of B cells. FIG. 6G shows illustrative cell distribution and staining from a population of macrophages/monocytes. FIG. 6H shows illustrative cell distribution and staining from a population of PBMCs.

FIG. 7 includes immunofluorescent images of cells that have been labeled with multiple fluorescent-molecule associated binding reagents and illustrating spatial changes indicative of T cell activation.

FIG. 8 includes immunofluorescent images of macrophages three days after stimulation (as shown) and labeled using the CellPainting Membrane (CPM) palette.

FIG. 9 includes immunofluorescent images of macrophages five days after stimulation (as shown) and labeled using the CellPainting Membrane (CPM) palette.

FIG. 10 includes immunofluorescent images showing macrophage polarization and CD68 expression.

FIG. 11 includes immunofluorescent images showing macrophage polarization and expression of mannose-receptor and calprotectin expression.

FIGS. 12A-12D includes immunofluorescent images showing labeled mitochondria.

FIG. 13A is a graph showing the intensity of signal from an anti-acetyl-Histone H3 (Lys27) binding reagent after treatment with each compound for 24, 48, and 72 hours in a histone modification assay. FIG. 13B is a graph showing the intensity of signal from an anti-acetyl-Histone H3 (Lys27) binding reagent/Hoechst ratio after treatment with each compound for 24, 48, and 72 hours in a histone modification assay. FIG. 13C is a graph showing the intensity of signal from Tri-Methyl-Histone H3 Lys27 after treatment with each compound for 24, 48, and 72 hours in a histone modification assay. FIG. 13D is a graph showing the intensity of signal from Tri-Methyl-Histone H3 Lys27/Hoechst ration after treatment with each compound for 24, 48, and 72 hours in a histone modification assay.

FIG. 14 is a graph showing the intensity of signal from a phalloidin stain 24 hours after compound treatment in a cytoskeletal modification assay.

FIG. 15A are images showing naïve, M1, and M2 macrophages. FIG. 15B is a visualization plot of cell type, where blue spots indicate M1 cell type, red spots indicate naïve cell type, and orange spots indicate M2 cell type. FIG. 15C are images of macrophage cell state after 5 day culture using field level imaging. FIG. 15D are images of macrophage cell state after 3 day culture using single cell masks.

FIG. 16A shows IL2 concentration in young and old PBMCs 24 and 48 hours after stimulation. FIG. 16B shows IL2 concentration in young and old T-cells 24 and 48 hours after stimulation.

FIG. 17A shows percentage of CD25 positive T-cell activation in young and adult T cells 24 and 48 hours after stimulation. FIG. 17B shows percentage of CD25 positive T-cell activation in young and adult PBMC cells 24 and 48 hours after stimulation. FIG. 17C shows IFNγ production in young and old T cells 24 and 48 hours after stimulation. FIG. 17D shows IFNγ production in young and old PBMC cells 24 and 48 hours after stimulation.

FIG. 18A contains images of young PBMCs after 24 hours using the CPM panel. FIG. 18B contains images of young PBMCs after 48 hours using the CPM panel.

FIG. 19 shows correlations between texture in the ConcanvalinA stain are correlated with age.

FIG. 20 shows that Mitotracker® intensity and entropy increases with age.

FIG. 21 is a plot of the percentage difference from control to condition in T-cells, showing a decrease in percentage of T-cells with MF59.

FIG. 22 is a plot of the percentage difference from control to condition in natural killer cells, showing an increase in percentage of natural killer cells with MF59.

FIG. 23 is a series of plots showing different levels of IL-6, IL-8, MCP1, and TNF-alpha between young and old cell samples.

FIG. 24 are illustrative cells in a mixed lymphocyte population.

FIG. 25A are representative images of isolated T cells, B cells, NK cells, and monocytes from human bulk PBMCs using immunomagnetic negative selection and stained with Hoechst (blue), ConA (red), Phalloidin (green), WGA (far red). The boxed areas represent the single cell shown in the zoomed image. Scale bar=10 μm (overview) and 5 μm (zoom). FIG. 25B shows normalized averages for a subset of morphologic features that differentiate cell populations. ConA, concanavalin A; NK, natural killer; PBMC, peripheral blood mononuclear cell; WGA, wheat germ agglutinin.

FIG. 26A are images of PBMCs and cells predicted to be T cells, B cells, NK cells, noninfected monocytes, or dead cells or infected monocytes. The cells were stained with the Cell Painting Palette 2 (Hoechst, blue; Phalloidin, green; MitoTracker, red), WGA, and ConA, and the predictions were derived from a model trained on image embeddings. Scale bar=10 μm. FIG. 26B is a principal component (PC) analysis plot of 1200 morphologic features computed on single cells within bulk PBMCs exposed to rVSV-AG-mCherry. PCs were generated using the average feature value, then colored by cell type labels from a model trained on image embeddings of cell types. FIG. 26C shows the predicted PBMC composition of 24 donors exposed to rVSV-ΔG-mCherry at 10× MOI. The predictions were derived from a model trained on image embeddings computed from single-cell crops, labeled by cell type. ConA, concanavalin A; MOI, multiplicity of infection; NK, natural killer; PBMC, peripheral blood mononuclear cell; PC, principal component; rVSV-ΔG-mCherry, recombinant vesicular stomatitis virus expressing a red fluorescent construct; WGA, wheat germ agglutinin.

FIG. 27A to-FIG. 27C are plots showing the correlation between the predicted composition of PBMCs exposed to rVSV-ΔG-mCherry and the composition determined by flow cytometry analysis for (FIG. 27A) T cells, (FIG. 27B) NK cells, and (FIG. 27C) infected and dead myeloid cells. NK, natural killer; PBMC, peripheral blood mononuclear cell; rVSV-ΔG-mCherry, recombinant vesicular stomatitis virus expressing a red fluorescent construct.

FIG. 28A shows mean cell composition of 24 donors; cells were untreated or exposed to rVSV-ΔG-mCherry at 0.1×, 1×, and 10×MOI for 24 h. FIG. 28B Cytokine levels in the supernatant of untreated versus virally exposed PBMCs (rVSV-ΔG-mCherry at 10×MOI for 24 h) from 80 donors. The box and whisker plots indicate the median and quartiles. IFN, interferon; IL, interleukin; MCP1, monocyte chemoattractant protein 1; MOI, multiplicity of infection; NK, natural killer; PBMC, peripheral blood mononuclear cell; rVSV-ΔG-mCherry, recombinant vesicular stomatitis virus (VSV) expressing a red fluorescent construct; TNF, tumor necrosis factor.

FIG. 29A shows examples of immune cell clustering and the point pattern of PBMCs stained with Cell Painting Palette 1 (Hoechst, blue; Phalloidin, green; ConA, red). FIG. 29B is a graphic representation of the spatial distribution of lymphocytes and monocytes in a field of view. FIG. 29C and FIG. 29D shows the distribution of cell-to-cell interaction scores using spatial statistical analysis to model non-Poisson multitype point distributions for uninfected and rVSV-ΔG-mCherry-infected PBMCs (at 10×MOI). FIG. 29C summarizes lymphocyte-to-monocyte interaction. FIG. 29D summarizes monocyte-to-monocyte interaction. Box and whisker plots show median and quartiles of the distribution for samples from 24 donors. Statistical significance (via the 2-tailed t test) is indicated. MOI, multiplicity of infection; PBMC, peripheral blood mononuclear cell; rVSV-ΔG-mCherry, recombinant vesicular stomatitis virus (VSV) expressing a red fluorescent construct.

FIG. 30A presents cross-validation performance of a single cell model trained on monocyte image embeddings to predict the different cellular phenotypes of no viral dose (naïve, 96 wells), low viral dose (0.1×MOI, 96 wells), medium viral dose (1×MOI, 96 wells), and high viral dose (10×MOI, 96 wells) aggregated by taking the mean of fields in a well. FIG. 30B Average cross-validation performance of a single cell model trained on T-cell image embeddings to predict the different cellular phenotypes of no viral dose (naïve) and high viral dose (10×MOI). AUC, area under the receiver operating characteristic curve; MOI, multiplicity of infection.

FIG. 31A shows the percentage of uninfected and rVSV-ΔG-mCherry-infected (at 10×MOI for 24 h) monocytes after viral exposure. FIG. 31B shows the percentages of T cells identified from uninfected and rVSV-ΔG-mCherry-infected (at 10′ MOI) PBMCs, presented according to donor age. Statistical significance between younger and older donors after viral infection is indicated. The shading around the trend lines indicates the 95% CIs of the fitted lines. MOI, multiplicity of infection; rVSV-ΔG-mCherry, recombinant vesicular stomatitis virus expressing a red fluorescent protein.

FIG. 32A and FIG. 32B show cytokine levels from the supernatant of uninfected (FIG. 32A) and (FIG. 32B) rVSV-ΔG-mCherry-infected (at 10×MOI for 24 h) PBMCs obtained from 89 donors from younger and older adult populations. FIG. 32C shows the correlation between cytokine levels and percentage of T cells for PBMCs exposed to rVSV-ΔG-mCherry at 10×MOI. IFN, interferon; IL, interleukin; MCP1, monocyte chemoattractant protein 1; MOI, multiplicity of infection; PBMC, peripheral blood mononuclear cell; rVSV-ΔG-mCherry, recombinant vesicular stomatitis virus expressing a red fluorescent construct; TNF, tumor necrosis factor.

FIG. 33A show cross-validation performance of a single cell model trained on T-cell image embeddings to predict the difference between immune responses in cells from younger and older donors. FIG. 33B show the weighted probability of samples from older donors when the samples were obtained from a different experiment than the one used for training. FIG. 33C is a comparison of several features from the multi-phenotype aging profile between younger and older donors. FIG. 33D shows normalized averages for features measuring the intensity distribution of mitochondria membrane potential in T cells from older (o) and younger (y) donors exposed to rVSV-ΔG-mCherry at a 10×MOI. AUC, area under the receiver operating characteristic curve; MOI, multiplicity of infection. MAD, mitochondrial-associated protein degradation; rVSV-ΔG-mCherry, recombinant vesicular stomatitis virus expressing a red fluorescent construct; STD, standard deviation.

FIG. 34A correlates immune aging score and COVID-19 cytokine score plotted for the top 40 therapies from our high throughput screen of 3400 therapies. Ranking is defined by the sum of the immune aging score and the COVID-19 cytokine score. Top therapies are shown in blue and orange, triptonide is shown in green. FIG. 34B A ranking of an immune aging score for top 40 therapies (x-axis).

FIGS. 35A-35D shows cells that were obtained from three older donors and treated with different concentrations of triptonide. The P values for each donor were combined using the Fisher exact test. FIG. 35A shows cytokine levels from the supernatant of rVSV-ΔG-mCherry-infected (at 10×MOI) PBMCs. Asterisks indicate statistical significance relative to untreated control cells. The values are normalized by total cell count per well. Reticularity measurement of mitochondria (FIG. 35B) and aging scores (FIG. 35C) in T cells. Horizontal bars represent the distribution of untreated controls from younger and older donors; the solid line represents the median and the lower and upper dashed lines represent the 25th and 75th quartiles, respectively. The line graph represents the median and 95% CI for treated cells, with statistical significance relative to untreated control cells indicated by asterisks. FIG. 35D On-age and off-age scores for T cells treated with either triptonide or dimethyl fumarate. Distributions for younger and older control (untreated) cells are plotted using a Gaussian kernel density estimation. Statistical significance relative to untreated control cells is indicated by the following: *P<0.05; **P<0.001; ***P<0.0001. IL, interleukin; MCP1, monocyte chemoattractant protein 1; MOI, multiplicity of infection; PBMC, peripheral blood mononuclear cell; TNF, tumor necrosis factor.

FIG. 36 show representative flow plots for cell composition discrimination. Events were first gated on FSC/SSC profile to distinguish dead cells and debris from live myeloid and live lymphoid cells. Myeloid morphology cells were then stratified by CD14 and CD11c expression to distinguish monocytes (CD14+). Lymphoid cells were first stratified by CD3 and CD19 expression to distinguish T cells (CD3+) and B cells (CD19+). Double-negative cells from the lymphoid morphology fraction were used to distinguish NK cells (CD3− CD19− CD56+).

FIG. 37A includes a Spatial Kolmogorov-Smirnov plot to identify inconsistencies with a homogeneous Poisson distribution. FIG. 37B shows significant departures from an expected distribution (homogeneous) which may indicate a heterogeneous distribution that is not independent of the spatial landscape. CSR, complete spatial randomness.

FIG. 38 shows cytokine levels from the supernatant of PBMCs obtained from 89 older and younger donors and exposed to rVSV-ΔG-mCherry at 1×MOI. IFN, interferon; IL, interleukin; MCP, monocyte chemoattractant protein 1; MOI, multiplicity of infection; PBMC, peripheral blood mononuclear cell; rVSV-ΔG-mCherry, recombinant vesicular stomatitis virus expressing a red fluorescent construct; TNF, tumor necrosis factor.

FIG. 39A and FIG. 39B show power log-log metrics computed on the Hoechst channel for (FIG. 39A) a plate that passed quality control and (FIG. 39B) a plate flagged for quality control due to technical artifacts.

FIG. 40A and FIG. 40B are examples of single cell images that are automatically labeled as (FIG. 40A) passing and (FIG. 40B) failing quality control.

FIG. 41 illustrates a computer system that is programmed or otherwise configured to implement methods provided herein.

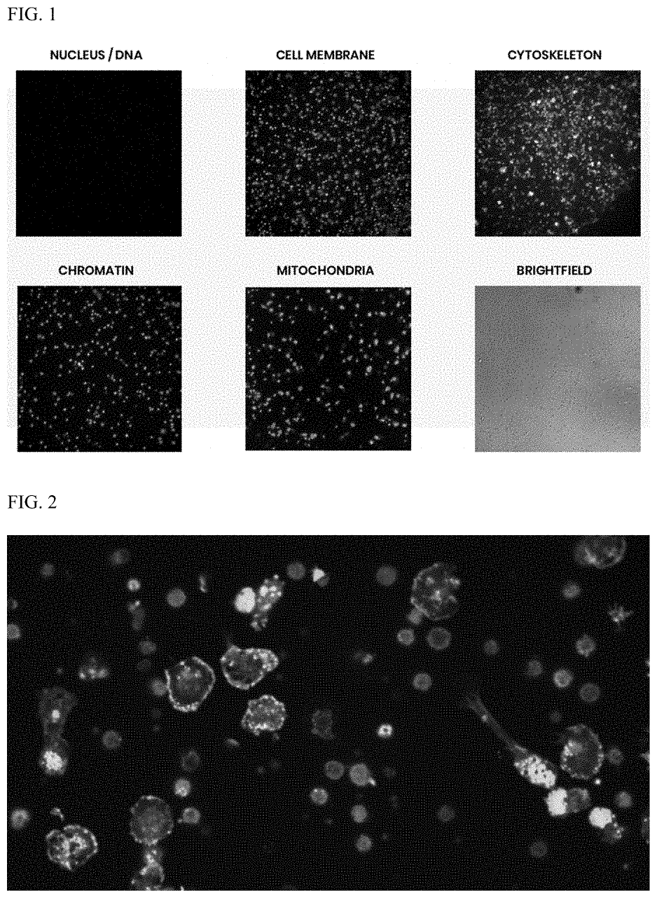

### DETAILED DESCRIPTION OF THE INVENTION

The aging process is the most important risk factor for morbidity, mortality, and progression of severe infectious disease. While the COVID-19 pandemic has drawn attention to the vulnerability of aging adults, the heightened risk faced by these patients is not limited to times of pandemic. Older adults face an increased risk of death from seasonal influenza and sepsis, and the ever-increasing aging population across the globe presents a challenge to protect this at-risk population. To help address this challenge, described herein is a system that produces a multi-phenotype aging profile by combining high-throughput imaging and machine learning algorithms. This is a novel way to study immune cell responses to viral infection that allows for the rapid characterization of these responses in multiple dimensions (e.g., cell composition, cell-to-cell interactions, cellular features, cytokine production, and hidden complexities) and enables screening for therapies that can have an impact on the older immune response to viral infections.

The present disclosure is based, in part, on the discovery of methods and systems for identifying an in vitro cell's state, function, and/or predicted age. These methods are useful in, at least, drug discovery for identifying drugs that promote longevity, improve immune function, and/or treat disease.

In some embodiments of the present disclosure, cells are labeled with a plurality of binding agents (e.g. antibodies) each associated with a different fluorescent molecule. The specific combination, patterns, intensities, and localizations of fluorescent signals together provides a morphological signature and/or functional signature. The morphological signature and/or functional signature is analyzed by various machine learning techniques and compared to trained models to identify the cells' state, function, and/or predicted age. Such an understanding of a cells' state, function, and/or predicted age can be used in drug discovery. Here, a change in the cells' state, function, and/or predicted age following contact with a potential drug can be accurately determined; thereby, identifying a drug that provides a desired effect, e.g., increase in cytokine release, a reduction in the cell's predicted age, activation of a functional trait, a morphological change, a fate change from an inactive cell type to an active one.

The present disclosure uses new and inventive combinations of assay methodologies to determine an in vitro cell's morphological signature and/or functional signature. Characterizing these signatures is facilitated by machine learning techniques which help create models that are useful for identifying the in vitro cell's state, function, and/or predicted age and for characterizing changes in the cell upon contact with a potential drug. Overall, the methods are inexpensive and with high-throughput, provide high information content, are reliable and robust, are interpretable to provide useful information, measure whole cell functions, and are ideal for machine learning. And, unlike current technologies, in general, the present disclosure provides high content imaging with superior information content and at a lower cost. Together, the methods of the present disclosure provide for the efficient discovery of drugs and methods that promote longevity, improve immune function, and/or treat disease.

Methods for Identifying an In Vitro Cell's State, Function, and/or Predicted Age

An aspect of the present disclosure is a method for identifying an in vitro cell's state, function, and/or predicted age. The method comprising: contacting an in vitro cell with a first binding reagent capable of recognizing and binding a first marker of the cell and contacting the cell with an at least second binding reagent capable of recognizing and binding an at least second marker of the cell; determining the in vitro cell's morphological signature and/or functional signature based on the intensity of a signal associated with the first binding reagent and the intensity of a signal associated with the at least second binding reagent; and identifying the cell's state, function, and/or predicted age based upon the morphological signature and/or functional signature.

In some embodiments, the in vitro cell's morphological signature comprises the cell's size and/or the cell's shape and/or morphological properties (e.g., eccentricity, form factor, and solidity). In some embodiments, the in vitro cell's functional signature comprises one or more of the presence or absence of a marker characteristic of a specific cell organelle, the presence or absence of a marker characteristic of a specific cell type, the presence or absence of a marker characteristic of a specific cell activity, the presence or absence of a marker characteristic of an active cell, the presence or absence of a marker characteristic of an inactive cell, and/or the presence or absence of a marker indicating gene expression, and/or the ability of the in vitro cell to interact with another cell or fragment of another cell. In some embodiments, the first marker and/or the at least second marker is located on or in the cell's nucleus, cell membrane, cytoplasm, cytoskeleton, chromatin, mitochondrion, and/or extracellular matrix. In some embodiments, the first binding reagent and/or the at least second binding reagent are each respectively directly or indirectly labeled with a first fluorescent molecule and/or an at least second fluorescent molecule. In some embodiments, the at least second marker comprises a third marker, a fourth marker, a fifth marker, a sixth marker, a seventh marker, an eighth marker, a ninth marker, a tenth marker, or more markers. In some embodiments, determining the in vitro cell's morphological signature and/or functional signature further comprises contacting the cell with a third binding reagent, a fourth binding reagent, a fifth binding reagent, a sixth binding reagent, a seventh binding reagent, and/or an eighth binding reagent each respectively capable of recognizing and binding a third marker, a fourth marker, a fifth marker, a sixth marker, a seventh marker, an eighth marker, a ninth eighth marker, and/or an at least tenth marker. In some embodiments, the third binding reagent, the fourth binding reagent, the fifth binding reagent, the sixth binding reagent, the seventh binding reagent, the eighth binding reagent, the ninth binding reagent, and/or the at least tenth binding reagent are each respectively directly or indirectly labeled with a third fluorescent molecule, a fourth fluorescent molecule, a fifth fluorescent molecule, a sixth fluorescent molecule, a seventh fluorescent molecule, an eighth fluorescent molecule, a ninth fluorescent molecule and/or an at least tenth fluorescent molecule.

In some embodiments, the intensity of a signal associated with the first binding reagent and the intensity of a signal associated with the at least second binding reagent comprises visualization of the cell or a portion thereof by light microscopy, e.g., fluorescent microscopy. In some embodiments, the intensity of a signal associated with the first binding reagent and the intensity of a signal associated with the at least second binding reagent are produced by each of the first fluorescent molecule and/or the at least second fluorescent molecule. In some embodiments, determining an in vitro cell's morphological signature and/or functional signature further comprises normalizing the signal intensity produced by each of the first fluorescent molecule and/or the at least second fluorescent molecule. In some embodiments, the in vitro cell's morphological signature and/or functional signature is calculated based upon the normalized signal intensity produced by each of the first fluorescent molecule and/or the at least second fluorescent molecule.

In some embodiments, the in vitro cell is obtained from a mammal, e.g., a human, mouse, rat, guinea pig, dog, cat, horse, cow, pig, rabbit, sheep, or non-human primate, such as a monkey, chimpanzee, or baboon. In some embodiments, the mammal is a non-rodent. In some embodiments, the mammal is a human.

In some embodiments, the human is an adult human. In some embodiments, the human has an age in a range of from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old, or older.

In some embodiments, the in vitro cell is obtained from a tissue sample from the mammal. The tissue sample may comprise, or be derived from, a tissue biopsy, blood, blood plasma, extracellular fluid, dried blood spots, cultured cells, culture media, or discarded tissue. In some embodiments, the in vitro cell is obtained from whole blood. In some embodiments, the in vitro cell is obtained from a fraction of whole blood, e.g., comprising white blood cells. A fraction comprising white blood cells may comprise hematopoietic stem cells, T cells, B Cell, Natural Killer (NK) cells, Monocytes (including macrophages). The in vitro cell may be isolated from a fraction of whole blood, for example, an in vitro cell of one type (e.g., B cells) may be isolated from other type of cell (e.g., NK cells).

In some embodiments, the in vitro cell's predicted age is determined by a machine learning technique selected from convolutional neural networks and gradient boosted trees to compute the in vitro cell's age (e.g., as a continuous value between 0 and 100). For example, the machine learning technique may comprise a supervised machine learning algorithm or supervised machine learning classifier configured to analyze a morphological signature, a functional signature, a marker, etc. of an in vitro cell. In some embodiments, a predicted age of the in vitro cell that is over 65 indicates that the cell is dying, old, and/or senescent. In some embodiments, a predicted age of the in vitro cell that is under 30 indicates that the in vitro cell is young, healthy, and/or active.

In some embodiments, the marker indicating gene expression is methylated DNA.

In some embodiments, the marker characteristic of a specific cell type is differentially expressed by a T cell (e.g., memory T cell or an effector T cell), a B cell, an Natural Killer (NK) cell, a monocyte/macrophage (e.g., an M1 macrophage or by an M2 macrophage), and a monocyte/dendritic cell.

In some embodiments, the in vitro cell's morphological signature and/or functional signature comprises characterizing the cell's mitochondrial shape using one or more of spatial pattern analysis, texture analysis, intensity changes, and convolutional neural networks.

In some embodiments, the in vitro cell's size and/or the cell's shape and/or morphological properties (e.g., eccentricity, form factor, and solidity) is quantified using convolutional neural networks.

In some embodiments, the marker characteristic of an active cell identifies T cell activation.

In some embodiments, the marker characteristic of an inactive cell identifies T cell exhaustion.

In some embodiments, the marker characteristic of a specific cell type is differentially expressed by an M1 macrophage and by an M2 macrophage. In some embodiments, the marker characteristic of an M1 macrophage is Calprotectin and/or the marker characteristic of an M2 macrophage is Mannose Receptor. In some embodiments, the marker is identified by an antigen binding domain that recognizes Calprotectin or Mannose Receptor. In some embodiments, a method is provided for predicting whether a macrophage has a M1 or M2 phenotype using a CPM or CPE or Cell Painting Pallet 4 staining procedure as described elsewhere herein.

In some embodiments, the cell's ability to interact with another cell or fragment of another cell is quantified using a spatial pattern analysis method.

In some embodiments, the in vitro cell's morphological signature and/or functional signature comprises a characterization based upon the presence or absence of cell motility, genomic instability, telomere attrition, epigenetic alterations, loss of proteostasis, deregulated nutrient-sensing, mitochondrial dysfunction, cellular senescence, stem cell exhaustion, and/or altered intercellular communication. In some embodiments, intercellular communication mediated by cytokines is a functional signature characterized using a method described herein. As a non-limiting example, PBMCs are stimulated and cytokine release is quantified. In some embodiments, PBMCs are activated with LPS from different sources, with expected cytokine release of: IFNg, IL6, IL10, IL12 and TNFa. In some embodiments, PBMCs are activated with PMA and lonomycin, with expected cytokine release of IL2, IFNg, TNFa, RANTES and φP. In some embodiments, PBMCs are activated with PWM, with expected cytokine release of IFNg, TNFa, TNFβ, IL2 (IL4), and IL10. Non-limiting examples of markers characteristic of cellular communication mediated by cytokines include: IL-1a, IL-1b, IL-2, IL-4, IL-5, IL-6, IL-10, IL-12, IL-13, IL-15, IL-17, IL-23, IFNγ, TNFα, TNFβ, IL-8, IL-12p70, and any combination thereof. An exemplary panel of markers comprises IL-1a, IL-1b, IL-2, IL-4, IL-5, IL-6, IL-10, IL-12, IL-13, IL-15, IL-17, IL-23, IFNγ, TNFα, and TNFβ. Another exemplary panel of markers comprises IL-1a, Il-1b, IL-2, IL-4, IL-5, IL-6, IL-8, IL-10, IL-12p70, IL-13, IL-15, IL-17, IL-23, IFNγ, TNFα, and TNFβ. In some embodiments, methods and systems disclosed herein use fluorescence microscopy to determine biological features of a subject. In some cases, the features are of the immune system. The features may be used to provide insight into specific biological function. Categories for evaluation include: compositional—portions of individual cell populations present within the whole, spatial—the physical relationship between different cell types, and functional—changes in biological mechanisms which result in significant actions. Compositional: See Examples 1 and 2. Spatial: For cell to cell interactions, see Example 3. For antigen presentation, see Example 4. Functional: For cytokines, see Example 5. For M1/M2 macrophages, see Example 6. For T cell fate, see Example 7. For T cell exhaustion, see Example 9.

In some embodiments, the in vitro cell's signature comprises a spatial biological feature. As a first example, the biological feature comprises cell sorting. As another example, the biological feature comprises cell type composition. As another example, the biological feature comprises cell sorting and cell type composition.

In some embodiments, the in vitro cell's signature comprises cell type composition. Non-limiting examples of markers for identification of cell types include CD3 (expressed on T cells), CD19 (expressed on B cells), CD56 (expressed on NK cells), CD14 (expressed on macrophages and expressed on subtypes of monocytes), and CD11c (highly expressed on dendritic cells and expressed on some monocytes (intermediate and nonclassical)). T cells can be characterized by CD3+, CD19−, CD14−, CD11c−. B cells can be characterized by CD3−, CD19+, CD56−, CD14−, CD11c−. NK cells can be characterized by CD3−, CD56+, CD19−, CD14−, CD11c−. Macrophages and monocytes can be characterized by CD3−, CD19−, CD14+. Dendritic cells can be characterized by CD3−, CD19−, CD11c+. In certain instances, NK cells are CD19+, CD56+ and T cells are CD11c+, CD3+. Methods and palettes described herein may be utilized to identify cell type. In some embodiments, a palette comprises CellPainting Membrane (CPM), CellPainting Epigenetic (CPE), Cell Specific (CPS), CellPainting Membrane+Cell Specific (CPMS), or any combination of markers of the aforementioned palettes, and combinations of palettes. As an example, the palette comprises CPM. As another example, the palette comprises CPE. As another example, the palette comprises CPS. As another example, the palette comprises CPMS. As another example, the palette comprises markers selected from CPE and from CPS. In some embodiments, the method comprises performing an assay of Example 2.

In some embodiments, the in vitro cell's signature comprises a spatial biological feature. As a first example, the biological feature comprises cell to cell interaction. As another example, the biological feature comprises antigen presentation. As another example, the biological feature comprises cell to cell interaction and antigen presentation.

In some embodiments, the in vitro cell's signature comprises characterization of antigen presentation. T cells are activated by the binding of T cell receptors (TCRs) to antigen-loaded major histocompatibility complexes on antigen-presenting cells (APCs), resulting in the formation of TCR “microclusters”, which coalesce in the immunological synapse (IS), thereby allowing the delivery of effector functions. Actin cytoskeletal rearrangements regulating the membrane architecture of T cells are important during initial cell-cell interactions through the formation of actin-rich protrusions. These, in turn, allow the formation of “close contacts” between T cells and APCs, favoring the transient interactions of proteins required for signaling. Changes to cytoskeleton, rearrangement of T cell mitochondria to the location of the immunological synapse, and TCR microclusters may be visualized by fluorescence microscopy using a herein-disclosed CellPainting palette, or any combination of markers from a CellPainting palette, described herein. For instance, cells are contacted with one or more markers from CellPainting Membrane (CPM), CellPainting Epigenetic (CPE), Cell Specific (CPS), and CellPainting Membrane+Cell Specific (CPMS), and imaged accordingly. In some embodiments, the method comprises performing an assay of Example 4.

In some embodiments, the in vitro cell's signature comprises characterization of cell to cell interaction. The interaction between T cells and professional antigen-presenting cells (APCs), including dendritic cells and macrophages, is an essential step in triggering an adaptive (T cell) immune response. APCs present foreign antigens via major histocompatibility complex (MHC) receptors to CD3 receptors on T cells (TCRs) which stimulate the cells to seek out specific pathogens using effector functions. The first step in this interaction is the scanning of MHC receptors by T cells, which is influenced by the motility of individual cell populations. T cells scan the surfaces of APCs in search of antigen peptides bound to MHC proteins. Methods and palettes herein may be utilized to quantifying this interaction, which may be useful for understanding the effects of immunomodulatory drugs. In some embodiments, using spatial point pattern analysis, statistical models of the distance between various cell types in a PBMC mixture are built using the methods herein. In some embodiments, a palette comprises CellPainting Membrane (CPM), CellPainting Epigenetic (CPE), Cell Specific (CPS), CellPainting Membrane+Cell Specific (CPMS), any combination of markers of the aforementioned palettes, and combinations of palettes. As an example, the palette comprises CPM. As another example, the palette comprises CPE. As another example, the palette comprises CPM and CPE. In some embodiments, the method comprises performing an assay of Example 3.

In some embodiments, the in vitro cell's morphological signature and/or functional signature comprises a characterization based upon the presence, absence, an/or quantification of one or more of the following parameters: T-cell PMH, T-Cell PMH: On Age, T-Cell PMH: Off Age, NK PMH, Lymphocyte PMH, Monocyte PMH, All-Cell PMH, T-Cell CPH, T-Cell CPH: On Age, T-Cell CPH: Off Age, NK CPH, Lymphocyte CPH, Monocyte CPH, All-Cell CPH, T-Cell Viral, Monocytes Viral, % NK, NK Count, % Lymphocyte, Lymphocyte Count, % Monocyte, Monocyte Count, % Loaded Monocyte, Loaded Monocyte Count, % T-Cell, T-Cell Count, T-Cell Mito Texture, T-Cell Concentric Phalloidin, T-Cell Reticular Mitochondria, and a cytokine (e.g., MCP1, IL-6, TNF-alpha, IL-1, IL-8, IL-17A, IL-4, IL-10, IL-2, IFN-gamma). In some embodiments, parameters characterized for cell age prediction include one or more of: T-cell PMH, T-Cell PMH: On Age, T-Cell PMH: Off Age, NK PMH, Lymphocyte PMH, Monocyte PMH, All-Cell PMH, T-Cell CPH, T-Cell CPH: On Age, T-Cell CPH: Off Age, NK CPH, Lymphocyte CPH, Monocyte CPH, All-Cell CPH, T-Cell Viral, and Monocytes Viral. In some embodiments, parameters characterized for PBMC composition include one or more of: % NK, NK Count, % Lymphocyte, Lymphocyte Count, % Monocyte, Monocyte Count, % Loaded Monocyte, Loaded Monocyte Count, % T-Cell, and T-Cell Count. In some embodiments, parameters characterized for cell structural features include one or more of: T-Cell Mito Texture, T-Cell Concentric Phalloidin, and T-Cell Reticular Mitochondria. In some embodiments, parameters characterized for cytokine profiling include one or more of: MCP1, IL-6, TNF-alpha, IL-1, IL-8, IL-17A, IL-4, IL-10, IL-2, and IFN-gamma. The term PMH above refers to Phalloidin, MitoTracker, and Hoechst stains that the model was trained upon: the term CPH above refers to ConA, Phalloidin, and Hoechst stains that the model was trained upon. To better understand a shift in T-cell age score after treatment, image embeddings of T cells were used to compute an “on-age” score, measuring the distance from young controls, and a complementary “off-age” score, measuring the magnitude of the change in the orthogonal direction to age distances.

In some embodiments, the first binding reagent and/or the at least second binding reagent is a lectin. In some embodiments, the lectin is Wheat Germ Agglutinin (WGA) or Concanavalin (ConA).

In some embodiments, the first binding reagent and/or the at least second binding reagent comprises an antigen binding domain. In some embodiments, the binding reagent that comprises an antigen binding domain is an antibody. In some embodiments, the binding reagent that comprises an antigen binding domain recognizes total Histone H3, Acetyl-Histone H3, or Tri-Methyl-Histone H3. In some embodiments, the binding reagent that comprises an antigen binding domain recognizes CD3, CD11c, CD14, CD16, CD19, CD45, CD56, or CD68.

Examples of binding reagent comprises an antigen binding domain include a single-chain antibody (scFv); a recombinant camelid heavy-chain-only antibody (VHH); a shark heavy-chain-only antibody (VNAR); a microprotein; a darpin; an anticalin; an adnectin; an aptamer; an Fv; an Fab; an Fab′; and an F(ab′)2; and an antibody or antigen binding domain thereof from an IgA (including subclasses IgA1 and IgA2), IgD, IgE, IgG (including subclasses IgG1, IgG2, IgG3, and IgG4), or IgM Fc domain, optionally a human Fc domain, or a hybrid and/or variant thereof.

In some embodiments, the first binding reagent and/or the at least second binding reagent is a nuclear stain, e.g., Hoechst 33342, or a mitochondrial stain, e.g., a MitoTracker®.

In some embodiments, the first binding reagent and/or the at least second binding reagent is Phalloidin. Phalloidin tightly and selectively bind to F-actin (e.g., in a cell's cytoskeleton). When attached to a fluorescent tag, Phalloidin visualize a cell's cytoskeleton.

In some embodiments, the first binding reagent and/or the at least second binding reagent is capable of determining if a cell is alive or dead (e.g., one or more of DAPI, propidium iodide (PI), and a Zombie dye).

In some embodiments, the first binding reagent, the second binding reagent, the third binding reagent, the fourth binding reagent, and/or the fifth binding reagent each comprises one of Hoechst 33342; Wheat Germ Agglutinin (WGA); Phalloidin; Concanavalin (ConA); and a binding reagent capable of determining if a cell is alive or dead (e.g., one or more of DAPI, propidium iodide (PI), and a Zombie dye). In some embodiments, the method further comprises determine the in vitro cell's morphological signature using brightfield microscopy. The combination of these five binding reagents along with brightfield imaging may be referred to herein as the CellPainting Membrane (CPM) palette.

In some embodiments, the first binding reagent, the second binding reagent, the third binding reagent, the fourth binding reagent, and/or the fifth binding reagent each comprises one of Hoechst 33342; an antigen binding domain that recognizes Acetyl-Histone H3; an antigen binding domain that recognizes Tri-Methyl-Histone H3; MitoTracker® Orange; and a binding reagent capable of determining if a cell is alive or dead (e.g., one or more of DAPI, propidium iodide (PI), and a Zombie dye). In some embodiments, the method further comprises determine the in vitro cell's morphological signature using brightfield microscopy. The combination of these five binding reagents along with brightfield imaging may be referred to herein as the CellPainting Epigenetic (CPE) palette.

In some embodiments, the first binding reagent, the second binding reagent, the third binding reagent, the fourth binding reagent, the fifth binding reagent, and/or the sixth binding reagent each comprises one of Hoechst 33342; an antigen binding domain that recognizes CD3; an antigen binding domain that recognizes CD19; an antigen binding domain that recognizes CD14; an antigen binding domain that recognizes CD11c; and an antigen binding domain that recognizes CD56. In some embodiments, the method further comprises determine the in vitro cell's morphological signature using brightfield microscopy. The combination of these six binding reagents along with brightfield imaging may be referred to herein as the Cell Specific (CPS) palette.

In some embodiments, the first binding reagent, the second binding reagent, the third binding reagent, the fourth binding reagent, and/or the fifth binding reagent each comprises one of Hoechst 33342; an antigen binding domain that recognizes CD3; Phalloidin; Concanavalin (ConA); and an antigen binding domain that recognizes CD11c. In some embodiments, the method further comprises determine the in vitro cell's morphological signature using brightfield microscopy. The combination of these five binding reagents along with brightfield imaging may be referred to herein as the CellPainting Membrane+Cell Specific (CPMS) palette.

In some embodiments, the first binding reagent, the second binding reagent, the third binding reagent, the fourth binding reagent, and/or the fifth binding reagent each comprises one of Hoechst 33342; Wheat germ agglutinin (WGA); MitoTracker® Orange; Concanavalin (ConA); and Phalloidin. In some embodiments, the method further comprises determine the in vitro cell's morphological signature using brightfield microscopy. The combination of these five binding reagents along with brightfield imaging may be referred to herein as the CPMM palette.

In some embodiments, the first binding reagent, the second binding reagent, the third binding reagent, and/or the fourth binding reagent each comprises one of Hoechst 33342; Wheat germ agglutinin (WGA); Phalloidin; and Concanavalin (ConA). In some embodiments, the method further comprises determine the in vitro cell's morphological signature using brightfield microscopy. The combination of these four binding reagents along with brightfield imaging may be referred to herein Cell Painting Palette 1.

In some embodiments, the first binding reagent, the second binding reagent, the third binding reagent, and/or the fourth binding reagent each comprises one of Hoechst 33342 (e.g., using a Hoechst fluorophore); an antigen binding domain that recognizes CD16 (e.g., using a FITC fluorophore); an antigen binding domain that recognizes CD14 (e.g., using a Texas Red fluorophore); and a lectin capable of identifying cell morphology, e.g., Wheat Germ Agglutinin (WGA) or Concanavalin (ConA) (e.g., using Cy5 fluorophore). In some embodiments, the method further comprises determine the in vitro cell's morphological signature using brightfield microscopy. The combination of these four binding reagents along with brightfield imaging may be referred to herein Cell Painting Palette 3. For example, Cell Painting Palette 3 may comprise a combination of 250 nM Hoechst 33342, 1:50 anti-mouse CD16, 1:50 anti-rabbit CD14, and WGA or ConA.

In some embodiments, the first binding reagent, the second binding reagent, the third binding reagent, and/or the fourth binding reagent each comprises one of Hoechst 33342 (e.g., using a Hoechst fluorophore); an antigen binding domain directed against Calprotectin (e.g., using a FITC(488) fluorophore); an antigen binding domain directed against mannose receptor antibody (e.g., using a Texas Red(568) fluorophore); and a lectin capable of identifying cell morphology, e.g., Wheat Germ Agglutinin (WGA) or Concanavalin (ConA) (e.g., using Cy5 fluorophore). In some embodiments, the method further comprises determine the in vitro cell's morphological signature using brightfield microscopy. The combination of these four binding reagents along with brightfield imaging may be referred to herein Cell Painting Palette 4. For example, Cell Painting Palette 4 may comprise a combination of 250 nM Hoechst 33342, 1:50 anti-mouse Calprotectin antibody, 1:800 anti-rabbit mannose receptor antibody, and WGA or ConA.

In some embodiments, the first binding reagent, the second binding reagent, the third binding reagent, the fourth binding reagent, and/or the fifth binding reagent each comprises one of Hoechst 33342; Wheat germ agglutinin (WGA); MitoTracker® Orange or mCherry; Concanavalin (ConA); and Phalloidin (iFluor700). In some embodiments, the method further comprises determine the in vitro cell's morphological signature using brightfield microscopy. For cells infected with rVSV-ΔG-mCherry, the mCherry signal may be detected. The combination of these binding reagents along with brightfield imaging (and/or mCherry) may be referred to herein Cell Painting Palette 2.

In some embodiments, the first binding reagent, the second binding reagent, the third binding reagent, the fourth binding reagent, and/or the fifth binding reagent, or higher numbered binding reagents comprise any combination of markers selected from the aforementioned palettes.

In some embodiments, the in vitro cell's morphological signature and/or functional signature is determined by measurements of the cell membrane, the cell's epigenetic signature, the cell's mitochondrial signature, and cell specific protein markers.

In some embodiments, the method further comprises contacting the in vitro cell with an agent known to affect a cell's morphological signature and/or functional signature. In some embodiments, the agent known to affect a cell's morphological signature and/or functional signature comprises one or more of Vesicular Stomatitis virus (replication deficient VSV); lipopolysaccharide (LPS); Phytohaemagglutinin (PHA); ConcanavalinA (ConA); Pokeweed Mitogen (PWM); Carbonyl cyanide 4-(trifluoromethoxy)phenylhydrazone (FCCP); phorbol myristate acetate (PMA) with Ionomycin; trichostatin A (TSA); C646; 2-Hydroxyglutarate; Colchicine; Cytochalasine A; Jasplakinolide; Paclitaxol (Taxol); Phalloidin; and/or a receptor/antigen blocking antibody; e.g., an anti-MHC antibody.

In some embodiments, the in vitro cell is a peripheral blood mononuclear cell (PBMC). Using PBMC provides numerous advantages, e.g., relative to isolated cell. First, using PBMCs reduces costs, as they are less expensive than isolated cells; this savings allow for a scaling up the number assays that can be performed (e.g., during training and validations) which may significantly increase success of the resulting models. Second, using PBMCs reduces wetlab stress in that they do not require significant manipulations which may affect the cell's function and/or activity, which may result a process-based confounder. Third, in vivo, PBMCs naturally contact and communicate with each other; thus, in vitro models using PBMCs approximate natural conditions and, thereby, may provide greater clinical relevance. Fourth, similarly, an important action of cells in the immune system (which includes PBMCs) is communication and interaction between cells; in vitro PMBCs retain the ability to communication and interaction and this can be measured and characterized when developing an immune profiling platform. Fifth, PBMCs comprise a variety of subtypes and sub-subtypes: PBMCs include B cells and T cells; T cells can be divided into cytotoxic T cells, regulatory T cells, and T helper cells; and T helper cells can be subdivided into, at least, T helper1, T helper2, T helper17, and T helper22. Modeling activity, function, and morphology of subtypes of PBMC provide a better understanding of the in vivo immune system. And, Sixth, it is reasonable that there are key cell types (and subtypes) that are relevant to human disease (e.g., disease prevention); the herein disclosed methods segment PBMCs and can help identify these key cell types' state and function.

In some embodiments, identifying the cell's state, function, and/or predicted age predicts T cell activation, T cell fate, and/or T cell exhaustion. In some embodiments, identifying the cell's state, function, and/or predicted age further predicts T cell activation, T cell fate, and T cell exhaustion when the identifying comprises use of a supervised machine learning algorithm. Non-limiting examples of hallmarks for exhaustion of CD8+ T cells include: co-expression of multiple inhibitory receptors such as PD-1, CTLA-4, LAG-3, TIM-3, 2B4/CD244/SLAMF4, CD160, and/or TIGIT; loss of IL-2 production, proliferative capacity, ex vivo cytolytic activity; impairment of production of TNF-alpha, IFN-gamma, and/or cc (beta) chemokines; degranulation; expression of high levels of Granzyme B; poor responsiveness to IL-7 and/or IL-15 (drive memory T cell antigen-dependent proliferation long after antigen elimination); and cell death (e.g., may be due to overstimulation). Non-limiting examples of hallmarks for exhaustion of CD4+ T cells include: co-expression of multiple inhibitory receptors such as PD-1, CTLA-4, LAG-3, TIM-3, 2B4/CD244/SLAMF4, CD160, and/or TIGIT; loss of IL-2 production, proliferative capacity, ex vivo cytolytic activity; impairment of production of TNF-alpha, IFN-gamma, and/or cc (beta) chemokines; altered expression of GATA-3, Bcl-6, and/or Helios; similarity to a T Follicular Helper (Tth) cell phenotype (e.g., surface markers such as CD4, CXCR5, ICOS, and/or PD-1; secreted cytokines such as IL-4, IL-6, and/or IL-21; transcription factors such as Bcl-6, IRF4, and/or STAT4); and earlier manifestation of dysfunction compared to CD8+ exhausted T cells (Tex cells). Any combination of the hallmark markers listed may be used to evaluate T cell exhaustion phenotype. In an exemplary method, the in vitro cell is contacted with a binding reagent specific for one or more of the hallmarks. For instance, the binding reagent comprises a fluorophore attached to an agent that binds to the hallmark (e.g., an antibody like an anti-interleukin attached to a fluorophore).

In some embodiments, identifying the cell's state, function, and/or predicted age further provides for identification of therapeutically-relevant subsets of immune cells. In some embodiments, the identification of therapeutically-relevant subsets of immune cells comprises use of an unsupervised machine learning algorithm selected from t-SNE, Autoencoder, principal component analysis, and/or k-means clustering.

In some embodiments, identifying the cell's state, function, and/or predicted age further predicts cytokine and chemokine release by a PBMC. In some embodiments, the prediction of cytokine and chemokine release by a PBMC comprises use of a supervised machine learning algorithm.

In some embodiments, the cell's function is immune function. In some embodiments, methods provided herein for identification of a cell's function include methods for identifying immune cell function. For instance, the method comprises measurement of immune functions and/or phenotypes at the whole cell level of an immune cell. The methods may be performed on any clinically relevant population of immune cells. The methods may also be used to discover, de novo, populations which are most pertinent to target indications. Once a population is chosen, the method can be utilized to characterize how immune function and phenotypes change in response to a therapeutic intervention, e.g., such as a test agent described herein. Immune cells utilized in the methods described herein include PBMCs. In some embodiments, the methods include fluorescent microscopy methods described herein. In some embodiments, the methods are low-cost and provide high information content, as compared to existing technologies for immune profiling. In addition, the methods may be high-throughput, reliable, robust, interpretable, measure whole cell function, are ideal for machine learning, or any combination thereof.

In some embodiments, methods provided herein for identification of a cell's state, function, and/or predicted age include methods for discovering and targeting aging mechanisms which cause a decreased response to vaccines. This may be useful in developing vaccines for the elderly, who have shown decreased response to traditional vaccination strategies.

In some embodiments, a method for identification of a cell's state, function and/or predicted age comprises imaging bulk PBMCs and segmenting cell populations. The image profiles can be compared with profiles for existing cells populations, as well as used to discover new cell populations. Non-limiting examples of image profiles for existing populations include T cells, B cells, monocytes, macrophages, dendritic cells, and natural killer cells. The images may be analyzed to identify biological features of the cells. Biological features may include prediction of cell age and/or quantification of immune cell function. Biological features include cell functions such as, without limitation, M1/M2 macrophage, T cell activation, T cell exhaustion, T cell fate, cell to cell interaction, and phagocytosis. Profiling of cell functions may be achieved using high content imaging of cellular organelles related to key functions. For instance, imaging may include staining of nucleus, DNA, cell membrane, cytoskeleton, chromatin, mitochondria, and/or use of brightfield imaging. Other biological features include clinical, spatial, and compositional features. Clinical features can be used to image cells post-vaccine or cells having inflammation activity and/or infected with a virus (e.g., influenza virus, SARS-CoV-2). Spatial features include cell to cell interactions and antigen presentation. Composition features include cell composition and subpopulation discovery. The biological features may be interpreted to identify functional and disease changes in the context of age. For instance, age versus cell to cell interaction, age versus phagocytosis, age versus cell activation, and/or age versus cell exhaustion. In some embodiments, images related to the biological features are used to computationally model aging along with one or more other hallmarks of aging, such as genomic instability, telomere attrition, epigenetic alterations, loss of proteostasis, deregulated nutrient-sensing, mitochondrial dysfunction, cellular senescence, stem cell exhaustion, and/or altered intercellular communication. In some embodiments, the biological features are interpreted to characterize efficacy of therapies, such as the test agents described herein.

Aging increases risk of infection that can cause severe disease and death. The cause of this increased risk is not well understood, but it is known that the immune system undergoes a multitude of changes as it ages. Some of these changes manifest themselves in the innate immune system. Dysregulation of inflammatory responses called “inflamm-aging” may disrupt the innate antiviral response by increasing the resting level of circulating proinflammatory cytokines. Changes in the adaptive immune system may impair immune function, ranging from relative reductions in naïve T cells and relative increases in memory/effector T-cell populations to alterations in signaling pathways, epigenetics, and effector functions that have all been associated with age. Collectively, these age-related alterations in the immune system are referred to as “immunosenescence.” The immune system maintains an elegant balance in its response to various events. This means that even though immunosenescence and inflamm-aging have been linked with pathology, both processes may also have beneficial effects. Maintaining a balance between the beneficial effects of these aging processes while also targeting those that are detrimental requires an ability to measure and model the complex interplay of multiple phenotypes and immune states. Such a model may be a powerful system for finding potential beneficial therapies in older adults.

In some embodiments, the methods described herein are utilized to study age-related immune diseases and conditions. These diseases and conditions include reduced vaccine response in the elderly, IPF, COPD, cardiovascular disease, and atherosclerosis. The overall aging process leads to a decreased ability to respond to stressors and maintain homeostasis. Commonly, murine models of aging are utilized to investigate age-related changes in the immune system and the C57BL/6 mouse is considered to be one of the most studied animal models. Indeed, this mouse shares many important processes with the human immune system and in aging biology. During the biological process of aging many immune system components are impacted. Age-related DNA damage in the bone marrow is associated with increased release of immature cells into the blood and a bias for differentiation toward myeloid lineage cells over lymphoid lineage cells from hematopoietic stem cells. The innate immune system (specifically neutrophils, macrophages, and dendritic cells) exhibits decreased migration and chemotaxis, as well as decreased phagocytosis and changes in population frequencies in both aging humans and mice. Similarly, the adaptive immune system exhibits decreased function with aging. In both mice and humans, thymic involution and dysfunction lead to decreased naïve T cell output with aging. In addition, T cell function and memory T cell generation are negatively impacted by aging and antibody quality is also reduced. In summary, the aged immune systems of both humans and mice exhibit delayed and reduced responsiveness with regards to both innate and adaptive aspects resulting in an overall poor response to an infectious challenge.

An exemplary method comprises imaging cells that exhibit age related immune phenotypes and analyzing the images. Immune cells may be imaged to identify changes in the immune cells of the composition of the immune cells. Immune cells may be imaged to identify hallmarks of inflammation, which include changes in chemical regulators of initiation, maintenance, and cessation of immune response resulting in low grade inflammation, as well as levels of blood inflammatory markers. The microenvironment of the immune cells may be analyzed. For instance, to evaluate changes in where the immune cells live, such as in lymphoid and nonlymphoid tissues. With aging, there are alterations in lymph node structure that affects T cell and B responses and native cell pool. Lymph node size also decreases with age and older lymph nodes tend to have fibrotic changes. Non-limiting examples of innate immune cells that may be imaged and analyzed using the methods described herein include neutrophils, monocytes, macrophages, and dendritic cells (e.g., monocyte-derived dendritic cells and myeloid/plasmacytoid dendritic cells). Effects of aging on neutrophils include decreased chemotaxis, phagocytosis; decreased TLR1-induced action; and decreased NET formation. Effects of aging on monocytes include decreased TLR-induced pro-inflammatory cytokines; decreased TLR-induced co-stimulation; decreased TLR1, 4 expression; increased TLR5 expression, Flg-conj vaccination; increased STAT3 phosphorylation; and increased basal IL-10. Effects of aging on macrophages include decreased DC-SIGN signaling; increased STAT1 phosphorylation, and increased TLR3 expression. Effects of aging on monocyte-derived dendritic cells include increased LPS, ssRNA-induced cytokines; decreased WNV-induced type I IFN; decreased phagocytosis, migration; and decreased PI3-K activity. Effects of aging on myeloid/plasmacytoid dendritic cells include increased basal cytokine levels, decreased TLR-induced cytokines, and decreased TLR expression.

In some embodiments, a method for identification of a cell's state, function, and/or predicted age is performed to improve vaccine response, for instance, in the elderly. In some cases, elderly subjects include subjects about 65 years or older. In some cases, elderly subjects are characterized as elderly based on phenotypic characterizations of cells, e.g., PBMCs.

The tremendous burden of infection on older adults is not limited to times of pandemic. Influenza is consistently problematic in older adults with increased risk for serious complications and hospitalization. In addition, approximately 90% of flu-related deaths occur in this population, with influenza and pneumonia being the eighth leading cause of death among persons over 65 years of age in the United States. Even when death is avoided, older adults have an increased risk for secondary complications and morbidities from flu infection. Depending on how successful the WHO predicts the influenza strains causing seasonal epidemics, the produced vaccines show efficacy rates between 60% and 90%. However, vaccine effectiveness in adults aged 65 and older is usually significantly lower, ranging from an average of 28% protection against fatal and nonfatal complications (with large dispersion), 39% protection against typical influenza-like illness, and 49% protection against disease with confirmed virus infection. Influenza vaccine effectiveness is a significant problem in elderly as compared to young individuals and is associated with high rates of complicated illness including pneumonia, heart attacks, and strokes in the >65-year-old population. A variety of phenotypes of elderly cells are implicated in elderly vaccine response biology. In some embodiments, one or more of these phenotypes of a subject are imaged and analyzed using a method described herein. Non-limiting phenotypes of monocytes and/or dendritic cells include dysregulated monocyte/macrophage production of IL-10 (predicts poor antibody response and impaired vaccine responses in older adults), intracellular production of TNF-α and IL-6 following vaccination (diminished substantially in classic and CD14+CD16+ monocyte populations from older adults when compared with young adults), release of IFNs and pro-inflammatory cytokines (lower capacity of plasmacytoid dendritic cells (pDCs) from elderly subjects to release IFNs and pro-inflammatory cytokines has been associated with a reduced response to influenza vaccine), changes in TLR-induced expression of costimulatory molecules in monocytes and of proinflammatory cytokines in primary myeloid dendritic cells (mDCs) and pDCs (associated with impaired influenza vaccine antibody response), basal cytokine production (substantial elevations in basal cytokine production were found in mDCs and pDCs, implying that such inflammatory dysregulation may contribute to impaired responses to newly encountered antigens), tissue context, cellular activation state (age-associated increases in TLR-induced cytokine production have been found in monocyte-derived DCs obtained following treatment with IL-4 plus GM-CSF), thymic activity, and dendritic cell numbers (low thymic activity and dendritic cell numbers have been associated with immune response to primary viral infection in the elderly). Non-limiting phenotypes of T cells include IL-6 production (aged CD4 T cells require high levels of IL-6 from antigen presenting dendritic cells for response); IFNγ:IL-10 ratios, levels of the cytolytic mediator granzyme B (in influenza-challenged PBMCs, correlates with increased risk of influenza in vaccinated older adults), and cognate helper function of naïve CDR(+) T cells (cognate age-related reductions in humoral responses due to defects in the cognate helper function of naïve CD4(+) T cells from aged individuals). Non-limiting phenotypes of B cells include, serum levels of TNF-α (the ability to generate a vaccine-specific antibody response is negatively correlated with levels of serum TNF-α, unstimulated B cells from elderly make higher levels of TNF-α than those from young individuals); cytomegalovirus (CMV)-serology (CMV-seropositivity has been shown to have a negative effect on influenza vaccine-specific antibody responses); expression of activation-induced cytidine deaminase (AID); (AID is a measure of optimal B-cell responses and its decreased expression in B cells from elderly individuals has been shown to lead to a reduced ability to generate higher affinity protective antibodies); and antibody repertoire (Influenza and pneumococcal vaccine-induced expansion of B cells with short and hydrophilic IgH CDR3 regions is lower in older individuals; impaired anti-pneumococcal IgM and IgA responses correlates with the spectratypes for their IgM- and IgA-expressing B cells; elderly adults have less de novo somatic hypermutations in immunoglobulin variable genes; elderly individuals have less adaptability in their antibody responses to influenza).

Additionally, between 2009 and 2014, adults 260 years of age accounted for 70% of the sepsis cases reported in the United States. The mortality rate for sepsis was 16% for patients 60-79 years and 18% for patients >80 years of age. In another study, patients >80 years of age with sepsis had an in-hospital mortality rate of 47%. Therefore, the aging population is devasted by infectious disease at all times, not just during a pandemic.

In some embodiments, methods and systems are provided that characterize the immune system of an elderly subject, which may be compared to the immune system of a young subject. In some embodiments, an elderly or old subject is at least about 60 years old, at least about 65 years old, at least about 70 years old, or at least about 75 years old. In some embodiments, a young subject is about 40 years old or younger, about 35 years old or younger, about 30 years old or younger, about 25 years old or younger, or about 20 years old or younger. In some embodiments, a young subject is from about 1, 2, 3, 4, 5, 6, 7, 8, 9, 10, 15, 18, or 20 years old to about 40, 35, 30, 25, or 25 years old. Non-limiting exemplary methods and systems are described in the Examples, e.g., Examples 16-21.

Methods and palettes herein may be utilized in a system for eliciting the differences between cells from the young and old in the context of innate activation, monocytes to dendritic cell differentiation, and antigen uptake. Readouts from the methods can be used to teach a computational model to accurately identify characteristics that define a young and an old immune response.

In some embodiments, systems herein can distinguish and/or model differences between young and old response to viruses in immune cells, which is useful for discovering and targeting the aging mechanisms that decrease response to vaccine and viruses with age. In some embodiments, a screen is performed with tens, hundreds, thousands, or millions of therapeutics to profile therapeutics having an impact on immune response. In some embodiments, images are analyzed to determine which therapeutics make immune cells like PBMCs from elderly subjects look more like young immune cells. The elderly cells may be treated with the therapeutics in the presence of viral stimulants. Such therapeutic compounds identified are potentially clinically relevant for use in the elderly population.

The increased risk of hospitalization or death due to infection in older adults has been exemplified by the pandemic of coronavirus disease 2019 (COVID-19) caused by severe acute respiratory syndrome coronavirus 2 (SARS-CoV-2) infection. The disease has disproportionally affected older adults by increasing morbidity and mortality. A study of patients hospitalized with SARS-CoV-2 infection demonstrated that age at admission was associated with a greater mortality risk than other factors such as obesity and chronic cardiac disease (age 70-79 years [HR, 9.6]; age >80 years [HR, 13.6]; obesity [HR, 1.4]; chronic cardiac disease [HR, 1.3]). In China, the case fatality rate was 0.4% for patients age 40-49 years and 15% for patients >80 years of age: in Italy, 96% of COVID-19 deaths occurred in people >60 years of age, while only 1% of patients under the age of 50 years died.

In some embodiments, a method for identification of a cell's state, function, and/or predicted age is performed to improve vaccine response, for instance, in the elderly, where the vaccine is for SARS-CoV-2, which causes COVID-19 disease. There are several categories of repurposing and novel therapies being tested for each stage of the COVID-19 disease. Significant amount of investment is focused on developing preventative therapies which invoke mechanisms of the host's immune system to prevent the virus from gaining a foothold. The most important of these is a vaccine which gives the body an acquired immunity to the infection. Other preventative measures also include antibodies which neutralize the virus by blocking interactions with a receptor, or binding to a viral capsid in a manner that inhibits uncoating of the genome. Antivirals which disrupt various mechanisms of the virus are being developed to be deployed at multiple disease stages. Viral targets range from the RNA replication mechanisms to blocking binding of the host cell receptors which enable viral entry. In addition to this, anti-inflammatory therapies which target pro-inflammatory cytokines are being evaluated for their ability to reduce inflammation and tissue damage at the severe stage of the disease. In some embodiments, the approaches provided herein are different when compared to each of these. The systems herein appreciate the immense clinical burden that COVID-19 has on the elderly and the immune related dysfunction shown to be a central tenet of severe disease progression. The dysfunction of the elderly immune system may be one of the main drivers of COVID-19's devastating impact on this demographic, and as such, therapeutics identified using the systems and methods here target these age related immune mechanisms.

In some embodiments, a monocyte system herein is used to target key monocyte function, which is critical to normal disease progression. This system may be used for repurposing existing therapies for specific use in elderly populations. A therapy that can hasten, improve, and/or boost the initial delayed, dysfunctional response of monocytes in the elderly can potentially prevent the transition to severe/late stage disease, which for diseases like COVID-19, may drastically reduce the number of deaths. In some embodiments, provided is a monocyte system that models the dysfunctional/delayed response to coronavirus and the aggravation of that response by inherit dysfunction in the elderly innate immune system. This model system may be used to screen for safe, repurposable drugs which boost the initial innate immune response for the treatment of disease, such as COVID-19.

In some embodiments, a viral system herein models elderly response to high viral load and the adaptive immune (e.g., T-cell, NK cell, B-cell) system response to a cytokine storm produced by monocytes. In some embodiments, the model system is useful for screening safe, repurposable drugs which decrease inflammation, rejuvenate the elderly response to viruses, and/or improve the lymphopenia.

In some embodiments, systems herein can distinguish and/or model the differences between vaccine response in the young and elderly. In an example, the system may be used to screen for vaccines that induce elderly immune cells to respond more like the young immune cells. In another example, the system may be used to discover and target immune aging mechanisms causing decreased response to vaccines. Thus, the system may be useful for screening for vaccines that may be efficacious in the elderly. Any available vaccine may be screened, for instance, vaccines against influenza vaccines, SARS-CoV-2, diphtheria, tetanus, pertussis, hepatitis B, poliomyelitis, and Haemophilus influenzae type b. The vaccine system may be combined with adjuvant systems, or components thereof, described herein for improving vaccine response in the elderly.

An exemplary method for improving vaccine response using a system described herein includes imaging immune cells (e.g., PBMCs) after exposure to vaccines; segmenting T-cells and dendritic cells; quantifying innate activation, T-cell priming and T-cell activation; screening possible interventions for hits; and testing hits in a mouse model for elderly vaccine response to identify interventions for human clinical trials.

In some embodiments, systems herein can distinguish and/or model differences in immune response between different adjuvants. In some embodiments, systems herein can distinguish and/or model differences in immune response between different doses of the same adjuvant. In some embodiments, systems herein can distinguish and/or model differences in immune response between adjuvants and control (no adjuvant treatment). The model may be used to classify adjuvants. Exemplary adjuvants that can be used in these models include, without limitation, MF59, AS01, and GLA. In some embodiments, a model compares immune response to adjuvant vs. control, and predicts if a small molecule induces a phenotype that looks like any of the clinically proven adjuvants. In some embodiments, a model compares immune response to AS01 vs. MF59 vs. GLA vs. control, and predicts if a small molecules looks more like a specific adjuvant. In some embodiments, a model compares immune response to AS01 high vs. AS01 low vs. MF59 high vs. MF59 low vs. GLA high vs. GLA low vs. control, and predicts if a small molecule looks more like the high or low concentration for a specific adjuvant.

In some embodiments, provided herein is a method for improving elderly vaccine response comprising stimulating cells with a virus, such as Influenza or SARS-CoV-2, and imaging PBMCs. Cell populations are segmented into relevant populations, e.g., T cells, Th1 cells, and/or dendritic cells. Images are analyzed to measure clinically relevant biological features such as clinically relevant functions, e.g., T cell activation, cell to cell interaction, antigen presentation, and/or young versus old phenotypes. Potential interventions are screened, which may include thousands of agents, to identify functional hits and aging hits. In vivo screening of potential interventions may be performed using a mouse model of elderly vaccine response to identify interventions for use in clinical trials.

In the above aspect or in any of the above embodiments, the method further comprising identifying the state, function, and/or predicted age of an at least second cell. The method comprising: contacting an at least second in vitro cell with a first binding reagent capable of recognizing and binding a first marker of the cell and contacting the at least second cell with an at least second binding reagent capable of recognizing and binding an at least second marker of the cell; determining the at least second in vitro cell's morphological signature and/or functional signature for the at least second cell based on the intensity of a signal associated with the first binding reagent and the intensity of a signal associated with the at least second binding reagent; and identifying the at least second cell's state, function, and/or predicted age based upon the morphological signature and/or functional signature.

In some embodiments, cells and cell culture materials are used to promote cell adhesion, e.g., for cells that are not naturally adherent. In some embodiments, the cells, during the initial cell plating preparation, are kept in cell culture medium depleted from protein supplements and/or plated onto substrates that are treated to promote adhesion.

Methods for Identifying a Drug Capable of Changing a Cell's State, Function, and/or Predicted Age

Another aspect of the present disclosure provides a method identifying a drug capable of changing a cell's state, function, and/or predicted age. The method comprising: contacting a first in vitro cell with a test agent; determining a change in the first in vitro cell's state, function, and/or predicted age; and identifying the test agent as a drug capable of changing a cell's state, function, and/or predicted age based upon the change in the first in vitro cell's state, function, and/or predicted age relative to a second in vitro cell that was not contacted with the test agent. In this aspect, the change in the cell's state, function, and/or predicted age is determined by performing at least: contacting the first in vitro cell with a first binding reagent capable of recognizing and binding a first marker of the first in vitro cell and contacting the first in vitro cell with an at least second binding reagent capable of recognizing and binding an at least second marker of the first in vitro cell; determining the first in vitro cell's morphological signature and/or functional signature based on the intensity of a signal associated with the first binding reagent and the intensity of a signal associated with the at least second binding reagent; and identifying the first in vitro cell's state, function, and/or predicted age based on the morphological signature and/or functional signature.

In some embodiments, the test agent is a small molecule, biologic (e.g., an animal extract, plant extract, or cellular extract), antibody or an antigen binding fragment, and/or a nucleic acid (e.g., a gene therapy molecule), or a combination thereof.

In some embodiments, the second in vitro cell that is not contacted with the test agent may be a historical control. As used herein, a historical control is a cell having similar features as the in vitro cell but has not been treated with a test agent. A historical control cell will have been contacted with the first binding reagent capable of recognizing and binding the first marker of the cell and the at least second binding reagent capable of recognizing and binding the at least second marker of the cell and the intensities of signal associated with the first binding reigned and at least second binding reagents were measured and normalized.

In some embodiments, the first in vitro cell's morphological signature comprises the first in vitro cell's size and/or the first in vitro cell's shape and/or morphological properties (e.g., eccentricity, form factor, and solidity). In some embodiments, the first in vitro cell's functional signature comprises one or more of the presence or absence of a marker characteristic of a specific cell organelle, the presence or absence of a marker characteristic of a specific cell type, the presence or absence of a marker characteristic of a specific cell activity, the presence or absence of a marker characteristic of an active cell, the presence or absence of a marker characteristic of an inactive cell, the presence or absence of a marker indicating gene expression, and/or the ability of the in vitro cell to interact with another cell or fragment of another cell. In some embodiments, the first marker and/or the at least second marker is located on or in the first in vitro cell's nucleus, cell membrane, cytoplasm, cytoskeleton, chromatin, mitochondrion, and/or extracellular matrix. In some embodiments, the first binding reagent and/or the at least second binding reagent are each respectively directly or indirectly labeled with a first fluorescent molecule and/or an at least second fluorescent molecule. In some embodiments, the at least second marker comprises a third marker, a fourth marker, a fifth marker, a sixth marker, a seventh marker, an eighth marker, a ninth marker, a tenth marker, or more markers. In some embodiments, determining the first in vitro cell's morphological signature and/or functional signature further comprises contacting the first in vitro cell with a third binding reagent, a fourth binding reagent, a fifth binding reagent, a sixth binding reagent, a seventh binding reagent, and/or an eighth binding reagent each respectively capable of recognizing and binding the third marker, the fourth marker, the fifth marker, the sixth marker, the seventh marker, the eighth marker, the ninth eighth marker, and/or the at least tenth marker. In some embodiments, the third binding reagent, the fourth binding reagent, the fifth binding reagent, the sixth binding reagent, the seventh binding reagent, the eighth binding reagent, the ninth binding reagent, and/or the at least tenth binding reagent are each respectively directly or indirectly labeled with a third fluorescent molecule, a fourth fluorescent molecule, a fifth fluorescent molecule, a sixth fluorescent molecule, a seventh fluorescent molecule, an eighth fluorescent molecule, a ninth fluorescent molecule and/or an at least tenth fluorescent molecule.

In some embodiments, the intensity of a signal associated with the first binding reagent and the intensity of a signal associated with the at least second binding reagent comprises visualization of the first in vitro cell or a portion thereof by light microscopy, e.g., fluorescent microscopy. In some embodiments, the intensity of a signal associated with the first binding reagent and the intensity of a signal associated with the at least second binding reagent are produced by each of the first fluorescent molecule and/or the at least second fluorescent molecule. In some embodiments, determining the first in vitro cell's morphological signature and/or functional signature further comprises normalizing the signal intensity produced by each of the first fluorescent molecule and/or the at least second fluorescent molecule. In some embodiments, the first in vitro cell's morphological signature and/or functional signature is calculated based upon the normalized signal intensity produced by each of the first fluorescent molecule and/or the at least second fluorescent molecule.

In some embodiments, the first in vitro cell's predicted age is determined by a machine learning technique selected from convolutional neural networks and gradient boosted trees to compute the first in vitro cell's age (e.g., as a continuous value between 0 and 100). For example, the machine learning technique may comprise a supervised machine learning algorithm or supervised machine learning classifier configured to analyze a morphological signature, a functional signature, a marker, etc. of an in vitro cell. In some embodiments, an age of the first in vitro cell that is over 65 indicates that the cell is dying, old, and/or senescent. In some embodiments, an age of the first in vitro cell's that is under 30 indicates that the cell is young, healthy, and/or active.

In some embodiments, the marker indicating gene expression is methylated DNA.

In some embodiments, the marker characteristic of a specific cell type is differentially expressed by a T cell (e.g., memory T cell or an effector T cell), a B cell, an Natural Killer (NK) cell, a monocyte/macrophage (e.g., an MI macrophage or by an M2 macrophage), and/or a monocyte/dendritic cell.

In some embodiments, the first in vitro cell's morphological signature and/or functional signature comprises characterizing the first in vitro cell's mitochondrial shape using one or more of spatial pattern analysis, texture analysis, intensity changes, and convolutional neural networks.

In some embodiments, the first in vitro cell's size and/or the first in vitro cell's shape and/or morphological properties (e.g., eccentricity, form factor, and solidity) are quantified using convolutional neural networks.

In some embodiments, the marker characteristic of an active cell identifies T cell activation.

In some embodiments, the marker characteristic of an inactive cell identifies T cell exhaustion.

In some embodiments, the marker characteristic of a specific cell type is differentially expressed by an M1 macrophage and by an M2 macrophage. In some embodiments, the marker characteristic of an M1 macrophage is Calprotectin and/or the marker characteristic of an M2 macrophage is Mannose Receptor. In some embodiments, the marker is identified by an antigen binding domain that recognizes Calprotectin or Mannose Receptor.

In some embodiments, the first in vitro cell's ability to interact with another cell or fragment of another cell is quantified using a spatial pattern analysis method.

In some embodiments, the first in vitro cell's morphological signature and/or functional signature comprises a characterization based upon the presence or absence of cell motility, genomic instability, telomere attrition, epigenetic alterations, loss of proteostasis, deregulated nutrient-sensing, mitochondrial dysfunction, cellular senescence, stem cell exhaustion, and/or altered intercellular communication.

In some embodiments, the first binding reagent and/or the at least second binding reagent is a lectin. In some embodiments, the lectin is Wheat Germ Agglutinin (WGA) or Concanavalin (ConA).

In some embodiments, the first binding reagent and/or the at least second binding reagent comprises an antigen binding domain. In some embodiments, the binding reagent that comprises an antigen binding domain is an antibody. In some embodiments, the binding reagent that comprises an antigen binding domain recognizes total Histone H3, Acetyl-Histone H3, or Tri-Methyl-Histone H3. In some embodiments, the binding reagent that comprises an antigen binding domain recognizes CD3, CD11c, CD14, CD16, CD19, CD45, CD56, or CD68.

In some embodiments, the first binding reagent and/or the at least second binding reagent is a nuclear stain, e.g., Hoechst 33342, or a mitochondrial stain, e.g., a MitoTracker®.

In some embodiments, the first binding reagent and/or the at least second binding reagent is Phalloidin.

In some embodiments, the first binding reagent and/or the at least second binding reagent is capable of determining if a cell is alive or dead (e.g., one or more of DAPI, propidium iodide (PI), and a Zombie dye).

In some embodiments, the first binding reagent, the second binding reagent, the third binding reagent, the fourth binding reagent, and/or the fifth binding reagent each comprises one of Hoechst 33342, Wheat Germ Agglutinin (WGA); Phalloidin; Concanavalin (ConA); and/or a binding reagent capable of determining if a cell is alive or dead (e.g., one or more of DAPI, propidium iodide (PI), and a Zombie dye). In some embodiments, the method further comprises determine the first in vitro cell's morphological signature using brightfield microscopy. The combination of these five binding reagents along with brightfield imaging may be referred to herein as the CellPainting Membrane (CPM) palette.

In some embodiments, the first binding reagent, the second binding reagent, the third binding reagent, the fourth binding reagent, and/or the fifth binding reagent each comprises one of Hoechst 33342; an antigen binding domain that recognizes Acetyl-Histone H3; an antigen binding domain that recognizes Tri-Methyl-Histone H3; MitoTracker® Orange; and/or a binding reagent capable of determining if a cell is alive or dead (e.g., one or more of DAPI, propidium iodide (PI), and a Zombie dye). In some embodiments, the method further comprises determine the in vitro cell's morphological signature using brightfield microscopy. The combination of these five binding reagents along with brightfield imaging may be referred to herein as the CellPainting Epigenetic (CPE) palette.

In some embodiments, the first binding reagent, the second binding reagent, the third binding reagent, the fourth binding reagent, the fifth binding reagent, and/or the sixth binding reagent each comprises one of Hoechst 33342; an antigen binding domain that recognizes CD3; an antigen binding domain that recognizes CD19; an antigen binding domain that recognizes CD14; an antigen binding domain that recognizes CD11c; and/or an antigen binding domain that recognizes CD56. In some embodiments, the method further comprises determining the first in vitro cell's morphological signature using brightfield microscopy. The combination of these six binding reagents along with brightfield imaging may be referred to herein as the Cell Specific (CPS) palette.

In some embodiments, the first binding reagent, the second binding reagent, the third binding reagent, the fourth binding reagent, and/or the fifth binding reagent each comprises one of Hoechst 33342; an antigen binding domain that recognizes CD3; Phalloidin; Concanavalin (ConA); and/or an antigen binding domain that recognizes CD11c. In some embodiments, the method further comprises determining the first in vitro cell's morphological signature using brightfield microscopy. The combination of these five binding reagents along with brightfield imaging may be referred to herein as the CellPainting Membrane+Cell Specific (CPMS) palette.

In some embodiments, the first binding reagent, the second binding reagent, the third binding reagent, the fourth binding reagent, and/or the fifth binding reagent each comprises one of Hoechst 33342; WGA; MitoTracker® Orange; Concanavalin (ConA); and/or Phalloidin. In some embodiments, the method further comprises determining the first in vitro cell's morphological signature using brightfield microscopy. The combination of these five binding reagents along with brightfield imaging may be referred to herein as the CPMM palette.

In some embodiments, the first in vitro cell's morphological signature and/or functional signature is determined by measurements of the cell membrane, the cell's epigenetic signature, the cell's mitochondrial signature, and cell specific protein markers.

In some embodiments, the method further comprises contacting the first in vitro cell with an agent known to affect a cell's morphological signature and/or functional signature. In some embodiments, the agent known to affect a cell's morphological signature and/or functional signature comprises one or more of Vesicular Stomatitis virus (replication deficient VSV); lipopolysaccharide (LPS); Phytohaemagglutinin (PHA); ConcanavalinA (ConA); Pokeweed Mitogen (PWM); Carbonyl cyanide 4-(trifluoromethoxy)phenylhydrazone (FCCP); phorbol myristate acetate (PMA) with Ionomycin; trichostatin A (TSA); C646; 2-Hydroxyglutarate; Colchicine; Cytochalasine A; Jasplakinolide; Paclitaxol (Taxol); Phalloidin; and/or a receptor/antigen blocking antibody; e.g., an anti-MHC antibody.

In some embodiments, the first in vitro cell is a peripheral blood mononuclear cell (PBMC).

In some embodiments, identifying the first in vitro cell's state, function, and/or predicted age further predicts T cell activation, T cell fate, and T cell exhaustion, wherein the identifying comprises use of a supervised machine learning algorithm.

In some embodiments, identifying the first in vitro cell's state, function, and/or predicted age further comprises identifying therapeutically-relevant subsets of immune cells. In some embodiments, the identification of therapeutically-relevant subsets of immune cells comprises use of an unsupervised machine learning algorithm selected from t-SNE, Autoencoder, principal component analysis, and/or k-means clustering.

In some embodiments, identifying the first in vitro cell's state, function, and/or predicted age further comprises predicting cytokine and chemokine release by a PBMC. In some embodiments, predicting the cytokine and chemokine release by a PBMC comprises use of a supervised machine learning algorithm.

Another aspect of the present disclosure provides a drug identified by a method for identifying a drug capable of changing a cell's state, function, and/or predicted age.

Another aspect of the present disclosure provides a pharmaceutical composition for treating a disease or disorder associated with aging comprising a therapeutically-effective amount of the drug.

Another aspect of the present disclosure provides a high throughput method for identifying a drug capable of changing a cell's state, function, and/or predicted age, comprising: providing a sample of cells into each of a plurality of wells in a multi-well plate; contacting cells in a first subset of cells in the multi-well plate with a first test agent; determining a change in state, function, and/or predicted age of cells in the first subset of cells; and identifying the test agent as a drug capable of changing a cell's state, function, and/or predicted age based on the change in state, function, and/or predicted age of cells in the first subset relative to cells in a second subset of cells in the multi-well plate that were not contacted with the test agent. In this aspect, the change in state, function, and/or predicted age is determined by performing at least: contacting cells in the first subset with a first binding reagent capable of recognizing and binding a first marker and contacting cells in the first subset with an at least second binding reagent capable of recognizing and binding an at least second marker; determining the morphological signature and/or functional signature of cells in the first subset based on the intensity of a signal associated with the first binding reagent and the intensity of a signal associated with the at least second binding reagent; and identifying the state, function, and/or predicted age of cells in first subset based upon the morphological signature and/or functional signature.

In some embodiments, the cells in the second subset were contacted with a control and/or cells in the second subset were contacted with a second test agent. In some embodiments, the first test agent is a small molecule, biologic (e.g., an animal extract, plant extract, or cellular extract), antibody or an antigen binding fragment, and/or a nucleic acid (e.g., a gene therapy molecule), or a combination thereof and/or the second test agent is a small molecule, biologic (including an animal extract, plant extract, or a cellular extract), antibody or an antigen binding fragment, a nucleic acid (e.g., a gene therapy molecule), or a combination thereof.

High throughput methods of the present disclosure are facilitated using commercially-available automated systems for manipulating and imaging a multi-well plate.

In the high throughput methods, any number of samples may be multiplexed. For example, a multiplexed analysis may contain samples from 2, 3, 4, 5, 6, 7, 8, 9, 10, 11, 12, 13, 14, 15, 16, 17, 18, 19, about 20, about 25, about 30, about 35, about 40, about 45, about 50, about 55, about 60, about 65, about 70, about 75, about 80, about 85, about 90, about 95, about 100, or more than 100 initial samples. The different samples may be segregated to different areas (e.g., wells) of a solid support.

In some embodiments, cells and cell culture materials are used to promote cell adhesion, e.g., for cells that are not naturally adherent. In some embodiments, the cells, during the initial cell plating preparation, are kept in cell culture medium depleted from protein supplements and/or plated onto substrates that are treated to promote adhesion.

Another aspect of the present disclosure provides a drug identified by a high throughput method for identifying a drug capable of changing a cell's state, function, and/or predicted age.

Another aspect of the present disclosure provides a pharmaceutical composition for treating a disease or disorder associated with aging comprising a therapeutically-effective amount of the drug.

In some embodiments, provided herein is a system for producing a multi-phenotype aging profile to measure immunosenescence. Older adults have immune dysregulation that leads to increased infection, illness, and death, for reasons that are not well understood. In some embodiments, the system combines the power of high-throughput robotics laboratory and machine learning algorithms to build a multi-phenotype aging profile that models the dysfunctional response to viral infection in older adults. In an exemplary embodiment, the system utilizes machine learning to measure one or more signatures of immunosenescence. Signatures of immunosenescence may include one or more of: compositional (measure changes in cell composition), cell-to-cell interaction (capture the physical interaction between cells), organelle structure (profile the change in cellular morphology), cytokines (measure intercellular signaling), and hidden complexities (unbiased machine learning of complex, non-obvious aging phenotypes). A system that measured signatures of immunosenescence across these five categories was used to test thousands of therapies for their ability to reverse the observed defects of the aging immune system. The top therapies are subject to further investigation, with the goal of translation into clinical practice. A non-limiting exemplary system is described in Examples 27 to 31.

### Test Agents

Aspects of the present disclosure include methods and systems for identifying a drug capable of changing a cell's state, function, and/or predicted age. The methods comprise: contacting a first in vitro cell or a first subset of cells with a test agent; determining a change in the first in vitro cell's or the first subset of cell's state, function, and/or predicted age; and identifying the test agent as a drug capable of changing a cell's state, function, and/or predicted age based upon the change in the first in vitro cell's or the first subset of cell's state, function, and/or predicted age relative to a second in vitro cell or second subset of cells that was not contacted with the test agent.

In some embodiments, the test agent is a small molecule, biologic (including an animal extract, plant extract, or cellular extract), antibody or an antigen binding fragment, and/or a nucleic acid (e.g., a gene therapy molecule), or a combination thereof.

In some embodiments, the test agent is a small molecule. Non-limiting examples of small molecules that may be used in methods and compositions of the present disclosure include Abatacept, Abiraterone, Acetaminophen/hydrocodone, Amphetamine mixed salts, Aripiprazole, Atazanavir, Atorvastatin, Avonex (REBIF), Budesonide, Budesonide/formoterol, Buprenorphine, Capecitabine, Celecoxib, Ciclosporin ophthalmic emulsion, Cinacalcet, Dabigatran, Darbepoetin alfa, Darunavir, Dexlansoprazole, Doxycycline, Duloxetine, Elvitegravir/cobicistat/emtricitabine/tenofovir, Emtricitabine/Rilpivirine/Tenofovir disoproxil fumarate, Emtricitabine/tenofovir/efavirenz, Enoxaparin, Epoetin alfa, Esomeprazole, Eszopiclone, Etanercept (ENBREL), Everolimus, Ezetimibe, Ezetimibe/simvastatin, Fenofibrate, Filgrastim, fingolimod, Fluticasone propionate, Fluticasone/salmeterol, Glatiramer, Imatinib, Insulin aspart, Insulin detemir, Insulin glargine (LANTUS), Insulin lispro, Ipratropium bromide/salbutamol, Ledipasvir/sofosbuvir, Lenalidomide, Levothyroxine, Lidocaine, Liraglutide, Lisdexamfetamine, Memantine, Methylphenidate, Metoprolol, Mometasone, Olmesartan, Oseltamivir (TAMIFLU), Oxycodone, Pegfilgrastim (NEULASTA), Pemetrexed, Pneumococcal conjugate vaccine, Pregabalin, Quetiapine, Rabeprazole, Raloxifene, Raltegravir, Rivaroxaban, Rosuvastatin, Salbutamol, Sevelamer, Sildenafil, Sitagliptin/metformin, Sofosbuvir, Solifenacin, Tadalafil, Telaprevir, Tenofovir/emtricitabine, Testosterone gel, Tiotropium bromide, Valproate, Valsartan, and Zostavax.

In some embodiments, the test agent is a biologic. A biologic is a therapeutic product that is produced from living organisms or contain components of living organisms. Non-limiting examples of biologics that may be used in methods and compositions of the present disclosure include abatacept (Orencia), abobotulinumtoxinA (Dysport), aflibercept (Eylea), agalsidase beta (Fabrazyme), albiglutide (Tanzeum), aldesleukin (Proleukin), alglucosidase alfa (Myozyme, Lumizyme), alteplase (Cathflo Activase, Activase), anakinra (Kineret), asfotase alfa (Strensiq), asparaginase (Elspar), asparaginase Erwinia chrysanthemi (Erwinaze), becaplermin (Regranex), belatacept (Nulojix), collagenase (Santyl), collagenase Clostridium histolyticum (Xiaflex), dulaglutide (Trulicity), ecallantide (Kalbitor), elosulfase alfa (Vimizim), epoetin alfa (Epogen/Procrit), etanercept (Enbrel), etanercept-szzs (Ereizi), follitropin alpha (Gonal f), galsulfase (Naglazyme), glucarpidase (Voraxaze), iaronidase (Aldurazyme), idursulfase (Elaprase), incobotulinumtoxinA (Xeomin), interferon alfa-2b (Intron A), interferon alfa-n3 (Alferon N Injection), interferon beta-1a (Avonex), interferon beta-1a (Rebif), interferon beta-1b (Betaseron), interferon beta-1b (Extavia), interferon gamma-1b (Actimmune), methoxy polyethylene glycol-epoetin beta (Mircera), metreleptin (Myalept), ocriplasmin (Jetrea), onabotulinumtoxinA (Botox), oprelvekin (Neumega), palifermin (Kepivance), parathyroid hormone (Natpara), pegaptanib (Macugen), pegaspargase (Oncaspar), pegfilgrastim (Neulasta), peginterferon alfa-2a (Pegasys), peginterferon alfa-2b (PegIntron, Sylatron), peginterferon beta-1a (Plegridy), pegloticase (Krystexxa), rasburicase (Elitek), reteplase (Retavase), Rilonacept (Arcalyst), rimabotulinumtoxinB (Myobloc), romiplostim (Nplate), sargramostim (Leukine), sebelipase alfa (Kanuma), tenecteplase (TNKase), and ziv-aflibercept (Zaltrap).

In some embodiments, the biologic is an animal extract.

In some embodiments, the biologic is a plant extract.

In some embodiments, the biologic is a cellular extract.

In some embodiments, the test agent is an antibody or an antigen binding fragment thereof. Non-limiting examples of antibodies (or an antigen binding fragment thereof) that may be used in methods and compositions of the present disclosure include 3f8, 8h9, Abagovomab, Abciximab (REOPRO), Abituzumab, Abrezekimab, Abrilumab, Actoxumab, Adalimumab (HUMIRA amjevita), Adecatumumab, Ado-Trastuzumab Emtansine, Ado-Trastuzumab Emtansine (KADCYLA), Aducanumab, Afasevikumab, Afelimomab, Alacizumab pegol, Alefacept (AMEVIVE), Alemtuzumab, Alemtuzumab (CAMPATH), Alirocumab (PRALUENT), Alpelisib (PIQRAY), Altumomab pentetate, Amatuximab, Anatumomab mafenatox, Andecaliximab, Anetumab ravtansine, Anifrolumab, Anrukinzumab (IMA-638), Apolizumab, Aprutumab ixadotin, Arcitumomab, Ascrinvacumab, Aselizumab, Atezolizumab (TECENTRIQ), Atidortoxumab, Atinumab, Atorolimumab, Avelumab (BAVENCIO), Axicabtagene Ciloleucel (YESCARTA), Azintuxizumab vedotin, Bapineuzumab, Basiliximab (SIMULECT), Bavituximab, Bcd-100, Bectumomab, Begelomab, Belantamab mafodotin, Belimumab (BENLYSTA), Bemarituzumab, Benalizumab, Bedimatoxumab, Bermekimab, Bersanlimab, Bertilimumab, Besilesomab, Bevacizumab (AVASTIN), Bezlotoxumab (ZINPLAVA), Biciromab, Bimagrumab, Bimekizumab, Birtamimab, Bivatuzumab mertansine, Bleselumab, Blinatumomab (BLINCYTO), Blontuvetmab, Blosozumab, Bococizumab, Brazikumab, Brentuximab Vedotin (ADCETRIS), Briakinumab, Brodalumab (SILIQ), Brolucizumab, Brontictuzumab, Burosumab, Cabiralizumab, Camidanlumab tesirine, Camrelizumab, Canakinumab (ILARIS), Cantuzumab mertansine, Cantuzumab ravtansine, Caplacizumab, Caplacizumab-yhdp (CABLIVI), Capromab pendetide, Carlumab, Carotuximab, Catumaxomab, Cbr96-doxorubicin immunoconjugate, Cedelizumab, Cemiplimab, Cemiplimab-rwlc (LIBTAYO), Cergutuzumab amunaleukin, Certolizumab pegol (CIMZIA), Cetrelimab, Cetuximab (ERBITUX), Cibisatamab, Cirmtuzumab, Citatuzumab bogatox, Cixutumumab, Claudiximab, Clazakizumab, Clenoliximab, Clivatuzumab tetraxetan, Codrituzumab, Cofetuzumab pelidotin, Coltuximab ravtansine, Conatumumab, Concizumab, Cosfroviximab, Cr6261, Crenezumab, Crizanlizumab, Crotedumab, Cusatuzumab, Dacetuzumab, Daclizumab (ZINBRYTA, ZENAPAX), Dalotuzumab, Dapirolizumab pegol, Daratumumab (DARZALEX), Dectrekumab, Demcizumab, Denintuzumab mafodotin, Denosumab (PROLIA, XGEVA), Depatuxizumab mafodotin, Derlotuximab biotin, Detumomab, Dezamizumab, Dinutuximab (UNITUXIN), Diridavumab, Domagrozumab, Dorlimomab aritox, Dostarlimab, Drozitumab, Ds-8201, Duligotuzumab, Dupilumab, Durvalumab (IMFINZI), Dusigitumab, Duvortuxizumab, Ecromeximab, Eculizumab (SOLIRIS), Edobacomab, Edrecolomab, Efalizumab (RAPTIVA), Efungumab, Eldelumab, Elezanumab, Elgemtumab, Elotuzumab (EMPLICITI), Elsilimomab, Emactuzumab, Emapalumab-lzsg (GAMIFANT), Emibetuzumab, Emicizumab, Enapotamab vedotin, Enavatuzumab, Enfortumab vedotin, Enlimomab pegol, Enoblituzumab, Enokizumab, Enoticumab, Ensituximab, Epitumomab cituxetan, Epratuzumab, Eptinezumab, Erenumab, Erlizumab, Ertumaxomab, Etaracizumab, Etigilimab, Etrolizumab, Evinacumab, Evolocumab (REPATHA), Exbivirumab, Fanolesomab, Faralimomab, Faricimab, Farletuzumab, Fasinumab, Fbta05, Felvizumab, Fezakinumab, Fibatuzumab, Ficlatuzumab, Figitumumab, Firivumab, Flanvotumab, Fletikumab, Flotetuzumab, Folfiri-Bevacizumab, Folfiri-Cetuximab, Fontolizumab, Foralumab, Foravirumab, Fremanezumab, Fresolimumab, Frovocimab, Frunevetmab, Fulranumab, Futuximab, Galcanezumab, Galiximab, Gancotamab, Ganitumab, Gantenerumab, Gatipotuzumab, Gavilimomab, Gedivumab, Gemtuzumab Ozogamicin (MYLOTARG), Gevokizumab, Gilvetmab, Gimsilumab, Girentuximab, Glembatumumab vedotin, Golimumab (SIMPONI, SIMPONI ARIA), Gomiliximab, Gosuranemab, Guselkumab, Ianalumab, Ibalizumab, Ibi308, lbritumomab Tiuxetan (ZEVALIN), Icrucumab, Idarucizumab (PRAXBIND), Ifabotuzumab, Igovomab, ladatuzumab vedotin, Imalumab, Imaprelimab, Imciromab, Imgatuzumab, Inclacumab, Indatuximab ravtansine, Indusatumab vedotin, Inebilizumab, Inflectra (REMICADE), Infliximab (REMICADE), Infliximab-dyyb (INFLECTRA), Inolimomab, Inotuzumab Ozogamicin (BESPONSA), Intetumumab, Iomab-b, Ipilimumab (YERVOY), Iratumumab, Isatuximab, Iscalimab, Istiratumab, Itolizumab, Lxekizumab (TALTZ), Keliximab, Labetuzumab, Lacnotuzumab, Ladiratuzumab vedotin, Lampalizumab, Lanadelumab, Landogrozumab, Laprituximab emtansine, Larcaviximab, Lebrikizumab, Lemalesomab, Lendalizumab, Lenvervimab, Lenzilumab, Lerdelimumab, Leronlimab, Lesofavumab, Letolizumab, Lexatumumab, Libivirumab, Lifastuzumab vedotin, Ligelizumab, Lilotomab satetraxetan, Lintuzumab, Lirilumab, Lodelcizumab, Lokivetmab, Loncastuximab tesirine, Lorvotuzumab mertansine, Losatuxizumab vedotin, Lucatumumab, Lulizumab pegol, Lumiliximab, Lumretuzumab, Lupartumab amadotin, Lutikizumab, Mapatumumab, Margetuximab, Marstacimab, Maslimomab, Matuzumab, Mavrilimumab, Mepolizumab (NUCALA), Metelimumab, Milatuzumab, Minretumomab, Mirikizumab, Mirvetuximab soravtansine, Mitumomab, Modotuximab, Mogamulizumab, Mogamulizumab-kpkc (POTELIGEO), Monalizumab, Morolimumab, Mosunetuzumab, Motavizumab, Moxetumomab pasudotox, Moxetumomab Pasudotox-tdfk (LUMOXITI), Muromonab-cd3, Nacolomab tafenatox, Namilumab, Naptumomab estafenatox, Naratuximab emtansine, Narnatumab, Natalizumab (TYSABRI), Navicixizumab, Navivumab, Naxitamab, Nebacumab, Necitumumab (PORTRAZZA), Nemolizumab, Neod001, Nerelimomab, Nesvacumab, Netakimab, Nimotuzumab, Nirsevimab, Nivolumab, Nivolumab (OPDIVO), Nofetumomab merpentan, Obiltoxaximab (ANTHIM), Obinutuzumab (GAZYVA), Ocaratuzumab, Ocrelizumab, Odulimomab, Ofatumumab (ARZERRA), Olaratumab (LARTRUVO), Oleclumab, Olendalizumab, Olokizumab, Omalizumab (XOLAIR), Omburtamab, Oms721, Onartuzumab, Ontuxizumab, Onvatilimab, Opdivo (NIVOLUMAB), Opicinumab, Oportuzumab monatox, Oregovomab, Orticumab, Otelixizumab, Otilimab, Otlertuzumab, Oxelumab, Ozanezumab, Ozoralizumab, Pagibaximab, Palivizumab (SYNAGIS), Pamrevlumab, Panitumumab (VECTIBIX), Pankomab, Panobacumab, Parsatuzumab, Pascolizumab, Pasotuxizumab, Pateclizumab, Patritumab, Pdr001, PEG-Intron (Peginterferon Alfa-2b), Pembrolizumab (KEYTRUDA), Pemetrexed Disodium, Pemtumomab, Perakizumab, Pertuzumab (PERJETA), Pexelizumab, Pidilizumab, Pinatuzumab vedotin, Pintumomab, Placulumab, Plerixafor, Plozalizumab, Pogalizumab, Polatuzumab vedotin, Polatuzumab Vedotin-piiq (POLIVY), Ponezumab, Porgaviximab, Prasinezumab, Prezalizumab, Priliximab, Pritoxaximab, Pritumumab, Pro 140, Quilizumab, Racotumomab, Radretumab, Rafivirumab, Ralpancizumab, Ramucirumab, Ramucirumab (CYRAMZA), Ranevetmab, Ranibizumab (LUCENTIS), Ravagalimab, Ravulizumab, Ravulizumab-cwvz (ULTOMIRIS), Raxibacumab, Refanezumab, Regavirumab, Regn-eb3, Relatlimab, Remtolumab, Reslizumab (CINQAIR), Rilotumumab, Rinucumab, Risankizumab, Rituximab (RITUXAN), Rituximab (TRUXIMA), Rituximab and Hyaluronidase Human (RITUXAN HYCELA), Rivabazumab pegol, Rmab, Robatumumab, Roledumab, Romilkimab, Romosozumab, Rontalizumab, Rosmantuzumab, Rovalpituzumab tesirine, Rovelizumab, Rozanolixizumab, Ruplizumab, Sa237, Sacituzumab govitecan, Samalizumab, Samrotamab vedotin, Sarilumab, Satralizumab, Satumomab pendetide, Secukinumab (COSENTYX), Selicrelumab, Seribantumab, Setoxaximab, Setrusumab, Sevirumab, Sgn-cdl9a, Shp647, Sibrotuzumab, Sifalimumab, Siltuximab (SYLVANT), Simtuzumab, Siplizumab, Sirtratumab vedotin, Sirukumab, Sofituzumab vedotin, Solanezumab, Solitomab, Sonepcizumab, Sontuzumab, Spartalizumab, Stamulumab, Sulesomab, Suptavumab, Sutimlimab, Suvizumab, Suvratoxumab, Tabalumab, Tacatuzumab tetraxetan, Tadocizumab, Talacotuzumab, Talizumab, Tamtuvetmab, Tanezumab, Taplitumomab paptox, Tarextumab, Tavolimab, Tefibazumab, Telimomab aritox, Telisotuzumab vedotin, Tenatumomab, Teneliximab, Teplizumab, Tepoditamab, Teprotumumab, Tesidolumab, Tetulomab, Tezepelumab, Tgn1412, Tibulizumab, Tigatuzumab, Tildrakizumab, Timigutuzumab, Timolumab, Tiragotumab, Tislelizumab, Tisotumab vedotin, Tnx-650, Tocilizumab (ACTEMRA), Tomuzotuximab, Toralizumab, Tosatoxumab, Tositumomab, Tovetumab, Tralokinumab, Trastuzumab (HERCEPTIN), Trastuzumab and Hyaluronidase-oysk (HERCEPTIN HYLECTA), Trastuzumab emtansine, Trbs07, Tregalizumab, Tremelimumab, Trevogrumab, Tucotuzumab celmoleukin, Tuvirumab, Ublituximab, Ulocuplumab, Urelumab, Urtoxazumab, Ustekinumab (STELARA), Utomilumab, Vadastuximab talirine, Vanalimab, Vandortuzumab vedotin, Vantictumab, Vanucizumab, Vapaliximab, Varisacumab, Varlilumab, Vatelizumab, Vedolizumab, Veltuzumab, Vepalimomab, Vesencumab, Visilizumab, Vobanlizumab, Volociximab, Vonlerolizumab, Vopratelimab, Vorsetuzumab mafodotin, Votumumab, Vunakizumab, Xentuzumab, Xmab-5574, Zalutumumab, Zanolimumab, Zatuximab, Zenocutuzumab, Ziralimumab, Zolbetuximab (IMAB362), and Zolimomab aritox.

In some embodiments, the test agent is a nucleic acid (e.g., a gene therapy molecule). The nucleic acid may be DNA (e.g., plasmid DNA and linear DNA) or is RNA (e.g., mRNA, antisense RNA, miRNA, siRNA, and gRNA). In some embodiments, the nucleic acid comprises a viral vector. RNA may be a small interfering RNA (siRNA), a microRNA (miRNA), a small hairpin RNA (shRNA), a messenger RNA (mRNA), an anti-sense nucleic acid (asRNA), and/or a guide RNA (gRNA). The nucleic acid may encode a gene-editing protein. A gene-editing protein recognizes, binds to, and/or creates a single- or double-stranded break in a gene's DNA sequence and reduces transcription of the gene. The gene-editing protein may be a CRISPR-associated protein 9 (Cas9), a Transcription Activator-Like Effector Nucleases (TALEN), or a Zinc Finger Nuclease (ZFN). Use of such gene-editing proteins may be considered a gene therapy.

Yet another aspect of the present disclosure is a drug capable of changing a cell's state, function, and/or predicted age that is identified by a herein-described method.

### Method for Reducing an In Vitro Cell's Predicted Age

In another aspect, the present disclosure provides an in vitro method for reducing a cell's predicted age. The method comprising contacting a cell with the drug capable of changing a cell's state, function, and/or predicted age that is identified by a herein-described method.

In some embodiments, a drug capable of changing a cell's state, function, and/or predicted age that is identified by a herein-described method is contacted with an in vitro cell prior to in vivo uses of the drug. Such in vivo uses may help identify dosages suitable for in vivo uses, possible side effects of the drug predicted to occur with in vivo uses, favorable/unfavorable excipients and formulations, and so forth.

The drug may be provided in vitro with an additional compound or agent to determine if the drug has a synergistic or antagonistic effect in context of the additional compound or agent.

Non-limiting exemplary methods for reducing an in vitro cell's predicted age are described in Examples 27 to 31. In some embodiments, reducing an in vitro cell's predicted age comprises restoring one or more aspects of the viral immune response of an older adult to a younger state. As an example, restoring comprises treating the older adult with a test compound or agent determined to restore the one or more aspects of the viral immune response for the older adult. In some embodiments, provided are methods of increasing the viral immune response of the older adult by administering a test compound. In one embodiment, the test compound is triptonide.

### Pharmaceutical Compositions and Administrations

In an aspect, the present disclosure provides a pharmaceutical composition for treating a disease or disorder associated with aging comprising a therapeutically-effective amount of a drug capable of changing a cell's state, function, and/or predicted age identified by a herein-described method.

The term therapeutically effective amount is meant the amount of a drug which can provide a desired therapeutic benefit, e.g., increasing lifespan, promoting longevity, and/or preventing, reducing the severity of, or delaying the onset of various aging-associated conditions.

A composition of the present disclosure may be formulated to be suitable for in vivo administration to a mammal. Such compositions can optionally comprise a suitable amount of a pharmaceutically acceptable excipient so as to provide the form for proper administration. Pharmaceutical excipients can be liquids, such as water or saline. In addition, auxiliary, stabilizing, thickening, lubricating, and coloring agents can be used. The pharmaceutically acceptable excipients are sterile when administered to a subject. Water is a useful excipient when any composition described herein is administered intravenously. In some embodiments, the compositions described herein are suspended in a saline buffer (including, without limitation Ringer's, TBS, PBS, HEPES, HBSS, and the like). Saline solutions and aqueous dextrose and glycerol solutions can also be employed as liquid excipients, specifically for injectable solutions. Suitable pharmaceutical excipients also include starch, glucose, lactose, sucrose, glycerol monostearate, sodium chloride, dried skim milk, glycerol, propylene, glycol, water, ethanol and the like. Any composition described herein, if desired, can also comprise pH buffering agents.

Dosage forms suitable for parenteral administration (e.g., intravenous injection or infusion, intraarterial injection or infusion, intramuscular injection, intraperitoneal injection, subcutaneous injection, and intra-arterial injection or infusion) include, for example, solutions, suspensions, dispersions, emulsions, and the like.

The dosage of any herein-disclosed composition can depend on several factors including the characteristics of a subject to be administered. Examples of characteristics include species, sex, age, weight, size, health, and/or disease status. Moreover, the dosage may depend on whether the administration is the first time the subject received a composition of the present disclosure or if the subject has previously received a composition of the present disclosure. Additionally, pharmacogenomic (the effect of genotype on the pharmacokinetic, pharmacodynamic or efficacy profile of a composition) information about a particular subject may affect dosage used. Furthermore, the exact individual dosages can be adjusted somewhat depending on a variety of factors, including the specific composition being administered, the time of administration, the route of administration, the nature of the formulation, and the rate of excretion. Some variations in the dosage can be expected.

Moreover, the dosage depends on the specific composition administered.

### Aging-Associated Conditions

A drug identified by a herein-disclosed method and pharmaceutical compositions comprising the same treat, prevent, reduce the severity of, and/or delay the onset of various aging-associated conditions, e.g., chronic diseases and disabilities/conditions of aging. Illustrative aging-associated conditions include age-related macular degeneration (AMD), Alzheimer's disease, arthritis (including osteoarthritis), atherosclerosis and cardiovascular disease, benign prostatic hyperplasia (BPH), bone atrophy, cancer, cataracts, constipation, decrease in visual acuity, decrease in overall energy, delirium, dementia, depression, diminished peripheral vision, glaucoma, greater risk of heat stroke or hypothermia, hearing loss, hypertension, increased susceptibility to infection (including influenza, SARS-CoV-2, sepsis and pneumonia), memory loss, metabolic syndrome, muscle atrophy (including sarcopenia), osteoporosis, reduced metabolism (including increased risk for obesity), reduced reflexes and coordination including difficulty with balance, respiratory disease, shingles, type 2 diabetes, urologic changes (including incontinence), whitening or graying of hair, and wrinkling and sagging skin (including loss of skin elasticity). Aged non-human subjects experience similar, homologous, and/or equivalent aging-associated conditions.

### Computer-Implemented Methods

In some embodiments, the methods of the present disclosure identify an in vitro cell's state, function, and/or predicted age, in part, using computer-implemented methods that analyze, characterize, and evaluate the in vitro cell's morphological signature and/or functional signature.

In some embodiments, the computer-implemented methods utilize machine learning techniques (e.g., deep learning; machine vision, classical machine learning such as linear regression, gradient boosting, random forests, linear regression, logistic regression, naïve Bayes classifier, support vector machines; and others) to learn from raw data. Models may be built that handle each sample type and its associated preparation process well such that the machine learning system can gradually improve itself via repeated experimentation and evaluation until the resulting models, weights, hyperparameters, and feature extractors are capable of successfully predicting an in vitro cell's state, function, and/or predicted age for a given sample and/or conditions. Different sample types and data generation operations may require different machine learning techniques. For example, data produced via imaging may be suitable for machine vision techniques and models to identify correlations and signal in microscopic images. Data produced via epigenetic sequencing may be suitable for genomic machine learning techniques and models. These approaches share underlying fundamentals (for example, both may use optimization methods such as gradient descent), but the technological approaches used may differ depending on the data type (e.g., images vs. sequencing data). Similarly, the exact model structure may differ depending on the source of the sample (e.g., blood sample vs. cultured cells), and the learned weights of the model may differ depending on many of the processes used during data gathering (e.g., which lab and what sets of equipment and workflows were used to acquire, prepare, and process the samples). Over time, these techniques and models may become more generalized and more and more shareable across multiple sample types and preparation processes.

The machine learning techniques may comprise a neural network. The neural network may include a convolutional neural network (CNN). The CNN may comprise a deep convolutional neural network (DCNN) (e.g., comprising multiple layers). The DCNN may comprise a cascaded deep convolutional neural network. Alternatively, the CNN may comprise a simplified CNN or a shallow CNN (e.g., comprising one feature layer and/or one hidden layer). The methods of the present disclosure may comprise a combination of deep learning architectures and/or other machine learning techniques, cascaded or chained together. For example, these cascades of machine learning architectures may be configured to generate intermediate results that are better processed by later parts in the chain or cascade, or to help interpret partial results coming from different parts of the chain or cascade. For example, deep learning architectures may comprise any combination of one or multiple CNN, recurrent neural networks, and/or generative adversarial networks. In addition, machine learning techniques such as gradient boosting, random forests, linear regression, logistic regression, naïve Bayes classifier, and/or support vector machines may be incorporated into the methods of the present disclosure.

The machine learning techniques may also be trained using training datasets using suitable methods appropriate for the one or more types of machine learning architectures (and sample types) used in the method of the present disclosure. As examples, the morphological signature and/or functional signature of cells of known ages can be used to create a training datasets, the morphological signature and/or functional signature of cells from subjects having a known disease or disorder can be used to create training sets, the morphological signature and/or functional signature of cells treated with a known test agent can be used to create training sets; the morphological signature and/or functional signature of cells having a known cell state (e.g., a M1 vs M2-type macrophage or memory T cell vs an effector T cell) can be used to create training sets; the morphological signature and/or functional signature of cells having a known cell function (e.g., a cytokine producing cell vs an inactive cell or a cell capable of interacting with another cell (e.g., phagocytosing) vs a cell that does not interact with other cells) can be used to create training sets.

The machine learning techniques may comprise a software module performing image preprocessing of sample data. The image preprocessing may include, for example, normalization, image enhancement, image correction, contrast enhancement, brightness enhancement, filtering with one or more filters, transformation, increasing image resolution, decreasing image resolution, increasing bit resolution, decreasing bit resolution, increasing image size, decreasing image size, increasing field-of-view, decreasing field-of-view, background subtraction, noise or artifact subtraction, image subtraction, lossy compression, or lossless compression. The normalization may include, for example, normalization of signal noise, sequencing reads, image channel balancing/combination, quality filtering, color balancing, color normalization, or a combination thereof. The normalization may include, for example, normalization of image format, image slice spacing, image intensity, image contrast, image saturation, image size, image orientation, or other image data properties of the image data. The plurality of sample data may be segmented into a plurality of 2D slices. The detection structure may employ the convolutional neural network (CNN) to screen locations in a portion of or each of the plurality of 2D slices of the segmented sample data. For example, the CNN may use a sliding window methodology to identify the features and classify the features.

Relatedly, methods for normalizing images using illumination correction methods are described in, at least, Singh el al., “Pipeline for illumination correction of images for high-throughput microscopy” J Microsc. 2014 December; 256(3). 231-236, the contents of which is incorporated by reference in its entirety. For example, Data-driven or retrospective illumination correction in which the background variation in each image can be independently corrected by subtracting a smooth version of the raw image may be used. However, more preferably, retrospective methods that estimate an illumination correction function (ICF) by combining information across multiple images are more robust. The ICF is calculated by averaging all images in an experimental batch (usually, all images for a particular channel from a particular multi-well plate), followed by smoothing using a median filter, e.g., 500 pixels. Then, each image is corrected by dividing it by the ICF.

In some embodiments, the value of the median filter is chosen by starting with filter dimensions approximately 25% that of the image, then increasing the dimensions in increments of about 10/6 of the image size until the ICFs appear smooth overall; the presence of bright ‘blotches’ in the ICF indicates that the local cellular intensities still dominate the global illumination pattern or that artefacts are present. In some embodiments, the value of the median filter is chosen by using a fixed number, such as a 256-pixel median filter over a 1080p image. The ICF approach may utilize freely available open-source software, for example CellProfiler (Carpenter et al., 2006, Genome Biology, 7(10), R100; Kamentsky et al., 2011, Bioinformatics, 27(8), 1179-80; the contents of each of which is incorporated by reference in its entirety) so that it can be readily and routinely applied to large numbers of images from high-throughput microscopy experiments. For microtiter plate-based imaging experiments, ICFs may vary across plates; thus, ICF may be determined on a plate-by-plate basis.

Further, features (e.g., fluorescent signals) are extracted from each cell in an image (e.g., a field of view) may be normalized using a reference distribution defined by control cells. The image-based profile may be given by computing the mean for each feature across all the cells of a specific condition (e.g., cell type, cell of specific passage number, and cell treated with a test agent). See, Ljosa et al., 2013, J. Biomol. Screening, 18(10) 1321-1329, the contents of which is incorporated by reference in its entirety. Alternatively, the features (e.g., fluorescent signals) are extracted from each cell in an image (e.g., a field of view) may be median-averaged for the cells in a given field.

In some embodiments, the background variation in each image can be additionally corrected by subtracting a smoothed version of the raw image.

The machine learning techniques may comprise a data pre-processing operation, which alters the input images in various ways so as to aid the training of the models to better extract the features (e.g., fluorescent signals and morphological properties) which are relevant to the biological signal of the cell. These alterations may include, but not be limited to, rotating the image 90°, rotating the image 180°, rotating the image 270°, inverting the image left or right, and inverting the image up or down. These alterations aim to remove any nuisance signals present in the image that may be irrelevant to the biological signal of the cells being analyzed.

A convolutional neural network (CNN) useful in methods of the present disclosure may have 2 to 64 convolutional layers and 1 to 8 fully connected layers. For example, the CNN may have 2, 4, 8, 16, 32, 64, or more convolutional layers and 1, 2, 3, 4, 5, 6, 7, 8, or more fully connected layers. The sliding window may have less than about 4,000 pixels by less than about 4,000 pixels, less than about 2,000 pixels by less than about 2,000 pixels, less than about 1,000 pixels by less than about 1,000 pixels, less than about 512 pixels by less than about 512 pixels, less than about 256 pixels by less than about 256 pixels, less than about 128 pixels by less than about 128 pixels, less than about 64 pixels by less than about 64 pixels, less than about 32 pixels by less than about 32 pixels, less than about 16 pixels by less than about 16 pixels, less than about 8 pixels by less than about 8 pixels, or less than about 4 pixels by less than about 4 pixels. For example, the sliding window may have a first dimension (e.g., along the x-axis) or less than 100, less than 500, less than 1,000, less than 2,000, less than 3,000, less than 4,000, or less than 5,000 pixels; and may have a second dimension (e.g., along the y-axis) or less than 100, less than 500, less than 1,000, less than 2,000, less than 3,000, less than 4,000, or less than 5,000 pixels. The CNN may comprise a neural network instance selected randomly from a plurality of neural network instances. The second CNN may comprise a neural network instance selected randomly from a plurality of neural network instances.

The machine learning techniques may comprise a software module performing post-processing of one or a plurality of refined locations. For example, the post-processing may include characterizing centroid location, volume, shape, intensity, density, transparency, regularity, or a combination thereof.

A computer program operating the machine learning techniques may include instructions, executable by the processor, to apply to the first sample data a network structure, such as a recurrent neural network structure or a general adversarial network structure. The results obtained from applying the machine learning detection structure may be combined with results obtained from applying the network structure to determine an in vitro cell's state, function, and/or predicted age. The computer program may include instruction, executable by the processor, to apply to the first sample data a machine learning technique, such as gradient boosting, random forests, linear regression, logistic regression, naïve Bayes classifier, and support vector machines. The results obtained from applying the machine learning detection structure may be combined with results from applying the machine learning technique.

Specific examples of computer-implemented methods used in the present disclosure include at least the following: determination of a cell's ability to interact with another cell or fragment of another cell which is quantified using a spatial pattern analysis method; determination of an in vitro cell's size and/or the cell's shape and/or morphological properties (e.g., eccentricity, form factor, and solidity) which is quantified using convolutional neural networks; determination of an in vitro cell's morphological signature and/or functional signature which comprises characterizing the cell's mitochondrial shape (e.g., using one or more of spatial pattern analysis, texture analysis, intensity changes) using convolutional neural networks; quantification of cell-to-cell interactions using spatial pattern analyses; quantification cell shape, nucleus shape, and/or mitochondrial shape based upon, at least, spatial pattern analysis, texture analysis, and fluorescent intensity using convolutional neural networks; predictions of a cell's old or young age based on assay profiling of a broad range of cell organelles based upon trained convolutional neural networks; determination of the cell's predicted age which is determined by a machine learning technique selected from convolutional neural networks and gradient boosted trees; determination of cell subtype or state, such as the determination of the cell subtype composition of a heterogeneous group of immune cells; identification of therapeutically-relevant subsets of immune cells using an unsupervised machine learning algorithm selected from t-SNE, Autoencoder, principal component analysis, and/or k-means clustering; and prediction of cytokine and chemokine release by a PBMC using a supervised machine learning algorithm (e.g., based upon trained neural networks and/or gradient boosted trees).

In some embodiments of the present disclosure, a microscope detects, at least, fluorescent intensity signals associated with the binding reagent from a plurality of cells and across multiple fields of view. The computer-implemented methods are capable of computationally segmenting the individual cells based on their cell membrane, nucleus, and/or cytoskeleton. Accordingly, the methods of the present disclosure are capable of determining an individual in vitro cell's morphological signature and/or functional signature for identifying the individual in vitro cell's state, function, and/or predicted age.

The terminology used herein is for the purpose of describing particular cases only and is not intended to be limiting.

As used herein, unless otherwise indicated, the terms “a”, “an” and “the” are intended to include the plural forms as well as the single forms, unless the context clearly indicates otherwise.

The terms “comprise”, “comprising”, “contain,” “containing,” “including”, “includes”, “having”, “has”, “with”, or variants thereof as used herein, generally are inclusive in a manner similar to the term “comprising.”

The term “preventing,” as used herein, generally refers to avoiding the occurrence of a disease or disorder and/or reducing the likelihood of acquiring the disease or disorder. By treating is meant, at least, ameliorating or avoiding the effects of a disease or disorder, including reducing a sign or symptom of the disease or disorder.

The term “about” or “approximately” means within an acceptable error range for the particular value as determined by one of ordinary skill in the art, which will depend in part on how the value is measured or determined, e.g., the limitations of the measurement system. For example, “about” can mean 10% greater than or less than the stated value. In another example, “about” can mean within 1 or more than 1 standard deviation, per the practice in the given value. Where particular values are described in the application and claims, unless otherwise stated the term “about” should be assumed to mean an acceptable error range for the particular value.

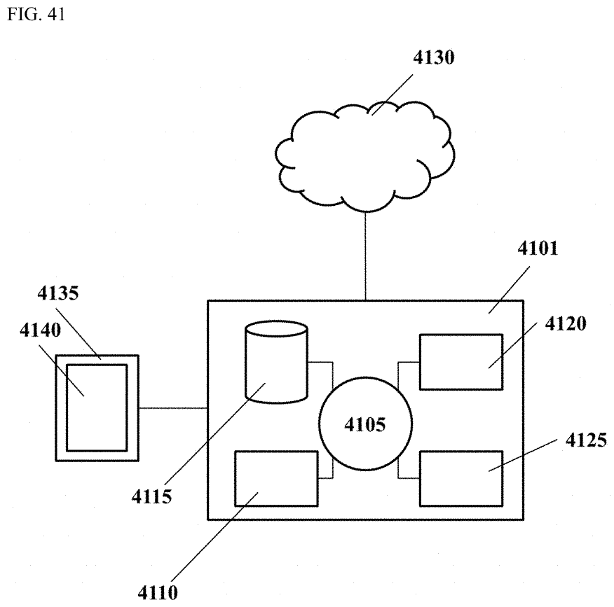

The present disclosure provides computer systems that are programmed to implement methods of the disclosure. FIG. 41 shows a computer system 4101 that is programmed or otherwise configured to, for example, determine morphological signatures of a cell, determine functional signatures of a cell, identify a cell's state, function, and/or predicted age based on the morphological signature and/or functional signature, and identify a test agent as a drug capable of changing a cell's state, function, and/or predicted age.

The computer system 4101 can regulate various aspects of analysis, calculation, and generation of the present disclosure, such as, for example, determining morphological signatures of a cell, determining functional signatures of a cell, identify a cell's state, function, and/or predicted age based on the morphological signature and/or functional signature, and identifying a test agent as a drug capable of changing a cell's state, function, and/or predicted age. The computer system 4101 can be an electronic device of a user or a computer system that is remotely located with respect to the electronic device. The electronic device can be a mobile electronic device.

The computer system 4101 includes a central processing unit (CPU, also “processor” and “computer processor” herein) 4105, which can be a single core or multi core processor, or a plurality of processors for parallel processing. The computer system 4101 also includes memory or memory location 4110 (e.g., random-access memory, read-only memory, flash memory), electronic storage unit 4115 (e.g., hard disk), communication interface 4120 (e.g., network adapter) for communicating with one or more other systems, and peripheral devices 4125, such as cache, other memory, data storage and/or electronic display adapters. The memory 4110, storage unit 4115, interface 4120 and peripheral devices 4125 are in communication with the CPU 4105 through a communication bus (solid lines), such as a motherboard. The storage unit 4115 can be a data storage unit (or data repository) for storing data. The computer system 4101 can be operatively coupled to a computer network (“network”) 4130 with the aid of the communication interface 4120. The network 4130 can be the Internet, an internet and/or extranet, or an intranet and/or extranet that is in communication with the Internet.

The network 4130 in some cases is a telecommunication and/or data network. The network 4130 can include one or more computer servers, which can enable distributed computing, such as cloud computing. For example, one or more computer servers may enable cloud computing over the network 4130 (“the cloud”) to perform various aspects of analysis, calculation, and generation of the present disclosure, such as, for example, determining morphological signatures of a cell, determining functional signatures of a cell, identify a cell's state, function, and/or predicted age based on the morphological signature and/or functional signature, and identifying a test agent as a drug capable of changing a cell's state, function, and/or predicted age. Such cloud computing may be provided by cloud computing platforms such as, for example, Amazon Web Services (AWS), Microsoft Azure, Google Cloud Platform, and IBM cloud. The network 4130, in some cases with the aid of the computer system 4101, can implement a peer-to-peer network, which may enable devices coupled to the computer system 4101 to behave as a client or a server.

The CPU 4105 may comprise one or more computer processors and/or one or more graphics processing units (GPUs). The CPU 4105 can execute a sequence of machine-readable instructions, which can be embodied in a program or software. The instructions may be stored in a memory location, such as the memory 4110. The instructions can be directed to the CPU 4105, which can subsequently program or otherwise configure the CPU 4105 to implement methods of the present disclosure. Examples of operations performed by the CPU 4105 can include fetch, decode, execute, and writeback.

The CPU 4105 can be part of a circuit, such as an integrated circuit. One or more other components of the system 4101 can be included in the circuit. In some cases, the circuit is an application specific integrated circuit (ASIC).

The storage unit 4115 can store files, such as drivers, libraries and saved programs. The storage unit 4115 can store user data, e.g., user preferences and user programs. The computer system 4101 in some cases can include one or more additional data storage units that are external to the computer system 4101, such as located on a remote server that is in communication with the computer system 4101 through an intranet or the Internet.

The computer system 4101 can communicate with one or more remote computer systems through the network 4130. For instance, the computer system 4101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 4101 via the network 4130.

Methods as described herein can be implemented by way of machine (e.g., computer processor) executable code stored on an electronic storage location of the computer system 4101, such as, for example, on the memory 4110 or electronic storage unit 4115. The machine executable or machine readable code can be provided in the form of software. During use, the code can be executed by the processor 4105. In some cases, the code can be retrieved from the storage unit 4115 and stored on the memory 4110 for ready access by the processor 4105. In some situations, the electronic storage unit 4115 can be precluded, and machine-executable instructions are stored on memory 4110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 4101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 4101 can include or be in communication with an electronic display 4135 that comprises a user interface (UI) 4140 for providing, for example, morphological signatures of a cell, functional signatures of a cell, and a cell's state, function, and/or predicted age. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 4105. The algorithm can, for example, determine morphological signatures of a cell, determine functional signatures of a cell, identify a cell's state, function, and/or predicted age based on the morphological signature and/or functional signature, and identify a test agent as a drug capable of changing a cell's state, function, and/or predicted age.

Any aspect or embodiment described herein can be combined with any other aspect or embodiment as disclosed herein.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Modeling of Critical Immune States and Functions Using Cell Sorting

A sample of PBMC patient cells is sorted by cell type, stained with a panel of binding reagents, and classified based on morphological signature and/or functional signature using a trained model. This approach may be used for any cell type that can be identified phenotypically, even with a relatively complex cell surface market schema, e.g., up to about 8-12 channels. Up to four cell populations are sorted simultaneously from each sample. The sorting method includes sorting out dead cells. A benefit of cell sorting is that flow cytometry fluorescence and gating is well understood, particularly for certain markers described herein. In addition, there may be no staining optimization necessary for flow cytometry as certain markers can be checked through correlation with well-characterized FSC/SSC values, and by sorting specific cell types there is ground truth data on each well at a very high purity (95-99%). Further, instead of relying on just Brightfield and Hoechst to train cell identification, cells can be stained with an entire CellPainting palette.

Briefly, cell types are sorted and stained with using a herein-described palette (e.g., the CellPainting Membrane (CPM) palette, CellPainting Epigenetic (CPE) palette, Cell Specific (CPS) palette, and CellPainting Membrane+Cell Specific (CPMS)) on total PBMCs. Combinations of palettes may be used. For instance, a palette comprising features of the CPS palette and the CPM palette. A classifier is trained with sorted cell subsets. Subsequent experiments are performed using a palette on total PBMCs. In order to ensure that antibodies and fluorophores used in sorting are not visible after 24 hour culture and/or interfere with a CellPainting palette, an experiment is performed to determine whether fluorophores are visible after 24 hours of cell culture. PBMCs are first stained with a panel comprising: a stain to distinguish between live and dead cells (e.g., one or more of DAPI, propidium iodide (PI), and a Zombie dye), CD3, CD19, CD56, CD11c, and CD14. A sample is sorted on a flow cytometer to set the gating strategy for sorting. Remaining sample is thoroughly washed, plated in media, and incubated for 24 hours. The incubated sample is then sorted on the flow cytometer again to determine any residual fluorescence. An unstained sample is sorted in a similar manner to set a baseline.

If fluorescence is maintained after 24 hours, five PBMC samples are stained with the same panel (a stain to distinguish between live and dead cells (e.g., DAPI, PI, and a Zombie dye), CD3, CD19, CD56, CD11c, and CD14. The following populations are sorted from four samples each (gated on live singlets): CD3+ T cells, CD19+B cells, CD56+NK cells, CD11c+ Dendritic cells/monocytes, CD14+ Macrophages/monocytes. One of the populations is not tested for each of the samples on a rotating basis. The sorted populations are stained using a herein-described palette and data regarding the cell's morphological signature and/or functional signature are acquired.

### Example 2: Assays for Predicting PBMC Cell Composition

An imaging assay was developed to predict and label individual immune cells within a mixture of PBMCs. A model is trained using cell specific staining labels and PBMC cell types are predicted using a herein-described palette. The following markers were used to identify cell types: CD3 is expressed on T cells, CD19 is expressed on B cells, CD56 is expressed on NK cells (both CD56-bright and CD56-dim populations), CD14 is expressed on macrophages and expressed on subtypes of monocytes, CD11c is highly expressed on dendritic cells and expressed on some monocytes (intermediate and nonclassical). T cells can be characterized by CD3+, CD19−, CD14−, CD11c−. B cells can be characterized by CD3−, CD19+, CD56−, CD14−, CD11c−. NK cells can be characterized by CD3−, CD56+, CD19−, CD14−, CD11c−. Macrophages and monocytes can be characterized by CD3−, CD19−, CD14+. Dendritic cells can be characterized by CD3−, CD19−, CD11c+. There may be rare exceptions where NK cells are CD19+, CD56+ and T cells are CD11c+, CD3+.

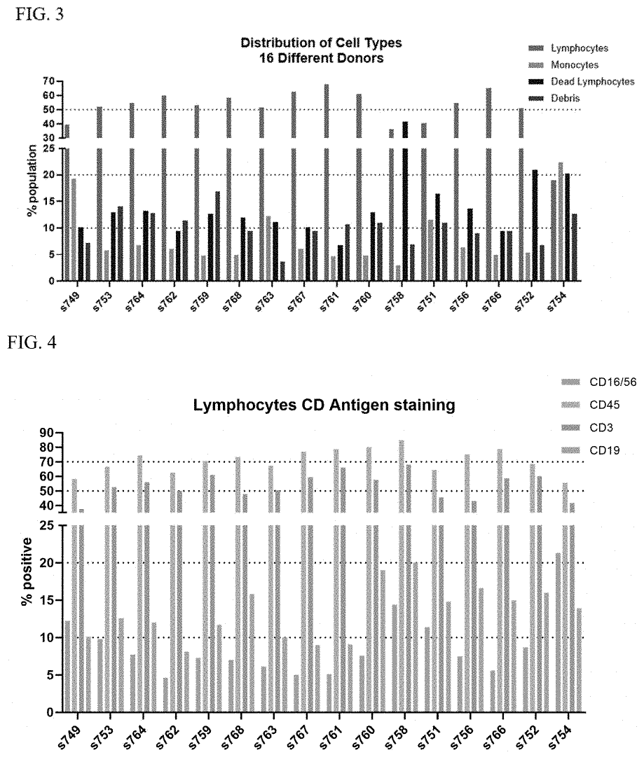

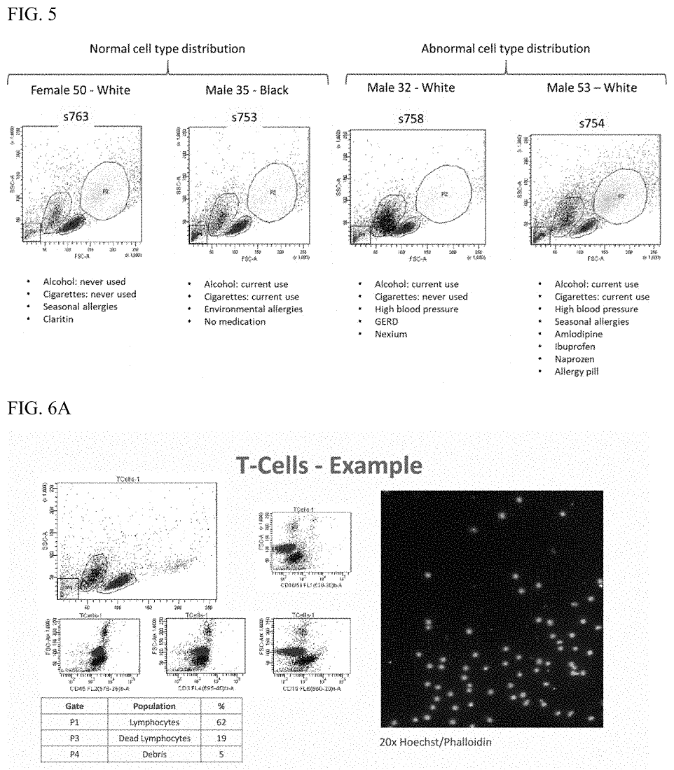

Flow cytometry was performed to measure the ground truth cell composition. About 100,000 PBMC cells/cm² or 10,000 PBMC cells per well were plated on a 384 well plate, for flow cytometry analysis at T24. Cells were detached with PBS/EDTA and stained for flow cytometry analysis using four-color flow kit/BioRad (4C007): PERCP conjugated CD3 (Mouse IgG1), FITC conjugated CD16/CD56 (Mouse IgG1, IgG2a), RPE conjugated CD45 (Mouse IgG2a), APC conjugated CD19 (Mouse IgG1). In certain experiments, flow detection and analysis panel were performed with a custom five-color flow kit comprising: CD3 PerCP (T Cells), APC anti-CD19 (B cells), PE anti-CD14 (macrophage/monocyte), APC-Cy7 anti-CD11c (dendritic cell/monocyte), and FITC anti-CD56 (NK cells). Monocytes, granulocytes, debris and dead lymphocyte fraction was determined by forward scatter and side scatter (FSC/SSC) as shown in FIG. 3. The distribution of CD16/56, CD45, CD3 and CD19 within lymphocytes are shown in FIG. 4. Cell distributions indicated as normal or abnormal cell type distribution among different donors are shown in FIG. 5.

PBMCs from sixteen donors were plated on 384 well plates at about 10,000 cells per well (240,000 cells per donor×2). The cells were stained with one or more of palettes: CellPainting Specific (CPS), CellPainting Membrane (CPM), CellPainting Epigenetic (CPE), and CellPainting Membrane+Cell Specific (CPMS). CPS comprises use of Brightfield microscopy, Hoechst stain, CD3 stain to identify T cells, CD19 stain to identify B cells, CD14 stain to identify macrophages/monocytes, CD11c stain to identify dendritic cells/monocytes, and CD56 stain to identify NK cells. CPM comprises use of Brightfield microscopy, Hoechst stain, wheat germ agglutinin (WGA) stain, phalloidin stain, Concanavalin A stain, and a stain to distinguish between live and dead cells (e.g., one or more of DAPI, propidium iodide (PI), and a Zombie dye). CPE comprises use of Brightfield microscopy, Hoechst stain, open chromatin stain H3K27ac, closed chromatin stain H3K27me3, MitoTracker® mitochondrial stain, and a stain to distinguish between live and dead cells. CPMS comprises use of Brightfield microscopy, Hoechst stain, CD3 stain to identify T cells, phalloidin stain, Concanavalin A stain, and CD11c stain to identify dendritic cells/monocytes. The experiment was performed with 2 plate replicates×3 (CPM, CPE, CPS, CPMS).

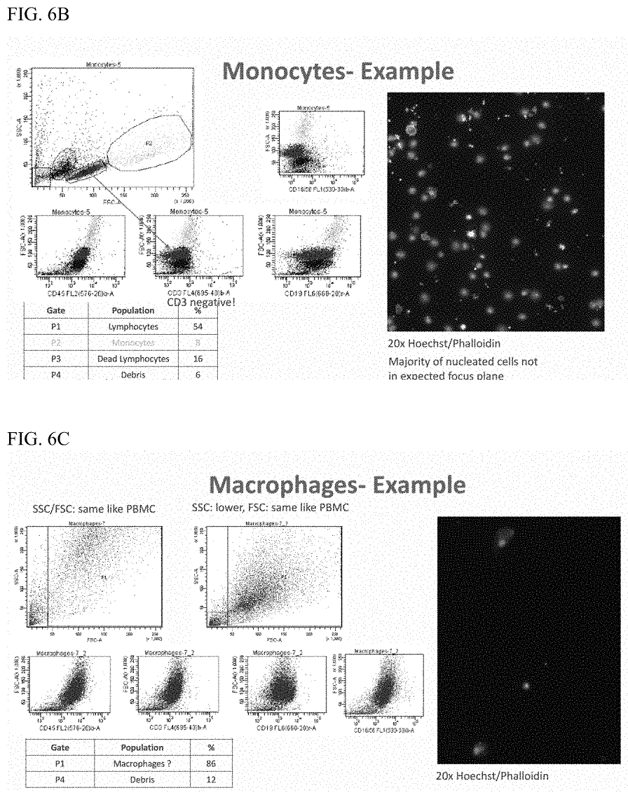

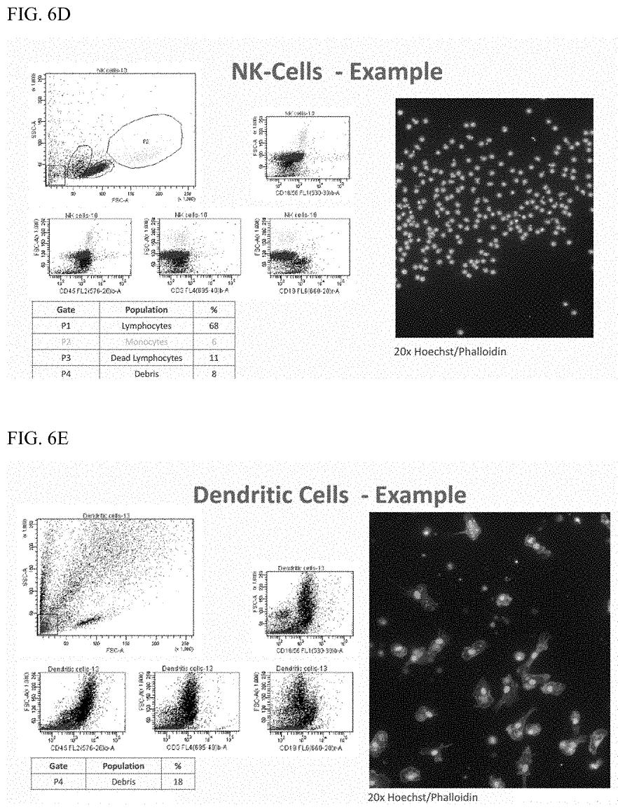

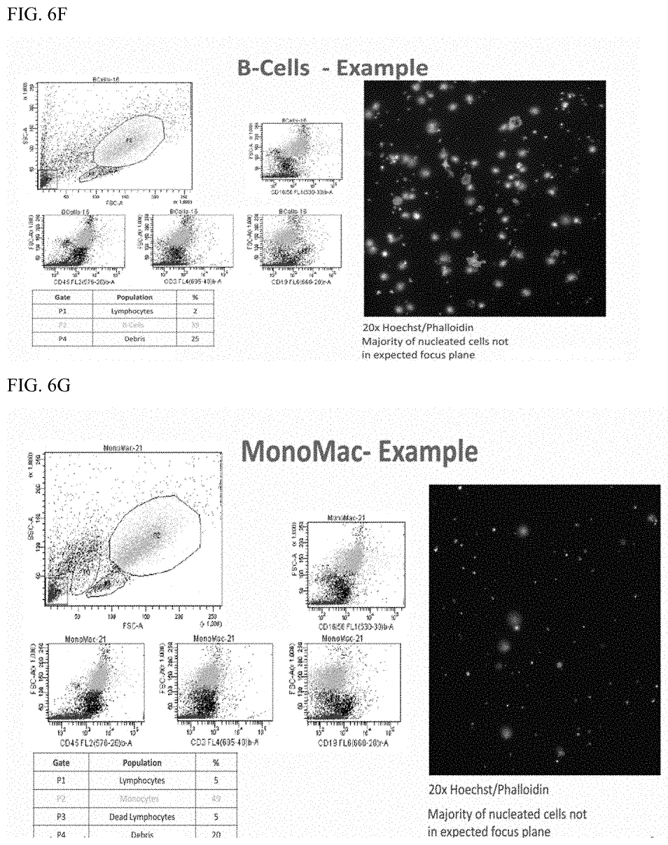

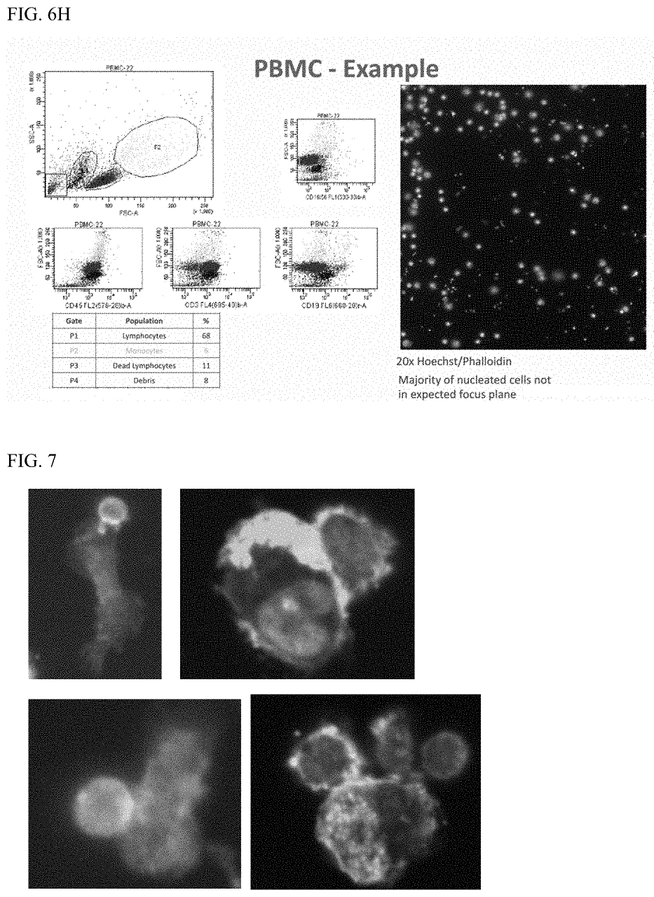

Isolated cells were stained using CPS, CPM, CPE, or CPMS. Three donors from each of the following cell type were stained: NK cell, monocyte, macrophage, dendritic cell, T cell, B cell, PBMC and monocyte/macrophage. FIG. 6A to FIG. 6H show exemplary cell distribution by flow cytometry and cell staining by fluorescent microscopy.

The cells were segmented using the palettes with the following priorities:

1. T cells/B cells/NK Cells vs Monocytes/Dendritic/Macrophages,

2. T cells/B cells/NK Cells vs Monocytes/Dendritic vs Monocyte/Macrophages,

3. T cells/B cells vs NK Cells vs Monocytes/Dendritic vs Monocyte/Macrophages,

4. T cells vs B cells vs NK Cells vs Monocytes/Dendritic vs Monocyte/Macrophages.

The following training and testing splits were performed: for cell specific strains as labels, train on Hoechst, Brightfield and CP nuclei features, and test on cell specific stains (CV fold), cell painting PBMCs (composition matches), and cell painting isolated cells. For wetlab isolated cells as labels, train on cell painting all stains and CP features, and test on cell specific strains (nuclei), isolated cells (CV fold), and cell painting PMBCs (composition matches). For example, sets as labels, train on cell painting all stains and CP features, and test on cell painting PBMCs all stains. For unsupervised clustering of cell painting data, train on clustering on all cells, and test on clustering within cell subtype.

### Example 3: Assays for Modeling Immune Cell States

The interaction between T cells and professional antigen-presenting cells (APCs), including dendritic cells and macrophages, is an essential step in triggering an adaptive (T cell) immune response. APCs present foreign antigens via major histocompatibility complex (MHC) receptors to CD3 receptors on T cells (TCRs) which stimulate the cells to seek out specific pathogens using effector functions. The first step in this interaction is the scanning of MHC receptors by T cells, which is influenced by the motility of individual cell populations. T cells scan the surfaces of APCs in search of antigen peptides bound to MHC proteins. Quantifying this interaction is critical for understanding the effects of immunomodulatory drugs. Using spatial point pattern analysis, statistical models of the distance between various cell types in a PBMC mixture can be built. The models may be useful for quantifying cell to cell interactions. The quantifications may be used to determine how cell interactions change in the presence of drugs, viruses, bacteria, age, and so forth.

| | un- | | migG | | anti | | anti | | un- | | migG | | anti | | anti | | un- | | migG | | anti | | anti | | 3 μg/ml ea |
|---|---|---|---|---|---|---|---|---|---|---|---|---|---|---|---|---|---|---|---|---|---|---|---|---|---|
| | treated | | Isotype | | HLA DR | | CD54 | | treated | | Isotype | | HLA DR | | CD54 | | treated | | Isotype | | HLA DR | | CD54 | | (3 h pre- |
| | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | incubation |
| A | 1 | 1 | 1 | 1 | 1 | 1 | 1 | 1 | 1 | 1 | 1 | 1 | 1 | 1 | 1 | 1 | 1 | 1 | 1 | 1 | 1 | 1 | 1 | 1 | |
| B | 2 | 2 | 2 | 2 | 2 | 2 | 2 | 2 | 2 | 2 | 2 | 2 | 2 | 2 | 2 | 2 | 2 | 2 | 2 | 2 | 2 | 2 | 2 | 2 | |
| C | 3 | 3 | 3 | 3 | 3 | 3 | 3 | 3 | 3 | 3 | 3 | 3 | 3 | 3 | 3 | 3 | 3 | 3 | 3 | 3 | 3 | 3 | 3 | 3 | |
| D | 4 | 4 | 4 | 4 | 4 | 4 | 4 | 4 | 4 | 4 | 4 | 4 | 4 | 4 | 4 | 4 | 4 | 4 | 4 | 4 | 4 | 4 | 4 | 4 | |
| E | 5 | 5 | 5 | 5 | 5 | 5 | 5 | 5 | 5 | 5 | 5 | 5 | 5 | 5 | 5 | 5 | 5 | 5 | 5 | 5 | 5 | 5 | 5 | 5 | |
| F | 6 | 6 | 6 | 6 | 6 | 6 | 6 | 6 | 6 | 6 | 6 | 6 | 6 | 6 | 6 | 6 | 6 | 6 | 6 | 6 | 6 | 6 | 6 | 6 | |
| G | 7 | 7 | 7 | 7 | 7 | 7 | 7 | 7 | 7 | 7 | 7 | 7 | 7 | 7 | 7 | 7 | 7 | 7 | 7 | 7 | 7 | 7 | 7 | 7 | |
| H | 8 | 8 | 8 | 8 | 8 | 8 | 8 | 8 | 8 | 8 | 8 | 8 | 8 | 8 | 8 | 8 | 8 | 8 | 8 | 8 | 8 | 8 | 8 | 8 | |
| I | 9 | 9 | 9 | 9 | 9 | 9 | 9 | 9 | 9 | 9 | 9 | 9 | 9 | 9 | 9 | 9 | 2 | 9 | 9 | 9 | 9 | 9 | 9 | 9 | |
| J | 10 | 10 | 10 | 10 | 10 | 10 | 10 | 10 | 10 | 10 | 10 | 10 | 10 | 10 | 10 | 10 | 10 | 10 | 10 | 10 | 10 | 10 | 10 | 10 | |
| K | 11 | 11 | 11 | 11 | 11 | 11 | 11 | 11 | 11 | 11 | 11 | 11 | 11 | 11 | 11 | 11 | 11 | 11 | 11 | 11 | 11 | 11 | 11 | 11 | |
| L | 12 | 12 | 12 | 12 | 12 | 12 | 12 | 12 | 12 | 12 | 12 | 12 | 12 | 12 | 12 | 12 | 12 | 12 | 12 | 12 | 12 | 12 | 12 | 12 | |
| M | 13 | 13 | 13 | 13 | 13 | 13 | 13 | 13 | 13 | 13 | 13 | 13 | 13 | 13 | 13 | 13 | 13 | 13 | 13 | 13 | 13 | 13 | 13 | 13 | |
| N | 14 | 14 | 14 | 14 | 14 | 14 | 14 | 14 | 14 | 14 | 14 | 14 | 14 | 14 | 14 | 14 | 14 | 14 | 14 | 14 | 14 | 14 | 14 | 14 | |
| O | 15 | 15 | 15 | 15 | 15 | 15 | 15 | 15 | 15 | 15 | 15 | 15 | 15 | 15 | 15 | 15 | 15 | 15 | 15 | 15 | 15 | 15 | 15 | 15 | |
| P | 16 | 16 | 16 | 16 | 16 | 16 | 16 | 16 | 16 | 16 | 16 | 16 | 16 | 16 | 16 | 16 | 16 | 16 | 16 | 16 | 16 | 16 | 16 | 16 | |

| | | |
|---|---|---|
| na [symbol] ve | 10 ng/ml LPS | VSV(2 μg/ml poly I:C |

two plates with 10k and 40k (30-40k) cells/well, CPM, CPE

Different conditions that simulate cell to cell interactions are set up in a 384 well plate. Separate conditions include: Vesicular stomatitis virus (replication deficient VSV), lipopolysaccharide (LPS), and normal culture (native), in the presence or absence of a MHC-II blocking antibody. The cells are stained with the CPM cell painting palette or the CPE cell painting palette, and cell to cell interactions are quantified across all conditions. An overview of the experimental set-up is shown in the table below.

Briefly, MHC-II antibodies are preincubated for three hours before overnight stimulation (anti-human-HLA-DR (G46-6; 3 μg/ml) (eBiosciences), anti-human-CD54 (HA58; 3 μg/ml) (BD Biosciences), mouse-IgG2a isotype control (C1.18.4; 1:300) (BioXCell). Recombinant VSV expressing GFP is added (10 μl) at a multiplicity of infection (MOI) of 10 for eighteen hours. Alternatively, viral infection is mimicked by stimulation with TLR3 ligand Poly I:C (2 μg/mL). LPS is added at 10 ng/ml LPS (Invivogen). The incubations are performed according to the above table using sixteen donors, and imaged.

### Example 4: Assays for Identifying Cell Function: T Cell Activation

Spatial changes in immune cells, such as T cell activation, may be assessed using an imaging assay provided herein. T cells are activated by the binding of T cell receptors (TCRs) to antigen-loaded major histocompatibility complexes on antigen-presenting cells (APCs), resulting in the formation of TCR “microclusters”, which coalesce in the immunological synapse (IS), thereby allowing the delivery of effector functions. Actin cytoskeletal rearrangements regulating the membrane architecture of T cells are important during initial cell-cell interactions through the formation of actin-rich protrusions. These, in turn, allow the formation of “close contacts” between T cells and APCs, favoring the transient interactions of proteins required for signaling. FIG. 7 is a panel of fluorescence microscopy images showing changes that occur during T cell activation such as substantial changes in the cytoskeleton, rearrangement of T cell mitochondria to the location of the immunological synapse, and TCR microclusters. The cells were imaged with four palettes:

(1) CellPainting Membrane (CPM)—4 FOV, 20× water lens, wide field (yields approx. 1500-2000 events (cells)), markers: Brightfield, Hoechst, WGA, Phalloidin, ConcanavalinA, Live/Dead;

(2) CellPainting Epigenetic (CPE)—4 FOV, 40× water lens, confocal 4×z-stack (yields approx. 400-500 events (cells)), markers: Brightfield, Hoechst, Open Chromatin (H3K27ac), Closed Chromatin (H3K27me3), Mitotracker®, Live/Dead:

(3) Cell Specific (CPS)—4 FOV, 20×water lens, wide field (yields approx. 1500-2000 events (cells)), markers: Brightfield, Hoechst, T Cells (CD3), B Cells (CD19), Monocyte/Macrophage (CD14), Monocyte/Dendritic Cell (CD11c), NK Cells (CD56); and

(4) CellPainting Membrane+Cell Specific (CPMS)—4 FOV, 20× water lens, wide field (yields approx. 1500-2000 events (cells)), markers: Brightfield, Hoechst, T Cells (CD3), Phalloidin, ConcanavalinA, Monocyte/Dendritic Cell (CD11c).

### Example 5: Assays for Identifying Cytokine-Mediated Intercellular Communication

Intercellular communication mediated by cytokines is a primary mechanism by which cells of the immune system communication. Measurement of this communication, in the context of other age-related changes, may provide a phenotyping tool useful for understanding one of the most important immune cell functions.

Here, PBMCs are stimulated and cytokine release is quantified. PBMCs are activated with LPS from different sources, with expected cytokine release of: IFNg, IL6, IL10, IL12 and TNFα. PBMCs are activated with PMA and lonomycin, with expected cytokine release of IL2, IFNg, TNFα, RANTES and TGFβ. PBMCs are activated with PWM, with expected cytokine release of IFNg, TNFα, TNFβ, IL2 (114), and IL10.

Briefly, PBMCs are thawed, resuspended in 10 ml of prewarmed culture medium, centrifuged at 138×g for 5 min, and medium is removed. The cells are resuspended in 4 ml of plating medium, the cells are centrifuged at 138×g for 5 min, and medium is removed, and the cells are resuspended in 10 ml of plating medium. Cells are diluted to a plating density of 4×10⁵ per ml. 25 μL of cell suspension (˜10,000 cells) are transferred to assay plates and the cells are incubated at 37° C. for half an hour.

The following PBMC stimulators are independently added to different wells: Phytohaemagglutinin (PHA) or ConcanavalinA (ConA), which is mainly for T cell proliferation; Pokeweed Mitogen (PWM), which is mainly for T- and B cell proliferation; or Lipopolysaccharide (LPS), which is mainly for B cell and monocyte activation. 25 μL of a stimulator is added to the plate (2× serum containing medium with 2×LPS (100 ng/mL final); 2×PMA (25 ng/mL)+2× Ionomycin (1 μg/mL final); 2×PWM (10 μg/mL final)+2×PHA (10 μg/mL final); or buffer alone). For time point 0, only 2× serum containing medium with 2×DMSO (0.2%) is added. The cells are incubated at 37° C. as appropriate (0, 8, 24 or 48 hours). The cells are then harvested fixed, and imaged.

### Example 6: Assays for Identifying Macrophages and Subtypes Thereof

Macrophages are a type of white blood cell that engulfs and digests cellular debris, foreign substances, microbes, cancer cells, and anything else that does not have surface expression of proteins specific to healthy cells. Such engulfment is known as phagocytosis. Macrophages also play a critical role in nonspecific defense (innate immunity) and help initiate specific defense mechanisms (adaptive immunity) by recruiting other immune cells such as lymphocytes. Macrophages also play an important anti-inflammatory role and can decrease immune reactions through the release of cytokines.

In the presence of inflammatory stimuli and danger signals, macrophages polarize toward the M1 state and release reactive species and inflammatory cytokines to fight pathogens. In contrast, a wound healing environment promotes polarization toward an M2 phenotype and leads to cellular processes that facilitate tissue repair. Macrophages exhibit different degrees of elongation when stimulated toward M1 or M2 phenotypes with cytokines in vitro. M1 macrophages exhibit smaller, more rounded cells with tightly packed dotted texture of actin. M2 macrophages exhibit larger, more irregular cell bodies with smoother actin staining and more distributed localized spots. Provided herein are methods and systems for identifying different macrophage phenotypes.

Macrophage type (MI vs M2) can change with age. These changes are diverse and, in general, may represent pro-inflammatory activation of cells with an alternatively activated (M2-like) phenotype. Impaired macrophage polarization in the elderly may dysregulate the development of the host response, making them more susceptible to infectious diseases. The aging microenvironment may be a key modulator of macrophage-elicited responses.

Monocytes (which differentiate into macrophages) can be polarized towards an M1 phenotype by IFNγ or LPS. The addition of granulocyte macrophage colony-stimulating factor (GM-CSF, which acts as a priming signal for macrophages during M1 polarization) augments the pro-inflammatory function of these cells. By contrast, M2 polarization can be achieved by the addition of IL-4 or IL-13. As with GM-CSF and M1 polarization, macrophage colony-stimulating factor (M-CSF) can enhance the anti-inflammatory function of M2 macrophages.

Cells are stained using the CellPainting Membrane (CPM) palette or the CellPainting Epigenetic (CPE) palette with a M1 or M2 marker in a schematic as shown below.

| | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | | |
|---|---|---|---|---|---|---|---|---|---|---|---|---|---|---|---|---|---|---|---|---|---|---|---|---|---|---|
| A | P1 | M1 | P2 | M2 | P3 | M3 | P1 | M1 | P2 | M2 | P3 | M3 | P1 | M1 | P2 | M2 | P3 | M3 | P1 | M1 | P2 | M2 | P3 | M3 | PBMC 1 | P1 |
| B | P1 | M1 | P2 | M2 | P3 | M3 | P1 | M1 | P2 | M2 | P3 | M3 | P1 | M1 | P2 | M2 | P3 | M3 | P1 | M1 | P2 | M2 | P3 | M3 | PBMC 2 | P2 |
| C | P1 | M1 | P2 | M2 | P3 | M3 | P1 | M1 | P2 | M2 | P3 | M3 | P1 | M1 | P2 | M2 | P3 | M3 | P1 | M1 | P2 | M2 | P3 | M3 | PBMC 3 | P3 |
| D | P1 | M1 | P2 | M2 | P3 | M3 | P1 | M1 | P2 | M2 | P3 | M3 | P1 | M1 | P2 | M2 | P3 | M3 | P1 | M1 | P2 | M2 | P3 | M3 | Monocyte 1 | M1 |
| E | P1 | M1 | P2 | M2 | P3 | M3 | P1 | M1 | P2 | M2 | P3 | M3 | P1 | M1 | P2 | M2 | P3 | M3 | P1 | M1 | P2 | M2 | P3 | M3 | Monocyte 2 | M2 |
| F | P1 | M1 | P2 | M2 | P3 | M3 | P1 | M1 | P2 | M2 | P3 | M3 | P1 | M1 | P2 | M2 | P3 | M3 | P1 | M1 | P2 | M2 | P3 | M3 | Monocyte 3 | M3 |
| G | P1 | M1 | P2 | M2 | P3 | M3 | P1 | M1 | P2 | M2 | P3 | M3 | P1 | M1 | P2 | M2 | P3 | M3 | P1 | M1 | P2 | M2 | P3 | M3 | | |
| H | P1 | M1 | P2 | M2 | P3 | M3 | P1 | M1 | P2 | M2 | P3 | M3 | P1 | M1 | P2 | M2 | P3 | M3 | P1 | M1 | P2 | M2 | P3 | M3 | untreated | untreated |
| I | P1 | M1 | P2 | M2 | P3 | M3 | P1 | M1 | P2 | M2 | P3 | M3 | P1 | M1 | P2 | M2 | P3 | M3 | P1 | M1 | P2 | M2 | P3 | M3 | MCSF | na [symbol] ve |
| J | P1 | M1 | P2 | M2 | P3 | M3 | P1 | M1 | P2 | M2 | P3 | M3 | P1 | M1 | P2 | M2 | P3 | M3 | P1 | M1 | P2 | M2 | P3 | M3 | LPS/INFg/mCSF | |
| K | P1 | M1 | P2 | M2 | P3 | M3 | P1 | M1 | P2 | M2 | P3 | M3 | P1 | M1 | P2 | M2 | P3 | M3 | P1 | M1 | P2 | M2 | P3 | M3 | IL-4/IL-13/mCSF | |
| L | P1 | M1 | | | | | | | | | | | | | | | | | | | | | | | | |

LPS is prepared at 20 ng/ml (6 μg), IFNγ is prepared at 20 ng/ml (1.2 μg), IL4 is prepared at 20 ng/ml (0.6 jig), IL13 is prepared at 10 ng/ml, and GM-CSF is prepared at 100 ng/ml. 2 plates are prepared in replicate and stained with CPM or CPE with M1/M2 marker. 10,000 cells are distributed per well, with 3 PBMC donors (PMBC 1, PBMC 2, PBMC 3) and 3 monocyte donors (Monocyte 1, Monocyte 2, Monocyte 3). Cells are untreated or treated with mCSF, LPS/IFNg/mCSF, or IL4/IL 13/mCSF.

These results are used for training an assay for predicting whether a macrophage has a M1 or M2 phenotype using a CPM or CPE staining procedure.

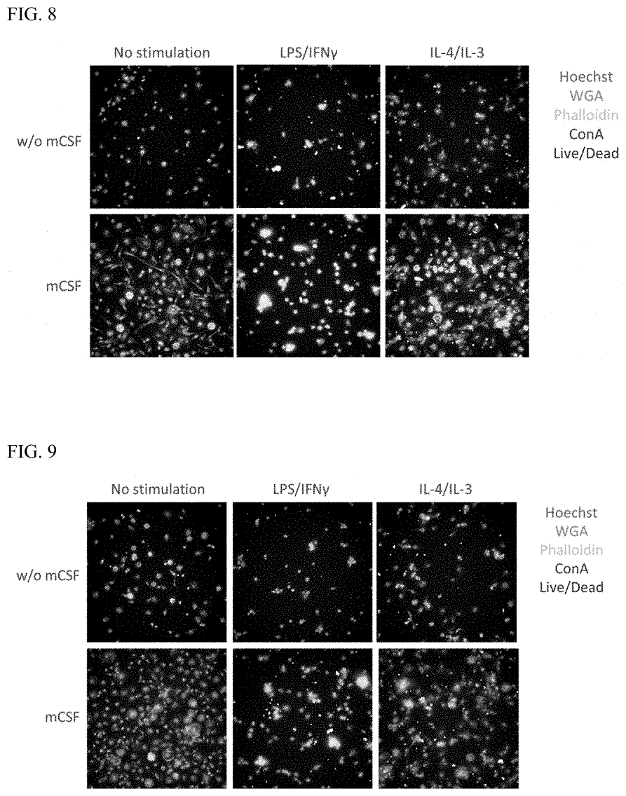

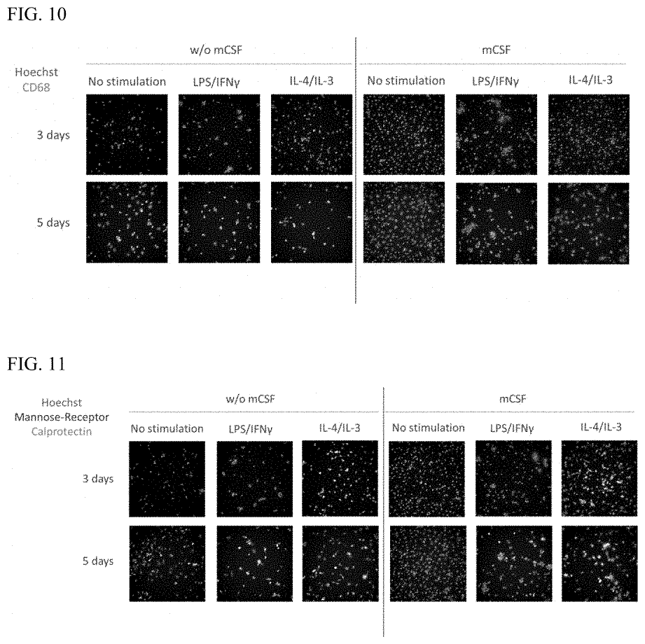

Cells stained and imaged using the CPM palette are shown in FIG. 8 and FIG. 9 after 3 days and 5 days, respectively. Expression of macrophage markers CD68, mannose-receptor (M2 polarization by IL4/IL13) and calprotectin (M1 polarization by IFN/LPS) are shown in FIG. 10 and FIG. 11.

Using the CPM palette on native cells and cells polarized to MI/M2, detection of the MI/M2 state was achieved with high accuracy at both the single cell and field level. In a subsequent experiment, the analysis was performed on production data with 16 donors. Classifiers from the production data were run on segmented macrophages from PBMC soup. An example of a map of the experimental set up is shown below.

| | | Monocyten s725 | | | | | | | | | | | | | | | | | | | | | | | | |
|---|---|---|---|---|---|---|---|---|---|---|---|---|---|---|---|---|---|---|---|---|---|---|---|---|---|---|
| 10000 cells/ | A | | | | | | | | | | | | | | | | | | | | | | | | | w/o mCSF |
| wells | B | | | | | | | | | | | | | | | | | | | | | | | | | |
| | C | | | | | | | | | | | | | | | | | | | | | | | | | |
| | D | | | | | | | | | | | | | | | | | | | | | | | | | |
| | E | | | | | | | | | | | | | | | | | | | | | | | | | |
| | F | | | | | | | | | | | | | | | | | | | | | | | | | |
| | G | | | | | | | | | | | | | | | | | | | | | | | | | |
| | H | | | | | | | | | | | | | | | | | | | | | | | | | |
| | I | | | | | | | | | | | | | | | | | | | | | | | | | w/MCSF |
| | J | | | | | | | | | | | | | | | | | | | | | | | | | |
| | K | | | | | | | | | | | | | | | | | | | | | | | | | |
| | L | | | | | | | | | | | | | | | | | | | | | | | | | |
| | M | | | | | | | | | | | | | | | | | | | | | | | | | |
| | N | | | | | | | | | | | | | | | | | | | | | | | | | |
| | O | | | | | | | | | | | | | | | | | | | | | | | | | |
| | P | | | | | | | | | | | | | | | | | | | | | | | | | |
| | | | | | | | | | | LPS and IFN-y | | | | | | | | IL-4 and IL-13 | | | | | | | | |
| | | | | | | | | | | M1 phenotype by IFN-y or LPS | | | | | | | | M2 polarisation by IL-4 and IL-13 | | | | | | | | |
| | | Monocyten s725 | | | | | | | | | | | | | | | | | | | | | | | | |
| | | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | |
| 10000 cells/ | A | | | | | | | | | | | | | | | | | | | | | | | | | w/o mCSF |
| wells | B | | | | | | | | | | | | | | | | | | | | | | | | | |
| | C | | | | | | | | | | | | | | | | | | | | | | | | | |
| | D | | | | | | | | | | | | | | | | | | | | | | | | | |
| | E | | | | | | | | | | | | | | | | | | | | | | | | | |
| | F | | | | | | | | | | | | | | | | | | | | | | | | | |
| | G | | | | | | | | | | | | | | | | | | | | | | | | | |
| | H | | | | | | | | | | | | | | | | | | | | | | | | | |
| | I | | | | | | | | | | | | | | | | | | | | | | | | | w/MCSF |
| | J | | | | | | | | | | | | | | | | | | | | | | | | | |
| | K | | | | | | | | | | | | | | | | | | | | | | | | | |
| | L | | | | | | | | | | | | | | | | | | | | | | | | | |
| | M | | | | | | | | | | | | | | | | | | | | | | | | | |
| | N | | | | | | | | | | | | | | | | | | | | | | | | | |
| | O | | | | | | | | | | | | | | | | | | | | | | | | | |
| | P | | | | | | | | | | | | | | | | | | | | | | | | | |

| | |
|---|---|
| LPS and IFN-y | IL-4 and IL-13 |
| M1 phenotype by IFN-y or LPS | M2 polarisation by IL-4 and IL-13 |

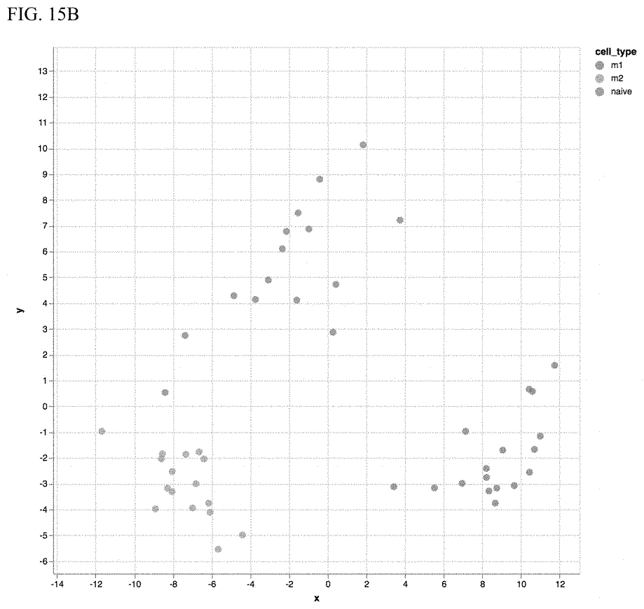

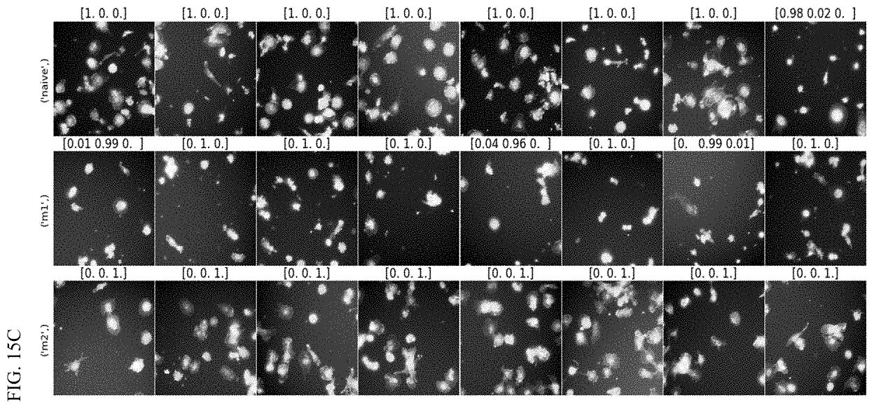

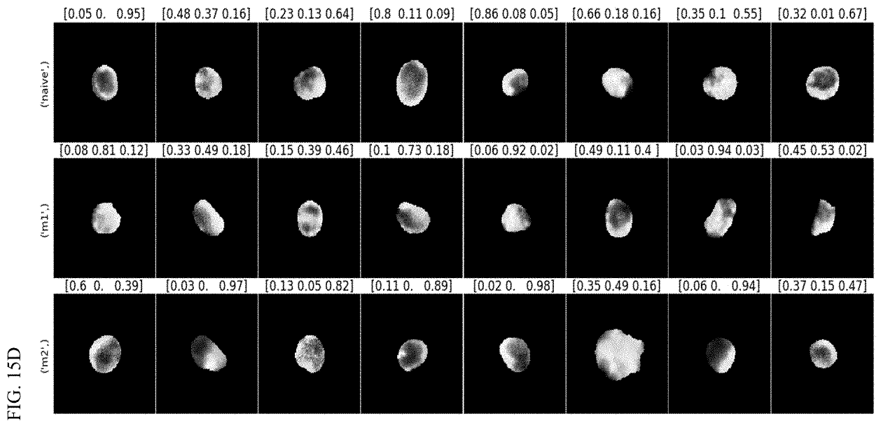

Images of M1 and M2 macrophages are shown in FIG. 15A. M1 macrophages smaller, more rounded cells. M2 macrophages are larger, more irregular cell bodies with smoother staining and more distributed localized spots. FIG. 15B is a visualization plot of cell type, where blue spots indicate M1 cell type, red spots indicate naïve cell type, and orange spots indicate M2 cell type. A training run was performed to visualize predictions and introspect a trained model. The classifier was trained to predict macrophage state after 5 day culture using field level images; prediction by [“cell type” ] is shown in FIG. 15C. The classifier was trained to predict macrophage state after 3 day culture using single cell masks; prediction by [“cell type” ] is shown in FIG. 151D.

### Example 7: Assays for Identifying T Cell Fates

Following activation, a naïve T cell transitions into an effector cell. Effector T cells are armed and activated to mount a response to an invasion. Memory T cells are a subset of infection- and cancer-fighting T cells that have previously encountered and responded to their cognate antigen; thus, the term “antigen-experienced” T cell is often applied. Such T cells can recognize foreign invaders, such as bacteria or viruses, as well as cancer cells. Memory T cells become “experienced” once they have encountered an antigen during an infection, encounter with cancer, or previous vaccination. Upon a second encounter with the antigen, memory T cells can reproduce to mount a faster and stronger immune response than the first time the immune system encountered the antigen, e.g., on a pathogen that infected the body.

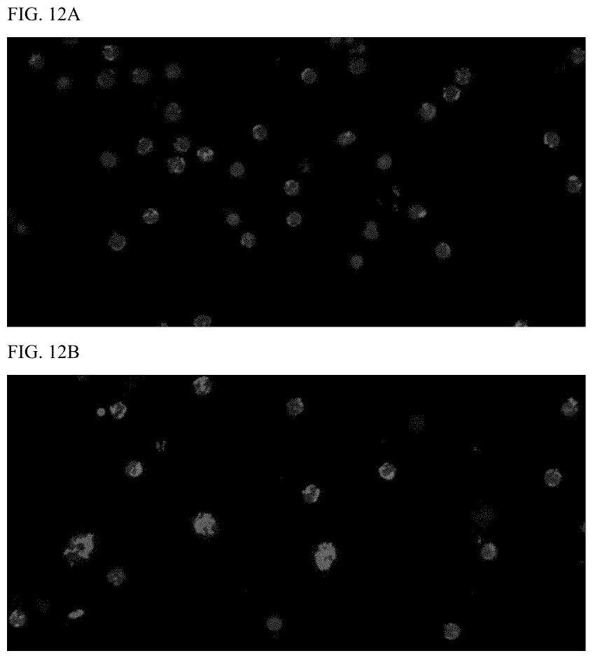

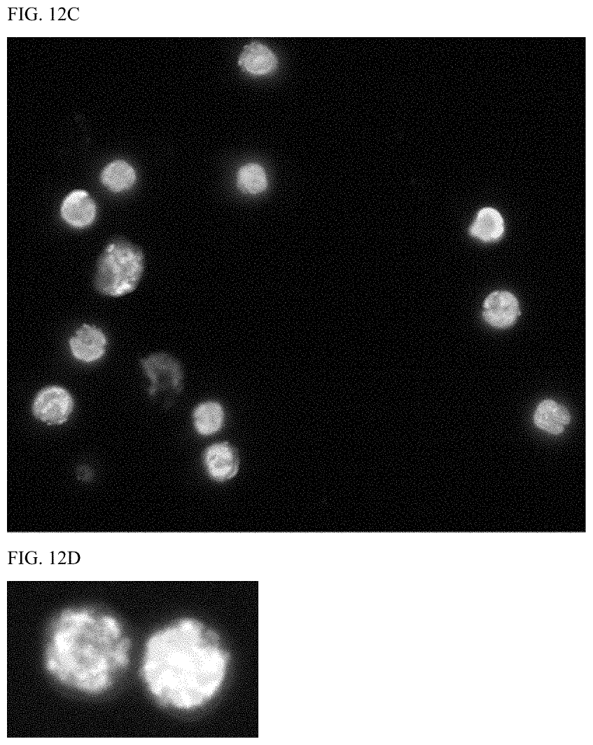

Effector T cells have small, distinct mitochondria dispersed in the cytoplasm, while memory T cells have densely packed, somewhat tubular, mitochondria. The individual mitochondria can be represented as subcellular points which can be analyzed and model using spatial point pattern analysis. These models can be helpful to understand T cell fate based on age and therapy. See, e.g., FIGS. 12A-12D.

### Example 8: Assays for Predicting Histone and Cytoskeletal Modifications

Modifications to histones and cytoskeleton may also be hallmarks of immune cell function and/or aging. PBMCs were treated with TSA (trichostatin A, an inhibitor of histone deacetylases), C646 (an inhibitor of histone acetyl transferase), 2-Hydroxyglutarate (suppresses dioxygenases involved in removing histone methylation), colchicine (inhibitor of polymerization and microtubule target), cytochalasin A (inhibitor of polymerization and actin target), jasplakinolide (enhancer of polymerization and actin target), paclitaxel (Taxol, inhibitor of depolymerization and microtubule target), or phalloidin (inhibitor of depolymerization and actin target), and stained and imaged using CellPainting Membrane (CPM) palette or CellPainting Epigenetic (CPE) palette.

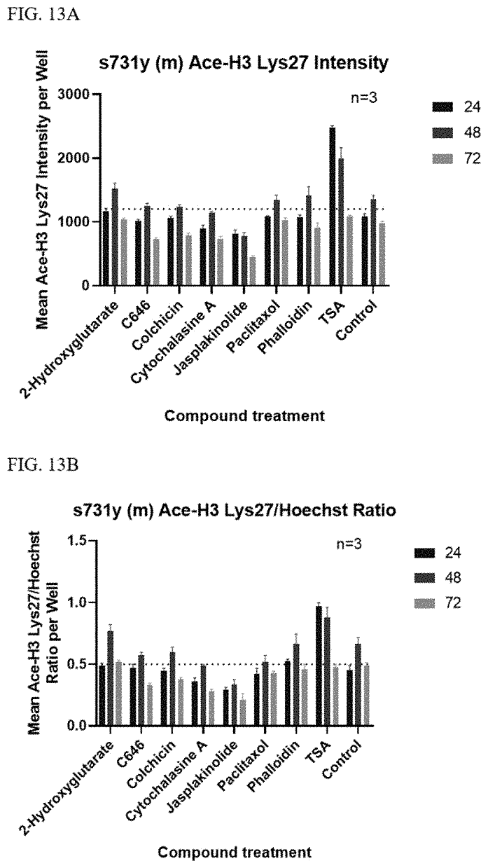

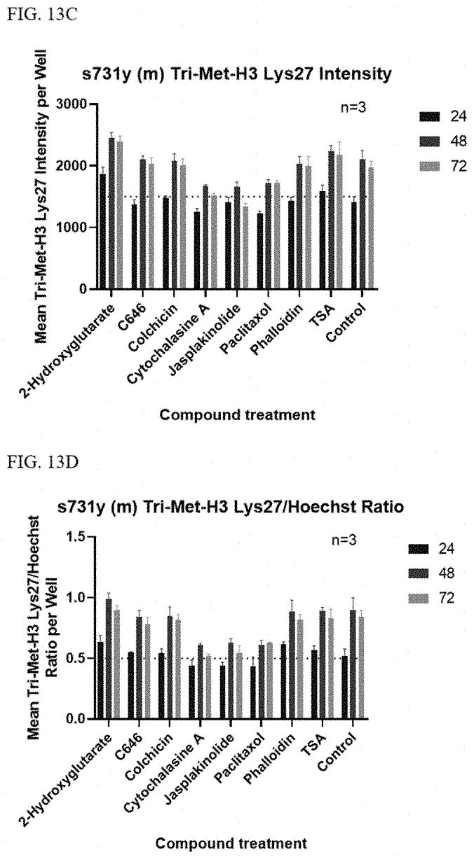

FIG. 13A to FIG. 13D show the intensity of signal from an anti-acetyl-Histone H3 (Lys27) binding reagent or Tri-Methyl-Histone H3 Lys27 after treatment with each compound for 24, 48, or 72 hours.

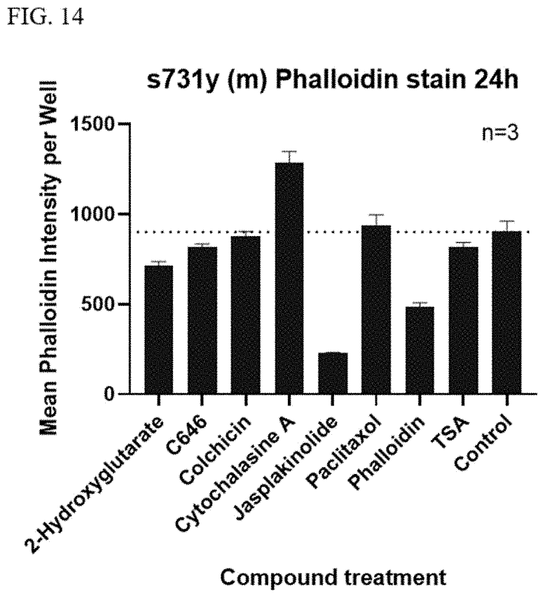

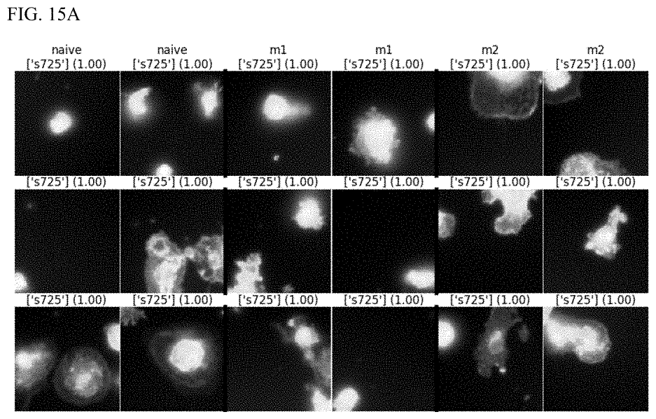

FIG. 14 shows the intensity of signal from a phalloidin stain 24 hours after compound treatment.

### Example 9: Assays for Evaluating T Cell Exhaustion

In chronic infections and cancer, T cells are exposed to persistent antigen and/or inflammatory signals. This scenario is often associated with the deterioration of T cell function: a state called “exhaustion”. Exhausted T cells lose robust effector functions, express multiple inhibitory receptors and are defined by an altered transcriptional program. T cell exhaustion is often associated with inefficient control of persisting infections and tumors, but revitalization of exhausted T cells can reinvigorate immunity.

Provided herein are assays for determining T cell exhaustion comprising staining T cells with the CellPainting Membrane (CPM) palette, the CellPainting Epigenetic (CPE) palette, the Cell Specific (CPS) palette, or the CellPainting Membrane+Cell Specific (CPMS) palette), and comparing the intensity of signals provided by the combination of markers within the stain to the intensity of signals produced by T cells of varying states of exhaustion.

Exhausted T cells may be defined by evaluation of IFNγ and/or IL-2, which are common markers for immune response. Phenotypic measurement of hallmarks of exhaustion in young versus old subjects versus functional stimulation is performed to evaluate exhaustion. Non-limiting examples of hallmarks for exhaustion of CD8+ T cells include: co-expression of multiple inhibitory receptors such as PD-1, CTLA-4, LAG-3, TIM-3, 2B4/CD244/SLAMF4, CD160, and/or TIGIT; loss of IL-2 production, proliferative capacity, ex vivo cytolytic activity; impairment of production of TNF-alpha, IFN-gamma, and/or cc (beta) chemokines; degranulation; expression of high levels of Granzyme B; poor responsiveness to IL-7 and/or IL-15 (drive memory T cell antigen-dependent proliferation long after antigen elimination); and cell death (e.g., may be due to overstimulation). Non-limiting examples of hallmarks for exhaustion of CD4+ T cells include: co-expression of multiple inhibitory receptors such as PD-1, CTLA-4, LAG-3, TIM-3, 2B4/CD244/SLAMF4, CD160, and/or TIGIT; loss of IL-2 production, proliferative capacity, ex vivo cytolytic activity; impairment of production of TNF-alpha, IFN-gamma, and/or cc (beta) chemokines; altered expression of GATA-3, Bcl-6, and/or Helios; similarity to a T Follicular Helper (Tfh) cell phenotype (e.g., surface markers such as CD4, CXCR5, ICOS, and/or PD-1; secreted cytokines such as IL-4, IL-6, and/or IL-21; transcription factors such as Bcl-6, IRF4, and/or STAT4); and earlier manifestation of dysfunction compared to CD8+ Tex cells.

### Example 10: Assays Evaluating In Vitro Cell Aging

To evaluate the age of PBMC cells from a subject, the following experiments are performed: the CellPainting Membrane (CPM) experiment, CellPainting Epigenetic (CPE) experiment, Cell Specific (CPS) experiment, and CellPainting Membrane+Cell Specific (CPMS) experiment, each with two 384-well plates per experiment, and two replicates per plate. There are 48 donors per plate, 96 donors per experiment, and eight replicates per donor on a 384 well plate. 10,000 cells per well are used (80,000 cells per donor per plate, 240,000 cells per donor per experiment). An overview of each experiment is provided below. Flow analysis is performed at 24 hours on each of the 96 donors.

For the CPM experiments, imaging is performed with the following parameters: 4 FOV, 20× water lens, wide field (yields approximately 1500-2000 events (cells)), Brightfield, Hoechst, WGA, Phalloidin, Concanavalin A, and a stain or other method for distinguishing between live and dead cells (e.g., one or more of DAPI, propidium iodide (PI), and a Zombie dye).

For the CPE experiments, imaging is performed with the following parameters: 4 FOV, 40× water lens, confocal 4×z-stack (yields approximately 400-500 events (cells)), Brightfield, Hoechst, Open Chromatin (H3K27ac), Closed Chromatin (H3K27me3), Mitotracker®, and a stain or other method for distinguishing between live and dead cells (e.g., one or more of DAPI, propidium iodide (PI), and a Zombie dye).

For the CPS experiments, imaging is performed with the following parameters: 4 FOV, 20× water lens, wide field (yields approximately 1500-2000 events (cells)), Brightfield, Hoechst, T Cells (CD3), B Cells (CD19), Monocyte/Macrophage (CD14), Monocyte/Dendritic Cell (CD11c), and NK Cells (CD56).

For the CPMS experiments, imaging is performed with the following parameters: 4 FOV, 20× water lens, wide field (yields approximately 1500-2000 events (cells)), Brightfield, Hoechst, T Cells (CD3), Phalloidin, ConcanavalinA, and Monocyte/Dendritic Cell (CD11c). Cell aging may be evaluated using one, two, or a combination of all three assays.

### Example 11: Assays for Evaluating Aging in Certain Cell Types

To evaluate cell aging in specific cell types from PBMCs, PBMCs are compared to known samples of young (from subjects under 30 years old) and old (from subjects over 50 years old). An aging segmentation experiment is performed on three samples each of young and old samples: T cells, B cells, monocytes, NK cells, macrophages, and dendritic cells to provide the training set of known samples.

### Example 12: Assays Characterizing Cell Aging and/or Immune Cell Function

The state, function, and/or age of a cell is determined using one or more in vitro assays described herein. Briefly, the assays comprise contacting the cell with a plurality of binding reagents each capable of recognizing and binding a marker of the cell and determining the cell's morphological signature and/or functional signature based on the intensity of the signal associated with each the binding reagent. The binding reagents include those provided in the palettes described in Example 2: the CellPainting Membrane (CPM) palette, the CellPainting Epigenetic (CPE) palette, the Cell Specific (CPS) palette, and the CellPainting Membrane+Cell Specific (CPMS) palette.

In a first assay, cell to cell interactions of the immune system (e.g., T cell interactions with APCs) are evaluated by staining cells with CPM or CPE, and quantifying any interactions by comparison between the resulting images and a panel of images from known cell to cell interactions (as performed in Example 3).

In another assay, T cell activation in a cell is evaluated by imaging spatial changes to the T cell. The cell is labeled with binding reagents capable of recognizing the cell's morphological signature, including: cytoskeletal changes, rearrangement of mitochondria, and the presence or absence of TCR microclusters.

In another assay, cytokine communication within a sample of PBMCs is determined by activating the PBMCs with stimulators (e.g., LPS, PHA or ConA, PWM). The signature of cytokines released is compared with signatures associated with known cytokine mediated intercellular communications.

In another assay, the change of a macrophage with age is determined by staining the macrophage using CPM or CPE staining procedure, and predicting whether the macrophage has a M1 or M2 phenotype using the training model described in Example 6.

In another assay, the state of a T cell is determined by sorting T cells from PBMCs using flow cytometry and/or cell imaging with a CellPainting or CellSpecific method as described in Example 1 and Example 2. T cells are first identified as CD3+ or as CD3+, CD19−, CD14−, and CD11c−. The T cells are contacted with binding reagents capable of recognizing and binding the following markers: CD3, CD19, CD14, and CD11c. The morphological signature of the T cells are determined to distinguish between effector T cells having small, distinct mitochondria dispersed in the cytoplasm, and memory T cells having densely packed, somewhat tubular mitochondria.

In another assay, histone and/or cytoskeletal modifications are evaluated by imaging a cell using CPM or CPE and comparing the imaged cell to cells having certain hallmarks of immune cell function and/or aging, such images generated using the method of Example 8.

Any of the aforementioned assays may be performed on one or more samples from a patient in order to characterize cell aging and/or immune function. The characterization of a stained sample may be performed using a computer trained using machine learning or the like with a data set comprising stained samples of known cell state, function, and/or predicted age.

### Example 13: Screening Methods for Identifying Agents for the Treatment of Cell Aging and/or Immune Cell Function

Test agents are evaluated as being capable of changing a cell's state, function, and/or predicted age using an in vitro assay described herein. The assays include contacting the cell with a test agent and a plurality of binding reagents each capable of recognizing and binding a marker of the cell; then determining the cell's morphological signature and/or functional signature based on the intensity of a signal associated with the binding reagents. The binding reagents include those provided in the palettes described in Example 2: the CellPainting Membrane (CPM) palette, the CellPainting Epigenetic (CPE) palette, the CellSpecific (CPS) palette, and the CellPainting Membrane+Cell Specific (CPMS) palette.

In a first assay, the ability of test agent to change cell to cell interactions is evaluated by contacting the cell with the test agent, staining the cells with CPM or CPE, and comparing the changes in images with and without the test agent. The images may be compared with a panel of images from known cell to cell interactions (as performed in Example 3).

The ability of a test agent to affect T cell activation is determined by contacting T cells with the test agent and binding reagents (e.g., binding reagents capable of recognizing the cell's morphological signature, including: cytoskeletal changes, rearrangement of mitochondria, and the presence or absence of TCR microclusters), imaging the cells, and comparing the imaged cells to known T cell activation states.

The ability of a test agent to alter immune cell communication is determined. PBMCs are contacted with test agent and then stimulated with a stimulator (e.g., LPS, PHA or ConA, PWM). The signature of cytokines released is compared with signatures associated with known cytokine mediated intercellular communications to evaluate the effect of test agent on cellular communication.

The ability of a test agent to prevent macrophage change associated with aging is determined by contacting a macrophage with the testing agent in vitro, followed by determining the morphology of the macrophage after stimulation with LPS, IFNγ, mCSF, IL4, IL13, and/or GM-CSF and imaging using CPM or CPE.

The ability of a test agent to affect T cell fate is determined by contacting a T cell with the testing agent in vitro, followed by determining a change in the cell's state by contacting the cell with a binding reagent capable of recognizing and binding a marker of the cell indicative of the cell fate. The marker is specific for the morphology of the mitochondria, where if the mitochondria is small and dispersed in the cytoplasm, the cell may be characterized as an effector T cell, and if the mitochondria is densely packed and somewhat tubular, the cell may be characterized as a memory T cell. The ability of the test agent to affect cell age using mitochondria morphology of cells with and without test agent treatment is evaluated.

The ability of a test agent to affect histone and/or cytoskeletal modifications is evaluated by contacting a test cell with the test agent in vitro and then imaging the cell using CPM or CPE. The image is compared to cells having certain hallmarks of immune cell function and/or aging, such images generated using the method of Example 8.

### Example 14: Preclinical Influenza Vaccine Model in Mice

A scalable Influenza vaccine model is developed. The model is used to evaluate the efficacy of known anti-influenza drugs in mice. The model is then used to identify a novel therapeutic for treatment of Influenza and/or a novel vaccine for Influenza. The treatment may be specific for elderly patients or any desired age group.

Experimental reagents include: PR8 recombinant nucleoprotein (vaccine analog with known dosing), mouse-adapted PR8 flu (a 1934 H1N1 flu strain discovered in Puerto Rico with well-established and known LD50), drug compounds of interest, young C57/B16 Mice (defined as 12-14 weeks of age), and old C57/B16 Mice (defined as 72-80 weeks of age). Animals are grouped with 10 mice per group as follows: young flu only control (virus), old flu only control (virus only), young vaccine control (virus+h1NP+PBS), old vaccine control (virus+h1NP+PBS), young experimental (virus+h1NP+drug of interest), and old experimental (virus+h1NP+drug of interest). Fourteen days prior to treatment with PR8 NP (Day-14), mice are started on drug treatment. Fourteen days later (Day 0), mice are treated with PR8 NP. On day 10, mice are given a second PR8 treatment. On day 28, mice are infected with PR8 influenza particles. On day 38, mice are sacrificed. Mice are evaluated for efficacy of drug treatment to prevent and/or treat Influenza challenge at various time points. This experiment is replicated to develop a, SARS-CoV-2 vaccine model in mice.

### Example 15: Aging Phenotypes of the Immune System Evaluated Using Fluorescent Microscopy

Two palettes were utilized to stain key cellular organelles to characterize aging phenotypes of the immune system:

CellPainting Palette I: cells were stained using CellPainting Palette I protocol to profile the morphological state and epigenetic landscape of PBMCs for classifying cells as young versus old. Fluorescent lectins like Wheat Germ Agglutinin (WGA) and Concanavalin (ConA) selectively bind to glycoprotein and glycolipids in biological members. Phalloidin has specificity for F-actin within cells. In this experiment, fluorescent reagents include: WGA, Alexa Fluor™ 488 conjugate (Invitrogen), Texas Red™-X Phalloidin (Invitrogen), Concanavalin A, Alexa Fluor™ 633 conjugate (Invitrogen), Acetyl-Histone H3 (Lys27) monoclonal mouse antibody (Invitrogen), Tri-Methyl-Histone H3 (Lys27) (C36B11) rabbit monoclonal antibody (Cell Signaling), goat anti-rabbit IgG (H+L) cross-adsorbed secondary antibody, Alexa Fluor 700 (Invitrogen), and goat anti-mouse IgG (H+L) cross-adsorbed secondary antibody, Alexa Fluor 514 (Invitrogen). Each component of CellPainting Palette was diluted as follows in Block buffer: WGA, Alexa Fluor™ 488 conjugate (1:2000), Texas RedT™-X Phalloidin (1:400), Concanavalin A, Alexa Fluor™ 633 conjugate (1:80), Acetyl-Histone H3 (Lys27) monoclonal mouse antibody (1:500), Tri-Methyl-Histone H3 (Lys27) (C36B11) rabbit monoclonal antibody (1:3000), and Hoechst 33342 (1:10000). Briefly, PBMCs were platted in an assay plate (384 well Screenstar COC Imaging plate, Greiner BioOne) and incubated with Block Buffer (DPBS supplemented with 2% w/v BSA, 0.2% w/v fish gelatin, 1% v/v antibiotic/antimycotic, and 5% (1:20) normal human serum). 25 μL CellPainting Palette diluted in block buffer as described above was added to the wells and incubated for 18 hours at 4° C. The wells were washed with PBS three times and incubated with secondary antibody for 1 hour at room temperature. The wells were washed with PBS complemented with antibiotic/antimycotic and imaged as shown below.

Image acquisition conducted with binning 1 and 16 FOV with 20× water objective NA 1.0

| Channel | Label | Excitation | Emission | Comment |
|---|---|---|---|---|
| 1 | Brightfield | Transmission | 655-760 | |
| 2 | Hoechst 33342 | 355-385 | 430-500 | Nuclei |
| 3 | Alexa 488 | 460-490 | 500-550 | WGA |
| 4 | Alexa 633 | 615-645 | 655-705 | ConA |
| 5 | Texas Red | 530-560 | 570-650 | Phalloidin |
| 6 | Alexa 700_narrow | 650-675 | 685-760 | Tri-Met H3 |
| | | | | (Lys 27) |
| 7 | Alexa 514 | 490-515 | 525-580 | Ace-H3 |
| | | | | (Lys 27) |

CellPainting Palette II: Cells were stained using CellPainting Palette II protocol to profile the morphological state (Hoechst, WGA, Phalloidin) and mitochondrial activity/morphology of PBMCs for classifying cells as young versus old. MitoTracker® Orange CMTMRos is utilized to stain mitochondria in live cells, its accumulation is dependent upon membrane potential. FCCP (carbonyl cyanide 4-(trifluoromethoxy)phenylhydrazone) is used as a positive control, and is an ionophore which uncouples the respiration chain (ATP synthesis) by transporting protons through the mitochondrial membrane without generating energy for oxidative phosphorylation. Briefly, PBMCs were platted in an assay plate (384 well Screenstar COC Imaging plate, Greiner BioOne), cells are stimulated with FCCP as a control for MitroTracker® staining and MitoTracker®. Cells were washed, fixed, and permeabilized. Cells were blocked and stained using CellPainting Palette I as described above. The cells were imaged as shown below.

Image acquisition conducted with binning 1 and 16FOV with 20× water objective NA 1.0

| Channel | Label | Excitation | Emission | Comment |
|---|---|---|---|---|
| 1 | Brightfield | Transmission | 655-760 | |
| 2 | Hoechst 33342 | 355-385 | 430-500 | Nuclei |
| 3 | Alexa 488 | 460-490 | 500-550 | WGA |
| 4 | Alexa647_narrow | 615-645 | 655-705 | Phalloidin |
| 5 | MitoTracker ® | 530-560 | 570-650 | Mitochondria |
| | Orange | | | |

### Example 16: Viral System as a Screen for Improved Elderly Vaccine Response

Activation and response of various immune cells change with age and cause a decreased vaccine response. Macrophage polarization, TLR, antigen uptake, and T-cell priming have all been shown to be dysfunctional and implicated in a decrease response to vaccine. Biochemical changes in the activation signal pathways of the elderly adaptive immune system result in poor effector functions. There are two approved vaccines which slightly boost the elderly vaccine response, MF59 and AS01. MF59 establishes an immunocompetent environment at the injection site which promotes recruitment of immune cells, including antigen presenting cells (APCs), that are facilitated to engulf antigen and transport it to draining lymph node (dLN) where the antigen is accumulated. In vitro studies showed that MF59 promotes the differentiation of monocytes to dendritic cells (Mo-DCs). AS01 is also used in the Shingrix vaccine to activate the innate immune system to prime the downstream T-cells and B-cells.

The present example provides a viral system to elicit the differences between young and old cells in the context of innate activation, monocyte to dendritic cell differentiation, and antigen uptake. Computational modeling is performed to measure the various differences in young and old cells during activation. Activation can be induced through various stimulates that are either polyclonal (non-specific) or antigen specific. Non-specific activation can be done with superantigens such as Staphylococcal enterotoxin B (SEB), model antigens, anti-CD3/CD28 beads, and CD3-engagers. Antigen specific activation can be triggered by influenza virus and vaccine but the T-cell response may be reduced. Upon stimulation, the response across all PBMCs can be measured by a primary readout of broad immunofluorescence imaging and secondary readouts of flow cytometry, cytokines, expression, and other traditional markers of activation. The primary and secondary readouts can be used to teach a computational model to accurately identify the characteristics that define a young and an old response.

Non-antigen specific stimulants. Staphylococcal enterotoxin B (SEB) from Staphylococcus aureus activates monocytes/macrophages and T lymphocytes by binding MHC class II molecules and specific Vβ regions of T-cell receptors. SEB may be ordered from Sigma Aldrich, S4881. Anti-CD3/CD28-coated beads (Miltenyi Biotec) artificially stimulate T-cell CD3/CD28 receptors, but do not affect monocytes. LPS stimulates CD14/TLR4/MD2 receptor on monocytes, dendritic cells, macrophages, and B cells. Poly I:C stimulates TLR3 receptor on B-cells, macrophages, and dendritic cells. PMA/Ionomycin (Stemcell Technologies) activates T-cells in combination with PMA to express cytokines; it bypasses T-cell receptors.

Antigen-specific stimulants. Vesicular stomatitis virus (VSV) stimulates CD14/TLR4 receptor on monocytes, dendritic cells, and macrophages. VSV-IFNβ exhibits multiple mechanisms of action, including direct cell killing, stimulation of an innate immune response, recruitment of CD8 T-cells, and depletion of T regulatory cells. VSV-IFNs also promotes the establishment of a CD8 T-cell response to endogenous tumor antigens. Other suitable viral stimulates that can be used in the model are influenza, severe acute respiratory syndrome coronavirus 2 (SARS-CoV-2), HIV, and Ebola viruses.

Assay Development. To determine if a given stimulus can successfully induce T-cell activation and/or monocyte/dendritic cell activation, a first assay was developed as follows: two donors each of PBMC and T-cells (young and old) were assayed at two timepoints (24 hours and 48 hours) with four stimuli and four controls, using a CPM and CPE readout (see previous examples for CPM and CPE description), IL-2 and IFNγ levels as determined by ELISA, CD25 flow cytometry, and regular flow cytometry 5 color. The stimuli were: (1) beads+CD3/28, (2) beads=CD3/28+LPS (10 ng/ml), (3) beads+CD3/28+poly I:C (2 μg/ml), and (4) beads+CD3/28+VSV 10×MOI. The controls were: (1) beads+naïve, (2) beads+LPS, (3) beads+poly I:C, and (4) beads+VSV. A plate map of the experimental set up is shown below.

| | | beads + CD3/CD28 | | | | | | beads | | | | | | beads + CD3/CD28 | | | | | | beads | | | | | | Cell | Trigger |
|---|---|---|---|---|---|---|---|---|---|---|---|---|---|---|---|---|---|---|---|---|---|---|---|---|---|---|---|
| | | | 2 | 3 | 4 | 5 | 6 | 7 | 8 | 9 | 10 | 11 | 12 | 11 | 14 | 15 | 16 | 17 | 18 | 19 | 20 | 21 | 22 | 23 | 24 | Type | Medium |
| 10k | A | | | | | | | | | | | | | | | | | | | | | | | | | T-cell (y) | VSV |
| cells | B | | | | | | | | | | | | | | | | | | | | | | | | | PBMC (y) | 10x |
| | C | | | | | | | | | | | | | | | | | | | | | | | | | T-cell (o) | MOPOI |
| | D | | | | | | | | | | | | | | | | | | | | | | | | | PBMC (o) | |
| | E | | | | | | | | | | | | | | | | | | | | | | | | | T-cell (y) | LPS |
| | F | | | | | | | | | | | | | | | | | | | | | | | | | PBMC (y) | 10 ng/ |
| | G | | | | | | | | | | | | | | | | | | | | | | | | | T-cell (o) | ml |
| | H | | | | | | | | | | | | | | | | | | | | | | | | | PBMC (o) | |
| | I | | | | | | | | | | | | | | | | | | | | | | | | | T-cell (y) | Poly I:C |
| | J | | | | | | | | | | | | | | | | | | | | | | | | | PBMC (y) | 2 μ/ |
| | K | | | | | | | | | | | | | | | | | | | | | | | | | T-cell (o) | ml |
| | L | | | | | | | | | | | | | | | | | | | | | | | | | PBMC (o) | |
| | M | | | | | | | | | | | | | | | | | | | | | | | | | T-cell (y) | naive |
| | N | | | | | | | | | | | | | | | | | | | | | | | | | PBMC (y) | |
| | O | | | | | | | | | | | | | | | | | | | | | | | | | T-cell (o) | |
| | P | | | | | | | | | | | | | | | | | | | | | | | | | PBMC (o) | |

Stimulate and timepoints to be used in the production run were chosen based on: (1) the amount of activation as measured through cytokines and expression, (2) largest morphological/phenotypic change based on the images, and (3) highest cell to cell interaction based on the images.

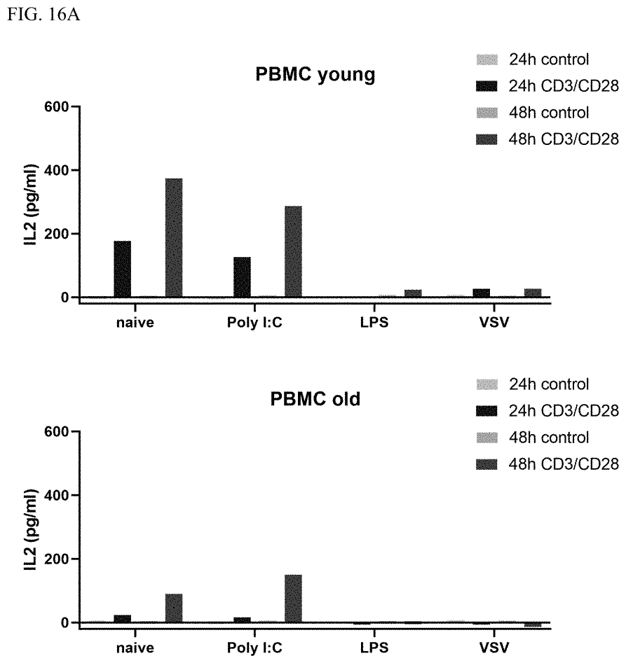

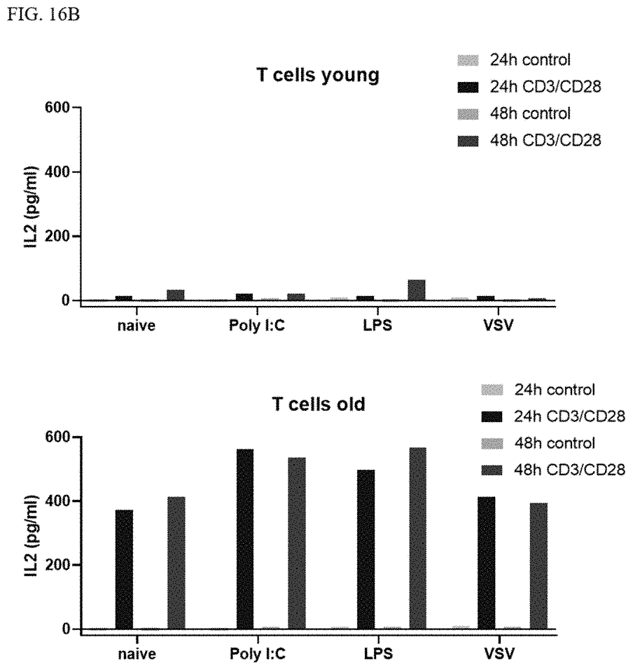

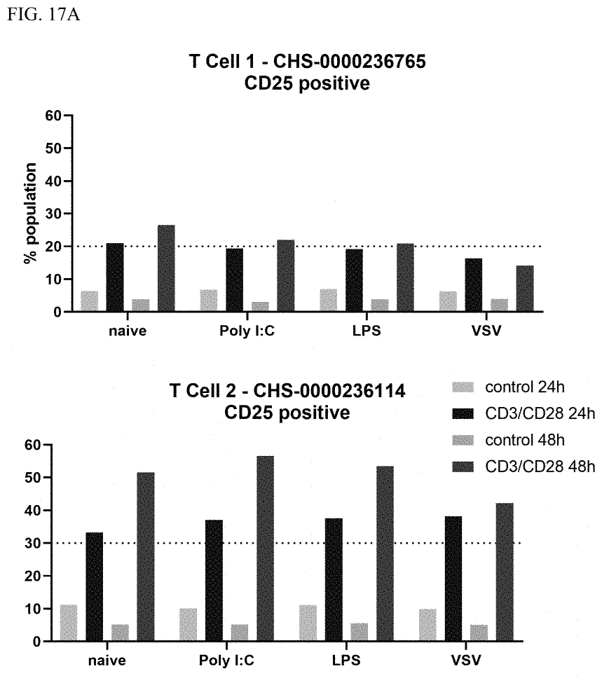

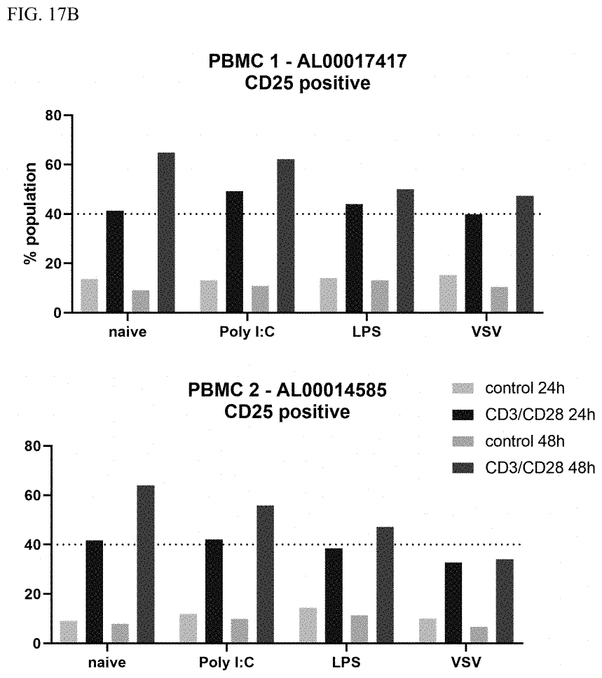

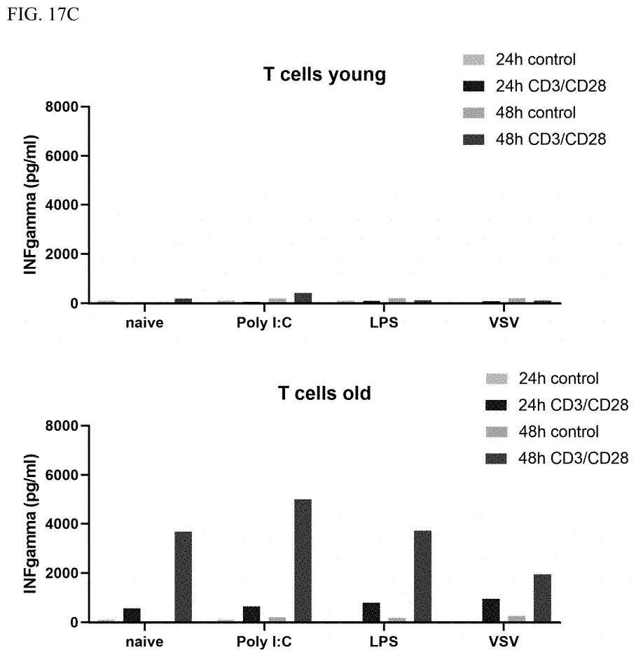

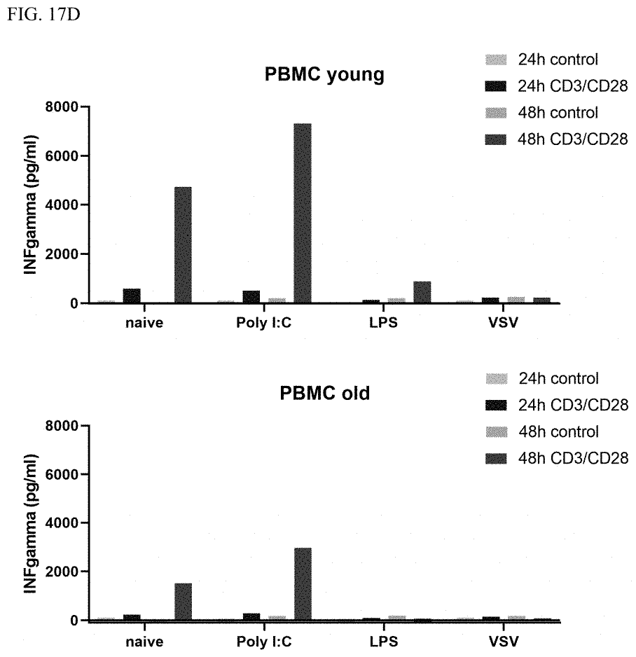

FIG. 16A shows IL2 concentration in young and old PBMCs 24 and 48 hours after stimulation. FIG. 16B shows IL2 concentration in young and old T-cells 24 and 48 hours after stimulation. FIG. 17A shows percentage of CD25 positive T-cell activation in young and adult T cells 24 and 48 hours after stimulation. FIG. 17B shows percentage of CD25 positive T-cell activation in young and adult PBMC cells 24 and 48 hours after stimulation. FIG. 17C shows IFNγ production in young and old T cells 24 and 48 hours after stimulation. FIG. 17D shows IFNγ production in young and old PBMC cells 24 and 48 hours after stimulation. FIG. 18A contains images of young PBMCs after 24 hours using the CPM panel. FIG. 18B contains images of young PBMCs after 48 hours using the CPM panel.

Based on an analysis of primary and secondary readouts of the various PBMC stimulation methods, the stimulation condition selected for the next step was VSV at 24 hours. IL-2, IFNγ, and CD25 are traditional, standard markers of activation. VSV stimulation suppresses the concentration of IL-2 and IFNγ. This suppression is a known biological mechanism which makes measuring activation through these means difficult. CD25 expression was increased in 24 hr VSV stimulation along with all other stimulates. However, CD25 expression in VSV was the lowest compared to all other stimulants.

A mathematical model was built to differentiate the different stimulates using the images and classify eight different stimulation states. For this experiment, the best performing states were VSV+anti-CD3/CD28 and VSV only at 48 hours. This may be caused by the strong phenotypes that both of these stimulates induce in the cells. The VSV expands the monocyte lineage cells and fluoresces the antigen that the cells ingest from the virus. The anti-CD3/CD28 causes the T-cells to clump together into large balls of activated T-cells. The performance improves when just VSV incubated for 48 hours is evaluated versus the control wells.

The results of this first assay indicates that using VSV as a stimulant allows for quantification of distinct imaging phenotypes across functions that are critical to vaccine response. Antigen uptake, TLR function and T-cell priming have all been shown to have various defects in the elderly innate immune system. Activating the cells using VSV and then imaging with the CPM and CPE assay allows us to profile these aspects of response. The next step performed was to look at the differences between young and old in a production run.

For T-cell activation, the anti-CD3/CD28 beads showed a strong phenotype in both the traditional and imaging analysis. All the markers were increased, activated T-cells aggregated closely in the images, it was easy to classify, and cell to cell interaction increased. However, this stimulant is artificial and acts through means which overstimulate the T-cells in an unnatural way.

Aging Model 1. This model was built to separate young activated cells from old activated cells. For instance, in a classifier comparing young vs. control (old), the classifier predicts if a given viral system (e.g., stimulant) induces a phenotype in elderly cells to respond like young activated cells. This may comprise performing a binary classification of the given viral system (e.g., stimulant) (e.g., into either an positive (young activated cells) category or a control (old activated cells) category).

Primary readouts used were CellPainting Membrane (CPM) and CellPainting Epigenetic (CPE). CPM parameters used were 4 FOV, 40× water lens, wide field, z-stack, 4 planes starting at—Sum with distance of 1.6 um, yields of approximately 400-500 events (cells), Brightfield, Hoechst, WGA, Phalloidin, and ConcanavalinA. CPE parameters used were 4 FOV, 40× water lens, confocal 4× z-stack starting at −1 um with distance of 1.0 um, yields of approximately 400-500 events (cells), Brightfield, Hoechst, open chromatin (H3K27ac), closed chromatin (H3K27me3), and Mitotracker®. Secondary readouts included quantification of IL-10, IFNγ, Granzyme B, and IL-1. The readouts were times at 24 hours, 48 hours, and 72 hours.

Samples and plating: Each stimulant was plated on its own plate. Only samples 35 years old or younger and 60 years old or older were used (49 samples 35 or younger and 43 samples 60 or older for a total of 92 donor tubes per experiment). Two plates per experiment for each of three stains (CPM, CPE, and CPMS) were used, with 1 replicate per plate, for a total of 12 plates. Each plate had 48 donors (mixed old and young), 96 PBMC donors per experiment, and 8 replicates per donor per plate. For each well, 10,000 cells were used, with 80,000 cells per donor per plate, and 240,000 cells per donor per experiment. A plate map is shown below with uneven for the young and even for the old.

| R | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 |
|---|---|---|---|---|---|---|---|---|---|---|---|---|---|---|---|---|---|---|---|---|---|---|---|---|
| A | 1 | 1 | 2 | 2 | 3 | 3 | 4 | 4 | 5 | 5 | 6 | 6 | 7 | 7 | 8 | 8 | 9 | 9 | 10 | 10 | 11 | 11 | 12 | 12 |
| B | 25 | 25 | 26 | 26 | 27 | 27 | 28 | 28 | 29 | 29 | 30 | 30 | 31 | 31 | 32 | 32 | 33 | 33 | 34 | 34 | 35 | 35 | 36 | 36 |
| C | 7 | 7 | 8 | 8 | 9 | 9 | 10 | 10 | 11 | 11 | 12 | 12 | 1 | 1 | 2 | 2 | 3 | 3 | 4 | 4 | 5 | 5 | 6 | 6 |
| D | 31 | 31 | 32 | 32 | 33 | 33 | 34 | 34 | 35 | 35 | 36 | 36 | 30 | 30 | 29 | 29 | 28 | 28 | 27 | 27 | 26 | 26 | 25 | 25 |
| E | 13 | 13 | 14 | 14 | 15 | 15 | 16 | 16 | 17 | 17 | 18 | 18 | 19 | 19 | 20 | 20 | 21 | 21 | 22 | 22 | 23 | 23 | 24 | 24 |
| F | 37 | 37 | 38 | 38 | 39 | 39 | 40 | 40 | 41 | 41 | 42 | 42 | 43 | 43 | 44 | 44 | 45 | 45 | 46 | 46 | 47 | 47 | 48 | 48 |
| G | 19 | 19 | 20 | 20 | 21 | 21 | 22 | 22 | 23 | 23 | 24 | 24 | 13 | 13 | 14 | 14 | 15 | 15 | 16 | 16 | 17 | 17 | 18 | 18 |
| H | 42 | 42 | 43 | 43 | 44 | 44 | 45 | 45 | 46 | 46 | 47 | 47 | 42 | 42 | 41 | 41 | 40 | 40 | 39 | 39 | 38 | 38 | 37 | 37 |
| I | 1 | 1 | 2 | 2 | 3 | 3 | 4 | 4 | 5 | 5 | 6 | 6 | 7 | 7 | 8 | 8 | 9 | 9 | 10 | 10 | 11 | 11 | 12 | 12 |
| J | 25 | 25 | 26 | 26 | 27 | 27 | 28 | 28 | 29 | 29 | 30 | 30 | 31 | 31 | 32 | 32 | 33 | 33 | 34 | 34 | 35 | 35 | 36 | 36 |
| K | 7 | 7 | 8 | 8 | 9 | 9 | 10 | 10 | 11 | 11 | 12 | 12 | 1 | 1 | 2 | 2 | 3 | 3 | 4 | 4 | 5 | 5 | 6 | 6 |
| L | 31 | 31 | 32 | 32 | 33 | 33 | 34 | 34 | 35 | 35 | 36 | 36 | 30 | 30 | 29 | 29 | 28 | 28 | 27 | 27 | 26 | 26 | 25 | 25 |
| M | 13 | 13 | 14 | 14 | 15 | 15 | 16 | 16 | 17 | 17 | 18 | 18 | 19 | 19 | 20 | 20 | 21 | 21 | 22 | 22 | 23 | 23 | 24 | 24 |
| N | 37 | 37 | 38 | 38 | 39 | 39 | 40 | 40 | 41 | 41 | 42 | 42 | 43 | 43 | 44 | 44 | 45 | 45 | 46 | 46 | 47 | 47 | 48 | 48 |
| O | 19 | 19 | 20 | 20 | 21 | 21 | 22 | 22 | 23 | 23 | 24 | 24 | 13 | 13 | 14 | 14 | 15 | 15 | 16 | 16 | 17 | 17 | 18 | 18 |
| P | 42 | 42 | 43 | 43 | 44 | 44 | 45 | 45 | 46 | 46 | 47 | 47 | 42 | 42 | 41 | 41 | 40 | 40 | 39 | 39 | 38 | 38 | 37 | 37 |

Results of the Aging Model 1—ConcanavalinA staining. There were noticeable differences in staining with concanavalin between young and old cells. FIG. 19 shows correlations between texture in the ConcanvalinA stain are correlated with age. ConcanavalinA stains for glycoproteins on the cell surfaces and intracellular structures. Glycosylation is increased in the elderly, which is hypothesized to contribute to dysfunctional T-cell activation, which critical for effective vaccine response.

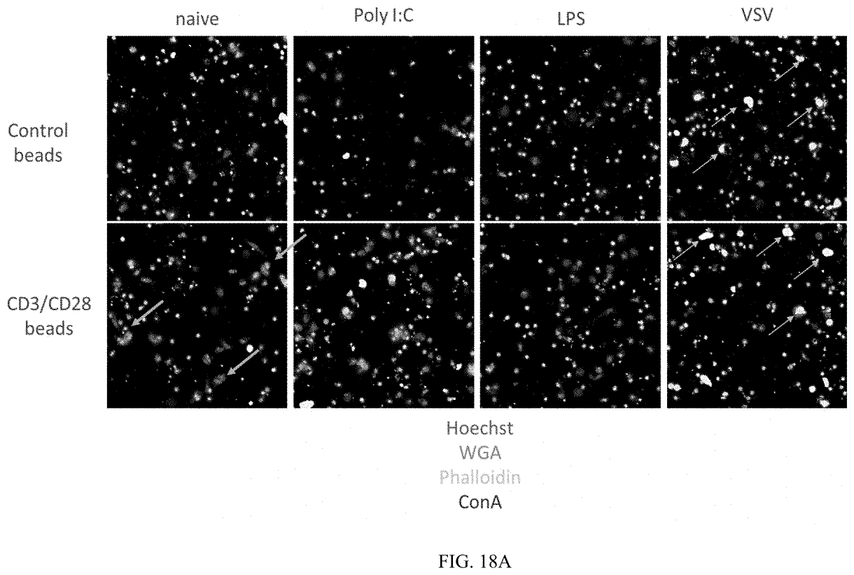

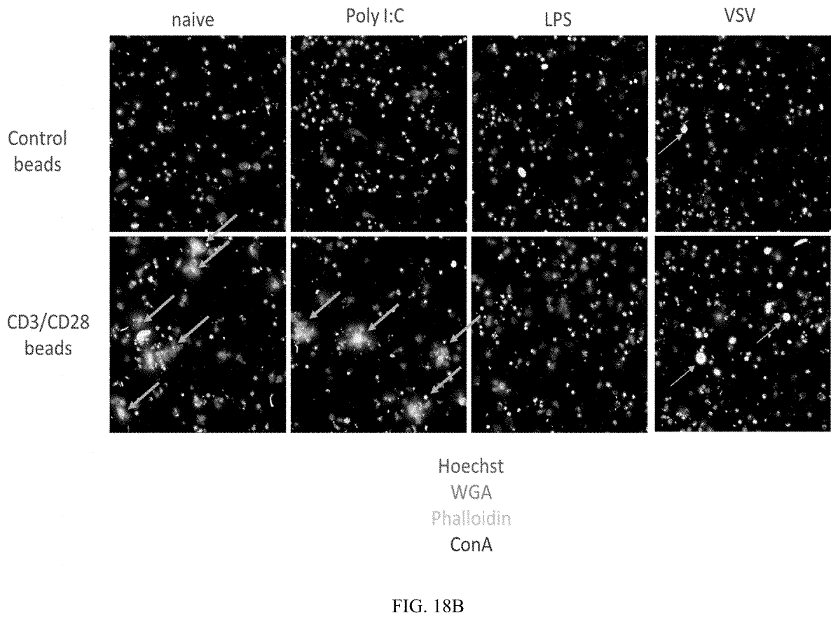

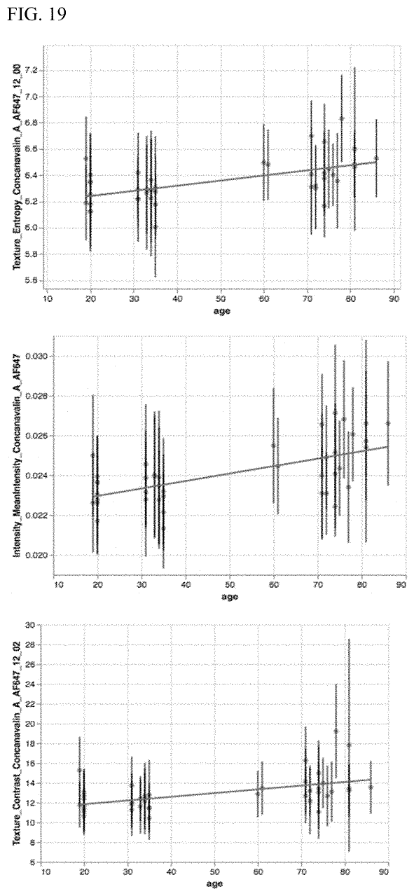

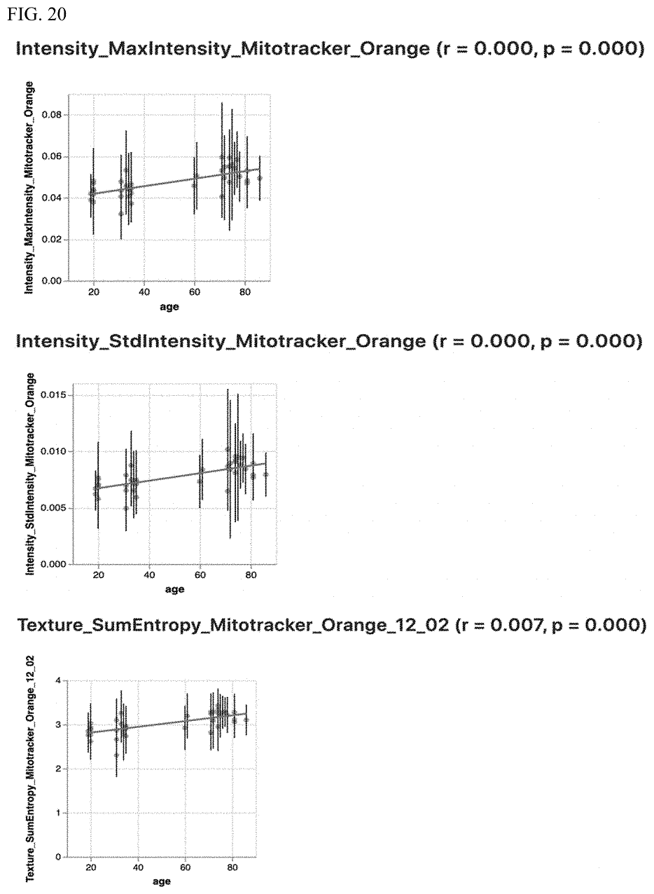

Results of the Aging Model 1—Mitotracker®. FIG. 20 shows that Mitotracker® intensity and entropy increases with age. The younger mitochondria is smaller, dispersed, and network-like, whereas the older mitochondria is larger, concentrated, and bulky. Mitochondria entropy is increased in the elderly, which may cause decreased viral recognition and T-cell activation leading to poor vaccine response.

AgingMode3. The cell staining palette in this model is CPMM, which includes Brightfield, Hoechst, WGA, Mitotracker®+VSV mCherry, ConA, and Phalloidin. Samples and plating: Each stimulant is plated on its own plate. Only samples 35 years old or younger and 60 years old or older are used (49 samples 35 or younger and 43 samples 60 or older for a total of 92 donor tubes per experiment). Two plates per experiment (CPMM) are used—two plates with 1×MOI (90 donors) and two plates with 10×MOI (90 donors), for a total of 4 plates. Each plate has 48 donors (mixed old and young), 92 PBMC donors per experiment, and 8 replicates per donor per plate. FirePlex Cytokine determination is performed for all plates. No flow cytometry is performed. A plate map is shown below with uneven for the young and even for the old.

| R | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 |
|---|---|---|---|---|---|---|---|---|---|---|---|---|---|---|---|---|---|---|---|---|---|---|---|---|
| A | 1 | 1 | 2 | 2 | 3 | 3 | 4 | 4 | 5 | 5 | 6 | 6 | 7 | 7 | 8 | 8 | 9 | 9 | 10 | 10 | 11 | 11 | 12 | 12 |
| B | 25 | 25 | 26 | 26 | 27 | 27 | 28 | 28 | 29 | 29 | 30 | 30 | 31 | 31 | 32 | 32 | 33 | 33 | 34 | 34 | 35 | 35 | 36 | 36 |
| C | 7 | 7 | 8 | 8 | 9 | 9 | 10 | 10 | 11 | 11 | 12 | 12 | 1 | 1 | 2 | 2 | 3 | 3 | 4 | 4 | 5 | 5 | 6 | 6 |
| D | 31 | 31 | 32 | 32 | 33 | 33 | 34 | 34 | 35 | 35 | 36 | 36 | 30 | 30 | 29 | 29 | 28 | 28 | 27 | 27 | 26 | 26 | 25 | 25 |
| E | 13 | 13 | 14 | 14 | 15 | 15 | 16 | 16 | 17 | 17 | 18 | 18 | 19 | 19 | 20 | 20 | 21 | 21 | 22 | 22 | 23 | 23 | 24 | 24 |
| F | 37 | 37 | 38 | 38 | 39 | 39 | 40 | 40 | 41 | 41 | 42 | 42 | 43 | 43 | 44 | 44 | 45 | 45 | 46 | 46 | 47 | 47 | 48 | 48 |
| G | 19 | 19 | 20 | 20 | 21 | 21 | 22 | 22 | 23 | 23 | 24 | 24 | 13 | 13 | 14 | 14 | 15 | 15 | 16 | 16 | 17 | 17 | 18 | 18 |
| H | 42 | 42 | 43 | 43 | 44 | 44 | 45 | 45 | 46 | 46 | 47 | 47 | 42 | 42 | 41 | 41 | 40 | 40 | 39 | 39 | 38 | 38 | 37 | 37 |
| I | 1 | 1 | 2 | 2 | 3 | 3 | 4 | 4 | 5 | 5 | 6 | 6 | 7 | 7 | 8 | 8 | 9 | 9 | 10 | 10 | 11 | 11 | 12 | 12 |
| J | 25 | 25 | 26 | 26 | 27 | 27 | 28 | 28 | 29 | 29 | 30 | 30 | 31 | 31 | 32 | 32 | 33 | 33 | 34 | 34 | 35 | 35 | 36 | 36 |
| K | 7 | 7 | 8 | 8 | 9 | 9 | 10 | 10 | 11 | 11 | 12 | 12 | 1 | 1 | 2 | 2 | 3 | 3 | 4 | 4 | 5 | 5 | 6 | 6 |
| L | 31 | 31 | 32 | 32 | 33 | 33 | 34 | 34 | 35 | 35 | 36 | 36 | 30 | 30 | 29 | 29 | 28 | 28 | 27 | 27 | 26 | 26 | 25 | 25 |
| M | 13 | 13 | 14 | 14 | 15 | 15 | 16 | 16 | 17 | 17 | 18 | 18 | 19 | 19 | 20 | 20 | 21 | 21 | 22 | 22 | 23 | 23 | 24 | 24 |
| N | 37 | 37 | 38 | 38 | 39 | 39 | 40 | 40 | 41 | 41 | 42 | 42 | 43 | 43 | 44 | 44 | 45 | 45 | 46 | 46 | 47 | 47 | 48 | 48 |
| O | 19 | 19 | 20 | 20 | 21 | 21 | 22 | 22 | 23 | 23 | 24 | 24 | 13 | 13 | 14 | 14 | 15 | 15 | 16 | 16 | 17 | 17 | 18 | 18 |
| P | 42 | 42 | 43 | 43 | 44 | 44 | 45 | 45 | 46 | 46 | 47 | 47 | 42 | 42 | 41 | 41 | 40 | 40 | 39 | 39 | 38 | 38 | 37 | 37 |

Compound testing with Aging Model 3. Cell plating is identical to the aging model. 14 test and 2 control compounds are analyzed. Old donors who at least 60 years old are tested with all 16 compounds. Young donors who are 35 years old or younger are treated with control (DMSO) only.

The table below shows the 14 test compounds.

| | CAS# | Name | iID | sID | Target |
|---|---|---|---|---|---|
| 1 | 1005342-46-0 | LCL-161 | i156015 | s156043 | IAP |
| 2 | 1009820-21-6 | CX-4945 | i152914 | s152942 | CK2 |
| 3 | 143664-11-3 | Elacridar | i153525 | s153553 | |
| 4 | 206361-99-1 | Darunavir | i152659 | s152687 | COVID Control |
| 5 | 585543-15-3 | Losmapimod | i155358 | s155386 | p38 |
| 6 | 1230487-00-9 | Siponimod | i153321 | s153349 | S1P |
| 7 | 796967-16-3 | Linifanib | i153734 | s153762 | VEGF, PDGFR. Flt3, and |
| | | | | | others |
| 8 | 1115-70-4 | Metformin | i153964 | s153992 | Gerotherapeutic |
| | | hydrochloride | | | |
| 9 | 155141-29-0 | Rosiglitazone Maleate | i153869 | s153897 | Gerotherapeutic |
| 10 | 2.09410-46-8 | Neflamapimod | i153520 | s153548 | p38 |
| 11 | 836683-15-9 | Acumapimod | i152085 | s152113 | p38 |
| 12 | 162359-55-9 | Fingolimod | i155410 | s155438 | S1P |
| 13 | 747-36-4 | Hydroxychloroquine | i155159 | s155187 | COVID Control |
| | | sulfate | | | |
| 14 | 781649-09-0 | Telcagepant/MK-0974 | i153046 | s153074 | CGRP |

Aging Model 4. The cell staining palette in this model is CPMM, which includes Brightfield, Hoechst, WGA, Mitotracker®+VSV mCherry, ConA, and Phalloidin.

Samples and plating. Each stimulant is plated on its own plate. Only samples 35 years old or younger and 60 years old or older are used (49 samples 35 or younger and 43 samples 60 or older, 89 donor tubes per experiment). Two plates per experiment with CPMM 10×MOI (89+2 donors). Two donors who are greater than 60 years old are included from high density iXCells. Each plate has 48 donors (mixed old and young), 89+2 PBMC donors per experiment, and 8 replicates per donor per plate. FirePlex Cytokine determination is performed for all plates. No flow cytometry is performed. A plate map is shown below with uneven for the young and even for the old.

Compound testing with Aging Model 4. 14 test compounds are tested at 0.25 μM and 5 μM. There are 2 DMSO controls, with DMSO on both plates in a different position. The table below shows the 14 test compounds.

| | CAS# | Name | iID | sID | Target |
|---|---|---|---|---|---|
| 1 | 1005342-46-0 | LCL-161 | i156015 | s156043 | IAP |
| 2 | 1009820-21-6 | CX-4945 | i152914 | s152942 | CK2 |
| 3 | 143664-11-3 | Elacridar | i153525 | s153553 | |
| 4 | 206361-99-1 | Darunavir | i152659 | s152687 | COVID Control |
| 5 | 585543-15-3 | Losmapimod | i155358 | s155386 | p38 |
| 6 | 1230487-00-9 | Siponimod | i153321 | s153349 | S1P |
| 7 | 796967-16-3 | Linifanib | i153734 | s153762 | VEGF, PDGFR. Flt3, and |
| | | | | | others |
| 8 | 1115-70-4 | Metformin | i153964 | s153992 | Gerotherapeutic |
| | | hydrochloride | | | |
| 9 | Project 7065 | Rapamycin | | | mTor |
| 10 | 2.09410-46-8 | Neflamapimod | i153520 | s153548 | p38 |
| 11 | 836683-15-9 | Acumapimod | i152085 | s152113 | p38 |
| 12 | 162359-55-9 | Fingolimod | i155410 | s155438 | S1P |
| 13 | 747-36-4 | Hydroxychloroquine | i155159 | s155187 | COVID Control |
| | | sulfate | | | |
| 14 | 781649-09-0 | Telcagepant/MK-0974 | i153046 | s153074 | CGRP |

Although the present example utilizes VSV as a stimulate, in some embodiments other viruses are used as a stimulate, including the SARS-CoV-2.

The described systems can distinguish between young and old response to viruses in many immune cell types. The experiments performed showed that T-cell composition decreases when viral load increases, with elderly T-cell composition decreasing faster in response to the virus than the in the young samples. Mitochondrial entropy is increased in the elderly, which is hypothesized to decrease viral recognition and T-cell action leading to poor response. Furthermore, glycosylation is increased in the elderly, which is hypothesized to contribute to dysfunctional T-cell action that is critical for effective response. In addition, elderly viral cytokine response showed significant differences from the young. Because the systems can distinguish between young and old response to viruses in immune cells, the system can be used to discover and target the aging mechanisms which cause decreased response to vaccine and viruses. Using high throughput screening, thousands of therapeutics can be profiled to build a database of impact on immune response.

### Example 17: Screen for Improved Elderly Viral Response

A library of 5000 compounds is screened at two concentrations, 1 μM and 10 μM, or 0.5 μM and 5 μM. Another library or libraries may be screened that include one or more of: approved small molecule drugs, approved immunomodulator biologics, other approved biologics, or other small molecule drugs. For instance, a library may include all approved small molecule drugs.

Compounds are spotted in a 384 Greiner small volume polypropylene plate (#784201) in two different volumes and filled with trigger medium during the cell attachment step. Compounds are spotted in Meander by row, ensuring that both concentrations of the compound are applied to the same donor.

Cells from 42 subjects aged 60 or older (“elderly”) are tested. PBMCs are exposed to VSV-G mCherry at 10× multiplicity of infection (MOI). The supernatant is harvested after 24 hours of incubation before the cells are fixed for CPMM or Mitotracker® live staining. The supernatant is kept at −80° C. for all experiments. The supernatant is tested for quantification of cytokine levels (IFNγ, IL-1β, IL-2, IL-4, IL-6, IL-8, IL-10, IL-17A, MCP1, TNFα). Cells are imaged using the CPMM palette, which includes Brightfield, Hoechst, WGA, Mitotracker®+VSV mCherry, ConA, and Phalloidin.

The images are analyzed to determine which compounds make PBMCs from the elderly subjects look more like young PBMCs in the presence of viral stimulants. These compounds are selected as potentially clinically relevant. For instance, in a classifier comparing young vs. control, the classifier predicts if a given compound induces a phenotype that looks like young PBMCs. This may comprise performing a binary classification of the compound (e.g., into either an positive (young) category or a control category).

An overview of the phenotypes and models evaluated is shown in the table below.

| | |
|---|---|
| Readout | Canonical model link |

Composition

| | |
|---|---|
| T-cell percentage | Model: VSV, mongo |
| NK cell percentage | |
| Loaded monocyte percentage | |
| Lymphocyte/monocyte ratio | |

Aging models

| | |
|---|---|
| T-cells | AUC: 0.81 CPH Model |
| | AUC: 0.77 PMH Model |
| NK cells | AUC: 0.65 CPH Model |
| | AUC: 0.60 PMH Model |
| All lymphocytes | AUC: 0.81 CPH Model |
| | AUC: 0.76 PMH Model |
| All monocytes | AUC: 0.61 CPH Model |
| | AUC: 0.53 PMH Model |
| All cells | AUC: 0.78 CPH Model |
| | AUC: 0.71 PMH Model |
| Viral Load | |

Glycosylation/ConA texture + entropy

| | |
|---|---|
| T-cells | AUC: 0.59 |
| NK cells | AUC: 0.55 |

Mitochondrial texture + entropy

| | |
|---|---|
| T-cells | AUC: 0.66 |
| NK cells | AUC: 0.54 |

Adjuvant phenotype

| | |
|---|---|
| T-cells | CPH model |
| All lymphocytes | CPH model |
| All monocytes | CPH model |
| T-cell morphology/irregularity | |
| Cell-to-cell interaction | |
| [WIP] Apoptotic cell <-> T-cell | |
| interaction | |

Results from the model are shown in the table below.

### Concanavalin Phalloidin and Hoechst

| | | | | | | Transfer |
|---|---|---|---|---|---|---|
| | 2/20 Aging | 3/29 Aging | 3/29 Aging | Combined | Transfer 2/20 | 2/20 -> 3/29 |
| Cell Types | Model | Model (10x) | Model (1x) | Aging Model | ->3/29 (10x) | (1x) |
| T-cells | AUC: 0.81 | AUC: 0.68 | AUC: 0.68 | AUC: 0.77 | p~0.00002 | p~0.00002 |
| | pics (fold0) | (pics fold3) | | | | |
| NK cells | AUC: 0.65 | AUC: 0.59 | AUC: 0.61 | AUC: 0.62 | p~0.021 | p~0.010 |
| All lymphocytes | AUC: 0.81 | AUC: 0.68 | AUC: 0.70 | AUC: 0.76 | p~0.00003 | p~0.00003 |
| All monocytes | AUC: 0.61 | AUC: 0.53 | AUC: 0.55 | AUC: 0.59 | p~0.089 | p~0.549 |
| All cells | AUC: 0.78 | AUC: 0.65 | AUC: 0.70 | AUC: 0.73 | p~0.002 | p~0.004 |

### Mitotracker®, Phalloidin, and Hoechst

| | 3/29 Aging Model | 3/29 Aging |
|---|---|---|
| Cell Types | (10×) | Model (1×) |
| T-cells | AUC: 0.78 | AUC: 0.72 |
| NK cells | AUC: 0.60 | AUC: 0.61 |
| All lymphocytes | AUC: 0.76 | AUC: 0.74 |
| All monocytes | AUC: 0.53 | AUC: 0.54 |
| All cells | AUC: 0.71 | AUC: 0.73 |

Green=Stat. sig. in all folds

### Example 18: Vaccine System as a Screen for Improved Elderly Vaccine Response

A system is designed to model the differences between vaccine response in the young and elderly. This system is used to screen for vaccines that may be efficacious in the elderly. Any available vaccine may be screened, for instance, vaccines against influenza vaccines, SARRS-CoV-2, diphtheria, tetanus, pertussis, hepatitis B, poliomyelitis, and Haemophilus influenzae type b, e.g., Infanrix Hexa. This system may be combined with adjuvant systems, or components thereof, described herein for improving vaccine response in the elderly. For instance, in a classifier comparing efficacious vs. control, the classifier predicts if a given vaccine induces a phenotype in elderly immune cells to respond like young immune cells (e.g., which indicates that the vaccine is efficacious). This may comprise performing a binary classification of the vaccine (e.g., into either an efficacious (young) category or a control category).

Innate Immune Activation and Recruitment Macrophages serve as the first line of immune system defense against pathogens that breach the epithelial barrier. They recognize molecular structures produced by microbial pathogens called pathogen-associated molecular patterns (PAMPs). Upon recognition, they initiate inflammation which results in many cellular and tissue changes in the surrounding area. Generally, it is accepted that directly after virus recognition, macrophages endocytose the invading pathogens and polarize to the MI-like phenotype. One of the most important changes resulting from the inflammatory environment created by the macrophages is the rapid recruitment of circulating neutrophils and monocytes through the secretion of cytokines TNF, IL-1, and IL-6 and chemokines. The neutrophils are recruited to focus on phagocytosing the invaders while the monocytes enter an inflammatory state pushing them to differentiate into dendritic cells.

There are different macrophage and inflammatory phenotypes in the elderly versus young subjects, which are detected in the present system to help identify vaccines for the elderly. For instance, the plasticity of macrophage polarization is dysfunctional in the elderly. Age-induced changes in macrophages are diverse and, in general, may represent pro-inflammatory activation of cells with an alternative activated (M2-like) phenotype. In older patients, inflammatory monocytes produce significantly less inflammatory cytokines, whose decreased production is strongly associated with vaccine antibody response. Further, an accumulation of the non-classical monocytes, in conjunction with higher levels of plasma TNF-α and IL-8 are observed in the elderly. These factors may contribute to inflamm-aging and age-related inflammatory conditions, such as atherosclerosis and osteoarthritis.

Dendritic cell activation and migration. After infection, monocyte derived dendritic cells start secreting inflammatory cytokines and phagocytosing pathogens in the inflamed area. The ingested pathogens are processed into antigens which are presented on MHC molecules. Once a monocyte derived dendritic cell is activated by an antigen, it migrates to the lymph nodes where the naïve T-cells are waiting to be primed by the presented antigen. The robust activation requires the combination of signals from the antigen, costimulatory molecules, and cytokines. The T-cells role across the surrounding cells, interacting with many different antigen presenting cells (APCs) until a match is made between MHC molecules. This match forms an immunological synapse which, along with costimulatory signals, activates a targeted effector response in the T-cell. Other APCs such as monocytes, macrophages, and B-cells can present antigens to activate T-cells which drives T-cell helper functions. The specialization of APCs in response to different vaccines is taken into consideration when designing vaccines for the elderly. The response of different DC subsets to vaccine antigens and adjuvants may be determined to obtain information about the induction of adaptive immunity. For example, to improve vaccine response to, for example, fluzone in the elderly, it may be beneficial to design adjuvants that activate monocytes.

T-cell activation, differentiation, and proliferation. Naïve and memory T-cells exist in a quiescent state, poised to proliferate and differentiate upon antigen stimulation during priming by APCs. Maintaining quiescence is vital to retain self-renewal potential and differentiation plasticity throughout life. In quiescence, cell division and growth are downregulated, cells are arrested in their cell cycle and they have low metabolic and mammalian target of rapamycin complex (mTORC) activity, resulting in reduced ribosome biogenesis and protein synthesis. Disruption of quiescence has been implicated in T-cell aging. Additional disruption to proper activation and effector function of aged T-cells include signaling pathways, poor APC priming, improper cytoskeleton rearrangement, exhaustion, and reduced naïve T-cells.

B-cell activation and and body production. The primary read-out for almost all vaccinations is the induction of protective antigen specific antibodies. The induction of vaccine-specific antibodies can be mediated by follicular or extrafollicular B cell responses, which provide long-term or short-term protection, respectively. Although short-term responses provide rapid antigen-specific antibody production, the cells generated from these interactions display poor survival. Thus, the generation of long-lived antibody-producing cells is essential for an effective vaccine response. In order to achieve long-lived protective antibody responses, B cells must undergo class switch recombination, somatic hypermutation and plasma cell differentiation, all of which require the help of a specialized T cell subset termed T follicular helper cells. These precursor TFH cells interact with local B cells to undergo full maturation into bona fide TFH cells, which again interact with B cells within germinal centers. This germinal center interaction induces the production of high affinity antibodies as well as the release of activated memory TFH cells from the tissue back into the blood. During aging, multiple changes in this pathway occur, including alterations naïve CD4 and TFH cell frequencies within the blood, reductions in TFH-B cell interactions and increases in TFR cells—which in turn lead to lower production of antigen-specific antibodies and activated TFH cells.

Vaccine System and Model. The vaccine system described herein can measure one or more of these various phenotypic modifications at the tissue and cellular level. The system may also be used to screen for vaccines that induce elderly immune cells to respond more like the young immune cells, and discover and target immune aging mechanisms causing decreased response to vaccines. For instance, in a classifier comparing young vs. control, the classifier predicts if a given vaccine induces a phenotype in elderly immune cells to respond like young immune cells. This may comprise performing a binary classification of the vaccine (e.g., into either a positive (young) category or a control category).

An exemplary method is provided for improving vaccine response using a system described herein. The method includes imaging PBMCs after exposure to vaccines; segmenting T-cells and dendritic cells; quantifying innate activation, T-cell priming and T-cell activation; screening possible interventions for hits; and testing hits in a mouse model for elderly vaccine response to identify interventions for human clinical trials.

### Example 19: Adjuvant System as a Screen for Improved Elderly Vaccine Response

Adjuvants stimulate an innate immune response by creating a local inflammatory environment that increases antigen uptake and presentation. One strategy to enhance immunogenicity of vaccines is the addition of adjuvants in order to develop vaccines for populations and pathogens for which traditional vaccines do not provide adequate protection. Most studies evaluating novel adjuvants for the elderly focus on influenza in order to achieve one or more of the following improvements: First, higher antibody responses and thereby protective antibody responses in a higher percentage of vaccines. Secondly, broader antibody recognition, which confers cross-protection against strain variants not included in the vaccine; this aspect is relevant for seasonal influenza vaccines, but particularly for pandemic vaccines enabling the development of prepandemic vaccines (vaccines for potentially pandemic strains, which might evolve until they cause a pandemic). Third, antigen dose sparing, which is also particularly relevant for pandemic situations, when it is crucial to produce a large number of doses in a very short period of time. Fourth, induction of protective mucosal immunity. The ultimate goal of next-generation adjuvanted influenza vaccines is to confer higher clinical efficacy and effectiveness, particularly for vulnerable populations, such as the elderly.

Adjuvants are critical interventions for boasting the elderly innate immune response to vaccines. They focus on enhancing the crosstalk between innate and adaptive immunity that leads to the induction of protective responses to a given pathogen. They enhance antigen uptake at the injection site and the major target cell types may be monocytes, macrophages, and dendritic cells. These activated cells then may secrete a complex of chemokines to recruit more immune cells to the injection site, which helps to form an immunocompetent environment for enhanced antigen transportation to the draining lymph nodes. A variety of APCs with different antigen processing ways will lead to a more competent immune response, including the increased engulfing of antigen and the accumulation of antigens in the draining lymph nodes contributing to the facilitated transportation of the APCs. Monocytes are essential for the innate response to pathogens as they play important roles in the inflammatory response. Monocytes differentiate to dendritic cells in inflammatory situations. These monocyte derived dendritic cells are an essential component of a robust vaccine response. They are recruited to the site of vaccination where they uptake antigens, transport them to the lymph nodes, and use the antigens to activate the adaptive immune system along with the assistance of co-stimulatory molecules. Dendritic cells are peculiar innate cells that can be directly stimulated by pathogen-associated molecular patterns and present pathogen-derived antigens to specific T cells in the lymph nodes draining the infection site. In turn, activated T cells provide help to the different cells of the adaptive immunity compartment. The type of adaptive immune response is specific to the original pathogen and depends in part on the way DCs integrate the various signals received during the initial recognition by pathogen recognition receptors. In the elderly, the ability of the monocyte derived dendritic cells to be recruited to the vaccination site, uptake antigens, migrate to the lymph nodes, and properly prime the adaptive immune system is significantly reduced.

Building a model which can accurately predict the phenotype of a successful adjuvant may be extremely valuable. Historically, adjuvants have proven insanely difficult to develop with most originating from luck followed by decades of persistent development. A model may be used to develop a systemized approach to finding new adjuvants which improve elderly response. The present system described seeks to provide this approach.

The present system allows for screening to identify superior adjuvants for improving elderly vaccine response. For instance, adjuvants can be identified that are synthetic, allowing for easier manufacturing. In addition, adjuvants can be identified that are personalized for the particular antigen.

Adjuvants. Exemplary adjuvants for use in the system include: emulsions, virosomes, and immune-stimulatory complexes such as liposomes, saponins, and toll-like receptor (TLR) agonists. An exemplary adjuvant is AS01 comprising monophosphoryl lipid A (MPL) and saponin (QS-21), which is licensed for use with malaria and herpes zoster vaccines. Another exemplary adjuvant is MF59 comprising an oil-in-water emulsion of squalene oil, which has provided greater efficacy in preventing influenza. Another exemplary adjuvant is AS03, an emulsion that increases titers and has been used during pandemics. Additional adjuvants that are undergoing testing in vaccines for the elderly include imiquimod (TLR agonist) and glucopyranosyl lipid A (TLR4 agonist and emulsion). Another exemplary adjuvant that may be used is Freud's Adjuvant (F5881).

Phenotypes. The phenotypes associated with MF59 treatment include (1) increased chemokine secretion, (2) monocyte differentiation to immature dendritic cells (DCs), (3) higher number of antigen-presenting cells, and (4) ATP release to boost T-cell mitochondrial response. The phenotypes associated with AS01 include (1) enhanced number of DCs and monocytes in the draining lymph node, (2) monocytes and neutrophils were the main cells carry the antigen, followed by DCs, (3) enhanced differentiation of monocytes into DCs, leading to a more heterogeneous DC population and eventually a more efficient antigen presentation to T-cells, and (4) a broad profile of activated APCs: DCs resident in the lymph node, DCs that migrated from the injected muscle, and monocyte-derived DCs.

MF59 assay design. MF59 and GLA adjuvants were used in this assay. The MF59 (Addavax, Invivogen) was dosed at 50-100 μL per mouse (1.5-2.5 mL blood volume) or 250 μL per rabbit (58-580 mL blood volume). The GLA (Monophosphoryl Lipid A, TLR agonist, Enzo) was dosed at 100 ng/mL (for two concentrations, e.g., 50 ng/mL and 250 ng/mL). Each adjuvant was plated on its own plate, with each plate having half of the wells with stimulant and half with controls. The cells were stained separately with 3 palettes: CPM, CPE, and CPMS, for a total of three plates per adjuvant. 12 PBMC samples per plate were included related to subjects between ages 40-50. A design of the plate is shown below.

| | | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 |
|---|---|---|---|---|---|---|---|---|---|---|---|---|---|---|---|---|---|---|---|---|---|---|---|---|---|
| untreated, 12 wells each | A | 1 | 1 | 2 | 2 | 3 | 3 | 4 | 4 | 5 | 5 | 6 | 6 | 7 | 7 | 8 | 8 | 9 | 9 | 10 | 10 | 11 | 11 | 12 | 12 |
| low concentration, 10 wells each | B | 1 | 1 | 2 | 2 | 3 | 3 | 4 | 4 | 5 | 5 | 6 | 6 | 7 | 7 | 8 | 8 | 9 | 9 | 10 | 10 | 11 | 11 | 12 | 12 |
| high concentration, 10 wells each | C | 1 | 1 | 2 | 2 | 3 | 3 | 4 | 4 | 5 | 5 | 6 | 6 | 7 | 7 | 8 | 8 | 9 | 9 | 10 | 10 | 11 | 11 | 12 | 12 |
| | D | 1 | 1 | 2 | 2 | 3 | 3 | 4 | 4 | 5 | 5 | 6 | 6 | 7 | 7 | 8 | 8 | 9 | 9 | 10 | 10 | 11 | 11 | 12 | 12 |
| | E | 1 | 1 | 2 | 2 | 3 | 3 | 4 | 4 | 5 | 5 | 6 | 6 | 7 | 7 | 8 | 8 | 9 | 9 | 10 | 10 | 11 | 11 | 12 | 12 |
| | F | 1 | 1 | 2 | 2 | 3 | 3 | 4 | 4 | 5 | 5 | 6 | 6 | 7 | 7 | 8 | 8 | 9 | 9 | 10 | 10 | 11 | 11 | 12 | 12 |
| | G | 1 | 1 | 2 | 2 | 3 | 3 | 4 | 4 | 5 | 5 | 6 | 6 | 7 | 7 | 8 | 8 | 9 | 9 | 10 | 10 | 11 | 11 | 12 | 12 |
| | H | 1 | 1 | 2 | 2 | 3 | 3 | 4 | 4 | 5 | 5 | 6 | 6 | 7 | 7 | 8 | 8 | 9 | 9 | 10 | 10 | 11 | 11 | 12 | 12 |
| | I | 7 | 7 | 8 | 8 | 9 | 9 | 10 | 10 | 11 | 11 | 12 | 12 | 1 | 1 | 2 | 2 | 3 | 3 | 4 | 4 | 5 | 5 | 6 | 6 |
| | J | 7 | 7 | 8 | 8 | 9 | 9 | 10 | 10 | 11 | 11 | 12 | 12 | 1 | 1 | 2 | 2 | 3 | 3 | 4 | 4 | 5 | 5 | 6 | 6 |
| | K | 7 | 7 | 8 | 8 | 9 | 9 | 10 | 10 | 11 | 11 | 12 | 12 | 1 | 1 | 2 | 2 | 3 | 3 | 4 | 4 | 5 | 5 | 6 | 6 |
| | L | 7 | 7 | 8 | 8 | 9 | 9 | 10 | 10 | 11 | 11 | 12 | 12 | 1 | 1 | 2 | 2 | 3 | 3 | 4 | 4 | 5 | 5 | 6 | 6 |
| | M | 7 | 7 | 8 | 8 | 9 | 9 | 10 | 10 | 11 | 11 | 12 | 12 | 1 | 1 | 2 | 2 | 3 | 3 | 4 | 4 | 5 | 5 | 6 | 6 |
| | N | 7 | 7 | 8 | 8 | 9 | 9 | 10 | 10 | 11 | 11 | 12 | 12 | 1 | 1 | 2 | 2 | 3 | 3 | 4 | 4 | 5 | 5 | 6 | 6 |
| | O | 7 | 7 | 8 | 8 | 9 | 9 | 10 | 10 | 11 | 11 | 12 | 12 | 1 | 1 | 2 | 2 | 3 | 3 | 4 | 4 | 5 | 5 | 6 | 6 |
| | P | 7 | 7 | 8 | 8 | 9 | 9 | 10 | 10 | 11 | 11 | 12 | 12 | 1 | 1 | 2 | 2 | 3 | 3 | 4 | 4 | 5 | 5 | 6 | 6 |

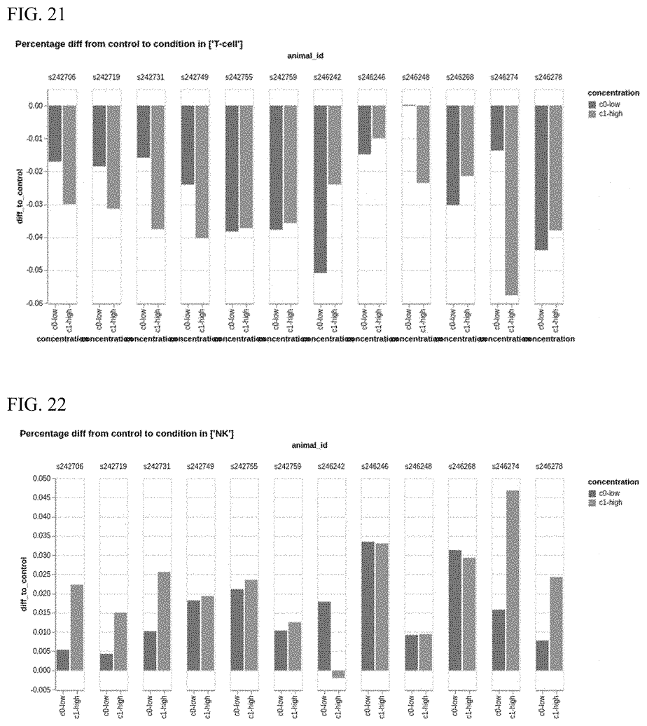

FIG. 21 is a plot of the percentage difference from control to condition in T-cells, showing a decrease in percentage of T-cells with MF59. FIG. 22 is a plot of the percentage difference from control to condition in natural killer cells, showing an increase in percentage of natural killer cells with MF59.

Cell-to-cell interactions were modeled with respect to adjuvant condition (control, 1:500, and 1:50). No significant relationship between condition and cell-to-cell interactions was found for lymphocyte—lymphocyte and lymphocyte—monocyte interactions. A significant relationship was found between conditions and cell-to-cell interactions was found for monocytes—monocyte interactions. There was a sharp and significant increase in monocyte to monocyte clustering with increased adjuvant concentration. A similar relationship was found when only looking at T-cells and macrophage/dendritic cells. The macrophage/dendritic cells exhibited a significant increase in clustering with increased adjuvant. Adjuvants were not shown to increase the cell-to-cell interaction of T-cells with either other T-cells or with macrophage/dendritic cells. No evidence of well effects were observed in this data. Point-point patterns exhibited homogeneous distributions. A summary of MF59 data is shown in the Table below.

| Model | Interactions | Conditios | Strauss Value |
|---|---|---|---|
| Lymphocytes-Monocytes | Lymphocytes-Monocytes | Control | 0.6429 |
| Lymphocytes-Monocytes | Lymphocytes-Lymphocytes | Control | 1.2342 |
| Lymphocytes-Monocytes | Monocytes-Monocytes | Control | 1.3160** |
| Lymphocytes-Monocytes | Lymphocytes-Monocytes | 1:500 | 0.4761 |
| Lymphocytes-Monocytes | Lymphocytes-Lymphocytes | 1:500 | 1.0910 |
| Lymphocytes-Monocytes | Monocytes-Monocytes | 1:500 | 1.5915** |
| Lymphocytes-Monocytes | Lymphocytes-Monocytes | 1:50 | 1.2912 |
| Lymphocytes-Monocytes | Lymphocytes-Lymphocytes | 1.50 | 0.6252 |
| Lymphocytes-Monocytes | Monocytes-Monocytes | 1:50 | 1.9522*** |
| T-Cells-Macrophage/Dendritic | T-Cells-Macrophage/Dendritic | Control | 0.6479 |
| T-Cells-Macrophage/Dendnitic | T-Cells-T-Cells | Control | 1.3442 |
| T-Cells-Macrophage/Dendritic | Macrophage/Dendritic- | Control | 1.3861** |
| | Macrophage/Dendritic | | |
| T-Cells-Macrophage/Dendntic | T-Cells-Macrophage/Dendntic | 1:500 | 1.2064 |
| T-Cells-Macrophage/Dendritic | T-Cells-T-Cells | 1:500 | 0.4597 |
| T-Cells-Macrophage/Dendritic | Macrophage/Dendritic- | 1:500 | 1.8125** |
| | Macrophage/Dendritic | | |
| T-Cells-Macrophage/Dendntic | T-Cells-Macrophage/Dendnitic | 1:50 | 1.3683 |
| T-Cells-Macrophage/Dendritic | T-Cells-T-Cells | 1:50 | 0.6154 |
| T-Cells-Macrophage/Dendntic | Macrophage/Dendritic- | 1:50 | 2.1392** |
| | Macrophage/Dendritic | | |

**w/ statistically significant model coefficient p < 0.01

AS01 assay design. ASOl adjuvant was used in this assay. The AS01 comprised MLPA (S. Minnesota) at 10 ng/mL-1 μg/mL in mDC cells, and QuilA (Saponin) at 10 μg/mL in Hela cells. The AS01 was dosed at a low dose (10 ng/ml MLPA+5 pig/ml Saponin), or a high dose (1 g/ml MLPA+10 μg/ml Saponin). Each adjuvant was plated on its own plate, with each plate having half of the wells with stimulant and half with controls. The cells were stained separately with 3 palettes: CPM, CPE, and CPMS, for a total of three plates per adjuvant. 12 PBMC samples per plate are included related to subjects between ages 40-50. A design of the plate is shown below.

| | | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 |
|---|---|---|---|---|---|---|---|---|---|---|---|---|---|---|---|---|---|---|---|---|---|---|---|---|---|
| untreated, 12 wells each | A | 1 | 1 | 2 | 2 | 3 | 3 | 4 | 4 | 5 | 5 | 6 | 6 | 7 | 7 | 8 | 8 | 9 | 9 | 10 | 10 | 11 | 11 | 12 | 12 |
| low concentration, 10 wells each | B | 1 | 1 | 2 | 2 | 3 | 3 | 4 | 4 | 5 | 5 | 6 | 6 | 7 | 7 | 8 | 8 | 9 | 9 | 10 | 10 | 11 | 11 | 12 | 12 |
| high concentration, 10 wells each | C | 1 | 1 | 2 | 2 | 3 | 3 | 4 | 4 | 5 | 5 | 6 | 6 | 7 | 7 | 8 | 8 | 9 | 9 | 10 | 10 | 11 | 11 | 12 | 12 |
| | D | 1 | 1 | 2 | 2 | 3 | 3 | 4 | 4 | 5 | 5 | 6 | 6 | 7 | 7 | 8 | 8 | 9 | 9 | 10 | 10 | 11 | 11 | 12 | 12 |
| | E | 1 | 1 | 2 | 2 | 3 | 3 | 4 | 4 | 5 | 5 | 6 | 6 | 7 | 7 | 8 | 8 | 9 | 9 | 10 | 10 | 11 | 11 | 12 | 12 |
| | F | 1 | 1 | 2 | 2 | 3 | 3 | 4 | 4 | 5 | 5 | 6 | 6 | 7 | 7 | 8 | 8 | 9 | 9 | 10 | 10 | 11 | 11 | 12 | 12 |
| | G | 1 | 1 | 2 | 2 | 3 | 3 | 4 | 4 | 5 | 5 | 6 | 6 | 7 | 7 | 8 | 8 | 9 | 9 | 10 | 10 | 11 | 11 | 12 | 12 |
| | H | 1 | 1 | 2 | 2 | 3 | 3 | 4 | 4 | 5 | 5 | 6 | 6 | 7 | 7 | 8 | 8 | 9 | 9 | 10 | 10 | 11 | 11 | 12 | 12 |
| | I | 7 | 7 | 8 | 8 | 9 | 9 | 10 | 10 | 11 | 11 | 12 | 12 | 1 | 1 | 2 | 2 | 3 | 3 | 4 | 4 | 5 | 5 | 6 | 6 |
| | J | 7 | 7 | 8 | 8 | 9 | 9 | 10 | 10 | 11 | 11 | 12 | 12 | 1 | 1 | 2 | 2 | 3 | 3 | 4 | 4 | 5 | 5 | 6 | 6 |
| | K | 7 | 7 | 8 | 8 | 9 | 9 | 10 | 10 | 11 | 11 | 12 | 12 | 1 | 1 | 2 | 2 | 3 | 3 | 4 | 4 | 5 | 5 | 6 | 6 |
| | L | 7 | 7 | 8 | 8 | 9 | 9 | 10 | 10 | 11 | 11 | 12 | 12 | 1 | 1 | 2 | 2 | 3 | 3 | 4 | 4 | 5 | 5 | 6 | 6 |
| | M | 7 | 7 | 8 | 8 | 9 | 9 | 10 | 10 | 11 | 11 | 12 | 12 | 1 | 1 | 2 | 2 | 3 | 3 | 4 | 4 | 5 | 5 | 6 | 6 |
| | N | 7 | 7 | 8 | 8 | 9 | 9 | 10 | 10 | 11 | 11 | 12 | 12 | 1 | 1 | 2 | 2 | 3 | 3 | 4 | 4 | 5 | 5 | 6 | 6 |
| | O | 7 | 7 | 8 | 8 | 9 | 9 | 10 | 10 | 11 | 11 | 12 | 12 | 1 | 1 | 2 | 2 | 3 | 3 | 4 | 4 | 5 | 5 | 6 | 6 |
| | P | 7 | 7 | 8 | 8 | 9 | 9 | 10 | 10 | 11 | 11 | 12 | 12 | 1 | 1 | 2 | 2 | 3 | 3 | 4 | 4 | 5 | 5 | 6 | 6 |

Flow cytometry was performed as follows. Twelve donors were assayed with three conditions each (native, low dose, and high dose), for a total of 36 samples. A custom 5 color flow kit from BD Biosciences was utilized: T-cells (CD3)—PERCP, B-cells (CD19)—APC, monocyte/macrophage (CD14)—PE, monocyte/dendritic cell (CD11c)—APC-Cy7, and NK cells (CD56)—FITC.

Cell-to-cell interactions were modeled with respect to adjuvant condition (control, 10 ng/mL MPLA-SM/5 μg/mL Quil-A, and 1 μg/mL MPLA-SM/10 μg/mL Quil-A). No significant relationship between condition and cell-to-cell interactions was found for lymphocyte—lymphocyte and lymphocyte—monocyte interactions. Similar to the cell-to-cell interactions for MF59, a significant relationship was found between conditions and cell2 cell interactions was found for monocytes—monocyte interactions (p=3.03E−14)), and macrophage/dendritic—macrophage/dendritic interactions (p=1.22e−09). This increase in monocyte/macrophage/dendritic cell interactions was seen in both concentrations of adjuvant, but there was not a significant difference in cell-to-cell interactions between these two concentrations. Also similar to previous findings for MF59, the AS01 adjuvant was not shown to increase the cell-to-cell interaction of T-cells with either other T-cells or with macrophage/dendritic cells. No evidence of well effects were observed in this data. Point-point patterns exhibited homogeneous distributions. A summary of MF59 data is shown in the Table below.

### AS01 CPM Plate

| Model | Interactions | Condition | Value |
|---|---|---|---|
| Lymphocytes-Monocytes | Lymphocytes-Monocytes | Control | 0.6171 |
| Lymphocytes-Monocytes | Lymphocytes-Lymphocytes | Control | 1.3437 |
| Lymphocytes-Monocytes | Monocytes-Monocytes | Control | 0.9438** |
| Lymphocytes-Monocytes | Lymphocytes-Monocytes | 10 ng/mL MPLA-SM/5 μg/mL Quil-A | 0.5820 |
| Lymphocytes-Monocytes | Lymphocytes-Lymphocytes | 10 ng/mL MPLA-SM/5 μg/mL Quil-A | 1.2607 |
| Lymphocytes-Monocytes | Monocytes-Monocytes | 10 ng/mL MPLA-SM/5 μg/mL Quil-A | 1.2454** |
| Lymphocytes-Monocytes | Lymphocytes-Monocytes | 1 μg/mL MPLA-SM/10 μg/mL Quil-A | 0.6727 |
| Lymphocytes-Monocytes | Lymphocytes-Lymphocytes | 1 μg/mL MPLA-SM/10 μg/mL Quil-A | 1.3738 |
| Lymphocytes-Monocytes | Monocytes-Monocytes | 1 μg/mL MPLA-SM/10 μg/mL Quil-A | 1.2745** |
| T-Cells-Macrophage/Dendritic | T-Cells-Macrophage/Dendritic | Control | 0.5751 |
| T-Cells-Macrophage/Dendritic | T-Cells-T-Cells | Control | 1.5421 |
| T-Cells-Macrophage/Dendritic | Macrophage/Dendritic-Macrophage/Dendritic | Control | 1.1944** |
| T-Cells-Macrophage/Dendritic | T-Cells-Macrophage/Dendritic | 10 ng/mL MPLA-SM/5 μg/mL Quil-A | 0.5318 |
| T-Cells-Macrophage/Dendritic | T-Cells-T-Cells | 10 ng/mL MPLA-SM/5 μg/mL Quil-A | 1.4004 |
| T-Cells-Macrophage/Dendritic | Macrophage/Dendritic-Macrophage/Dendritic | 10 ng/mL MPLA-SM/5 μg/mL Quil-A | 1.5514** |
| T-Cells-Macrophage/Dendritic | T-Cells-Macrophage/Dendritic | 1 μg/mL MPLA-SM/10 μg/mL Quil-A | 0.5728 |
| T-Cells-Macrophage/Dendritic | T-Cells-T-Cells | 1 μg/mL MPLA-SM/10 μg/mL Quil-A | 1.5264 |
| T-Cells-Macrophage/Dendritic | Macrophage/Dendritic-Macrophage/Dendritic | 1 μg/mL MPLA-SM/10 μg/mL Quil-A | 1.5785** |

**w/ statistically signigicant difference in control-adjuvant interactions (p < 0.01)

MF59 and XV01 assay. MF59 and ASOl adjuvants are used in this assay. The MF59 (Addavar, Invivogen) is dosed at 50-100 μL per mouse (1.5-2.5 mL blood volume) or 250 μL per rabbit (58-580 mL blood volume), dilutions are at 1:50 and 1-500. The ASOl is dosed at a low dose (10 ng/ml MLPA+5 μg/ml Saponin), or a high dose (1 μg/ml MLPA+10 μg/ml Saponin). Each adjuvant is plated on its own plate, with each plate having half of the wells with stimulant and half with controls. The cells are stained separately with 2 palettes: CPM and CPE. Per plate there are untreated and low and high doses of adjuvant. There are two total plates per adjuvant, two plates for compound controls (CPM, CPE), totaling six plates. 12 PBMC samples per plate are included related to subjects between ages 40-50. No flow cytometry is performed. An alternating, staggered plate layout similar to the aging model is used to validate results. A design of the plate is shown below.

| 384-Well | | | | | | | | | | | | | | | | | | | | | | | | |
|---|---|---|---|---|---|---|---|---|---|---|---|---|---|---|---|---|---|---|---|---|---|---|---|---|
| R | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 |
| A | 1 | 1 | 2 | 2 | 1 | 1 | 2 | 2 | 1 | 1 | 2 | 2 | 3 | 3 | 3 | 3 | 2 | 2 | 1 | 1 | 3 | 3 | 2 | 2 |
| B | 1 | 1 | 2 | 2 | 1 | 1 | 2 | 2 | 1 | 1 | 2 | 2 | 3 | 3 | 3 | 3 | 2 | 2 | 1 | 1 | 3 | 3 | 2 | 2 |
| C | 3 | 3 | 1 | 1 | 2 | 2 | 1 | 1 | 2 | 2 | 1 | 1 | 2 | 2 | 3 | 3 | 3 | 3 | 2 | 2 | 1 | 1 | 3 | 3 |
| D | 3 | 3 | 1 | 1 | 2 | 2 | 1 | 1 | 2 | 2 | 1 | 1 | 2 | 2 | 3 | 3 | 3 | 3 | 2 | 2 | 1 | 1 | 3 | 3 |
| E | 2 | 2 | 3 | 3 | 1 | 1 | 2 | 2 | 1 | 1 | 2 | 2 | 1 | 1 | 2 | 2 | 3 | 3 | 3 | 3 | 2 | 2 | 1 | 1 |
| F | 2 | 2 | 3 | 3 | 1 | 1 | 2 | 2 | 1 | 1 | 2 | 2 | 1 | 1 | 2 | 2 | 3 | 3 | 3 | 3 | 2 | 2 | 1 | 1 |
| G | 2 | 2 | 3 | 3 | 3 | 3 | 1 | 1 | 2 | 2 | 1 | 1 | 2 | 2 | 3 | 3 | 2 | 2 | 3 | 3 | 3 | 3 | 2 | 2 |
| H | 2 | 2 | 3 | 3 | 3 | 3 | 1 | 1 | 2 | 2 | 1 | 1 | 2 | 2 | 3 | 3 | 2 | 2 | 3 | 3 | 3 | 3 | 2 | 2 |
| I | 1 | 1 | 2 | 2 | 3 | 3 | 3 | 3 | 1 | 1 | 2 | 2 | 1 | 1 | 2 | 2 | 3 | 3 | 2 | 2 | 3 | 3 | 3 | 3 |
| J | 1 | 1 | 2 | 2 | 3 | 3 | 3 | 3 | 1 | 1 | 2 | 2 | 1 | 1 | 2 | 2 | 3 | 3 | 2 | 2 | 3 | 3 | 3 | 3 |
| K | 2 | 2 | 1 | 1 | 2 | 2 | 3 | 3 | 3 | 3 | 1 | 1 | 2 | 2 | 1 | 1 | 2 | 2 | 3 | 3 | 2 | 2 | 3 | 3 |
| L | 2 | 2 | 1 | 1 | 2 | 2 | 3 | 3 | 3 | 3 | 1 | 1 | 2 | 2 | 1 | 1 | 2 | 2 | 3 | 3 | 2 | 2 | 3 | 3 |
| M | 3 | 3 | 2 | 2 | 1 | 1 | 2 | 2 | 3 | 3 | 3 | 3 | 1 | 1 | 2 | 2 | 1 | 1 | 2 | 2 | 3 | 3 | 2 | 2 |
| N | 3 | 3 | 2 | 2 | 1 | 4 | 2 | 2 | 3 | 3 | 3 | 3 | 1 | 1 | 2 | 2 | 1 | 1 | 2 | 2 | 3 | 3 | 2 | 2 |
| O | 2 | 2 | 3 | 3 | 2 | 2 | 1 | 1 | 2 | 2 | 3 | 3 | 3 | 3 | 1 | 1 | 2 | 2 | 1 | 1 | 2 | 2 | 3 | 3 |
| P | 2 | 2 | 3 | 3 | 2 | 2 | 1 | 1 | 2 | 2 | 3 | 3 | 3 | 3 | 1 | 1 | 2 | 2 | 1 | 1 | 2 | 2 | 3 | 3 |

1 = native, 2 = adjuvant high 3 = adjuvant low

Summary. The adjuvant system described herein distinguished MF59 adjuvant (n=478) and control (n=472) PBMCs with 0.89 AUC. In addition, the adjuvant system distinguished ASOl adjuvant (n=480) and control (n=480) PBMCs with 0.90 AUC. MF59 was shown to significantly (p<0.0001) increase monocyte/dendritic cell interaction and clustering. MF59 increases mitochondrial activity, which is thought to be associated with its ability to increase ATP production in surrounding cells. AS01 was shown to change many key monocyte/dendritic cell organelle and structure.

The system may be used to model the phenotype of adjuvants clinically proven to improve vaccine response. This model may be used to screen and classify adjuvants. In some embodiments, the adjuvant classification comprises classifying a given small molecule into an adjuvant category from among a plurality of adjuvant categories. For instance, in a first level classifier comparing adjuvant vs. control, the classifier predicts if a small molecule induces a phenotype that looks like any of the clinically proven adjuvants. This may comprise performing a binary classification of the small molecule (e.g., into either an adjuvant category or a control or non-adjuvant category). In a second level classifier comparing AS01 vs. MF59 vs. GLA vs. control, the classifier predicts if a small molecules looks more like a specific adjuvant. This may comprise performing a categorical classification from among three or more categories of the small molecule (e.g., into an AS01 category, an MF59 category, a GLA category, or a control or non-adjuvant category). In a third level classifier comparing AS01 high vs. AS01 low vs. MF59 high vs. MF59 low vs. GLA high vs. GLA low vs. control, the classifier predicts if a small molecule looks more like the high or low concentration for a specific adjuvant. This may comprise performing a categorical classification from among five or more categories of the small molecule (e.g., into an AS01-high category, an AS01-low category, an MF59-high category, an MF59-low category, a GLA-high category, a GLA-low category, or a control or non-adjuvant category).

### Example 20: Monocyte System as a Screen for Improved Elderly Monocyte Function

The present example provides a system to model the dysfunctional innate immune response to virus, such as coronavirus (SARS-CoV-2), and the aggravation of that response by inherit dysfunction in the elderly innate immune system.

M1/M2 monocyte/macrophage. Monocytes/macrophages are a type of white blood cell of the immune system that engulfs and digests cellular debris, foreign substances, microbes, cancer cells, and anything else that does not have the type of proteins specific to healthy body cells on its surface in a process called phagocytosis. Besides phagocytosis, they play a critical role in nonspecific defense (innate immunity) and also help initiate specific defense mechanisms (adaptive immunity) by recruiting other immune cells such as lymphocytes. Beyond increasing inflammation and stimulating the immune system, monocytes/macrophages also play an important anti-inflammatory role and can decrease immune reactions through the release of cytokines. In the presence of inflammatory stimuli and danger signals, macrophages polarize toward the M1 state and release reactive species and inflammatory cytokines to fight pathogens. In contrast, a wound healing environment promotes polarization toward an M2 phenotype and leads to cellular processes that facilitate tissue repair. Macrophages exhibit different degrees of elongation when stimulated toward M1 or M2 phenotypes with cytokines in vitro. M1 macrophages were smaller, more rounded cells with tightly packed dotted texture of actin. M2 macrophages exhibited larger, more irregular cell bodies with smoother actin staining and more distributed localized spots.

Monocytes can be polarized towards the M1 phenotype by IFN-γ or LPS. The addition of granulocyte macrophage colony-stimulating factor (GM-CSF, which acts as a priming signal for macrophages during M1 polarization) augments the pro-inflammatory function of these cells. By contrast, M2 polarization can be achieved by the addition of IL-4. As with GM-CSF and M1 polarization, macrophage colony-stimulating factor (M-CSF) can enhance the anti-inflammatory function of M2 macrophages.

The plasticity of macrophage polarization is dysfunctional in the elderly. Age-induced changes in macrophages are diverse and, in general, may represent pro-inflammatory activation of cells with an alternatively activated (M2-like) phenotype. Using our high content assay and the experimental design below, a computational model is built to predict if a macrophage is more M1-like or M2-like then the model is used to find modulators of this state. For instance, in a first level classifier comparing MI-like vs. M2-like, the classifier predicts if a macrophage has a phenotype that looks like M1 or M2. This may comprise performing a binary classification of the macrophage (e.g., into either an M1-like category or an M2-like category).

M1/M2 monocyte/macrophage assay development. Monocytes from elderly subjects are exposed to coronavirus or model stimulants to see how they are different from the young response from the perspective of the M1/M2 phenotype. For instance, whether the monocytes from older subjects have a delayed and/or dysfunctional response. A screen can then be performed to identify drugs that improve this response. Human coronavirus 229E ATCC VR-740 and model stimulants are used. Additional regents used in the assay include LPS (20 ng/mL, Sigma, 6 μg), INFγ (20 ng/mL, R&D, 60 mL, 1.2 μg), IL-4 (20 ng/mL, Milltenyi Bio, 0.6 μg), IL-13 (10 ng/mL, R&D), and GM-CSF (100 ng/mL, 120 mL, 12 μg). The palette stain used includes Brightfield, Hoechst, WGA, Mitotracker, ConA, and Phalloidin. An exemplary plate is shown below.

| | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | | |
|---|---|---|---|---|---|---|---|---|---|---|---|---|---|---|---|---|---|---|---|---|---|---|---|---|---|---|
| A | P1 | M1 | P2 | M2 | P3 | M3 | P1 | M1 | P2 | M2 | P3 | M3 | P1 | M1 | P2 | M2 | P3 | M3 | P1 | M1 | P2 | M2 | P3 | M3 | PBMC 1 | P1 |
| B | P1 | M1 | P2 | M2 | P3 | M3 | P1 | M1 | P2 | M2 | P3 | M3 | P1 | M1 | P2 | M2 | P3 | M3 | P1 | M1 | P2 | M2 | P3 | M3 | PBMC 2 | P2 |
| C | P1 | M1 | P2 | M2 | P3 | M3 | P1 | M1 | P2 | M2 | P3 | M3 | P1 | M1 | P2 | M2 | P3 | M3 | P1 | M1 | P2 | M2 | P3 | M3 | PBMC 3 | P3 |
| D | P1 | M1 | P2 | M2 | P3 | M3 | P1 | M1 | P2 | M2 | P3 | M3 | P1 | M1 | P2 | M2 | P3 | M3 | P1 | M1 | P2 | M2 | P3 | M3 | Monocyte 1 | M1 |
| E | P1 | M1 | P2 | M2 | P3 | M3 | P1 | M1 | P2 | M2 | P3 | M3 | P1 | M1 | P2 | M2 | P3 | M3 | P1 | M1 | P2 | M2 | P3 | M3 | Monocyte 2 | M2 |
| F | P1 | M1 | P2 | M2 | P3 | M3 | P1 | M1 | P2 | M2 | P3 | M3 | P1 | M1 | P2 | M2 | P3 | M3 | P1 | M1 | P2 | M2 | P3 | M3 | Monocyte 3 | M3 |
| G | P1 | M1 | P2 | M2 | P3 | M3 | P1 | M1 | P2 | M2 | P3 | M3 | P1 | M1 | P2 | M2 | P3 | M3 | P1 | M1 | P2 | M2 | P3 | M3 | | |
| H | P1 | M1 | P2 | M2 | P3 | M3 | P1 | M1 | P2 | M2 | P3 | M3 | P1 | M1 | P2 | M2 | P3 | M3 | P1 | M1 | P2 | M2 | P3 | M3 | untreated | untreated |
| I | P1 | M1 | P2 | M2 | P3 | M3 | P1 | M1 | P2 | M2 | P3 | M3 | P1 | M1 | P2 | M2 | P3 | M3 | P1 | M1 | P2 | M2 | P3 | M3 | mCSF | naive |
| J | P1 | M1 | P2 | M2 | P3 | M3 | P1 | M1 | P2 | M2 | P3 | M3 | P1 | M1 | P2 | M2 | P3 | M3 | P1 | M1 | P2 | M2 | P3 | M3 | LPS/INFg/mCSF | |
| K | P1 | M1 | P2 | M2 | P3 | M3 | P1 | M1 | P2 | M2 | P3 | M3 | P1 | M1 | P2 | M2 | P3 | M3 | P1 | M1 | P2 | M2 | P3 | M3 | IL-4/IL-13/mCSF | |
| L | P1 | M1 | | | | | | | | | | | | | | | | | | | | | | | | |

Antigen update assay. Antigen update occurs in a monocyte population. In this assay, 24, 48 and 72 hours are used to see processing (and “presenting”) of the antigen can be targeted in a time dependent fashion. Processing of the antigens may happen through the endosomal/lysosomal pathway. Therefore the assay is designed to monitor co-localization of the antigens with lysosomal marker Rab9 and Lamp2. If co-localization is observed, this may qualify this as successful processing of the antigen in the lysosomes. Antigens will be chopped down to small peptides to be presented in the grove of the MHC2 complex. Antigens for the assay include SARS-CoV-2 Spike Glycoprotein (low dose at 0.5 μg/mL, high dose at 5 μg/mL) and influenza virus antigens (low dose at 0.5 μg/mL, high dose at 5 μg/mL). Adjuvants for the assay include ASO at a low dose of 10 ng/mL MLPA and 5 μg/mL Saponin. An exemplary layout of the plating is shown below.

Timepoints: 24, 48, 72 h

| | | | | | infA1 + | | SP1 + | | Dex + | | | | infA1 + | | SP1 + | | Dex + | | | | infA1 + | | SP1 + | | Dex + | Anti- | Stain |
|---|---|---|---|---|---|---|---|---|---|---|---|---|---|---|---|---|---|---|---|---|---|---|---|---|---|---|---|
| | | naive | AS01 | infA1 | A501 | SP1 | A501 | Dex | A501 | naive | A501 | infA1 | A501 | SP1 | A501 | Dex | A501 | naive | A501 | infA1 | A501 | SP1 | A501 | Dex | A501 | gen | Lysosome |
| | | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | conc. | antibody |
| 10000 | A | 1 | 1 | 1 | 1 | 1 | 1 | 1 | 1 | 1 | 1 | 1 | 1 | 1 | 1 | 1 | 1 | 1 | 1 | 1 | 1 | 1 | 1 | 1 | 1 | low | Rab9 |
| cells/ | B | 2 | 2 | 2 | 2 | 2 | 2 | 2 | 2 | 2 | 2 | 2 | 2 | 2 | 2 | 2 | 2 | 2 | 2 | 2 | 2 | 2 | 2 | 2 | 2 | dose | low |
| well | C | 1 | 1 | 1 | 1 | 1 | 1 | 1 | 1 | 1 | 1 | 1 | 1 | 1 | 1 | 1 | 1 | 1 | 1 | 1 | 1 | 1 | 1 | 1 | 1 | | Rab9 |
| | D | 2 | 2 | 2 | 2 | 2 | 2 | 2 | 2 | 2 | 2 | 2 | 2 | 2 | 2 | 2 | 2 | 2 | 2 | 2 | 2 | 2 | 2 | 2 | 2 | | high |
| | E | 1 | 1 | 1 | 1 | 1 | 1 | 1 | 1 | 1 | 1 | 1 | 1 | 1 | 1 | 1 | 1 | 1 | 1 | 1 | 1 | 1 | 1 | 1 | 1 | | LAMP2 |
| | F | 2 | 2 | 2 | 2 | 2 | 2 | 2 | 2 | 2 | 2 | 2 | 2 | 2 | 2 | 2 | 2 | 2 | 2 | 2 | 2 | 2 | 2 | 2 | 2 | | low |
| | G | 1 | 1 | 1 | 1 | 1 | 1 | 1 | 1 | 1 | 1 | 1 | 1 | 1 | 1 | 1 | 1 | 1 | 1 | 1 | 1 | 1 | 1 | 1 | 1 | | LAMP2 |
| | H | 2 | 2 | 2 | 2 | 2 | 2 | 2 | 2 | 2 | 2 | 2 | 2 | 2 | 2 | 2 | 2 | 2 | 2 | 2 | 2 | 2 | 2 | 2 | 2 | | high |
| | I | 1 | 1 | 1 | 1 | 1 | 1 | 1 | 1 | 1 | 1 | 1 | 1 | 1 | 1 | 1 | 1 | 1 | 1 | 1 | 1 | 1 | 1 | 1 | 1 | high | Rab9 |
| | J | 2 | 2 | 2 | 2 | 2 | 2 | 2 | 2 | 2 | 2 | 2 | 2 | 2 | 2 | 2 | 2 | 2 | 2 | 2 | 2 | 2 | 2 | 2 | 2 | dose | low |
| | K | 1 | 1 | 1 | 1 | 1 | 1 | 1 | 1 | 1 | 1 | 1 | 1 | 1 | 1 | 1 | 1 | 1 | 1 | 1 | 1 | 1 | 1 | 1 | 1 | | Rab9 |
| | L | 2 | 2 | 2 | 2 | 2 | 2 | 2 | 2 | 2 | 2 | 2 | 2 | 2 | 2 | 2 | 2 | 2 | 2 | 2 | 2 | 2 | 2 | 2 | 2 | | high |
| | M | 1 | 1 | 1 | 1 | 1 | 1 | 1 | 1 | 1 | 1 | 1 | 1 | 1 | 1 | 1 | 1 | 1 | 1 | 1 | 1 | 1 | 1 | 1 | 1 | | LAMP2 |
| | N | 2 | 2 | 2 | 2 | 2 | 2 | 2 | 2 | 2 | 2 | 2 | 2 | 2 | 2 | 2 | 2 | 2 | 2 | 2 | 2 | 2 | 2 | 2 | 2 | | low |
| | O | 1 | 1 | 1 | 1 | 1 | 1 | 1 | 1 | 1 | 1 | 1 | 1 | 1 | 1 | 1 | 1 | 1 | 1 | 1 | 1 | 1 | 1 | 1 | 1 | | LAMP2 |
| | P | 2 | 2 | 2 | 2 | 2 | 2 | 2 | 2 | 2 | 2 | 2 | 2 | 2 | 2 | 2 | 2 | 2 | 2 | 2 | 2 | 2 | 2 | 2 | 2 | | high |

Antigens
infA1 (H1N1): Low 0.5 μg/ml and high 5 μg/ml
SP1: Low 0.5 μg/ml and high 5 μg/ml
Dextran beads (stock 25 mg/ml): Low 5 μg/ml
Adjuvants
A501: low (10 ng/ml MLPA + 5 μg/ml Saponin)

### Example 21: Cytokine extraction from the systems described herein for image analysis

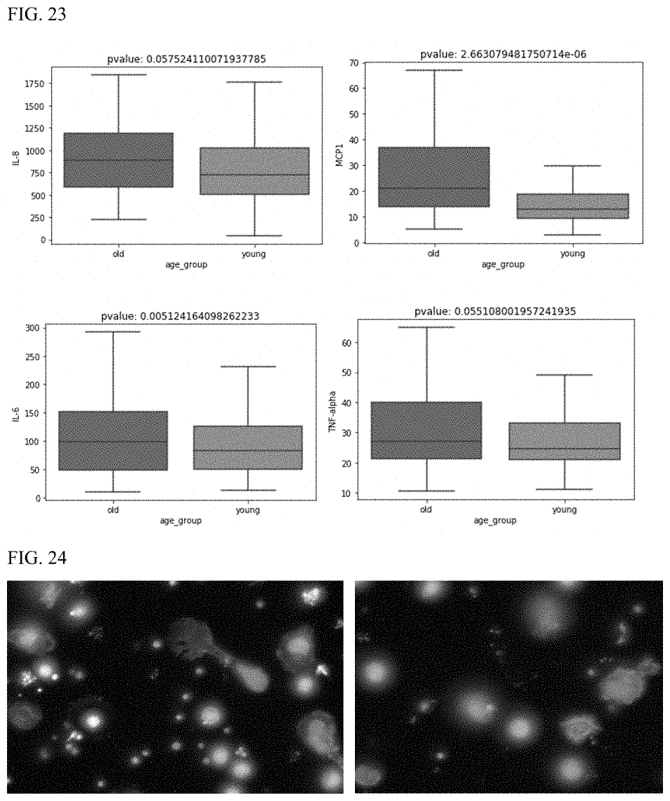

Cytokines from experiments described above may be measured using a FirePlex®-HT immunoassay from Abcam. Cytokines important in aging include, IFN-α, TNF-α, IL-6, IL-12, IL-8, IL-10, and IFN-γ. Human proteins measured using the immunoassay include those in FirePlex®-HT immunoassay, including GM-CSF, IFN-gamma, IL-1 beta, IL-10, IL12p70, IL-13, IL-17A, IL-1a, IL-2, IL-4, IL-5, IL-6, IL-8, IL-9, IP-10, MCP1, MIPIb, TNF-alpha, BCA1, C-reactive protein, EGF, Eotaxin, Eotaxin 2, Eotaxin 3, FGF1, Fractalkine, G-CSF, granzyme A, granzyme B, IFN-alphal, IFN-alpha2, IL-R1, IL-11, IL-12/EL-23 p40, IL-15, IL-18, IL-21, IL-22, IL-23, IL-31, IL-6R alpha, IL-7, M-CSF, MDC, MIG, MIP2/CXCL2, MIP3a/CCL20, RANTES, sTNF Rll, TARC, TNF-beta, VEGF R1. A pilot experiment was run, with a summary of results shown in FIG. 23. A difference in IL-6, IL-8, MCP1, and TNF-alpha was observed between young and old cell samples.

Any of the aforementioned assays may be performed on one or more samples from a patient in order to screen various interventions on cell aging and/or immune function. Such interventions include test agents such as therapeutics. Immune function includes diseases such as elderly vaccine response and chronic inflammation.

In any of the aforementioned assays, a second cell is not contacted with the test agent and serves as a control. A test agent is identified as a drug if it is capable of changing a cell's state, function, and/or predicted age based upon the change in the first in vitro cell's state, function, and/or predicted age relative to a second in vitro cell that was not contacted with the test agent.

### Example 22: COVID-19 Strategy Response

Worldwide, there are over four hundred thousand cases of confirmed COVID-19 cases and more than eighteen thousand deaths. Compared to those aged 30-59 years, those younger than 30 were 0.6 times less likely to die after developing symptoms while those older than 59 were 5.1 times more likely to die. The risk of symptomatic infection increased with age (for example, at about 4% per year among adults aged 30-60 years.

Several categories of repurposing and novel therapies may be tested for each stage of the COVID-19 disease. For example, preventative therapies may be developed which invoke mechanisms of the host's immune system to prevent the virus from gaining a foothold. Importantly, a vaccine is being developed which gives the body an acquired immunity to the infection. Other preventative measures also include antibodies which neutralize the virus by blocking interactions with a receptor, or binding to a viral capsid in a manner that inhibits uncoating of the genome. Antivirals which disrupt various mechanisms of the virus are being developed to be deployed at multiple disease stages. Viral targets range from the RNA replication mechanisms to blocking binding of the host cell receptors which enable viral entry. In addition to this, anti-inflammatory therapies which target pro-inflammatory cytokines are being evaluated for their ability to reduce inflammation and tissue damage at the severe stage of the disease.

Using systems and methods of the present disclosure, improved approaches are used to evaluate strategies for responding to COVID-19. Recognizing the immense clinical burden that COVID-19 has on the elderly and the immune related dysfunction shown to be a central tenet of severe disease progression, such approaches leverage the known dysfunction of the elderly immune system, which is one of the main drivers of COVID-19's devastating impact on this demographic. Therefore, therapies are developed which target these age-related immune mechanisms.

Clinically, the immune responses induced by SARS-CoV-2 infection may be two-phased. During the incubation and non-severe stages, a specific adaptive immune response is required to eliminate the virus and to preclude disease progression to severe stages. Therefore, strategies to boost immune responses at this stage are certainly important. For the development of a protective immune response at the incubation and non-severe stages, the host may be in good general health and an appropriate genetic background that elicits specific antiviral immunity. However, when a protective immune response is impaired, virus will propagate and massive destruction of the affected tissues will occur, especially in organs that have high ACE2 expression, such as intestine and kidney. The damaged cells induce innate inflammation in the lungs that is largely mediated by pro-inflammatory macrophages and granulocytes. Lung inflammation is the main cause of life-threatening respiratory disorders at the severe stage. Therefore, good general health may not be advantageous for patients who have advanced to the severe stage: once severe lung damage occurs, efforts may be made to suppress inflammation and to manage the symptoms. Further, the two-phase division is very important: the first immune defense-based protective phase (mild/early stage) and the second inflammation-driven damaging phase (severe/late stage). One system may search for therapies which boost immune responses during the mild/early stage, while another system may search for therapies which suppress it in the severe/late phase.

Using systems and methods of the present disclosure, multiple model systems were developed which can assess the ability of approved therapies to be beneficial at each of these disease stages. These approaches leverage various specific details of each disease stage and the system that was built to model it, as provided elsewhere herein. For prevention and vaccines, an adjuvant system was built, which finds better adjuvants to improve vaccine response and a vaccine system which models the elderly response to vaccines. For the early stage, a monocyte system was built, which targets key monocyte function which is critical to normal disease progression. For the severe/late stage, a viral system was built, which models the elderly response to high viral load and cytokine storms.

For example, a large screen is run on approved drugs in the viral system for severe/late stage repurposing therapies. Computational models and data are analyzed as part of this screen. Clinically relevant immune features are generated, phenotypes are annotated, and drug/target hits coming out of this screen are prioritized. The viral system may be used for modeling the elderly response to high viral load and cytokine storms. Also, the monocyte system may be used for mild/early stage repurposing therapies. Also, the monocyte system may be used because proper monocyte function is critical for preventing the progression to late stage disease.

Antiviral vaccines generally fall into one of the following types: inactive or live-attenuated viruses, virus-like particles, viral vectors, protein-based, DNA-based, and mRNA-based vaccines. Each of these methods induces acquired immunity to the virus in their own unique way. Inactive/live-attenuated viruses, viral vectors, and protein-based vaccines are traditional approaches which are used in many vaccines. mRNA and virus-like particle based approaches are the next-generation of vaccine technology and they hold an immense amount of promise. Many vaccines for coronavirus are being developed each using one of these different approaches.

While traditional and modern vaccine technology is being developed rapidly, the elderly response to these vaccines is not being accounted for. While the new virus-like particles and mRNA vaccines may produce an enhanced response, this has yet to be proven. Further, the elderly typically do not respond well to vaccines. Several methods such as high dose vaccines and adjuvants have been used to improve the elderly response to vaccines. Currently, developing specific methods to improve the elderly response to coronavirus vaccines is not a priority since the main vaccine has yet to be developed.

Using systems and methods of the present disclosure, repurposable methods are discovered which improve elderly response to vaccines. A phenotypic screening system (e.g., adjuvant system) has been developed, which can find better adjuvants. Also, a phenotypic screening system (e.g., vaccine system) of elderly vaccine response has been developed, which can find approved therapies that rejuvenate the response.

During the mild/early stages of disease, the viral infection has escalated to the point where the viral load stimulates an immune response which results in symptoms such as fever and cough. A healthy early stage response to the virus starts with recognition by the innate immune system and tissue resident cells. Upon recognition of the virus, the innate immune system creates an inflammatory environment in the tissue by producing chemokines and cytokines. These chemicals attract monocytes from the bloodstream to the site of infection. It is there where they differentiate into macrophages or dendritic cells (antigen presenting cells) which, along with neutrophils, fight the virus through mechanisms such as phagocytosis. Monocytes/macrophages also transition the infection site from acute inflammation to chronic inflammation with the continued production of cytokines. Phagocytosis of the virus by these antigen presenting cells leads to antigen uptake and presentation on the cell surface. The antigen presenting cells take the antigen from the site of inflammation to the lymph node where they activate the adaptive immune system. The T-cells and B-cells of the adaptive immune system respond by producing a massive virus specific army which only targets the viral infection. This army reduces the viral load and the activation of the innate immune system. Once cleared, the adaptive army transitions from an effector state to a memory state where it patrols the body for reinfection.

For COVID-19 patients, a dysfunctional monocyte response may be a significant driver of the transition from the mild/early stage to the severe/late stage. From a review of the causes and consequences of cytokine storm and immunopathology in patients with human coronavirus infections that predated the identification of COVID-19, the following may be identified as causes of an exuberant inflammatory response: rapid virus replication, hCoV infection of airway and/or alveolar epithelial cells, delayed type I Interferon (IFN) responses and monocyte/macrophage and neutrophil accumulation. Further, both SARS-CoV and MERS-CoV encode multiple proteins that antagonize IFN responses and that an early antagonism of the IFN response might delay or evade the innate immune response. Therefore, delayed IFN signaling may further orchestrate inflammatory monocyte/macrophage responses and sensitize T cells to apoptosis resulting in a further dysregulated inflammatory response. These inflammatory macrophages accumulate in the lungs and are a likely source of pro-inflammatory cytokines and chemokines associated with fatal disease induced by human coronavirus infections, such as SARS and COVID-19. Autopsy findings from patients with COVID-19 may mirror these findings.

Further, SARS-CoV infection may regulate immune-related genes in monocytes and/or macrophages, which may be important to the pathogenesis of SARS. As a cautionary tale of how anti-Spike antibody and monocyte/macrophage function may make COVID disease worse, it may be shown that anti-Spike IgG made SARS-CoV disease worse by switching macrophage from wound-healing to proinflammatory phenotype. Anti-Spike IgG fails to prevent viral entry. Instead, it binds to the virus, facilitating uptake by macrophages expressing FcR. This leads to macrophage stimulation and their production of proinflammatory cytokines (IL-6, IL-8, MCP1) and loss of tissue-repair cytokine (TGFb). Relevant to this finding is that in COVID-19 patients, the level of serum IgG against Spike protein correlates with older age, disease severity and lymphopenia. In profiling COVID-19 patients, infection induces readily detectable morphological and inflammation-related phenotypic changes in peripheral blood monocytes, the severity of which correlate with patient outcome. There are no detectable differences in the number of monocytes between patients with COVID-19 and normal healthy individuals, but there are significant morphological and functional differences, which are more pronounced in patients requiring prolonged hospitalization and ICU admission.

While the coronavirus causes innate immune system dysfunction on its own, its effect is compounded by the already dysfunctional state of the elderly immune system resulting in increased progression to the severe/late stage. For example, it may be shown that monocytes and macrophages have impaired type 1 IFN and ISG responses but intact initial cytokine responses. The dysfunction of the elderly innate immune system continues along many functional dimensions. The plasticity of monocyte/macrophage polarization is dysfunctional in the elderly. Age-induced changes in macrophages are diverse and, in general, may represent pro-inflammatory activation of cells with an alternatively activated (M2-like) phenotype. Recruitment of monocytes to the site of infection, pathogen recognition receptors, phagocytosis, and antigen uptake/presentation are all deregulated in the elderly.

This leads to a tremendous therapeutic opportunity during the mild/early stage of COVID-19. A therapy which can hasten/improve/boost the initial delayed, dysfunctional response of the monocytes can potentially prevent the transition to severe/late stage disease which may drastically reduce the number of deaths.

The monocyte system was developed to model the dysfunctional/delayed response to coronavirus and the aggravation of that response by inherit dysfunction in the elderly innate immune system. This model system may be used to screen for safe, repurposable drugs which boost the initial innate immune response.

Further, a proper immune response is key to preventing severe/late stage progression. As seen in the early stage, a dysfunctional immune response may result in onset of severe symptoms. When a protective immune response is impaired, virus will propagate and massive destruction of the affected tissues will occur, especially in organs that have high ACE2 expression, such as intestine and kidney. The damaged cells induce innate inflammation in the lungs that is largely mediated by pro-inflammatory monocytes/macrophages. Lung inflammation is a significant cause of life-threatening respiratory disorders at the severe stage. Therefore, good general health may not be advantageous for patients who have advanced to the severe stage: once severe lung damage occurs, efforts may be made to suppress inflammation and to manage the symptoms. The cytokine release syndrome seems to affect patients with severe conditions. The cytokine profiles of healthy, early stage, and late stage COVID-19 patients differ drastically with the late stage patients seeing strong increases in pro-inflammatory cytokines produced by monocytes.

In SARS-CoV-2 infected individuals, interleukin (IL)-6, IL-10, IL-2R, and TNFα surges during illness and declines during recovery. Patients requiring ICU admission have significantly higher levels of IL-6, IL-10 and TNFα and fewer CD4+ and CD8+ T cells. Further, the level of IL-6, IL-10 and tumor necrosis factor (TNF)-α inversely correlates with CD4+ and CD8+ T cell count, confirming animal studies that it is the cytokine storm which dampens adaptive immunity against SARS-CoV infection. IL-6, IL-8, IL-10, IL-1b, TNF-α all produced mostly by monocytes. The innate immune response to tissue damage caused by the virus may lead to acute respiratory distress syndrome (ARDS), in which respiratory failure is characterized by the rapid onset of widespread inflammation in the lungs and subsequent fatality. The symptoms of ARDS patients include short/rapid breathing, and cyanosis. Some severe patients admitted to intensive care units may require mechanical ventilators and those unable to breath have to be connected to extracorporeal membrane oxygenation to support life. CT images may reveal that there are characteristic white patches called “ground glass”, containing fluid in the lungs. Recent autopsies may show that the lungs are filled with clear liquid jelly, much resembling the lungs of wet drowning. Hyaluronan may be associated with ARDS; moreover, during SARS infection, the production and regulation of hyaluronan is defective. The levels of inflammatory cytokines (IL-1, TNF) are high in the lungs of COVID-19 patients and these cytokines are strong inducers of HA-synthase-2 (HAS2) in CD31+ endothelium, EpCAM+lung alveolar epithelial cells, and fibroblasts.

To address the cytokine storm, trials targeting IL-6 may be conducted. One, very small, uncontrolled, trial may show treatment with Tocilizumab to be effective against late stage COVID-19. Within a few days after treatment with Tocolizumab, the fever returned to normal and all other symptoms improved remarkably. Fifteen of the 20 patients (75.0%) may lower their oxygen intake and one patient may need no oxygen therapy. CT scans may manifest that the lung lesion opacity absorbed in 19 patients (90.5%). In other contexts, IL6 blockade may be successful in the cytokine storm caused by CAR T cells. It is not upstream of other cytokines so it may not be predicted whether other cytokines are affected. Conversely, it has a black box warning (highest warning) as follows: Serious infections leading to hospitalization or death including tuberculosis (TB), bacterial, invasive fungal, viral, and other opportunistic infections have occurred in patients receiving ACTEMRA. By targeting IL-6, viral load and other inflammatory cytokines are not being reduced, and the core mechanisms which cause the illness are not being addressed. Reducing viral load may not only reduce the immune stimulus, but also reduce the direct damage by the virus to pulmonary cells. Since elderly people have major problems in their naïve CD8 T cells they may have major problems in slowing down viral replication.

In the late severe/late stage of COVID-19 lymphopenia, reduced level of a certain type of blood cell, lymphocytes, may be an effective and reliable indicator of the severity and hospitalization in COVID-19 patients. Four potential mechanisms leading to lymphocyte deficiency. First, the virus may directly infect lymphocytes, resulting in lymphocyte death. Lymphocytes express the coronavirus receptor ACE2 and may be a direct target of viruses. Second, the virus may directly destroy lymphatic organs. Acute lymphocyte decline may be related to lymphocytic dysfunction, and the direct damage of novel coronavirus to organs such as thymus and spleen cannot be ruled out. This hypothesis may be confirmed by pathological dissection. Third, inflammatory cytokines continued to be disordered, perhaps leading to lymphocyte apoptosis. Basic research may confirm that tumor necrosis factor (TNF)α, interleukin (IL)-6, and other pro-inflammatory cytokines may induce lymphocyte deficiency. Fourth, inhibition of lymphocytes may occur by metabolic molecules produced by metabolic disorders, such as hyperlactic acidemia.

The viral system was developed to model the elderly response to very high viral load and the adaptive immune (T-cell, NK cell, B-cell) system response to a cytokine storm produced by monocytes. This model system may be used to screen for safe, repurposable drugs which decrease inflammation, rejuvenate the elderly response to viruses, and improve the lymphopenia.

### Example 23: Machine Learning Computational Models for Prediction of Phenotypes

Using systems and methods of the present disclosure, computational models are constructed for prediction of aging (e.g., young vs. old) phenotypes (e.g., induced in cells such as immune cells). These models are used to screen and classify potential compounds, such as viral systems, vaccines, adjuvants, monocytes, small molecules, etc. In some embodiments, the computational models comprise one or more trained algorithms, such as trained machine learning classifiers.

In some embodiments, the classification of the compound comprises classifying a given compound (e.g., small molecule) into a category from among a plurality of categories. As an example, in a first level classifier comparing efficacious vs. control, the classifier predicts if a given compound induces a phenotype that looks like any of one or more clinically proven compounds. This may comprise performing a binary classification of the given compound (e.g., into either an efficacious category, or a control or non-efficacious category).

As another example, in a second level classifier comparing compound A vs. compound B vs. control, the classifier predicts if a given compound looks more like a specific compound. This may comprise performing a categorical classification from among three or more categories of the small molecule (e.g., into a compound A category, a compound B category, or a control or non-efficacious category).

As another example, in a third level classifier comparing compound A-high vs. compound A-low vs. compound B-high vs. compound B-low vs. control, the classifier predicts if a given compound looks more like the high or low concentration for a specific compound. This may comprise performing a categorical classification from among five or more categories of the small molecule (e.g., into a compound A-high category, a compound A-low category, a compound B-high category, a compound B-low category, and a control or non-efficacious category).

Input data may be generated to produce training data sets for constructing trained machine learning classifiers as follows. Images are acquired from 384-well plates. For a typical immune experiment, four fields-of-view are acquired from each well, with four vertical Z-layers and five separate fluorescent channels for each field of view. The individual fluorescent channels may be varied to capture sets of broadly relevant cellular components or organelles. For example, Hoechst stains capture some sets of broadly relevant cellular components or organelles (e.g., the nucleus and nuclear morphology), while Mitotracker® stains capture other sets of broadly relevant cellular components or organelles (e.g., mitochondria), and Phalloidin stains capture other sets broadly relevant cellular components or organelles (e.g., actin filaments).

The acquired images are then run through a computational pre-processing pipeline. Next, the post-processed images are fed to a CellProfiler pipeline, which generates 1,256 features per cell. The computed features capture cell morphology (size, shape, etc.), stain intensity, stain texture, stain granularity, stain co-localization across channels, etc.

In parallel, every cell is run through a series of deep learning-based convolutional neural networks, which extra 1,024 unbiased features per channel. These deep learning embeddings are used for a variety of modeling tasks (e.g., cell subtype labeling).

Upon completion of the CellProfiler pipeline, the features are analyzed with respect to the label of interest (e.g., “control” vs. “treatment”, or “young” vs. “old”). Features that exhibit a significant difference across populations are summarized in an auto-generated report, which groups the significant features by type (e.g., morphology vs. staining intensity) and, further, by channel (e.g., Hoechst (for nuclei) vs. Mitotracker® (for mitochondria)). This summary serves as a distillation of the phenotypic differences between populations

In some embodiments, the machine learning classifier comprises gradient-boosted decision trees. The gradient-boosted decision trees may be trained over embeddings generated from pre-trained convolutional neural networks. The machine learning classifier comprising gradient-boosted decision trees may be configured to outperform other modeling techniques (such as random forests and SVMs) and other methods of feature generation (such as extraction of morphology or textural features with a tool like CellProfiler). For example, the use of gradient-boosted decision trees resulted in about 85% accuracy of classification vs. about 80% for random forests.

The machine learning classifier may operate on “embeddings” generated from deep learning models (CNNs) trained on millions of public images. Since these CNNs were trained on external data, they incorporate raw and unbiased visual properties of each cell, including morphology, texture, and intensity features. For example, peripheral blood mononuclear cells (PBMCs) in the systems fall into two broad buckets: lymphocytes, which include T-cells, B-cells, and natural killer (NK) cells; and monocytes, which include normal monocytes, macrophages, and dendritic cells. Generally speaking, differentiating between those two coarse classes is easier, and differentiating within those classes is more difficult as the differences are more subtle. The cell subtype classifiers are generally able to differentiate between all of those subtypes, but show somewhat higher confusion between the subtypes within the coarse categories (e.g. between B-cells and NK cells as compared to between B-cells and monocytes).

In some embodiments, training the machine learning classifier comprises performing various techniques for generating features and feeding them to the gradient-boosted model. Various backbone deep learning architectures (e.g., VGG3 and ResNet) may be used to construct the models. Further, various embedding output layers may be used to construct the models. The efficacy of data augmentation and pseudo-labeling techniques are demonstrated, in which the size of training dataset is able to be increased through computational techniques.

Embeddings may be generated as follows. Whenever a new plate of data is processed, deep learning-based embeddings are generated for every cell using pre-trained convolutional neural networks. These models serve as feature extractors, and the output embeddings are then used for downstream modeling (e.g., by feeding them as inputs to gradient-boosted decision tree-based models (e.g., using XGBoost)).

This approach of generating and training on deep learning-based embeddings was demonstrated to be an effective form of modeling, e.g., for the immune model. Such embeddings can be further optimized to improve the models and the embeddings on which they are trained. An approach to improve the deep learning embedding-based models is provided below, using the immune system and the problem structures engaged with therein as an example. Optimization and improvement of the models and the embeddings on which they are trained comprised data augmentation, architecture search, and parameter sweeps.

The data augmentation was performed with an aim to improve the embeddings and the embedding-based models, which is particularly valuable in the context of hand-labeled or manually-labeled data (e.g., ExampleSets). For example, such hand-labeled or manually-labeled data is used for cell subtype labeling, where hundreds or thousands of hand-labeled cells may be used for training.

The architecture search was performed with an aim to improve the embeddings and the embedding-based models by using different underlying models. For example, ResNet and InceptionV3 models were constructed and evaluated against a VGG model for single-cell embeddings. The manner in which performance changes when embeddings are obtained from different model output layers was investigated. Further, the manner in which performance changes when embeddings are obtained from multiple model output layers was investigated. For this process, the search space was limited to the pre-trained VGG, ResNet, and InceptionV3 models, and two output embedding layers (as well as the concatenation of the two) for each model.

The parameter sweeps were performed with an aim to improve the embeddings and the embedding-based models. After generating the single-cell embeddings, XGBoost models were trained on top of them. The manner in which performance changes when an exhaustive sweep is performed over the XGBoost parameter space, rather than relying on the defaults, was investigated.

Using the immune system as an example, there are a variety of problem structures beyond young-vs.-old aging models. To anchor these lines of research, first core problems were defined to benchmark against, including a cell subtype labeling model, a field-level aging model, a field-level, VSV-activated aging model, a single-cell, VSV-activated aging model, and an AS01 control-vs.-treatment model. Defining these benchmark problems in advance allowed these research questions to be framed as engineering challenges.

The results were obtained as follows. For the data augmentation, a noticeable, positive effect was demonstrated in the cell subtype labeling context. For example, validation accuracy with augmentation performed during training increased by 2.34% as compared to the same model with no augmentation performed during training. Significantly, an increase in the AUC was observed for the least-labeled class (B-cells), from 0.90 to 0.94. Therefore, data augmentation was incorporated into the cell subtype labeling procedure, and is applied to similar problem contexts.

For the architecture search, no single combination of backbone model and embedding layer was universally top-performing across the five problem types. However, the data demonstrated two trends. First, using embeddings from multiple layers (e.g., VGG layer-3 and VGG layer-5, rather than just VGG layer-3 or just VGG layer-5) generally outperforms embeddings from a single layer. Second, the ResNet embeddings generally, but not always, outperformed VGG and InceptionV3. Therefore, some embeddings do better than others on certain problems. The choice of the optimal or best performing embedding for a particular problem depends on many considerations. As a result, generating multiple embedding layers by default and/or migrating the code paths to use ResNet rather than VGG are evaluated for inclusion in computational models. Also, generating the embeddings by default, and automating the choice of embedding, which ultimately becomes a model parameter, may be done.

For the parameter sweeps, the efficacy of parameter optimization was assessed via an XGBoost optimization pipeline, which iteratively locks certain XGBoost parameters and sweeps over others in an attempt to identify the optimal parameter settings for a specific problem. The results and data obtained indicated that validation metrics (e.g., AUC and/or accuracy) can be reliably improved (e.g., by multiple percentage points) via parameter sweeps. Therefore, accuracy and AUC improvements were observed in various problem settings, by using optimized XGBoost parameters as compared to the default parameters. In each case, the parameters have been optimized using the validation set from the first fold, and then applied to every fold in-turn without further refinement.

Generally, the modeling techniques may rely on deep learning embeddings which are unbiased and, in some ways, opaque. However, the models can be also be “interrogated” to discover the signals pertinent to the classification task at hand. This can include, but is not limited to, iteratively using the model to make predictions on individual cells, and visualizing the “top cells” which activate a prediction. In this manner, the most significant differences can be visualized. As an example of such a change, concanavalin textural changes can be observed in aging (which is indicative of increased glycosylation in an elderly subject).

In some embodiments, the computational analysis comprises performing a cell-to-cell interaction analysis, which utilizes spatial statistics to model the spatial point patterns of different cell-types within a given window. This modeling provides a statistical basis for understanding the effect of different conditions on how cells spatially interact (clustering or inhibitory) with other cells.

After processing, individual fields are treated as unique spatial windows. The point location of individual cells are marked with cell-type classifications within these windows. This effectively translates an image of a field to a “marked point pattern.” A dataset is constructed using these marked point patterns, relevant spatial covariates (e.g. relative field location), and global covariates (e.g. donor identification number, well condition). This marked point pattern dataset is used to model the underlying point pattern process.

Spatial point pattern processes are modeled using 2 approaches. First, spatial point patterns of individual fields may be modeled with non-Poisson, multi-type point interactions for specific cell-types. From these individual point pattern models, cell-to-cell interaction parameters for specific cell-types-to-cell-type interactions (e.g., T-cell to Macrophage) are calculated for each field for subsequent analysis. Second, spatial point patterns of a selection of fields may be modeled using replicated spatial point pattern modeling. A series of stepwise replicated point pattern models with non-Poisson multi-type point interactions are constructed to analyze point pattern interactions of different cell-types with respect to conditions of interest. These replicated point pattern models may include a number of global and spatial covariates to model heterogenous and homogeneous point-patterns, varying interaction parameters describing interpoint interactions, and interactive interaction parameters describing the relationship of conditions of interest and cell-to-cell interactions. This modeling provides evidence of whether or not the conditions of interest alter the cell-to-cell interactions of targeted cell-types (e.g. monocyte to monocyte, T-cell to macrophage, etc.), and how cell to cell interactions change with these conditions (e.g. T-cells exhibiting increased clustering with macrophages under a given condition x).

### Example 24: The CPMM palette

There present disclosure includes multiple palettes for profiling of morphological state and quantification of dead cells of PBMCs for young vs. old classification. The CPMM palette is a modification of CellPainter Stain I (described elsewhere herein). Fluorescent lectins like Wheat Germ Agglutinin (WGA) and Concanavalin (ConA) selectively bind to glycoprotein and glycolipids in biological membranes. Phalloidin has a specificity for F-actin within cells. Mitochondria generate a potential across their membranes due to the activities of enzymes of the electron transport chain. MitoTracker® Orange CMTMRos is an orange-fluorescent dye that stains mitochondria in live cells and its accumulation is dependent upon membrane potential. The dye is well-retained after aldehyde fixation. Carbonyl cyanide 4-(trifluoromethoxy)phenylhydrazone (FCCP), used as a positive control, is an ionophore which uncouples the respiration chain (ATP synthesis) by transporting protons through the mitochondrial membrane without generating energy for oxidative phosphorylation

Using the CPMM palette image acquisition may be conducted with binning 1 and 4FOV with 40× water objective NA 1.0, non-confocal with z-stack of 4 planes starting at −2 μm with distance steps of 1.0 μm.:

| Channel | Label | Ex | Em | comment |
|---|---|---|---|---|
| 1 | Brightfield | Transmission | 655-760 | |
| 2 | Hoechst 33342 | 355-385 | 430-500 | Nuclei |
| 3 | Alexa 488 | 460-490 | 500-550 | WGA |
| 4 | MCTracker Orange | 530-560 | 570-650 | MitoTracker/ |
| | | | | mCherry |
| 5 | Alexa 633 | 615-645 | 655-705 | ConA |
| 6 | Alexa 700_narrow | 650-675 | 685-760 | Phalloidin 700 |

### Example 25: Use of the CPMM Palette with Frozen PBMCs

In this example, thawed frozen PBMCs were seeded into HTS assay plates, treated with a compound treatment, and fixed. The fixed cells were stained using the CPMM palette and images, including data export.

The following thawing procedure was used: 10 ml of prewarmed Culture Medium was added to a 15 ml tube; cell vials were quickly thawed at 37° C. (e.g. in a water bath) until thin layer of ice is still present, the cells were transferred to a centrifuge tube, centrifuged, resuspended in 10 ml culture medium.

The following plating procedure was used: cells were centrifuged and counted; cells were resuspended cold Plating Medium to a plating cell density of 0.4 Mio/ml (incl. correction factor 2.5×) to achieve approx. 10.000 cells/well; 350 μl of cell suspension was transferred into a well of a 96 Well MasterBlock (see layout) stored on ice (4° C.); the 96 Well Masterblock with cells moved on chilled position on Felix together with pre-warmed Assay Plate; plate was centrifuged, and incubated for the cells to adhere; after 30 min 10 μL TriggerMedium was added with our without stimulus/compound from DilutionPlate using Felix Script 406 “Transfer compound”; plate centrifuged and incubated for 24 h.

The cells were treated with MitoTracker® reagents for 30 minutes, washed, and fixed. The fixed cells were permeabilized, blocked and contated with the CPMM reagents, as mentioned in Example 24.

Images were acquired with binning 1 and 4FOV with 40× water objective NA 1.0, non-confocal with z-stack of 4 planes starting at −2 μm with distance steps of 1.0 μm.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

### Example 26: Cell Adhesion in a Mixed Lymphocyte (PBMC) Microscopic Assay

When imaging cells in culture, it is important that the cells remain adhered to a fixed substrate so that movements of the cells do not occur during image collection. However, many cell types are not normally adherent and instead remain in suspension. This example describes a novel approach which makes imaging non adherent cells, e.g., PBMC, possible.

Cell adhesion occurs between cells and neighboring cells or between the cell and (extracellular-) matrix. In a microscopic imaging assay, the matrix is typically the surface of a microtiter imaging plate where the cells can adhere. Cell adhesion occurs from the interactions between cell adhesion molecules, a family of transmembrane proteins located on the surface of the cell, and extracellular matrix proteins.

Here, cell culture materials are used to promote cell adhesion. This comprises tissue culture (TC)-treated polystyrene surfaces of microtiter plates (plasma ionization of polystyrene substrate); tissue culture-compatible coatings like amino acid multimers of poly-D lysine; collagen or extracellular matrix proteins; advanced proprietary tissue culture coatings like CellBind™, AdvancedTC™; cell glue like sea mussel protein extract and many more.

In some examples, to avoid unspecific cell activation in a mixed lymphocyte population, no protein/peptide-based tissue culture adhesion enhancers were employed. Instead cells were kept on ice after the thawing procedure during the initial cell plating preparation. Low temperature minimizes the auto-adherence from cell-to-cell (cell clumping) in the monocyte/macrophage population, when cells were kept in cell culture medium depleted from protein supplements like BSA/FBS (plating medium). Cell were transferred to TC-treated polystyrene imaging assay plates containing pre-warmed plating medium to quickly bring the temperature to 37° C. The Assay Plate was centrifuged 1 min at 124 g to accelerate cell sedimentation. Cell adhesion in the Assay Plate was allowed for 30 min in a TC incubator (37° C., 5% CO2). Since all proteins were deprived from the plating medium, cell adherence predominately occurred between the cell and the polystyrene surface of the Assay Plate. After 30 min, medium contain FCS was added to a final concentration of 10%.

FIG. 24 shows illustrative cells in a mixed lymphocyte population. The image to the left shows good cell adherence of monocyte population, defined cytoskeleton structure, whereas the image to the right shows poor cell adherence with many cells out of focus and no defined cytoskeleton structures.

### Example 27: Classification of Cell Types Based on Unbiased Morphological Analysis

One important phenotype of the multi-phenotype aging profile is cell composition. Traditional methods for measuring cell composition were unable to scale to the needs of the high-throughput system. Recent studies have shown that functional changes in cells can change their shape and that changes in the cell's shape can change its function. A scalable method for classifying immune cell types was designed within bulk PBMCs based on cell morphology. To highlight a diverse range of morphological changes, cells were stained with cell painting palettes (as disclosed herein) that broadly characterize a variety of cellular organelles such as the nucleus, cytoskeleton, mitochondria, and cell membrane, as described herein. To understand and verify the morphological differences between immune cell types, PBMCs were isolated from whole blood; T cells (CD3+), B cells (CD19+), natural killer (NK) cells (CD56+), and monocytes (CD14+) were further isolated based on customarily-defined cell surface markers. Isolated cells were plated, stained with Cell Painting Palette 1, and imaged.

### Cell Painting Palette 1.

| | | Excitation | Emission | |
|---|---|---|---|---|
| Channel | Label | (nm) | (nm) | Comment |
| 1 | Brightfield | Transmission | 655-760 | |
| 2 | Hoechst 33342 | 355-385 | 430-500 | Nuclei |
| 2 | Alexa 488 | 460-490 | 500-550 | Wheat germ |
| | | | | agglutinin |
| 4 | Texas Red | 530-560 | 570-650 | Phalloidin |
| 5 | Alexa 633 | 615-645 | 655-705 | Concanavalin A |

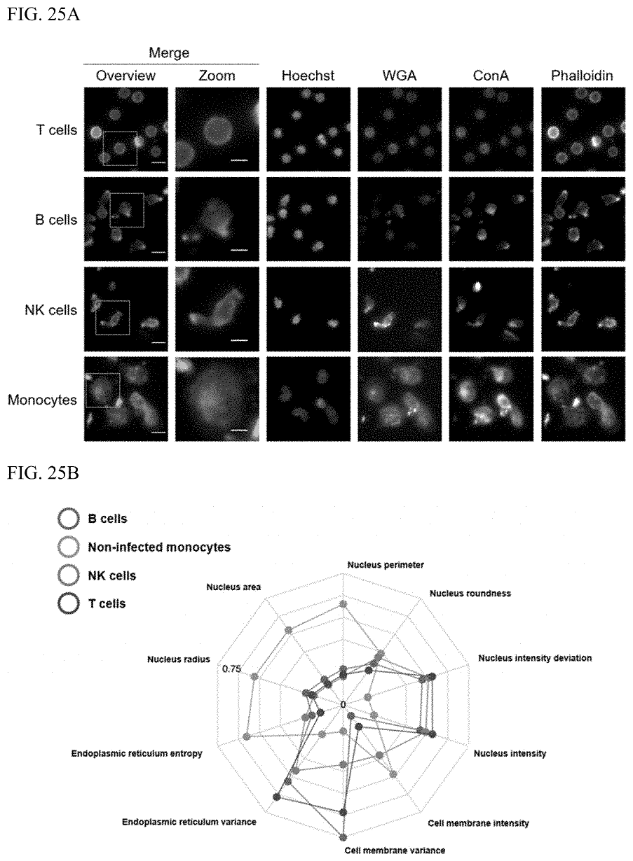

Within these images were observed and quantified notable differences in the shapes of the isolated immune cell populations. As shown in FIG. 25A, the T cells and B cells appeared most similar to each other; the T cells had a rounder and more uniform morphology, while the B cells had a more oval-shaped form with small actin caps on either end. The NK cells diverged from the tightly spherical form of other lymphocytes and showed more elongated and irregular forms. The monocytes were the populations with the largest cellular size encompassing podosomes, dendrites, and kidney-shaped nuclei (FIG. 25A). These data are quantified and summarized in FIG. 25B.

Using the observed phenotypic variation in isolated cells, a computational model was designed to classify single cells within a mixture of PBMCs. The model was built on PBMCs exposed to rVSV-ΔG-mCherry so that the multi-phenotype aging profile may accurately measure the compositional changes between young and old rVSV-infected PBMCs. These cells were then stained with Cell Painting Palette 2, which revealed similar organelles as Cell Painting Palette 1 but included a MitoTracker dye that overlapped with an mCherry protein that fluoresced upon expression.

### Cell Painting Palette 2.

| | | Excitation | Emission | |
|---|---|---|---|---|
| Channel | Label | (nm) | (nm) | Comment |
| 1 | Brightfield | Transmission | 655-760 | |
| 2 | Hoechst 33342 | 355-385 | 430-500 | Nuclei |
| 3 | Alexa 488 | 460-490 | 500-550 | Wheat germ agglutinin |
| 4 | MCTracker Orange | 530-560 | 570-650 | MitoTracker/mCherry |
| 5 | Alexa 633 | 615-645 | 655-705 | Concanavalin A |
| 6 | Alexa 700_narrow | 650-675 | 685-760 | Phalloidin iFluor700 |

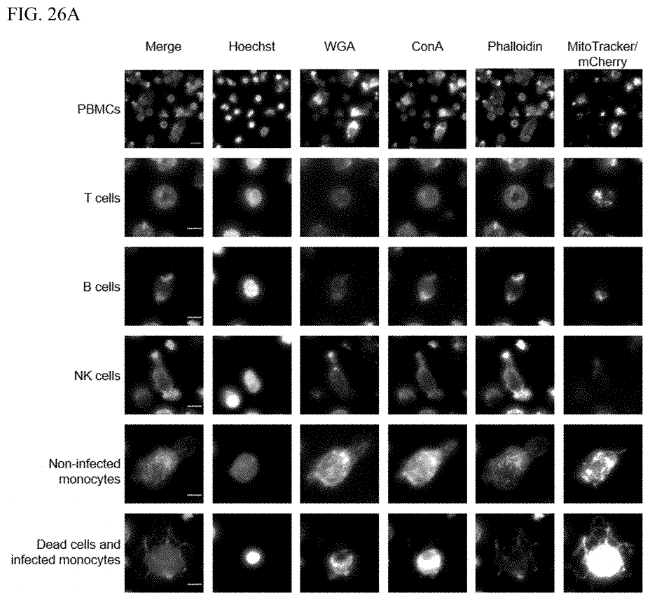

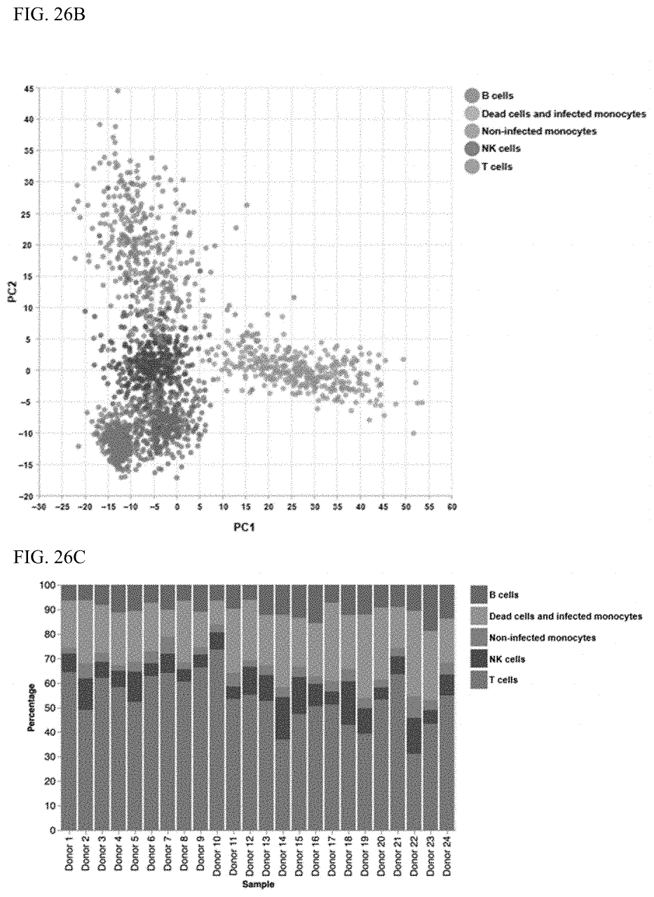

Since only the monocytes and macrophages were infected with the virus, they were the only cell types to show a stark mCherry signal representing infection (FIG. 26A). Image embeddings, a statistically optimized representation of an image, were generated on an expert-curated training set of >61,000 single cell images and then used to train a classification model. The model classified a single cell in an image of bulk PBMCs as a T cell, B cell, NK cell, noninfected monocyte, or dead cell and infected monocyte (FIG. 26A). To validate the accuracy of the classification, an expert-labeled test set of >10,000 single cell images was built from PBMCs. When the classifier was evaluated on this data set, the model obtained 91% accuracy. On the single cell images, a principal component analysis was run on several hundred features derived from CellProfiler that were not used to train the model. Coloring the first two principal components by the model-predicted classification label resulted in clearly identifiable clusters for each class (FIG. 261B). Because immune cell populations vary significantly between individuals, the model was run on PBMCs derived from several different individual donors to see if this variance may be captured by the classifier. The predicted cell compositions not only recapitulated the large inter-donor variance but also fell within the expected range of PBMC compositions (FIG. 26C). Since the classifier was consistently validated using these methods, its predictions were then compared to flow cytometry.

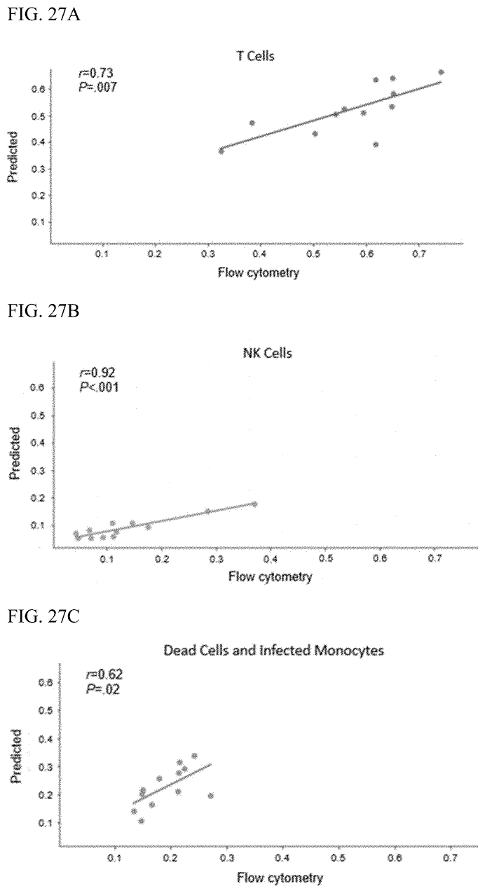

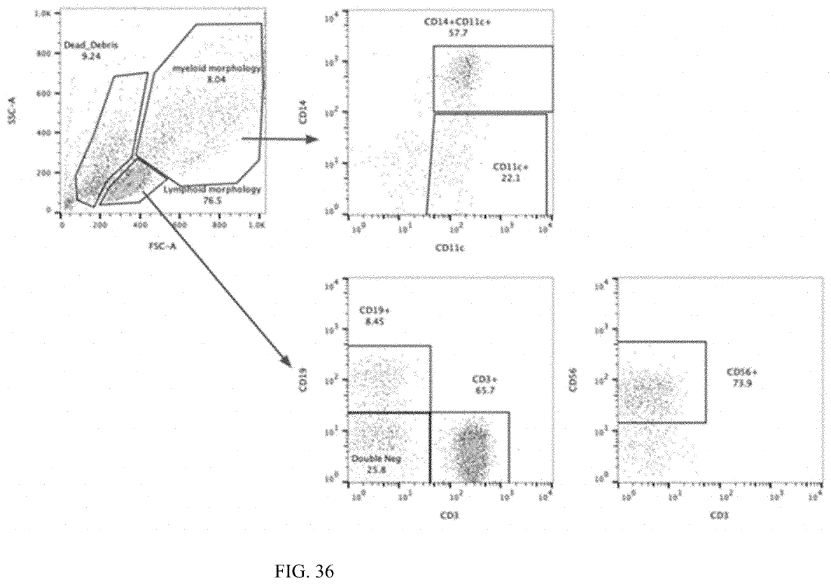

For comparison with flow cytometry, the cell composition predictions from the morphological model was correlated with those identified using flow cytometry (FIG. 36). The cell composition predictions from the model significantly correlated with those determined by flow cytometry for T cells (r=0.73; P=0.007) and NK cells (r=0.92; P<0.001). The dead cell and infected monocyte composition significantly correlated with the live and dead myeloid cell composition derived from flow cytometry (r=0.62; P=0.02; FIG. 27C). A B-cell marker was not included in the flow cytometry panel, so a correlation for that population was not calculated. While flow cytometry was able to accurately differentiate live and dead cells at different granularities, the morphological properties of dead cells in the images were very similar to rVSV-infected monocytes, making it difficult to differentiate live and dead infected cells. The cell type classification model developed and described herein provides a scalable, reproducible, and functionally meaningful method to incorporate cell composition and other phenotypic features into a multi-phenotype aging profile.

### Example 28. Modeling the Inflammatory Antiviral Immune Response

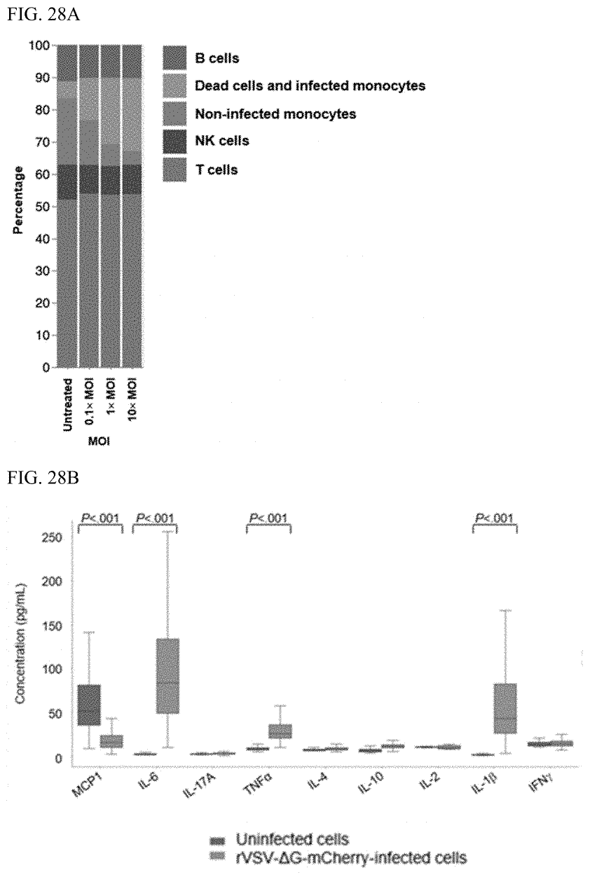

Dysregulated immune responses are a central driver of disease progression in severe infection of older adults. To develop aging profile features that specifically measure phenotypes related to viral infection, the responses to viral load were profiled in middle-aged (30-60 years old) PBMCs. As shown in FIG. 28A, notable shifts were observed in cellular compositions of virally infected PBMCs; specifically, there was a significant increase in dead cells and infected monocytes and a significant decrease in noninfected monocytes at 0.1×, 1×, and 10× multiplicity of infection (MOI; P<0.001). The NK cell population significantly decreased for all viral conditions (P<0.00l). The T-cell and B-cell populations did not undergo significant changes due to viral load. Proinflammatory cytokine levels from the entire PBMC population increased in response to viral infection, with significant increases in interleukin (IL)-6 (P<0.001), tumor necrosis factor (TNF)α (P<0.001), IL-103 (P<0.001), and IL-8 (P<0.001) at 10×MOI. Monocyte chemoattractant protein 1 (MCPI; P<0.001) significantly decreased upon viral exposure compared with untreated PBMCs (FIG. 28B). Given the clear changes in PBMC composition and cytokine production upon viral exposure, these features were incorporated into the multi-phenotype aging profile to understand how they differed with age.

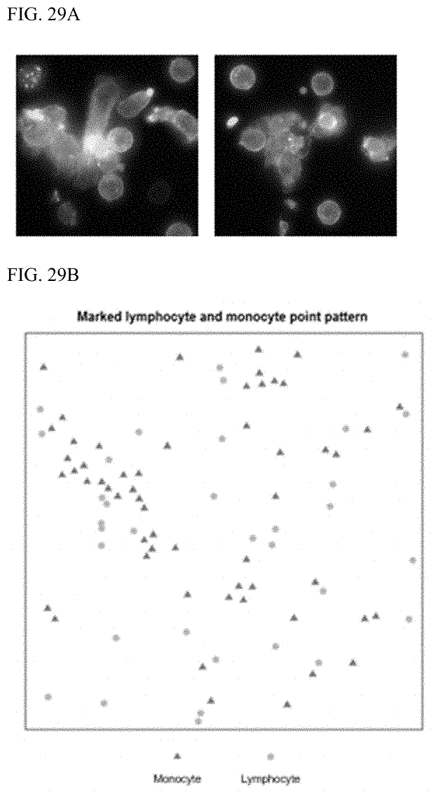

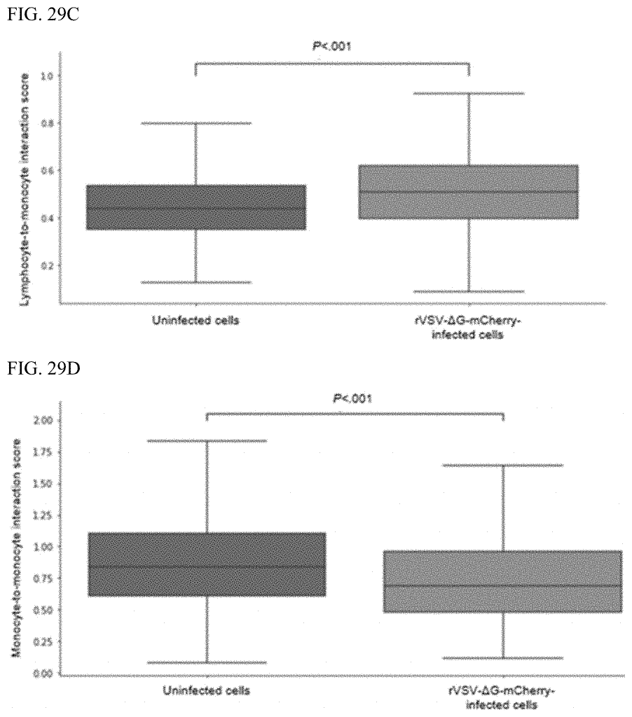

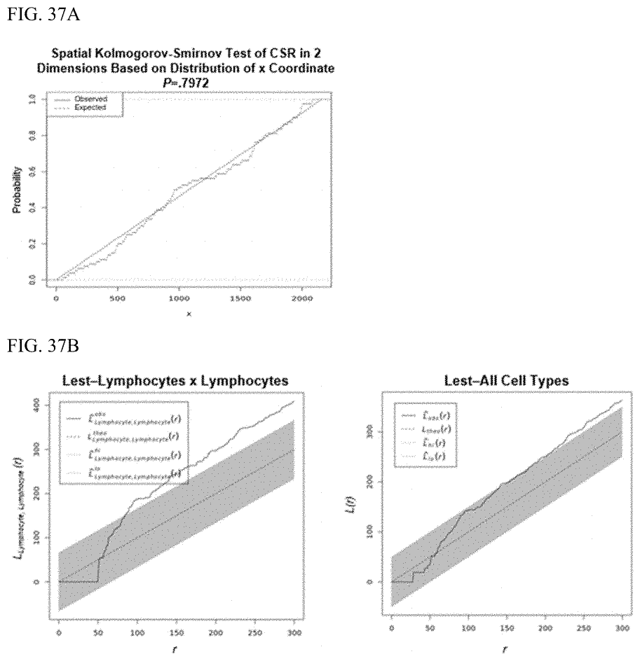

The inflammatory environment induced by viral exposure is also responsible for functional changes, such as physical interaction between cell types. For example, interactions between T cells and antigen-presenting cells lead to T-cell activation, as well as aggregation of macrophages, which contributes to cytokine production. The advantage of cell imaging is the generation of functionally-meaningful point patterns to model how interactions change with various perturbations. To this end, an interaction score was computed using spatial statistical analysis to model non-Poisson multitype point distributions of specific cell types (FIG. 29B). The point patterns were assessed for complete spatial randomness using quadrate tests and distance methods. Spatial point pattern distributions of PBMCs were found to be homogenously distributed with strong evidence of interpoint interactions (FIGS. 37A-B). Upon viral exposure, the interaction score that captured lymphocyte-to-monocyte aggregation significantly increased compared with the uninfected cells (P<0.001; FIG. 29C). In contrast, the monocyte-to-monocyte aggregation significantly decreased when exposed to rVSV (P<0.001; FIG. 29D). These interaction scores were also added as features to the multi-phenotype aging profile.

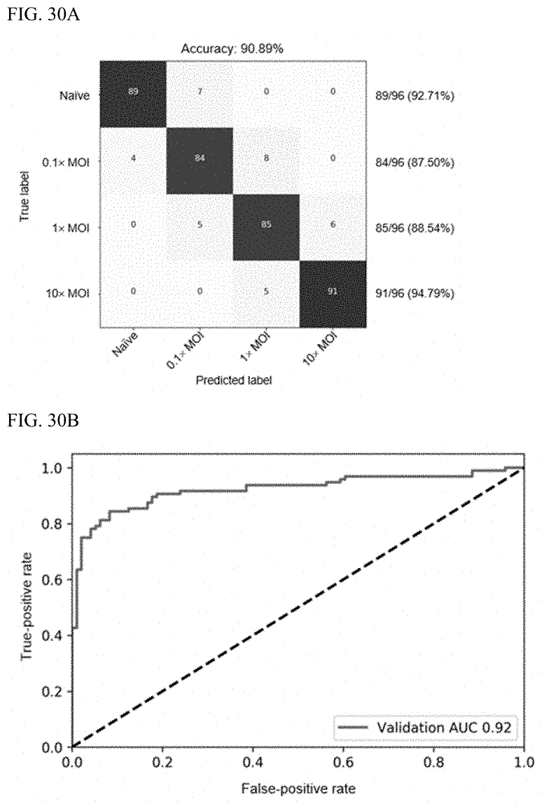

To more broadly measure how the immune response changes to different viral loads, machine learning algorithms were trained to learn the differences in the cellular immune response. Embeddings were generated on single cell images from PBMCs that were exposed to rVSV at different MOIs (0.1×, 1×, and 10×MOI) and stained with the Cell Painting Palette 2, as disclosed herein. The embeddings were then used to train a machine learning classifier to discriminate the cellular responses to rVSV at a range of MOIs. Four different MOIs were replicated across 24 donors and evenly distributed across a 384-well plate assay to minimize bias due to plate location. This resulted in 96 wells per condition. Stratified four-fold cross-validation was used to evaluate the classifier's performance on each well. When the model trained on only monocytes was evaluated, the classifier was able to differentiate between uninfected monocytes and monocytes exposed to 0.1×, 1×, and 10× viral MOIs with 91% accuracy across all four-folds (FIG. 30A). A model was trained on T cells that was unable to distinguish between the uninfected cells and the cells infected at 0.1× and 1×MOI conditions. However, the model learned significant differences between the T-cell response to 10×MOI cells and uninfected cells. The model was able to distinguish these conditions at an area under the receiver operating characteristic curve (AUC) of 0.92 across the 4-folds (FIG. 30B). These validated models of cell response to viral loads can be used to observe if the phenotype of older PBMCs looks more similar to the higher viral load phenotype compared with younger PBMCs.

### Example 29: Dysfunction in the Viral Immune Response in Aging Adults

The multi-phenotype aging profile was used to conduct a novel characterization of the differences between young and old viral immune responses across dozens of features.

### Multi-Phenotype Aging Profile for Viral Response

| Category | Metric |
|---|---|
| Compositional | % T cells |
| | % Dead cells and infected monocytes |
| | % Noninfected monocytes |
| | % NK cells |
| | % B cells |
| Cytokines | IFNγ, IL-1β, IL-2, IL-4, IL-6, IL-8, IL-10, IL-17A, MCP1, |
| | TNFα |
| Aging models | T cell |
| | NK cell |
| | Monocyte |
| | PBMCs |
| Cell-to-cell interaction | Monocyte ↔ T cell |
| | Monocyte ↔ Monocyte |
| | T cell ↔ T cell |
| Structural | Reticular mitochondria |
| | Concentric cytoskeleton |
| | Mitochondrial texture |
| Viral load models | Monocyte viral load response |
| | T-cell viral load response |

IFN, interferon; IL, interleukin; MCP1, monocyte chemoattractant protein 1; MOI, multiplicity of infection; NK, natural killer; PBMC, peripheral blood mononuclear cell; TNF, tumor necrosis factor.

The aging profiles for PBMCs from 49 younger (<35 years of age) donors and 40 older (>60 years of age) donors was generated. Demographics of the donors reflected the standard population of older adults, with older donors having a higher number of comorbidities, particularly hypertension, and taking more medications than younger donors.

### Demographics of Sample Donors

| | Younger | Older |
|---|---|---|
| | population | population |
| | (N = 49) | (N = 40) |
| Age, y | | |
| Mean (SD) | 26.4 (5.9) | 68.2 (7.4) |
| Median (range) | 27.0 (19-35) | 65.5 (60-86) |
| Weight, kg | | |
| Mean (SD) | 85.6 (22.7) | 76.9 (17.5) |
| Median (range) | 81.8 (52.3-186.4) | 72.0 (51.4-87.7) |
| Sex, male, n (%) | 13 (26.5) | 17 (42.5) |
| White, n (%) | 32 (65.3) | 37 (92.5) |
| No comorbidities, n (%) | 26 (53.1) | 6 (15.0) |
| Number of comorbidities,^{a} mean | 1.1 | 2.4 |
| Common comorbidities,^{b} n (%) | | |
| Hypertension | 2 (4.1) | 17 (42.5) |
| Gastroesophageal reflux | 4 (8.2) | 9 (22.5) |
| Seasonal allergies | 9 (18.4) | 7 (17.5) |
| Anxiety | 9 (18.4) | 5 (12.5) |
| High cholesterol | 0 | 6 (15.0) |
| Number of medications, mean | 1.5 | 3.8 |
| Alcohol use, n (%) | | |
| Current use | 23 (46.9) | 20 (50.0) |
| Previous use | 3 (6.1) | 2 (5.0) |
| No history | 22 (44.9) | 18 (45.0) |
| Unknown | 1 (2.0) | |
| Tobacco use, n (%) | | |
| Current use | 4 (8.2) | 8 (20.0) |
| Previous use | 4 (8.2) | 8 (20.0) |
| Never used | 41 (83.7) | 24 (60.0) |
| Viral status, negative, % | | |
| Hepatitis B virus | 100.0 | 100.0 |
| Hepatitis C virus | 100.0 | 100.0 |
| HIV | 100.0 | 100.0 |

^{a}Donors may have more than one comorbidity.
^{b}Incidence of ≥15% in either population.

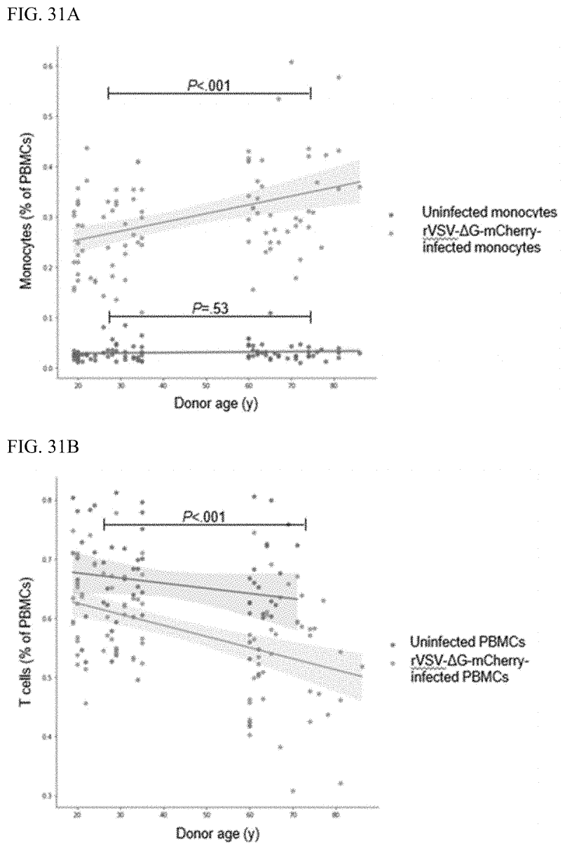

All 89 donors were plated across two 384-well plate assays, with the two age populations evenly distributed across the plate to account for plate position effects. The donors were exposed to rVSV at 10′ MOI and stained with the Cell Painting Palette 2, as disclosed herein. Compared with younger donors, PBMCs isolated from older donors had an increased percentage of dead cells and infected monocytes but showed no significant change in the percentage of noninfected monocytes (P<0.001 and P=0.53, respectively; FIG. 31A). Before viral infection, the percentage of T cells did not show a significant difference between younger and older donors (P=0.11). However, after viral infection, the T cells from older donors were significantly decreased compared with younger donors (P<0.001; FIG. 31B). This may be related to clinical lymphopenia, which has been observed in severely infected patients and is a predictive marker of infection severity in pneumonia, COVID-19, and sepsis. Without being bound by theory, the decreased T-cell population may recapitulate, at least in part, the lymphopenia observed in severely infected patients.

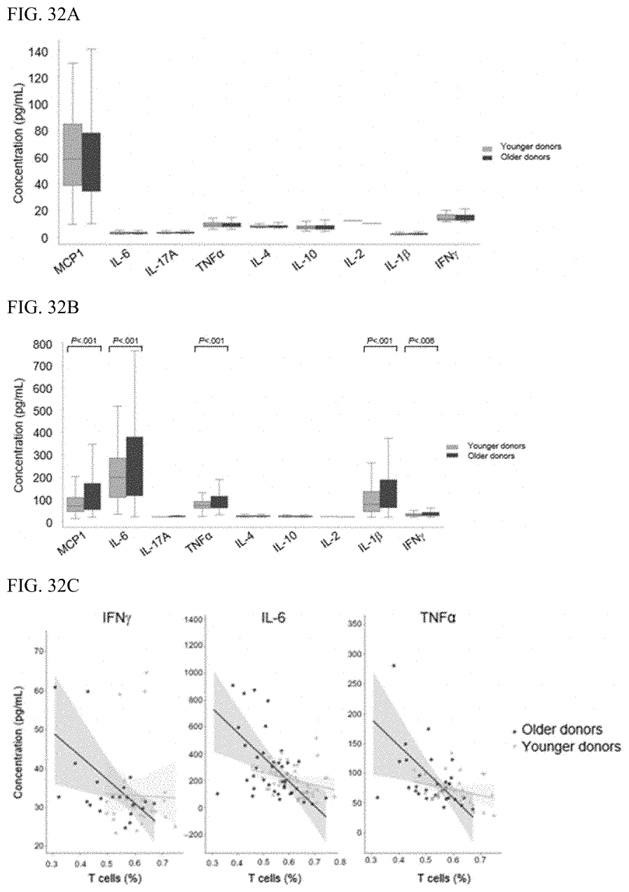

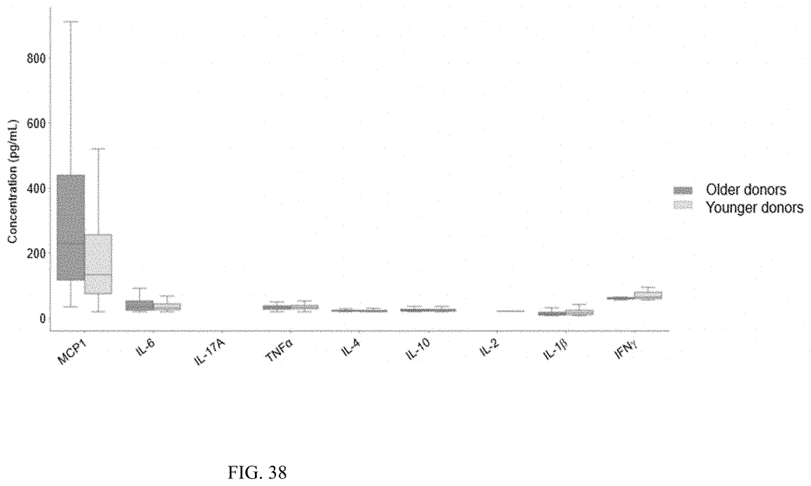

Decreased T-cell levels in older individuals may be driven by cytokine-induced apoptosis or pyroptosis. This has been demonstrated to occur via induction of proinflammatory cytokines (e.g., TNFα) and defects in the nuclear factor-κB (NF-κB) signaling cascade. To understand this relationship in aging, cytokine levels were compared in the multi-phenotype aging profiles of younger and older donors. No significant changes in cytokine production were observed between younger and older donors in uninfected PBMCs (FIG. 32A); there was also little difference in cytokine production of these cells when exposed to rVSV at 1×MOI (FIG. 38). To see significant differences in young and old cytokine production, the PBMCs were exposed to 10×MOI. After infection with rVSV at 10×MOI, the production of proinflammatory cytokines significantly increased in PBMCs from older donors compared with those from younger donors (IL-6 [P<0.001]; MCP1 [P<0.001]; TNFα [P<0.001]; IL-β [P<0.001]; interferon [IFN]γ [P<0.006]; FIG. 32B, and IL-8 [P<0.001]). These same cytokines have been implicated in the progression of hyperinflammation in severe sepsis, COVID-19, and pneumonia. Interestingly, the levels of IFNγ, TNFα, and IL-6 were also inversely correlated to the percentage of T cells in PBMCs exposed to rVSV at 10×MOI (IFN: older donors [r=−0.49; P=0.02], younger donors [r=−0.02; P=0.93]; TNFα: older donors [r=−0.60; P<0.001], younger donors [r=−0.30; P<0.09]; IL-6: older donors [r=−0.60; P<0.001], younger donors [r=−0.32; P<0.02]; FIG. 32C). Recent studies of patients with COVID-19 demonstrated an inverse relationship between peripheral T-cell numbers and serum IL-6 and TNFα levels in patients over 60 years of age. The decrease in T-cell percentages and increase in proinflammatory cytokines that were observed in PBMCs obtained from older donors may similarly indicate a potential set of mechanisms that may lead to the progression of severe infection. A better understanding of the mechanisms driving these differences between young and old donors may result in potential treatments and reduce progression in older adults.

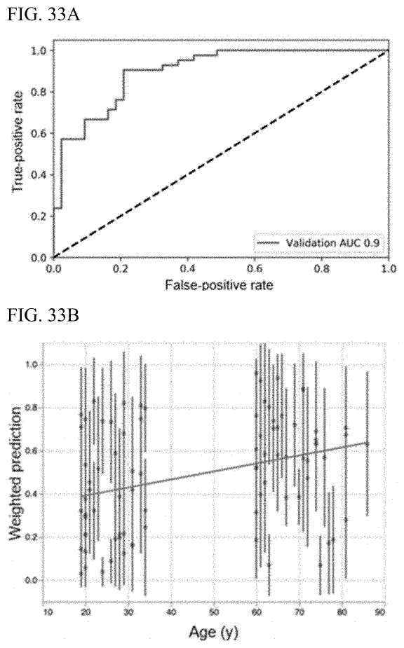

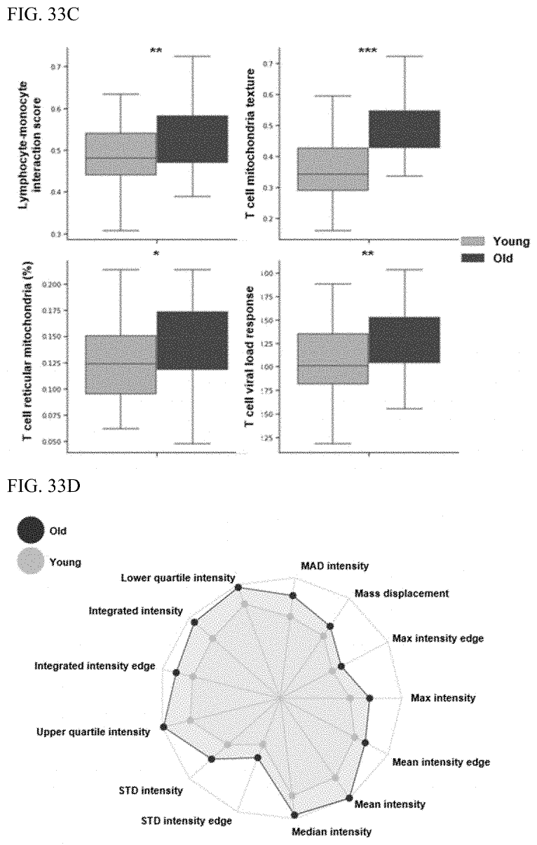

Next, image embeddings of T cells segmented from Cell Painting Palette 2-stained PBMCs were used to train an unbiased machine learning model to learn the hidden complexities contributing to the differences between the viral immune response in younger and older donors. Stratified four-fold cross-validation was used to evaluate the classifier's performance on each well. The model was able to differentiate between the antiviral T-cell response in cells obtained from younger and older donors at an AUC of 0.90 (FIG. 33A). The probability distribution of the test fold predictions was significantly different between younger and older populations (P=0.002). The experiment was repeated several times with continued significant performance. During repeat validation experiments, the model was capable of significantly separating the same samples from younger and older donors (P=0.002; FIG. 33B). The robustness of the model enables an accurate, unbiased measurement of age that incorporates many different dimensions of cellular functions. This powerful tool can help discover modulators of previously unknown aging mechanisms.

Cell composition, cytokines, and unbiased machine learning-driven aging models are only a fraction of the features in the multi-phenotype aging profile. The virus-related features in this aging profile also showed significant differences between young and old responses. The lymphocyte-to-monocyte interaction score, shown to increase with viral infection, was significantly increased in older donors compared with younger donors (FIG. 29C; FIG. 33C). The thus constructed T-cell viral load model is capable of predicting the T-cell response to the amount of viral load. The model predicts that the older donors respond to a higher viral load than the younger donors (FIG. 33C). Together, these data demonstrate that viral features within the multi-phenotype aging profile suggests that older PBMCs present increased viral phenotypes compared with younger PBMCs.

There are several features in the profile that provide a novel view of T-cell metabolism. The structural distribution of the mitochondria has been shown to be related to its function. Spherical mitochondria, elongated mitochondria, and fused/reticular mitochondria all drive specific metabolic functions. Clear differences were observed in the cellular distribution of mitochondria in T cells from younger donors and older donors after viral exposure. The intensity of MitoTracker staining, a measure of membrane potential, was significantly increased at the edge of the cell (P=0.03) in older populations; a significant increase in the standard deviation of mitochondrial distribution was also observed throughout the cell (P=0.003; FIG. 33D). These measurements were complemented by specific features that calculate the percentage of reticular mitochondria and changes in the overall texture of the mitochondria. The percentage of reticular mitochondria was significantly increased in older adults compared with younger adults, while a model of mitochondria texture also showed significant differences (P=0.04; P<0.001; FIG. 33C). These changes in mitochondrial structure between younger and older cells are consistent with reported mitochondrial mechanisms that have been linked to the age-related decline in T-cell senescence and function driving the age-related decline in T-cell function. Therapies that alter the mitochondrial behavior in cells from older donors, or any of the other signatures modeled in this system, may improve the response to infection in older individuals. The many significant differences between young and old viral immune responses are measurable by the dozens of features in the herein-disclosed multi-phenotype aging profile.

### Example 30: Evaluating the Multi-Phenotype Aging Profile of Approved Therapies

To find promising therapies, 3428 commercially-available bioactive compounds were evaluated by generating the multi-phenotype aging profile for each. Of the 3428 compounds, approximately 1900 have been approved for clinical use in at least one country. Young and old rVSV-infected PBMCs from 35 donors were seeded in 384-well plates, then the older donors were treated with a single therapy or left as an old untreated control. Each individual treatment and control well generated its own multi-phenotype aging profile containing dozens of age-related features. Every feature in the aging profile was classified as a “hit” or “miss” based on the magnitude of its distance from old untreated controls. All labeled features were combined into scores for each therapy's aging profile by weighting each feature based on its relevance to immune aging or COVID-19. For the COVID-19 cytokine score, the weights of cytokines known to be part of COVID-19 pathologies were increased. Therapies moving these cytokine levels in a beneficial direction may rank higher. For the immune aging score, the features were weighted based on the consistency and magnitude of the differences between young and old donors. Scoring was tailored to focus on any dysfunction in any specific disease by altering weights of the features. For example, a disease like cancer may have a score that weighted the cell-to-cell interaction features higher to find therapies that improve T-cell trafficking.

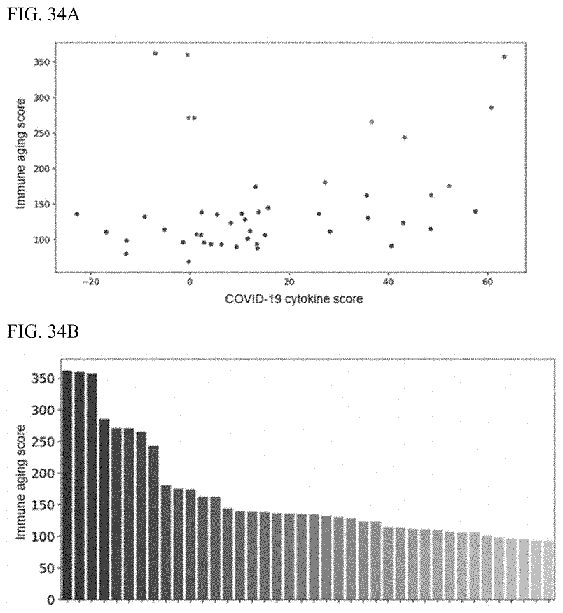

These immune aging and COVID-19 cytokine scores allowed for the evaluation of thousands of therapies for their therapeutic potential by measuring how each therapy affects dozens of age-related phenotypes. All therapies were ranked by the COVID-19 cytokine and immune aging scores, then the top 196 highest-scoring therapies were validated in a dose-response study. In the validation study, the beneficial effect was reproduced in 84% of the top compounds. The high rate of reproducibility means that the effect measured by the scores was quite robust. Each therapy's multi-phenotype aging profile also captured its unique diversity in comparison to all the other thousands of therapies. For example, some therapies showed beneficial potential because they made old T cells respond like younger T cells and changed the reticular shape of the mitochondria. Other therapies showed beneficial potential by reducing predicted viral load and decreasing levels of proinflammatory cytokines. Through this process, triptonide was identified as a therapy with potential for rejuvenating the aging immune system (FIGS. 34A-34B).

A significant number of the top hit compounds saw a compositional shift which was in the opposite direction of a young phenotype. For many of these compounds, dead cells and infected monocytes significantly increased while T cells significantly decreased. Without wishing to be bound by theory, this increase may have been mostly due to cells undergoing compound-induced apoptosis which were then labeled as dead cells and infected monocytes. While the T-cell machine learning aging models were not trained or evaluated on cells from this class, the ability of these models to capture subtle trends from high dimensional data leaves them vulnerable to bias by technical effects. This requires special tooling and attention to ensure known confounders, such as plate, donor, and batch effects, are not biasing the models and that proper awareness is brought to new potential confounders. The many features in the herein-disclosed multi-phenotype aging profile allows provides discovery of various compound effects leading to the identification of some features, such as apoptosis, which may be potential confounders. It is uncertain whether or not an increase in apoptosis impacted the model or whether the impact may be a negative artifact rather than a real beneficial aging phenotype. One hypothesis is that the increase in apoptosis caused differential cytokine release which induced an early stimulation of the innate immune system so that it was more prepared to mount a better viral response and resolution. The apoptotic and rejuvenating effects of the compounds may have combined to produce a stronger overall viral response.

### Example 31: Evaluation of Triptonide's Effect on the Multi-Phenotype Aging Profile of Old PBMCs

Triptonide, a diterpenoid from the medicinal herb Tripterygium wilfordii Hook F,45 had notable effects on virally-infected PBMCs from older donors. Tripterygium compounds demonstrated anti-inflammatory, immunosuppressive, and antiviral effects in animal models and in vitro studies. The anti-inflammatory effects of Tripterygium compounds target both T cells and macrophages. For T cells, activation is suppressed through inhibition of NF-κB transcription. For macrophages, IL-10, IL-6, and TNFα production are decreased and expression of the anti-inflammatory cytokine IL-37 is increased.

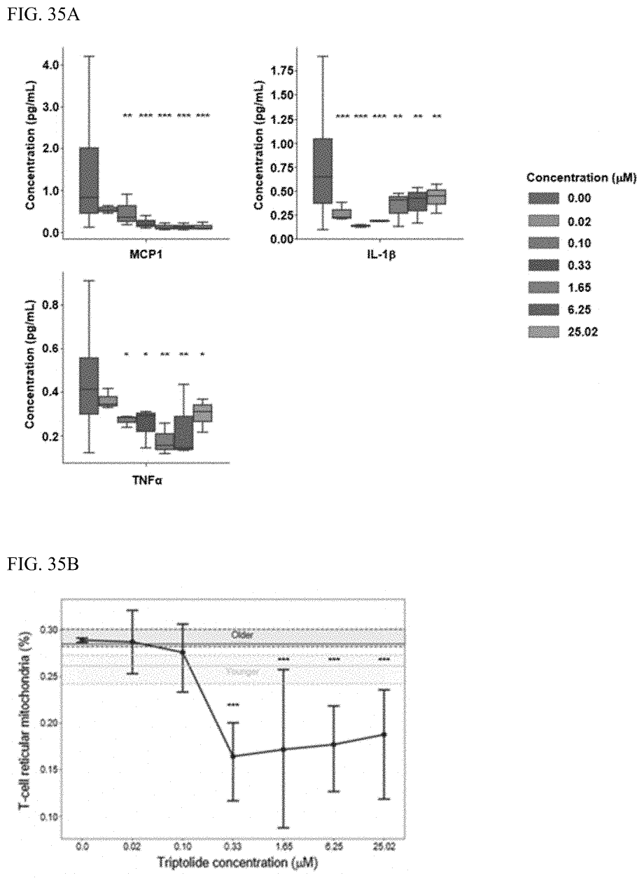

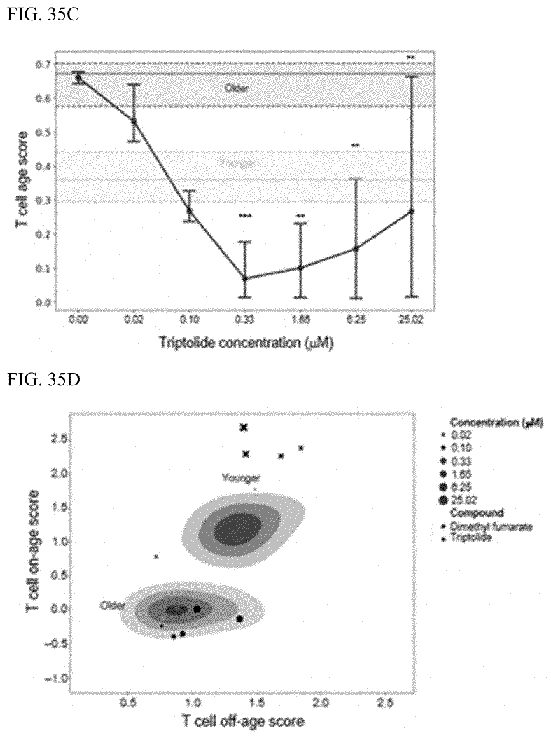

Similar significant decreases in proinflammatory cytokines produced by triptonide-treated PBMCs were observed relative to old untreated control PBMCs. MCP1, IL-1β, and TNFα all showed significant differences at one or more doses compared with controls (FIG. 35A). The most notable of these was MCP1, which showed a significant, dose-dependent reduction when compared to controls (0.1 μM [P=0.004]; 0.33 μM [P<0.001]; 1.65 μM [P<0.001]; 6.25 μM [P<0.001]; 25.02 μM [P<0.001]; FIG. 35A). These results validate the ability of the aging profile to identify immunomodulatory therapies.

Triptonide also caused the features measuring T-cell metabolism to shift from an older profile to a younger profile. There was a dose-dependent decrease in the percentage of reticular T-cell mitochondria, with lower doses showing levels closer to old controls and higher doses showing levels closer to young controls (FIG. 35B).

The predictions from the aging models also went through significant changes after treatment with triptonide. Compared with old untreated controls, old PBMCs treated with various doses of triptonide shifted the model's readout from an older phenotype to a younger phenotype (FIG. 35C). At a low dose, the T-cell age score of the treated PBMCs did not significantly change relative to old controls but significantly decreased upon the application of higher doses (0.02 μM [P=0.85]; 0.1 μM [P=0.16]; 0.33 μM [P<0.001]; 1.65 μM [P=0.001]; 6.25 μM [P=0.008]; 25.02 μM [P=0.005]; FIG. 35C). To better understand this shift in T-cell age score after treatment, image embeddings of T cells were used to compute an “on-age” score, measuring the distance from young controls, and a complementary “off-age” score, measuring the magnitude of the change in the orthogonal direction to age distances. The concept of on-age and off-age scores help distinguish compounds that are primarily driven by aging-related benefits, non-aging related benefits, or a mix of both. In other words, “off-age” effects are not definitively detrimental but show that the therapy induces effects that are not directly related to age. Several doses of triptonide significantly shifted the “on-age” score closer to untreated cells from younger donors and farther from that obtained using untreated cells from older donors (0.02 μM [P=0.27]; 0.1 μM [P<0.001]; 0.33 μM [P<0.001]; 1.65 μM [P<0.001]; 6.25 μM [P<0.001]; 25.02 μM [P<0.001]; FIG. 35D). Triptonide also induced significant “off-age” effects (0.02 μM [P=0.007]; 0.1 μM [P=0.43]; 0.33 μM [P<0.001]; 1.65 μM [P<0.001]; 6.25 μM [P<0.001]; 25.02 μM [P<0.001]; FIG. 35D). Another therapy, dimethyl fumarate, did not improve the “on-age” effects, as indicated by “on-age” scores that were very close to those of old untreated controls (FIG. 35D). Taken together, the multi-phenotype aging profile of old rVSV-infected PBMCs treated with multiple doses of Triptonide showed restored phenotypes in cytokines, reticular mitochondria, and the T-cell predicted age. A deeper understanding of the aging mechanisms behind triptonide may shed light into the dysfunction of immune viral responses in older adults.

The immunosuppressive and anti-inflammatory effects of Triplerygium compounds have also been linked to their ability to induce apoptosis in T cells. In these experiments, triptonide-treated PBMCs had a significant increase in the percentage of dead cells and infected monocytes and a decrease in the percentage of T cells when compared to vehicle-only controls. For the highest three concentrations, there was an average of 21% more dead and infected monocytes and 19% less T cells relative to vehicle-only controls. This was a common trend for many of the top scoring compounds.

### Examples 27 to 31 Used the Following Materials and Methods

Study Ethics and Donors

All study protocols were evaluated and approved by the WCG Institutional Review Board, and all donors provided informed consent. Eighty-nine healthy donors underwent screening at the Discovery Life Sciences Donor Clinic (Huntsville, Ala.). No donors were excluded based on comorbidities, alcohol use, smoking, or current medication.

Sample Collection and Cell Isolation

Whole blood was collected into EDTA tubes and then diluted with an equal volume of phosphate-buffered saline (PBS)+2% fetal bovine serum (FBS) and layered over Ficoll using SepMate™-50 tubes (STEMCELL Technologies Inc., Vancouver, Canada). Cells were centrifuged at 1200×g for 10 min at room temperature, and the top plasma layer was removed. PBMCs were collected, washed with PBS+2% FBS, and counted using acridine orange/propidium iodide using a Cellometer® Vision CBA (Nexcelom Bioscience, Lawrence, Mass., USA). PBMCs were cryopreserved in CryoStor® CS10 (BioLife Solutions, Bothell, Wash., USA), frozen using CoolCell® FTS30 freezing containers (BioCision, San Rafael, Calif., USA), and stored in the liquid nitrogen vapor phase until use.

Specific cell types were isolated from the PBMC fraction using the following kits (STEMCELL Technologies Inc.) per manufacturer's recommendations: EasySep™ Human T Cell Enrichment Kit (T cells): EasySep™ Human B Cell Enrichment Kit (B cells); EasySep™ Human NK Cell Enrichment Kit (NK cells); and EasySep™ Human Monocyte Enrichment Kit (monocytes). T cells (CD3⁺), B cells (CD19⁺), NK cells (CD56⁺), and monocytes (CD14⁺) were isolated based on their customarily defined cell surface markers. Isolated cells were counted using acridine orange/propidium iodide on a Cellometer Vision CBA and then cryopreserved as described above.

Assay Plate Layout

Each 384-well plate assay layout was designed to account for potential bias in experimental conditions due to plate location; conditions were plated such that every condition was included in every row or column. To generate data on isolated cell types, T cells, monocytes, macrophages, dendritic cells, B cells, NK cells, and PBMCs were evaluated as separate conditions. To evaluate effects due to viral load, untreated cells and cells infected at 0.1×, 1×, and 10×MOI were plated as different conditions (described below). To analyze differences between age groups, 48 individual donors were plated per assay plate, with eight replicates per donor. Two plates were required to evaluate all donors. Donors were stratified by age group (≤35 vs ≥60 years). The age groups were considered as separate conditions such that the interspersed plating resulted in a balance between younger and older donors across all plate locations.

Thawing and Plating Cells

Cells were thawed from the gas phase of liquid nitrogen by immersing the cryotube in a 37° C. water bath until a thin layer of ice was present. The cells were transferred to 10 mL of prewarmed culture medium (refer to Supplemental Methods for all media recipes) and immediately centrifuged for 10 min at 138×g. The cell pellet was gently washed with 10 mL of culture medium, resuspended in cold plating medium at a density of 4×10⁵ cells/mL, and 25 μL were added to the assay plate (filled with 15-μL prewarmed plating medium) using a 96-well dispensing head to achieve a final cell density of 10,000 cells per well. Cells were kept cool during the entire plating process. Assay plates were centrifuged for 1 min at 138×g and incubated for 30 min at 37° C. with 5% CO₂ to allow for cell adhesion to the plastic surface of the assay plate.

Vesicular Stomatitis Virus and Compound Exposure

After 30-min cell adhesion, 10 μL of 5× trigger medium (including rVSV-ΔG-mCherry, DMSO, test compound, and FBS) was added to the assay plate using a 384-well pipetting head to achieve a final concentration of rVSV-ΔG-mCherry at 10×MOI, 0.1% DMSO, 10% FBS, and 0.33 μM or 5.3 μM compound concentration. The assay plate was centrifuged for 1 min at 138×g and incubated for 24 h at 37° C. with 5% CO₂.

Live Cell Staining, Fixation, and Painting

After 24 h, cell supernatant was removed and evaluated to determine cytokine levels using the FirePlex®-HT assay system (Abcam, Cambridge, Mass., USA; described below). To label mitochondria in live cells, 50 μL/well of prewarmed 125 nM cell-permeant MitoTracker® solution (Thermo Fisher Scientific, Waltham, Mass., USA) was added to the assay plate and incubated at 37° C. with 5% CO₂. After 30 min incubation, cells were washed 2× with 50 L/well prewarmed culture medium. Cells were fixed through replacing 25 μL of the culture medium with 25 μL of prewarmed formaldehyde solution in culture medium (8% v/v). Cells were incubated at room temperature for 20 min before washing 3× with 50 μL/well PBS. Cells were then permeabilized with 50 μL/well permeabilization buffer (Supplemental Methods) for 5 min, then washed with 50 μL well PBS. Block buffer (50 μL/well; Supplemental Methods) was added to each well, and the assay plate was incubated for 1 h at room temperature.

For the cell painting assay, a CPMM palette was set up in block buffer. Block buffer was removed from the cells and 25 μL/well of the CPMM palette was added. After an overnight (˜18 h) incubation at 8° C., the assay plate was washed 4× with 50-μL/well PBS/acetic acid, and subjected to image acquisition. The plate was stored under refrigerated conditions for further use.

Imaging

Image acquisition was conducted on an Operetta CLS™ high-content analysis system (PerkinElmer, Waltham, Mass.) with binning 1, using a 40- water immersion objective in wide-field mode with Z-stack of 4 planes and distance steps of 1.0 μm. Four to 16 fields of view and 6 channels were captured.

Cytokine Detection

Cytokine levels in the cellular supernatant were evaluated using the FirePlex-HT assay system with the Human Cytokines FirePlex-HT Panel 1 (ab234897; Abcam). FirePlex-HT immunoassays quantify up to 10 protein analytes per sample from low sample inputs in 384-well plate format. The following cytokines were evaluated: IFNγ, IL-1β, IL-2, IL-4, IL-6, IL-8, IL-10, IL-17A, MCP1, and TNFα.

Flow Cytometry

Cells were plated as described above at a cell density of 1.5×10⁵ cells/cm² in 24-well microtiter plates and incubated for 24 h at 37° C. and 5% CO₂ All cells, including the floating cells, were harvested by adding 1 mL PBS/EDTA (1 mM) and incubating for 5 min at 37° C. before being transferred to a round-bottom flow cytometry tube. All remaining steps were carried out with the cells either kept on ice or at 4° C. Cells were washed once with 5 mL of cold FACS buffer (Supplemental Methods) and centrifuged at 216×g for 10 min, then stained with 100-μL antibody mix for 45 min protected from light. Cells were washed 2× with 5-mL FACS buffer, resuspended in 0.5-mL PBS, passed through a cell strainer, and analyzed using a FACSAria® I cell analyzer (Becton, Dickinson and Company, Franklin Lakes, N.J., USA). For VSV-treated experiments, monocytes were defined by medium to high forward scattered light (FSC) and side scattered light (SSC) morphology and CD11c⁺ CD14⁺. NK cells were defined by low FSC/SSC CD3⁻ CD56⁺, and T cells were defined by low FSC/SSC and CD3⁺. Within each defined cellular population, infected cells were segregated by mChery+ signal. Similar gating structures were used for initial identification of cellular subsets in the absence of VSV infection, with the addition of CD19⁺ FSC/SSC low events defined as B cells (see FIG. 36).

Image Processing

Initial data preprocessing was structured as a directed acyclic graph, with image transformation at each step in the graph execution; transformed images were passed along as inputs to the next step. For the first step, the input images, acquired as multiple confocal Z-layers per field of view, were projected onto a single field of view by taking the maximum value of each pixel across all Z-layers for a given field. The projected images were saved both at the original source resolution and at a down-sampled half-resolution. For each channel, an illumination correction function was computed using the half-resolution images and then applied to the full-resolution and half-resolution images to generate illumination-corrected versions of the images. For each channel, the mean and standard deviation of the pixel value distributions were computed over the projected images. The full-resolution and half-resolution images were then z-score normalized using these precomputed means and standard deviations of the plate for each channel to generate pre-normalized versions of the images.

Feature Generation

Using the normalized images, the data processing method continued to feature generation. A CellProfiler pipeline was generated by parameterizing on the relevant palette, magnification, and cells used for the experiment. CellProfiler uses the locations of each cell to generate over 1000 features per cell, including cell morphology, stain intensity, stain texture, stain granularity, and stain colocalization across channels. The CellProfiler pipeline was run over the full-resolution, illumination-corrected images to segment and generate thousands of image- and cell-level features. The location of each cell and nucleus on the plate, as well as a TIFF-based mask for each, was extracted from the CellProfiler output and converted to a format amenable to downstream processing. Pretrained convolutional neural networks were then used to extract single-cell deep-learning embeddings for every cell detected by CellProfiler in every channel. The individual cells were fed to the model using 88px single cell crops taken from the pre-normalized 2160×2160 pixel images. Embeddings were generated at multiple output layers in the pretrained model and then concatenated to create a single embedding vector for each cell.

Quality Control

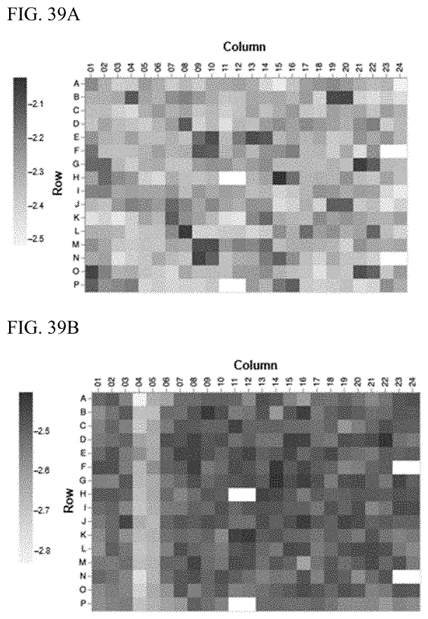

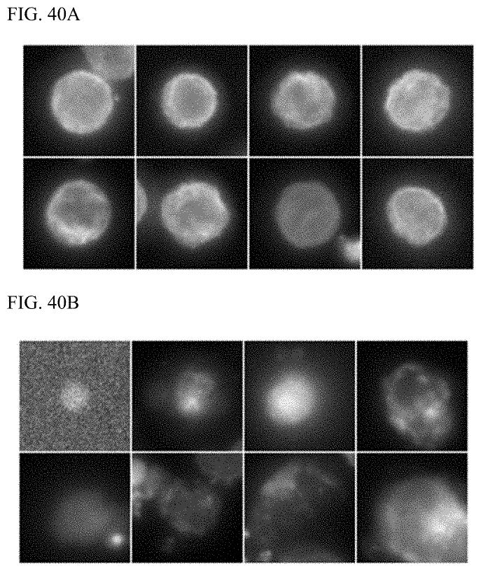

Every image, and each cell within the image, was run through a semi-automated quality control process. The image metrics computed by CellProfiler were visualized and analyzed per plate to identify technical artifacts that arose from issues such as location, operator error, and poor sample quality (FIGS. 39A-39B). Each plate was reviewed by an operator to check for quality across cell count, intensity, blur, focus, and outlier metrics. Plates that had strong negative or positive correlations between quality control metrics and plate location were marked for further review. Poor quality cells were identified by significant blur, poor staining, high background noise, or lack of definition (FIGS. 39A-39B).

Spatial Point Pattern Analysis

The individual cell points detected by CellProfiler were analyzed using spatial point pattern analysis. This modeling provides a statistical basis for understanding the effect of different conditions on how cells spatially interact (clustering or inhibitory) with other cells. After processing, individual fields were treated as unique spatial windows. The point locations of individual cells were marked with labels within these windows, effectively translating an image of a field to a marked point pattern. A data set was then constructed using these marked point patterns, relevant spatial covariates (e.g., relative field location), and global covariates (e.g., donor identification and well condition). This marked point pattern data set was used to model the underlying point pattern process. Spatial point patterns of individual fields were modeled with non-Poisson multitype point interactions for specific cell types. From these individual point pattern models, cell-to-cell interaction parameters for specific label-to-label interactions (e.g., T cell to macrophage) were calculated for each field for subsequent analysis. Spatial point patterns of a selection of fields were modeled using replicated spatial point pattern modeling. An additional series of stepwise replicated point pattern models with non-Poisson multitype point interactions were constructed to analyze point pattern interactions of different cell types with respect to conditions of interest. The replicated point pattern models may include a number of global and spatial covariates to model heterogeneous and homogeneous point patterns, varying interaction parameters describing interpoint interactions, and interactive interaction parameters describing the relationship of conditions of interest and cell-to-cell interactions. Interaction scores <1 indicated decreased clustering while interaction scores >1 indicated increased clustering. This modeling provides evidence of whether or not the conditions of interest alter the cell-to-cell interactions of targeted labels (e.g., monocyte to monocyte and T cell to macrophage), as well as how cell-to-cell interactions change with these conditions (e.g., T cells exhibiting increased clustering with macrophages under a given condition).

Classification

The embeddings and features created were used to train classifiers that predicted whether an immune response was indicative of a younger or older immune system. Data generation methods were optimized to reduce bias by using balanced plate layouts, robust quality control processes, automated liquid handling, and a large, diverse number of samples. Embeddings were generated for each single cell and then aggregated by taking the median of all cells within a field of view. The classification models were trained on these field-level embeddings using XGBoost, an implementation of gradient-boosted decision trees.

Two different methods were used to validate the performance, generalizability, and reproducibility of the classification models. The first method was described in the results above. The second method was stratified 4-fold cross-validation. The data was split into 4 folds such that all data from a single donor were isolated to one of the four folds and that each fold had the same proportion of younger and older donors. This stratification ensures that bias from donor and plate effects do not affect the test set. The model is trained and evaluated 4 times, with 3 folds used for training and the fourth fold used for testing. The performance of the model evaluated across all 4 test sets was averaged and reported as the cross-validation performance. The performance of the test folds was measured using the AUC aggregated at the donor level by taking the mean prediction across a sample's fields of view.

Materials and Equipment

| Name | Source | Product Number |
|---|---|---|
| RPMI 1640 w/o glutamine | Fisher Scientific | 31870-025 |
| FBS Gibco Value-FBS | Fisher Scientific | 10270098, LOT: |
| | | 42F6590K |
| HEPES | PAN-Biotech | P05-01100 |
| Pen/Strep: 10,000 units | Gibco | 15140-122 |
| L-Glutamine, 200 mM | Fisher Scientific | K0302 |
| Na-Pyruvate, 100 mM | Sigma | S8636 |
| Vitamins, 100× | Sigma | M6895 |
| Non-essential AA, 100× | Sigma | M7145 |
| 2-Mercaptoethanol 50 mM | Fisher Scientific | 31350-010 |
| Formaldehyde (FA) | Fisher Scientific | F/1501/PB15 |
| PBS: DPBS (w/o Ca²⁺, Mg²⁺) | PAN-Biotech | P04-36500 |
| 96-well MasterBlock PP 2 mL | Greiner BioOne | 780271 |
| Assay Plate-384-well high content | Corning | 4518 |
| imaging, low base | | |
| Dilution Plate-384-well small | Greiner BioOne | 784201 |
| volume plate | | |
| Supernatant Plate-384-well small | Greiner BioOne | 784201 |
| volume plate | | |
| 24-well plate | Greiner BioOne | 662160 |
| 15-mL tubes | Greiner BioOne | 188271 |
| 50-mL tubes | Greiner BioOne | 227261 |
| Polystyrene round-bottom tube | Falcon | 352008 |
| PBS: DPBS (w/o Ca²⁺, Mg²⁺) | PAN-Biotech | P04-36500 |
| BSA | Merck | 1.12017.0100 |
| Fish gelatin | Sigma | G7041 |
| Antibiotic/antimycotic | Gibco | 15240-062 |
| Triton X-100 | Roth | 3051.3 |
| Human serum, normal, 100 mL | Sigma- Aldrich | S1-100ML, LOT: 3280083 |
| VSV-ΔG-mCherry (8 × 10⁸ IFU/mL) | Creative Biogene | CBGAB0617-2 |
| | | Lot: 011020VB |
| Wheat germ agglutinin, Alexa | Invitrogen | W11261 |
| Fluor ™ 488 conjugate | | |
| Concanavalin A, Alexa Fluor ™ 633 | Invitrogen | C21402 |
| conjugate | | |
| MitoTracker orange CMTMRos | Invitrogen | M75I0 |
| Phalloidin-iFluor 700 conjugate | AAT Bioquest | 23129 |
| | | Lot: 239050 |
| Hoechst 33342 | Invitrogen | 62249 |
| APC-H7_MouseAntiHuman_CD3, | BD | 560176 |
| Lot 9172768 | | |
| PE_MouseAntiHuman_CD19, | BD | 555413 |
| Lot 9156749 | | |
| BB515MouseAntiHuman_CD56, | BD | 564488 |
| Lot 9309302 | | |
| BB700 Mouse Anti-Human CD11c, | BD | 748270 |
| Lot 9350423 | | |
| APC_MouseAntiHuman_CD14, | BD | 5553991 |
| Lot 9010704 | | |

AA, amino acid; BSA, bovine serum albumin; Ca, calcium; DPBS, Dulbecco’s phosphate-buffered saline; FBS, fetal bovine serum; IFU, infectious units, Mg, magnesium; Na, sodium; VSV-ΔG-mCherry, vesicular stomatitis virus expressing a red fluorescent construct.

| Type | Name, vendor |
|---|---|
| Felix-384-well liquid handler | Cybi-Felix w/384-well head, Analytik Jena |
| Echo550 | Labeyte |
| Operetta CLS | PerkinElmer |
| FACS | FACS Arial Sorter, Becton Dickinson |
| Multichannel dispenser | Multidrop Combi, Thermo Fisher Scientific |
| Plate shaker | Oribital shaker GLF, digital 3017 |
| Centrifuge | Thermo Multifuge 3L-R |
| Bioactive library | Assay. Works |

FACS, fluorescence-activated cell sorting.

CellProfiler Pipeline

(1) Segmenting the nuclei using the Hoechst channel, via Otsu or minimum cross entropy-based thresholding; (2) Segmenting the cell membrane using an appropriate channel, via minimum cross entropy-based thresholding, (3) Computing, over both the nuclei and cell membranes, a set of features to capture: (a) The size and shape of the objects of interest, (b) The texture of the objects of interest, across all channels, (c) The stain colocalization of the objects of interest, across all pairs of channels, (d) The granularity of the objects of interest, across all channels, (e) The intensity of the staining of the objects of interest, across all channels, (f) The intensity distribution of the staining of the objects of interest, across all channels, (g) A TIFF-based mask of the object, (4) Computing, over the entire field of view: (a) The intensity of the staining, across all channels, (b) The image correlation, power log-log slope, and other quality control metrics, across all channels.

General Experimental Notes

Fetal bovine serum (FBS) was heat inactivated at 56° C. for 30 min before use and stored frozen in small aliquots. An aliquot was thawed for each prepared bottle of medium

Medium was prepared fresh on the day of plating.

Trigger medium was filtered to eliminate precipitations in FBS.

Unless indicated, 5-mL serological pipettes were used throughout the protocol to minimize cell death.

5× Trigger medium contained 5×DMSO, 5× rVSV-ΔG-mCherry, 5× test compound, and 5×FBS to obtain a final DMSO concentration of 0.1%, rVSV-ΔG-mCherry at 10×MOL 0.33 μM or 5.3 μM compound, and 10% FBS.

All centrifugation steps were performed at room temperature (22° C.).

Thawing and wash steps were performed with warm culture medium (37° C.).

| Stock solution | Preparation | Storage; usage |
|---|---|---|
| MitoTrackerorange | Dissolved in DMSO to 1 mM | Stored at −20° C. |
| CMTMRos | | |
| Wheat germ agglutinin, Alexa | Dissolved in PBS to 1 mg/mL | Single use stored at −20° C. |
| Fluor ™ 488 conjugate 5 mg | | |
| Concanavalin A, Alexa | Dissolved in 0.1-M sodium | Single use stored at −20° C. |
| Fluor ™ 633 conjugate | bicarbonate (pH 8.3) to 1 | |
| | mg/mL | |
| Phalloidin-iFluor700 | Dissolved in 30-μL DMSO to | Stored at −20° C. |
| conjugate Lot 239050 | 1000× | |
| rVSV-ΔG-mCherry | 8 × 10⁸ IFU/mL | Stored at −80° C. |
| Bioactive library | 10 mM in DMSO | |

IFU, infectious units; PBS, phosphate-buffered saline; rVSV-ΔG-mCherry, recombinant vesicular stomatitis virus expressing a red fluorescent construct.

Culture medium

| Component | Concentration |
|---|---|
| RPMI 1640 | Base medium |
| FBS | 10% |
| HEPES | 25 mM |
| L-Glutamine, 200 mM | 2 mM |
| Na-Pyruvate, 100 mM | 1 mM |
| Vitamins, 100× | 0.4× |
| Non-essential AA, 100× | 1× |
| 2-Mercaptoethanol 50 mM | 50 μM |
| Pen/Strep: 10,000 units | 0.5× |

Plating medium

| Component | Concentration |
|---|---|
| RPMI 1640 | Base medium |
| HEPES | 25 mM |
| L-Glutamine, 200 mM | 2 mM |
| Na-Pyruvate, 100 mM | 1 mM |
| Vitamins, 100× | 0.4× |
| Non-essential AA, 100× | 1× |
| 2-Mercaptoethanol 50 mM | 50 μM |
| Pen/Strep: 10,000 units | 0.5× |

AA, amino acid; FBS, fetal bovine serum; Na, sodium.

5× Trigger medium^{a}

| Component | Concentration |
|---|---|
| FBS Gibco Value-FBS | 50% |
| rVSV-ΔG-mCherry | 50× MOI (1 × 10⁷ IFU/mL) |
| DMSO | 0.5% (accounting for compound DMSO) |

FBS, fetal bovine serum; IFU, infectious units; MOI, multiplicity of infection; rVSV-ΔG-mCherry, recombinant vesicular stomatitis virus expressing a red fluorescent construct.
^{a}Medium is filtered using 0.2-μm filter cups to eliminate precipitates present in the FBS.

Block buffer^{a}

| Component | Concentration |
|---|---|
| PBS | 1× supplemented with: |
| BSA | 2% (w/v) |
| Fish gelatin | 0.2% (w/v) |
| Antibiotic/antimycotic | 1% (v/v) (1:100) |
| Human serum, normal | 5% (v/v) (1:20) |

BSA, bovine serum albumin; PBS, phosphate-buffered saline.
^{a}Buffer is sterile filtered and frozen in aliquots.

Wash buffer

| Component | Concentration |
|---|---|
| PBS | 1× supplemented with: |
| Antibiotic/antimycotic | 1% (v/v) (1:100) |

PBS, phosphate-buffered saline.

Cell Painting Palette 2^{a}

| Component | Dilation |
|---|---|
| Wheat germ agglutinin, Alexa Fluor ™ 488 conjugate | 1:2,000 |
| Phalloidin iFluor 700 prediluted stock (1:10 in DMSO) | 1:10,000 |
| Concanavalin A, Alexa Fluor ™ 633 conjugate | 1:200 |
| Hoechst 33342 | 1:10,000 |

^{a}Diluted in block buffer.

Cell Painting Palette 3^{a}

| Component | Dilution |
|---|---|
| Hoechst 33342 | |
| Anti-CD (e.g., mouse) (e.g., FITC) | 1:50 |
| Anti-CD14 (e.g., rabbit) (e.g., Texas Red) | 1:50 |
| Wheat Germ Agglutinin (WGA) or Concanavalin | |
| (Con A) (e.g., Cy5) | |

^{a}Diluted in block buffer.

Cell Painting Palette 4^{a}

| Component | Dilution |
|---|---|
| Hoechst 33342 | |
| Anti-Calprotectin antibody (e.g., mouse) (e.g., FITC(488)) | 1:50 |
| Anti-Mannose receptor antibody (e.g,, rabbit) (e.g., Texas | 1:50 |
| Red(568)) | |
| Wheat Germ Agglutinin (WGA) or Concanavalin (Con A) | |
| (e.g., Cy5) | |

^{a}Diluted in block buffer.

FACS buffer

| Component | Concentration |
|---|---|
| DPBS | Supplemented with |
| Human serum | 5% |

DPBS, Dulbecco’s phosphate-buffered saline; FACS, fluorescence-activated cell sorting.

FACS antibody mix

| Component | Volume |
|---|---|
| FACS buffer | 100 μL |
| APC-H7_MouseAntiHuman_CD3, Lot 9172768 | 2.5 μL |
| PE_MouseAntiHuman_CD19, Lot 9156749 | 10 μL |
| BB515MouseAntiHuman_CD56, Lot 9309302 | 2.5 μL |
| BB700 Mouse Anti-Human CD11c, Lot 9350423 | 2.5 μL |
| APC_MouseAntiHuman_CD14, Lot 9010704 | 10 μL |

FACS, fluorescence-activated cell sorting.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

1-236. (canceled)

237. A computer-implemented method, the computer-implemented method comprising:
generating a training set comprising a first subset of images, the first subset of images including images of cells modified with one of a plurality of agents, and a second subset of images, the second subset of images including images of cells that have not been modified with an agent;
training a machine learned model using the training set, the machine-learned model configured to predict an effect of an agent on a state of a cell;
accessing a set of images, each image in the set of images including a cell, each cell associated with a first state;
applying the machine learned model to each image in the set of images to identify a set of candidate compounds predicted to modify a corresponding state of a corresponding cell from the first state of the cell to a second state; and
providing the set of candidate compounds to an entity associated with the set of images.

238. The method of claim 237, wherein the state of the cell is a predicted age of the cell, and wherein the machine-learned model is configured to predict an effect of an agent on the predicted age of a cell.

239. The method of claim 238, wherein the set of candidate compounds predicted to modify the corresponding state of the corresponding cell from the first state to the second state are predicted to reduce a predicted age of a cell by restoring one or more aspects of an immune response of the cell.

240. The method of claim 237, wherein the state of the cell is a function of the cell, and wherein the machine learned model is configured to predict an effect of an agent on the function of a cell.

241. The method of claim 240, wherein the function of the cell is the immune cell function of the cell, and wherein the agent modifies the immune cell function from a first function to a second function.

242. The method of claim 237, wherein the identified set of compounds is predicted to modify the corresponding state of the corresponding cell based on at least one of: a functional signature of the cell, a morphological signature of the cell, or a marker of the cell.

243. The method of claim 237, wherein generating the training set further comprises:
preprocessing one or more images in the first subset of images and one or more images in the second subset of images; and
wherein preprocessing an image includes at least one of: rotating the image, inverting the image left, inverting the image right, inverting the image up, or inverting the image down.

244. The method of claim 237, wherein applying the machine learned model to each image in the set of images further comprises:
preprocessing each image in the set of images; and
wherein preprocessing includes at least one of: normalization, image enhancement, image correction, contrast enhancement, brightness enhancement, filtering, transformation, adjusting image resolution, adjusting bit resolution, adjusting image size, adjusting field-of-view, background subtraction, image subtraction, or compression.

245. The method of claim 237, wherein training the machine learned model comprises:
accessing an initial set of weights;
initializing the machine learned model with the initial set of weights;
applying the machine learned model to the training set to generate a prediction of an effect of an agent on a state of a cell; and
updating the initial set of weights based on the predictions and a label associated with each image in the training set, the label indicating a known effect of the agent on a corresponding cell.

246. A system comprising:
a processor; and
a non-transitory computer-readable storage medium storing executing instructions that, when executed, cause the processor to perform steps comprising:
generating a training set comprising a first subset of images, the first subset of images including images of cells modified with one of a plurality of agents, and a second subset of images, the second subset of images including images of cells that have not been modified with an agent;
training a machine learned model using the training set, the machine-learned model configured to predict an effect of an agent on a state of a cell;
accessing a set of images, each image in the set of images including a cell, each cell associated with a first state;
applying the machine learned model to each image in the set of images to identify a set of candidate compounds predicted to modify a corresponding state of a corresponding cell from the first state of the cell to a second state; and
providing the set of candidate compounds to an entity associated with the set of images.

247. The system of claim 246, wherein the state of the cell is a predicted age of the cell, and wherein the machine learned model is configured to predict an effect of an agent on the predicted age of a cell.

248. The system of claim 247, wherein the set of candidate compounds predicted to modify the corresponding state of the corresponding cell from the first state to the second state are predicted to reduce a predicted age of a cell by restoring one or more aspects of an immune response of the cell.

249. The system of claim 246, wherein the state of the cell is a function of the cell, and wherein the machine learned model is configured to predict an effect of an agent on the function of a cell.

250. The system of claim 249, wherein the function of the cell is the immune cell function of the cell, and wherein the agent modifies the immune cell function from a first function to a second function.

251. The system of claim 246, wherein the identified set of compounds is predicted to modify the corresponding state of the corresponding cell based on at least one of: a functional signature of the cell, a morphological signature of the cell, or a marker of the cell.

252. The system of claim 246, wherein generating the training set further comprises:
preprocessing one or more images in the first subset of images and one or more images in the second subset of images; and
wherein preprocessing an image includes at least one of: rotating the image, inverting the image left, inverting the image right, inverting the image up, or inverting the image down.

253. The system of claim 246, wherein applying the machine learned model to each image in the set of images further comprises:
preprocessing each image in the set of images; and
wherein preprocessing includes at least one of: normalization, image enhancement, image correction, contrast enhancement, brightness enhancement, filtering, transformation, adjusting image resolution, adjusting bit resolution, adjusting image size, adjusting field-of-view, background subtraction, image subtraction, or compression.

254. The system of claim 246, wherein training the machine learned model comprises:
accessing an initial set of weights;
initializing the machine learned model with the initial set of weights;
applying the machine learned model to the training set to generate a prediction of an effect of an agent on a state of a cell; and
updating the initial set of weights based on the predictions and a label associated with each image in the training set, the label indicating a known effect of the agent on a corresponding cell.

255. A non-transitory computer-readable storage medium storing executable computer instructions that, when executed by a processor, causes the processor to perform steps comprising:
generating a training set comprising a first subset of images, the first subset of images including images of cells modified with one of a plurality of agents, and a second subset of images, the second subset of images including images of cells that have not been modified with an agent;
training a machine learned model using the training set, the machine-learned model configured to predict an effect of an agent on a state of a cell;
accessing a set of images, each image in the set of images including a cell, each cell associated with a first state;
applying the machine learned model to each image in the set of images to identify a set of candidate compounds predicted to modify a corresponding state of a corresponding cell from the first state of the cell to a second state; and
providing the set of candidate compounds to an entity associated with the set of images.

256. The non-transitory computer-readable storage medium of claim 255, wherein applying the machine learned model to each image in the set of images further comprises:
processing each image in the set of images; and
wherein processing includes at least one of: normalization, image enhancement, image correction, contrast enhancement, brightness enhancement, filtering, transformation, adjusting image resolution, adjusting bit resolution, adjusting image size, adjusting field-of-view, background subtraction, image subtraction, or compression.
